(19) United States
(12) Patent Application Publication
(10) Pub. No.: US 2018/0258437 A1
(43) Pub. Date: Sep. 13, 2018

(54) RECOMBINANT HOST CELLS AND METHODS FOR THE PRODUCTION OF L-ASPARTATE AND BETA-ALANINE

(71) Applicant: Lygos, Inc., Berkeley, CA (US)

(72) Inventor: Jeffrey A. Dietrich, Berkeley, CA (US)

(73) Assignee: Lygos, Inc., Berkeley, CA (US)

(21) Appl. No.: 15/976,861

(22) Filed: May 10, 2018

Related U.S. Application Data
(63) Continuation-in-part of application No. PCT/US2016/061578, filed on Nov. 11, 2016.
(60) Provisional application No. 62/504,290, filed on May 10, 2017.
(60) Provisional application No. 62/254,635, filed on Nov. 12, 2015.

(51) Int. Cl. C12N 15/81; C12N 9/06; C12N 9/00; C12P 13/20; C12P 13/06

(52) U.S. Cl. CPC C12N 15/81; C12N 9/0016; C12Y 104/01021; C12P 13/06; C12Y 604/01001; C12P 13/20; C12N 9/93

(57) ABSTRACT

Recombinant host cells, materials, and methods for the biological production of L-aspartate and/or beta-alanine under substantially anaerobic conditions.

### CROSS-REFERENCE TO RELATED APPLICATIONS

The present application claims priority to U.S. Provisional Patent Application No. 62/504,290, filed May 10, 2017, entitled “RECOMBINANT HOST CELLS AND METHODS FOR THE PRODUCTION OF L-ASPARTATE AND BETA-ALANINE,” and is a continuation-in-part of International Application No. PCT/2016/061578, filed Nov. 11, 2016, entitled “RECOMBINANT HOST CELLS AND METHODS FOR THE ANAEROBIC PRODUCTION OF L-ASPARTATE AND BETA-ALANINE,” which claims the benefit of and priority to U.S. Provisional Patent Application No. 62/254,635, filed Nov. 12, 2015, entitled “RECOMBINANT HOST CELLS AND METHODS FOR THE ANAEROBIC PRODUCTION OF L-ASPARTATE AND BETA-ALANINE,” the complete disclosures each of which is incorporated by reference herein in its entirety.

### GOVERNMENT INTEREST

This invention was made with government support under award number DE-EE0007565 awarded by the United States Department of Energy. The government has certain rights in the invention.

### REFERENCE TO SEQUENCE LISTING

This application contains a Sequence Listing submitted via EFS-web which is hereby incorporated by reference in its entirety for all purposes. The ASCII copy, created on May 10, 2018, is named Lygos RECOMBINANT HOST CELLS AND METHODS 5_10_2018 sequence listing_ST25.txt and is 238 KB in size.

### BACKGROUND OF THE INVENTION

The long-term economic and environmental concerns associated with the petrochemical industry has provided the impetus for increased research, development, and commercialization of processes for conversion of carbon feedstocks into chemicals that can replace those derived from petroleum feedstocks. One approach is the development of biorefining processes to convert renewable feedstocks into products that can replace petroleum-derived chemicals. Two common goals in improving a biorefining process include achieving a lower cost of production and reducing carbon dioxide emissions.

Aspartic acid (“L-aspartate”, CAS No. 56-84-8) is currently produced from fumaric acid, a non-renewable, petroleum-derived chemical feedstock. Likewise, beta-alanine (CAS No. 107-96-9) is produced from acrylamide, another non-renewable, petroleum feedstock.

The current, preferred route for industrial synthesis of L-aspartate and L-aspartate-derived compounds is based on fumaric acid. For example, an enzymatic process in which L-aspartate ammonia lyase catalyzes the formation of L-aspartate from fumaric acid and ammonia has been described (see “Amino Acids,” In: Ullmann's Encyclopedia of Industrial Chemistry, Wiley-VCH, Weinheim, New York (2002)).

The existing, petrochemical-based production routes to L-aspartate and beta-alanine are environmentally damaging, dependent on non-renewable feedstocks, and costly. Thus, there remains a need for methods and materials for biocatalytic conversion of renewable feedstocks into L-aspartate and/or beta-alanine and purification of biosynthetic L-aspartate and/or beta-alanine.

### SUMMARY OF THE INVENTION

In a first aspect, the present invention provides a recombinant host cell capable of producing L-aspartate and/or beta-alanine under substantially anaerobic conditions, the host cell comprising one or more heterologous nucleic acids encoding an L-aspartate pathway enzyme and optionally (in the case of beta-alanine producing host cells) an L-aspartate 1-decarboxylase. In one embodiment, the recombinant host cell has been engineered to produce L-aspartate and/or beta-alanine under substantially anaerobic conditions.

Any suitable host cell may be used in the practice of the methods of the present invention, and exemplary host cells useful in the compositions and methods provided herein include archaeal, prokaryotic, or eukaryotic cells. In an important embodiment, the recombinant host cell is a yeast cell. In certain embodiments, the recombinant yeast cells provided herein are engineered by the introduction of one or more genetic modifications (including, for example, heterologous nucleic acids encoding enzymes and/or disruption or deletion of native enzyme-encoding nucleic acids) into a Crabtree-negative yeast cell. In certain of these embodiments, the host cell belongs to the Pichia/Issatchenkia/Saturnispora/Dekkera clade. In certain of these embodiments, the host cell belongs to the genus selected from the group consisting of Pichia, Issatchenkia, or Candida. In certain embodiments, the host cell belongs to the genus Pichia, and in some of these embodiments the host cell is Pichia kudriavzevii.

Provided herein in certain embodiments are recombinant host cells having at least one active L-aspartate pathway from phosphoenolpyruvate or pyruvate to L-aspartate. In some embodiments wherein the host cell produces beta-alanine, the recombinant host cell further expresses an L-aspartate 1-decarboxylase. In certain embodiments, the recombinant host cells provided herein have an L-aspartate pathway that proceeds via phosphoenolpyruvate or pyruvate, and oxaloacetate intermediates. In many embodiments, the recombinant host cell comprises one or more heterologous nucleic acids encoding one or more L-aspartate pathway enzymes selected from the group consisting of phosphoenolpyruvate carboxylase, pyruvate carboxylase, phosphoenolpyruvate carboxykinase, and L-aspartate dehydrogenase wherein the heterologous nucleic acid is expressed in sufficient amounts to produce L-aspartate under substantially anaerobic conditions. In other embodiments, the recombinant host cell comprises one or more heterologous nucleic acids encoding one or more L-aspartate pathway enzymes selected from the group consisting of phosphoenolpyruvate carboxylase, pyruvate carboxylase, phosphoenolpyruvate carboxykinase, and L-aspartate dehydrogenase wherein the heterologous nucleic acid is expressed in sufficient amounts to produce L-aspartate under aerobic conditions. In one embodiment, the recombinant host cell comprises one or more heterologous nucleic acids encoding one or more L-aspartate pathway enzymes selected from the group consisting of pyruvate carboxylase and L-aspartate dehydrogenase, wherein the heterologous nucleic acid is expressed in sufficient amounts to produce L-aspartate under substantially anaerobic conditions. In one embodiment, the recombinant host cell comprises one or more heterologous nucleic acids encoding one or more L-aspartate pathway enzymes selected from the group consisting of pyruvate carboxylase and L-aspartate dehydrogenase wherein the heterologous nucleic acid is expressed in sufficient amounts to produce L-aspartate under aerobic conditions. In certain embodiments, the cell further comprises a heterologous nucleic acid encoding an L-aspartate 1-decarboxylase wherein said heterologous nucleic acid is expressed in sufficient amounts to produce beta-alanine under substantially anaerobic conditions.

In some embodiments, the recombinant host cell provided herein comprises a heterologous nucleic acid encoding an L-aspartate dehydrogenase. In certain embodiments, the recombinant host cell provided herein comprises a heterologous nucleic acid encoding Pseudomonas aeruginosa L-aspartate dehydrogenase (SEQ ID NO: 1) and is capable of producing L-aspartate and/or beta-alanine. In other embodiments, the recombinant host cell provided herein comprises a heterologous nucleic acid encoding Cupriavidus taiwanensis L-aspartate dehydrogenase (SEQ ID NO: 2) and is capable of producing L-aspartate and/or beta-alanine.

In various embodiments, the recombinant host cell further comprises a heterologous nucleic acid encoding an L-aspartate 1-decarboxylase and is capable of producing beta-alanine where cultured under suitable conditions. An L-aspartate 1-decarboxylase as used herein refers to any protein with L-aspartate decarboxylase activity, meaning the ability to catalyze the decarboxylation of L-aspartate to beta-alanine. In various embodiments, the recombinant host cell provided herein comprises one or more heterologous nucleic acids encoding an L-aspartate 1-decarboxylase selected from the group consisting of Bacillus subtilis L-aspartate 1-decarboxylase (SEQ ID NO: 5), Corynebacterium L-aspartate 1-decarboxylase (SEQ ID NO: 4), and/or Tribolium castaneum L-aspartate 1-decarboxylase (SEQ ID NO: 3) and is capable of producing beta-alanine.

In various embodiments, L-aspartate dehydrogenase enzymes suitable for use in accordance with the methods of the invention have L-aspartate dehydrogenase activity and comprise an amino acid sequence with at least 60%, at least 70%, at least 80%, at least 90%, or at least 95% sequence identity to SEQ ID NO: 14. In various embodiments, L-aspartate 1-decarboxylase enzymes suitable for use in accordance with the methods of the invention have L-aspartate 1-decarboxylase activity and comprise an amino acid sequence with at least 55%, at least 60%, at least 70%, at least 80%, at least 90%, or at least 95% sequence identity to SEQ ID NO: 15 and/or 16.

In a second aspect, the invention provides host cells genetically modified to delete or otherwise reduce the activity of endogenous proteins. Deletion or disruption of ethanol fermentation pathway(s) and nucleic acids encoding ethanol fermentation pathway enzymes is important for engineering a recombinant host cell capable of efficient production of L-aspartate and/or beta-alanine under substantially anaerobic conditions. In various embodiments, recombinant host cells comprising deletion or disruption of one or more nucleic acids encoding ethanol fermentation pathway enzymes decreases ethanol production by at least 55%, at least 60%, at least 70%, at least 90%, at least 95%, or at least 99% as compared to parental cells that do not comprise this genetic modification.

In various embodiments, the recombinant host cells comprise a deletion or disruption of one or more nucleic acids encoding an enzyme selected from the group consisting of pyruvate decarboxylase, alcohol dehydrogenase, and/or malate dehydrogenase.

In a third aspect, methods are provided herein for producing L-aspartate and/or beta-alanine by recombinant host cells of the invention. In certain embodiments, these methods comprise the step culturing a recombinant host cell described herein in a medium containing at least one carbon source and one nitrogen source under substantially anaerobic conditions such that L-aspartate is produced. In various embodiments, conditions are selected to produce an oxygen uptake rate of around 0-25 mmol/l/hr. In some embodiments, conditions are selected to produce an oxygen uptake rate of around 2.5-15 mmol/l/hr. In other embodiments, these methods comprise the step of culturing a recombinant host cell described herein in a medium containing at least one carbon source and one nitrogen source under aerobic conditions such that L-aspartate is produced.

### BRIEF DESCRIPTION OF THE FIGURES

FIG. 1 provides a schematic of the 1-aspartate pathway enzymes and 1-aspartate 1-decarboxylase enzymes provided by the invention. Conversion of oxaloacetate to 1-aspartate is catalyzed by 1-aspartate dehydrogenase (ec 1.4.1.21) and conversion of 1-aspartate to beta-alanine is catalyzed by 1-aspartate 1-decarboxylase (ec 4.1.1.11). Oxaloacetate-forming enzymes provided by the invention include pyruvate carboxylase (ec 6.4.1.1), phosphoenolpyruvate carboxylase (ec 4.1.1.31), and phosphoenolpyruvate carboxykinase (ec 4.1.1.49). Conversion of pyruvate to oxaloacetate is catalyzed by pyruvate carboxylase; conversion of phosphoenolpyruvate to oxaloacetate is catalyzed by phosphoenolpyruvate carboxylase and/or phosphoenolpyruvate carboxykinase.

### DETAILED DESCRIPTION OF THE INVENTION

The present invention provides recombinant host cells, materials, and methods for the biological production of L-aspartate and/or beta-alanine under substantially anaerobic conditions.

While the present invention is described herein with reference to aspects and specific embodiments thereof, those skilled in the art will recognize that various changes may be made and equivalents may be substituted without departing from the invention. The present invention is not limited to particular nucleic acids, expression vectors, enzymes, biosynthetic pathways, host microorganisms, or processes, as such may vary. The terminology used herein is for purposes of describing particular aspects and embodiments only, and is not to be construed as limiting. In addition, many modifications may be made to adapt a particular situation, material, composition of matter, process, process steps or steps, in accordance with the invention. All such modifications are within the scope of the claims appended hereto.

### Section 1: Definitions

In this specification and in the claims that follow, reference will be made to a number of terms that shall be defined to have the following meanings.

As used in the specification and the appended claims, the singular forms “a,” “an,” and “the” include plural referents unless the context clearly dictates otherwise. Thus, for example, reference to an “expression vector” includes a single expression vector as well as a plurality of expression vectors, either the same (e.g., the same operon) or different; reference to “cell” includes a single cell as well as a plurality of cells; and the like.

The term “accession number”, and similar terms such as “protein accession number”, “UniProt ID”, “gene ID”, “gene accession number” refer to designations given to specific proteins or genes. These identifiers describe a gene or enzyme sequence in publicly accessible databases, such as NCBI.

A dash (−) in a consensus sequence indicates that there is no amino acid at the specified position. A plus (+) in a consensus sequence indicates any amino acid may be present at the specified position. Thus, a plus in a consensus sequence herein indicates a position at which the amino acid is generally non-conserved; a homologous enzyme sequence, when aligned with the consensus sequence, can have any amino acid at the indicated “+” position.

As used herein, the term “express”, when used in connection with a nucleic acid encoding an enzyme or an enzyme itself in a cell, means that the enzyme, which may be an endogenous or exogenous (heterologous) enzyme, is produced in the cell. The term “overexpress”, in these contexts, means that the enzyme is produced at a higher level, i.e., enzyme levels are increased, as compared to the wild-type, in the case of an endogenous enzyme. Those skilled in the art appreciate that overexpression of an enzyme can be achieved by increasing the strength or changing the type of the promoter used to drive expression of a coding sequence, increasing the strength of the ribosome binding site or Kozak sequence, increasing the stability of the mRNA transcript, altering the codon usage, increasing the stability of the enzyme, and the like.

The terms “expression vector” or “vector” refer to a nucleic acid and/or a composition comprising a nucleic acid that can be introduced into a host cell, e.g., by transduction, transformation, or infection, such that the cell then produces (“expresses”) nucleic acids and/or proteins other than those native to the cell, or in a manner not native to the cell, that are contained in or encoded by the nucleic acid so introduced. Thus, an “expression vector” contains nucleic acids (ordinarily DNA) to be expressed by the host cell. Optionally, the expression vector can be contained in materials to aid in achieving entry of the nucleic acid into the host cell, such as the materials associated with a virus, liposome, protein coating, or the like. Expression vectors suitable for use in various aspects and embodiments of the present invention include those into which a nucleic acid sequence can be, or has been, inserted, along with any preferred or required operational elements. Thus, an expression vector can be transferred into a host cell and, typically, replicated therein (although, one can also employ, in some embodiments, non-replicable vectors that provide for “transient” expression). In some embodiments, an expression vector that integrates into chromosomal, mitochondrial, or plastid DNA is employed. In other embodiments, an expression vector that replicates extrachromasomally is employed. Typical expression vectors include plasmids, and expression vectors typically contain the operational elements required for transcription of a nucleic acid in the vector. Such plasmids, as well as other expression vectors, are described herein or are well known to those of ordinary skill in the art.

The terms “ferment”, “fermentative”, and “fermentation” are used herein to describe culturing microbes under conditions to produce useful chemicals, including but not limited to conditions under which microbial growth, be it aerobic or anaerobic, occurs.

The term “heterologous” as used herein refers to a material that is non-native to a cell. For example, a nucleic acid is heterologous to a cell, and so is a “heterologous nucleic acid” with respect to that cell, if at least one of the following is true: (a) the nucleic acid is not naturally found in that cell (that is, it is an “exogenous” nucleic acid); (b) the nucleic acid is naturally found in a given host cell (that is, “endogenous to”), but the nucleic acid or the RNA or protein resulting from transcription and translation of this nucleic acid is produced or present in the host cell in an unnatural (e.g., greater or lesser than naturally present) amount; (c) the nucleic acid comprises a nucleotide sequence that encodes a protein endogenous to a host cell but differs in sequence from the endogenous nucleotide sequence that encodes that same protein (having the same or substantially the same amino acid sequence), typically resulting in the protein being produced in a greater amount in the cell, or in the case of an enzyme, producing a mutant version possessing altered (e.g. higher or lower or different) activity; and/or (d) the nucleic acid comprises two or more nucleotide sequences that are not found in the same relationship to each other in the cell. As another example, a protein is heterologous to a host cell if it is produced by translation of RNA or the corresponding RNA is produced by transcription of a heterologous nucleic acid; a protein is also heterologous to a host cell if it is a mutated version of an endogenous protein, and the mutation was introduced by genetic engineering.

The term “homologous”, as well as variations thereof, such as “homology”, refers to the similarity of a nucleic acid or amino acid sequence, typically in the context of a coding sequence for a gene or the amino acid sequence of a protein. Homology searches can be employed using a known amino acid or coding sequence (the “reference sequence”) for a useful protein to identify homologous coding sequences or proteins that have similar sequences and thus are likely to perform the same useful function as the protein defined by the reference sequence. As will be appreciated by those of skill in the art, a protein having greater than 90% identity to a reference protein as determined by, for example and without limitation, a BLAST (blast.ncbi.nlm.nih.gov) search is highly likely to carry out the identical biochemical reaction as the reference protein. In some cases, two enzymes having greater than 20% identity will carry out identical biochemical reactions, and the higher the identity, i.e., 40% or 80% identity, the more likely the two proteins have the same or similar function. As will be appreciated by those skilled in the art, homologous enzymes can be identified by BLAST searching.

The terms “host cell” and “host microorganism” are used interchangeably herein to refer to a living cell that can be (or has been) transformed via insertion of an expression vector. A host microorganism or cell as described herein may be a prokaryotic cell (e.g., a microorganism of the kingdom Eubacteria) or a eukaryotic cell. As will be appreciated by one of skill in the art, a prokaryotic cell lacks a membrane-bound nucleus, while a eukaryotic cell has a membrane-bound nucleus.

The terms “isolated” or “pure” refer to material that is substantially, e.g. greater than 50% or greater than 75%, or essentially, e.g. greater than 90%, 95%, 98% or 99%, free of components that normally accompany it in its native state, e.g. the state in which it is naturally found or the state in which it exists when it is first produced.

As used herein, the term “nucleic acid” and variations thereof shall be generic to polydeoxyribonucleotides (containing 2-deoxy-D-ribose), polyribonucleotides (containing D-ribose), segments of polydeoxyribonucleotides, and segments of polyribonucleotides. “Nucleic acid” can also refer to any other type of polynucleotide that is an N-glycoside of a purine or pyrimidine base, and to other polymers containing nonnucleotidic backbones, provided that the polymers contain nucleobases in a configuration that allows for base pairing and base stacking, as found in DNA and RNA. As used herein, the symbols for nucleotides and polynucleotides are those recommended by the IUPAC-IUB Commission of Biochemical Nomenclature (Biochem. 9:4022, 1970). A “nucleic acid” may also be referred to herein with respect to its sequence, the order in which different nucleotides occur in the nucleic acid, as the sequence of nucleotides in a nucleic acid typically defines its biological activity, e.g., as in the sequence of a coding region, the nucleic acid in a gene composed of a promoter and coding region, which encodes the product of a gene, which may be an RNA, e.g. a rRNA, tRNA, or mRNA, or a protein (where a gene encodes a protein, both the mRNA and the protein are “gene products” of that gene).

The term “operably linked” refers to a functional linkage between a nucleic acid expression control sequence (such as a promoter, ribosome-binding site, and transcription terminator) and a second nucleic acid sequence, the coding sequence or coding region, wherein the expression control sequence directs or otherwise regulates transcription and/or translation of the coding sequence.

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

As used herein, “recombinant” refers to the alteration of genetic material by human intervention. Typically, recombinant refers to the manipulation of DNA or RNA in a cell or virus or expression vector by molecular biology (recombinant DNA technology) methods, including cloning and recombination. Recombinant can also refer to manipulation of DNA or RNA in a cell or virus by random or directed mutagenesis. A “recombinant” cell or nucleic acid can typically be described with reference to how it differs from a naturally occurring counterpart (the “wild-type”). In addition, any reference to a cell or nucleic acid that has been “engineered” or “modified” and variations of those terms, is intended to refer to a recombinant cell or nucleic acid.

The terms “transduce”, “transform”, “transfect”, and variations thereof as used herein refers to the introduction of one or more nucleic acids into a cell. For practical purposes, the nucleic acid must be stably maintained or replicated by the cell for a sufficient period of time to enable the function(s) or product(s) it encodes to be expressed for the cell to be referred to as “transduced”, “transformed”, or “transfected”. As will be appreciated by those of skill in the art, stable maintenance or replication of a nucleic acid may take place either by incorporation of the sequence of nucleic acids into the cellular chromosomal DNA, e.g., the genome, as occurs by chromosomal integration, or by replication extrachromosomally, as occurs with a freely-replicating plasmid. A virus can be stably maintained or replicated when it is “infective”: when it transduces a host microorganism, replicates, and (without the benefit of any complementary virus or vector) spreads progeny expression vectors, e.g., viruses, of the same type as the original transducing expression vector to other microorganisms, wherein the progeny expression vectors possess the same ability to reproduce.

As used herein, “L-aspartate” is intended to mean an amino acid having the chemical formula C₄H₅NO₄ and a molecular mass of 131.10 g/mol (CAS#56-84-8). L-aspartate as described herein can be a salt, acid, base, or derivative depending on the structure, pH, and ions present. The terms “L-aspartate”, “L-aspartic acid”, “L-aspartate”, and “aspartic acid” are used interchangeably herein.

As used herein, beta-alanine is intended to mean a beta amino acid having the chemical formula C₃H₆NO₂ and a molecular mass of 88.09 g/mol (CAS #107-95-9). Beta-alanine as described herein can be a salt, acid, base, or derivative depending on the structure, pH, and ions present. Beta-alanine is also referred to as “β-alanine”, “3-aminopropionic acid”, and “3-aminopropanoate”, and these terms are used interchangeably herein.

As used herein, the term “substantially anaerobic” when used in reference to a culture or growth condition is intended to mean the amount of oxygen is less than about 10% of saturation for dissolved oxygen in liquid media. The term is also intended to include sealed chambers of liquid or solid growth medium maintained with an atmosphere of less than about 1% oxygen.

### Section 2: Recombinant Host Cells for Production of L-Aspartate and Beta-Alanine

### 2.1 Host Cells

In one aspect, the invention provides a recombinant host cell capable of producing L-aspartate and/or beta-alanine under substantially anaerobic conditions, the host cell comprising one or more heterologous nucleic acids encoding a L-aspartate pathway enzyme and optionally (in the case of beta-alanine producing host cells) a L-aspartate 1-decarboxylase. In one embodiment, the recombinant host cell has been engineered to produce L-aspartate and/or beta-alanine under substantially anaerobic conditions. In another embodiment, the recombinant host cell natively produces L-aspartate and/or beta-alanine under substantially anaerobic conditions. In another embodiment, the recombinant host cell has been engineered to produce L-aspartate and/or beta-alanine under aerobic conditions.

Any suitable host cell may be used in practice of the methods of the present invention, and exemplary host cells useful in the compositions and methods provided herein include archaeal, prokaryotic, or eukaryotic cells.

### 2.1.1 Yeast Cells

In an important embodiment, the recombinant host cell is a yeast cell. Yeast cells are excellent host cells for construction of recombinant metabolic pathways comprising heterologous enzymes catalyzing production of small-molecule products. There are established molecular biology techniques and nucleic acids encoding genetic elements necessary for construction of yeast expression vectors, including, but not limited to, promoters, origins of replication, antibiotic resistance markers, auxotrophic markers, terminators, and the like. Second, techniques for integration/insertion of nucleic acids into the yeast chromosome by homologous recombination are well established. Yeast also offers a number of advantages as an industrial fermentation host. Yeast cells can generally tolerate high concentrations of organic acids and maintain cell viability at low pH and can grow under both aerobic and anaerobic culture conditions, and there are established fermentation broths and fermentation protocols. The ability of a strain to propagate and/or produce the desired product under substantially anaerobic conditions provides a number of advantages with regard to the present invention. First, this characteristic results in efficient product biosynthesis when the host cell is supplied with a carbohydrate carbon source. Second, from a process standpoint, the ability to run a fermentation under substantially anaerobic conditions decreases production cost.

In various embodiments, yeast cells useful in the method of the invention include yeasts of a genera selected from the non-limiting group consisting of Aciculoconidium, Ambrosiozyma, Arthroascus, Arxiozyma, Ashbya, Babjevia, Bensingtonia, Botryoascus, Botryozyma, Brettanomyces, Bullera, Bulleromyces, Candida, Citeromyces, Clavispora, Cryptococcus, Cystofilobasidium, Debaryomyces, Dekkara, Dipodascopsis, Dipodascus, Eeniella, Endomycopsella, Eremascus, Eremothecium, Erythrobasidium, Fellomyces, Filobasidium, Galactomyces, Geotrichum, Guilliermondella, Hanseniaspora, Hansenula, Hasegawaea, Holtermannia, Hormoascus, Hyphopichia, Issatchenkia, Kloeckera, Kloeckeraspora, Kluyveromyces, Kondoa, Kuraishia, Kurtzmanomyces, Leucosporidium, Lipomyces, Lodderomyces, Malassezia, Metschnikowia, Mrakia, Myxozyma, Nadsonia, Nakazawaea, Nematospora, Ogataea, Oosporidium, Pachysolen, Phachytichospora, Phaffia, Pichia, Rhodosporidium, Rhodotorula, Saccharomyces, Saccharomycodes, Saccharomycopsis, Saitoella, Sakaguchia, Saturnospora, Schizoblastosporion, Schizosaccharomyces, Schwanniomyces, Sporidiobolus, Sporobolomyces, Sporopachydermia, Stephanoascus, Sterigmatomyces, Sterigmatosporidium, Symbiotaphrina, Sympodiomyces, Sympodiomycopsis, Torulaspora, Trichosporiella, Trichosporon, Trigonopsis, Tsuchiyaea, Udeniomyces, Waltomyces, Wickerhamia, Wickerhamiella, Williopsis, Yamadazyma, Yarrowia, Zygoascus, Zygosaccharomyces, Zygowilliopsis, and Zygozyma, among others.

In various embodiments, the yeast cell is of a species selected from the non-limiting group consisting of Candida albicans, Candida ethanolica, Candida guilliermondii, Candida krusei, Candida lipolytica, Candida rnethanosorbosa, Candida sonorensis, Candida tropicalis, Candida utilis, Cryptococcus curvatus, Hansenula polymorpha, Issatchenkia orientalis, Kluyveromyces lactic, Kluyveromyces marxianus, Kluyveromyces thermotolerans, Komagataella pastoris, Lipomyces starkeyi, Pichia angusta, Pichia deserticola, Pichia galeiformis, Pichia kodamae, Pichia kudriavzevii (P. kudriavzevii), Pichia membranaefaciens, Pichia methanolica, Pichia pastoris, Pichia salictaria, Pichia stipitis, Pichia thermotolerans, Pichia trehalophila, Rhodosporidium toruloides, Rhodotorula glutinis, Rhodotorula graminis, Saccharomyces bayanus, Saccharomyces boulardi, Saccharomyces cerevisiae (S. cerevisiae), Saccharomyces kluyveri, Schizosaccharomyces pombe (S. pombe) and Yarrowia lipolytica. One skilled in the art will recognize that this list encompasses yeast in the broadest sense.

In certain embodiments, the recombinant yeast cells provided herein are engineered by the introduction of one or more genetic modifications (including, for example, heterologous nucleic acids encoding enzymes and/or the disruption or deletion of native nucleic acids encoding enzymes) into a Crabtree-negative yeast cell. In certain of these embodiments, the host cell belongs to the Pichia/Issatchenkia/Saturnispora/Dekkera clade. In certain of these embodiments, the host cell belongs to the genus selected from the group consisting of Pichia, Issatchenkia, or Candida. In certain embodiments, the host cell belongs to the genus Pichia, and in some of these embodiments the host cell is Pichia kudriavzevii.

In certain embodiments, the recombinant host cells provided herein are engineered by introduction of one or more genetic modifications into a Crabtree-positive yeast cell. In certain of these embodiments, the host cell belongs to the Saccharomyces clad. In certain of these embodiments, the host cell belongs to a genus selected from the group consisting of Saccharomyces, Hanseniaspora, and Kluyveromyces. In certain embodiments, the host cell belongs to the genus Saccharomyces, and in one of these embodiments the host cell is S. cerevisiae.

Members of the Pichia/Issatchenkia/Saturnispora/Dekkera or the Saccharomyces clade are identified by analysis of their 26S ribosomal DNA using the methods described by Kurtzman C. P., and Robnett C. J., (“Identification and Phylogeny of Ascomycetous Yeasts from Analysis of Nuclear Large Subunit (26S) Ribosomal DNA Partial Sequences”, Atonie van Leeuwenhoek 73(4):331-371; 1998). Kurtzman and Robnett report analysis of approximately 500 ascomycetous yeasts were analyzed for the extent of divergence in the variable D1/D2 domain of the large subunit (26S) ribosomal DNA. Host cells encompassed by a clade exhibit greater sequence identity in the D1/D2 domain of the 26S ribosomal subunit DNA to other host cells within the clade as compared to host cells outside the clade. Therefore, host cells that are members of a clade (e.g., the Pichia/Issatchenkia/Saturnispora/Dekkera or Saccharomyces clades) can be identified using the methods of Kurtzman and Robnett.

### 2.1.2 Other Host Cells

Recombinant host cells other than yeast cells are also suitable for use in accordance with the methods of the invention so long as the engineered host cell is capable of growth and/or product formation under substantially anaerobic conditions. Illustrative examples include various eukaryotic, prokaryotic, and archaeal host cells. Illustrative examples of eukaryotic host cells provided by the invention include, but are not limited to cells belonging to the genera Aspergillus, Crypthecodinium, Cunninghamella, Entomoplithora, Mortierella, Mucor, Neurospora, Pythium, Schizochytrium, Thraustochytrium, Trichoderma, Xanthophyllomyces. Examples of eukaryotic strains include, but are not limited to: Aspergillus niger, Aspergillus oryzae, Crypthecodinium cohnii, Cunninghamella japonica, Entomophthora coronata, Mortierella alpina, Mucor circinelloides, Neurospora crassa, Pythium ultimum, Schizochytrium limacinum, Thraustochytrium aureum, Trichoderma reesei and Xanthophyllomyces dendrorhous.

Illustrative examples of recombinant archaea host cells provided by the invention include, but are not limited to, cells belonging to the genera: Aeropyrum, Archaeglobus, Halobacterium, Methanococcus, Methanobacterium, Pyrococcus, Sulfolobus, and Thermoplasma. Examples of archae strains include, but are not limited to Archaeoglobus fulgidus, Halobacterium sp., Methanococcus jannaschii, Methanobacterium thermoautotrophicum, Thermoplasma acidophilum, Thermoplasma volcanium, Pyrococcus horikoshii, Pyrococcus abyssi, and Aeropyrum pernix.

Illustrative examples of recombinant prokaryotic host cells provided by the invention include, but are not limited to, cells belonging to the genera Agrobacterium, Alicyclobacillus, Anabaena, Anacystis, Arthrobacter, Azobacter, Bacillus, Brevibacterium, Chromatium, Clostridium, Corynebacterium, Enterobacter, Erwinia, Escherichia, Lactobacillus, Lactococcus, Mesorhizobium, Methylobacterium, Microbacterium, Pantoea, Phormidium, Pseudomonas, Rhodobacter, Rhodopseudomonas, Rhodospirillum, Rhodococcus, Salmonella, Scenedesmun, Serratia, Shigella, Staphylococcus, Strepromyces, Synnecoccus, and Zymomonas. Examples of prokaryotic strains include, but are not limited to Bacillus subtilis, Brevibacterium ammoniagenes, Bacillus amyloliquefacines, Brevibacterium ammoniagenes, Brevibacterium immariophilum, Clostridium beigerinckii, Corynebacterium glutamicum (C. glutamicum), Enterobacter sakazakii, Escherichia coli (E. coli), Lactobacillus acidophilus, Lactococcus lactis, Mesorhizobium loti, Pantoea ananatis (P. ananatis), Pseudomonas aeruginosa, Pseudomonas mevalonii, Pseudomonas pudita, Rhodobacter capsulatus, Rhodobacter sphaeroides, Rhodospirillum rubrum, Salmonella enterica, Salmonella typhi, Salmonella typhimurium, Shigella dysenteriae, Shigella flexneri, Shigella sonnei, and Staphylococcus aureus.

E. coli, C. glutamicum, and P. ananatis are particularly good prokaryotic host cells for use in accordance with the methods of the invention. E. coli is capable of growth and/or product (L-aspartate and/or beta-alanine) formation under substantially anaerobic conditions, is well-utilized in industrial fermentation of small-molecule products, and can be readily engineered. Unlike most wild type yeast strains, wild type E. coli can catabolize both pentose and hexose sugars as carbon sources. The present invention provides a wide variety of E. coli host cells suitable for use in the methods of the invention. In one embodiment, the recombinant host cell is an E. coli host cell. C. glutamicum is well utilized for industrial production of various amino acids. While generally regarded as a strict aerobe, wild type C. glutamicum is capable of growth under substantially anaerobic conditions if nitrate is supplied to the fermentation broth as an electron acceptor. If nitrate is not supplied, wild type C. glutamicum will not grow under substantially anaerobic conditions but will catabolize sugar and produce a range of fermentation products. In one embodiment, the recombinant host cell is a C. glutamicum host cell. Like E. coli, P. ananatis is also capable of growth under substantially anaerobic conditions; P. ananatis is also able to grow in a low pH environment, decreasing the amount of base that must be added during the fermentation in order to sustain organic acid (for example, aspartic acid) production. In one embodiment, the recombinant host cell is a P. ananatis host cell.

In some embodiments, the host cell is a microbe that is capable of growth and/or production of L-aspartate or beta-alanine under substantially anaerobic conditions. Suitable host cells may natively grow under substantially anaerobic conditions or may be engineered to be capable of growth under substantially anaerobic conditions.

Certain of these host cells, including S. cerevisiae, Bacillus subtilis, Lactobacillus acidophilus, have been designated by the Food and Drug Administration as Generally Regarded As Safe (or GRAS) and so are employed in various embodiments of the methods of the invention. While desirable from public safety and regulatory standpoints, GRAS status does not impact the ability of a host strain to be used in the practice of this invention; hence, non-GRAS and even pathogenic organisms are included in the list of illustrative host strains suitable for use in the practice of this invention.

### 2.2 L-Aspartate Pathway Enzymes and L-Aspartate 1-Decarboxylases

Provided herein in certain embodiments are recombinant host cells having at least one active L-aspartate pathway from phosphoenolpyruvate or pyruvate to L-aspartate. In some embodiments wherein the host cell produces beta-alanine, the recombinant host cell further expresses an L-aspartate 1-decarboxylase. A recombinant host cell having an active L-aspartate pathway as used herein produces active enzymes necessary to catalyze each metabolic reaction in a L-aspartate fermentation pathway, and therefore is capable of producing L-aspartate and/or beta-alanine in measurable yields and/or titers when cultured under suitable conditions. A recombinant host cell having an active L-aspartate pathway comprises one or more heterologous nucleic acids encoding L-aspartate pathway enzymes.

In certain embodiments, the recombinant host cells provided herein have a L-aspartate pathway that proceeds via phosphoenolpyruvate or pyruvate, and oxaloacetate intermediates. In many embodiments, the recombinant host cell comprises one or more heterologous nucleic acids encoding one or more L-aspartate pathway enzymes selected from the group consisting of phosphoenolpyruvate carboxylase, pyruvate carboxylase, phosphoenolpyruvate carboxykinase, and L-aspartate dehydrogenase wherein the heterologous nucleic acid is expressed in sufficient amounts to produce L-aspartate under substantially anaerobic conditions. In other embodiments, the recombinant host cell comprises one or more heterologous nucleic acids encoding one or more L-aspartate pathway enzymes selected from the group consisting of phosphoenolpyruvate carboxylase, pyruvate carboxylase, phosphoenolpyruvate carboxykinase, and L-aspartate dehydrogenase wherein the heterologous nucleic acid is expressed in sufficient amounts to produce L-aspartate under aerobic conditions. In certain embodiments, the cell further comprises a heterologous nucleic acid encoding an L-aspartate 1-decarboxylase wherein said heterologous nucleic acid is expressed in sufficient amounts to produce beta-alanine under substantially anaerobic conditions. Thus, one will recognize that recombinant host cells engineered for production of L-aspartate in accordance with the methods of the invention express an L-aspartate pathway, and recombinant host cells engineered for production of beta-alanine express, in addition to an L-aspartate pathway, a L-aspartate 1-decarboxylase.

In some embodiments, the recombinant host cell comprises one or more heterologous nucleic acids encoding one or more enzymes of an L-aspartate pathway. In some embodiments, the recombinant host cell comprises one or more heterologous nucleic acids encoding one L-aspartate pathway enzyme. In some embodiments, said one L-aspartate pathway enzyme is L-aspartate dehydrogenase. In other embodiments, said one L-aspartate pathway enzyme is pyruvate carboxylase. In other embodiments, said one L-aspartate pathway enzyme is phosphoenolpyruvate carboxylase. In still further embodiments, said one L-aspartate pathway enzyme is phosphoenolpyruvate carboxykinase. In various embodiments, the recombinant host cell comprises one or more heterologous nucleic acids encoding two L-aspartate pathway enzymes. In some embodiments, said two L-aspartate pathway enzymes are L-aspartate dehydrogenase and pyruvate carboxylase. In other embodiments, said two L-aspartate pathway enzymes are L-aspartate dehydrogenase and phosphoenolpyruvate carboxylase. In other embodiments, said two L-aspartate pathway enzymes are L-aspartate dehydrogenase and phosphoenolpyruvate carboxykinase. In various embodiments, the recombinant host cell comprises one or more heterologous nucleic acids encoding three L-aspartate pathway enzymes. In some embodiments, said three L-aspartate pathway enzymes are L-aspartate dehydrogenase, pyruvate carboxylase, and phosphoenolpyruvate carboxylase. In other embodiments, said three L-aspartate pathway enzymes are L-aspartate dehydrogenase, pyruvate carboxylase, and phosphoenolpyruvate carboxykinase. In other embodiments, said three L-aspartate pathway enzymes are L-aspartate dehydrogenase, phosphoenolpyruvate carboxylase, and phosphoenolpyruvate carboxykinase. In various embodiments, the recombinant host cell comprises one or more heterologous nucleic acids encoding all four L-aspartate pathway enzymes (i.e., L-aspartate dehydrogenase, pyruvate carboxylase, phosphoenolpyruvate carboxylase, and phosphoenolpyruvate carboxykinase). In certain embodiments, the recombinant host cell further comprises a heterologous nucleic acid encoding L-aspartate 1-decarboxylase.

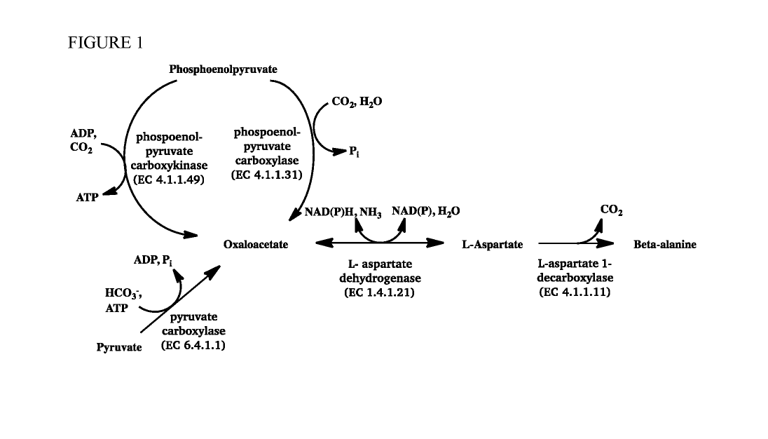

The recombinant host cells of the present invention include microbes that employ combinations of metabolic reactions for biosynthetically producing the compounds of the invention. The biosynthesized compounds can be produced intracellularly and/or secreted into the culture medium. The biosynthesized compounds produced by the recombinant host cells are L-aspartate and/or beta-alanine. The relationship of these compounds with respect to the metabolic reactions described herein are depicted in FIG. 1. In one embodiment, the recombinant host cell is engineered to produce L-aspartate under substantially anaerobic conditions. In another embodiment, the recombinant host cell is engineered to produce L-aspartate under aerobic conditions. In another embodiment, the recombinant host cell is engineered to produce beta-alanine under substantially anaerobic conditions.

The production of L-aspartate or beta-alanine via the biosynthetic pathways and recombinant host cells of the invention is particularly useful because L-aspartate and beta-alanine can be produced under substantially anaerobic conditions. Microorganisms generally lack the capacity to produce L-aspartate or beta-alanine (derived from L-aspartate using a L-aspartate 1-decarboxylase) under substantially anaerobic conditions. As described herein, the recombinant host cells of the invention are engineered to produce L-aspartate and/or beta-alanine when grown under substantially anaerobic conditions and supplied with a carbohydrate as the primary carbon source and an assimilable nitrogen source.

The L-aspartate pathway and L-aspartate 1-decarboxylase enzymes and nucleic acids encoding said enzymes may be endogenous or heterologous. In certain embodiments, the recombinant host cells provided herein comprise one or more heterologous nucleic acids encoding L-aspartate pathway and/or L-aspartate 1-decarboxylase enzymes. In certain embodiments, the recombinant host cell comprises a single heterologous nucleic acid encoding a L-aspartate pathway or L-aspartate 1-decarboxylase gene. In other embodiments, the cell comprises multiple heterologous nucleic acids encoding L-aspartate pathway and/or L-aspartate 1-decarboxylase enzymes. In these embodiments, the recombinant host cell may comprise multiple copies of a single heterologous nucleic acid and/or multiple copies of two or more heterologous nucleic acids. Recombinant host cells comprising multiple heterologous nucleic acids may comprise any number of heterologous nucleic acids.

In certain embodiments, the recombinant host cells provided herein comprise one or more endogenous nucleic acids encoding L-aspartate pathway and/or L-aspartate 1-decarboxylase enzymes. In certain of these embodiments, the cells may be engineered to express more of these endogenous enzymes. In certain of these embodiments, the endogenous enzyme being expressed at a higher level (produced at a higher amount as compared to a parental or control cell) may be operatively linked to one or more exogenous promoters or other regulatory elements.

In certain embodiments, the recombinant host cells provided herein comprise one or more endogenous nucleic acids encoding an L-aspartate pathway and/or L-aspartate 1-decarboxylase enzymes and one or more heterologous nucleic acids encoding L-aspartate pathway and/or L-aspartate 1-decarboxylase enzymes. In these embodiments, the recombinant host cells may have an active L-aspartate pathway and/or L-aspartate 1-decarboxylase that comprises one or more endogenous nucleic acids encoding L-aspartate pathway and/or L-aspartate 1-decarboxylase enzymes and one or more heterologous nucleic acids encoding L-aspartate pathway and/or L-aspartate 1-decarboxylase enzymes. In certain embodiments, the recombinant host cell may comprise both endogenous and heterologous nucleic acids encoding an L-aspartate pathway or L-aspartate 1-decarboxylase enzyme.

### 2.2.1 Oxaloacetate-Forming Enzymes

Three enzymes can be used to form oxaloacetate from the glycolytic intermediates phosphoenolpyruvate and/or pyruvate, and FIG. 1 provides a schematic showing the biosynthetic relationship of the three oxaloacetate-forming enzymes to the production of L-aspartate and beta-alanine. One oxaloacetate-forming enzyme provided by the invention is pyruvate carboxylase (EC 6.4.1.1), catalyzing conversion of pyruvate and hydrogen carbonate to oxaloacetate along with concomitant hydrolysis of adenosine triphosphate (ATP) to adenosine diphosphate (ADP). Another oxaloacetate-forming enzyme is phosphoenolpyruvate carboxylase (EC 4.1.1.31), catalyzing conversion of phosphoenolpyruvate and hydrogen carbonate to oxaloacetate along with concomitant release of phosphate. The third oxaloacetate-forming enzymes is phosphoenolpyruvate carboxykinase (EC 4.1.1.49), catalyzing formation of oxaloacetate from phosphoenolpyruvate and carbon dioxide along with concomitant formation of ATP from ADP. In various embodiments, the recombinant host cell comprises one or more heterologous nucleic acids encoding an oxaloacetate-forming enzyme selected from the group consisting of pyruvate carboxylase, phosphoenolpyruvate carboxylase, and phosphoenolpyruvate carboxykinase that results in increased production of L-aspartate and/or beta-alanine under substantially anaerobic conditions as compared to a parent cell not comprising said one or more heterologous nucleic acids. In various embodiments, the recombinant host cell comprises one or more heterologous nucleic acids encoding an oxaloacetate-forming enzyme selected from the group consisting of pyruvate carboxylase, phosphoenolpyruvate carboxylase, and phosphoenolpyruvate carboxykinase that results in increased production of L-aspartate and/or beta-alanine under aerobic conditions as compared to a parent cell not comprising said one or more heterologous nucleic acids.

Recombinant host cells of the invention engineered for production of L-aspartate and/or beta-alanine under substantially anaerobic conditions through increased expression of oxaloacetate-forming enzymes generally comprise one or more heterologous nucleic acids encoding at least one oxaloacetate-forming enzyme. In some embodiments, a recombinant host cell engineered for production of L-aspartate and/or beta-alanine under substantially anaerobic conditions comprises one or more heterologous nucleic acid encoding one oxaloacetate-forming enzyme. In other embodiments, a recombinant host cell engineered for production of L-aspartate and/or beta-alanine under substantially anaerobic conditions comprises heterologous nucleic acids encoding two oxaloacetate-forming enzymes. In yet a further embodiment, recombinant host cells of the invention engineered for production of L-aspartate and/or beta-alanine under substantially anaerobic conditions comprise heterologous nucleic acids encoding all three oxaloacetate-forming enzymes.

### 2.2.1.1 Pyruvate Carboxylase

One oxaloacetate-forming enzyme is pyruvate carboxylase, and in one embodiment, a recombinant host cell of the invention comprises one or more heterologous nucleic acids encoding a pyruvate carboxylase wherein said host cell is capable of producing L-aspartate and/or beta-alanine under substantially anaerobic conditions. In another embodiment, a recombinant host cell of the invention comprises one or more heterologous nucleic acids encoding a pyruvate carboxylase wherein said host cell is capable of producing L-aspartate and/or beta-alanine under aerobic conditions.

In some embodiments, a nucleic acid encoding pyruvate carboxylase is derived from a fungal source. Non-limiting examples of pyruvate carboxylase enzymes derived from fungal sources suitable for use in accordance with the methods of the invention include those selected from the group consisting of Aspergillus niger (UniProt ID: Q9HES8), Aspergillus terreus (UniProt ID: O93918), Aspergillus oryzae (UniProt ID:Q2UGL1; SEQ ID NO: 7), Aspergillus fumigatus (UniProt ID: Q4WP18), Paecilomyces variotii (UniProt ID: V5FWI7), P. kudriavzevii (referred to herein as PkPYC; SEQ ID NO: 58) and S. cerevisiae (UniProt ID: P11154) pyruvate carboxylase. In a specific embodiment, a recombinant host cell of the invention comprises one or more heterologous nucleic acids encoding Aspergillus oryzae pyruvate carboxylase (SEQ ID NO: 7) wherein said host cell is capable of producing L-aspartate and/or beta-alanine under substantially anaerobic conditions. In another specific embodiment, a recombinant host cell of the invention comprises one or more heterologous nucleic acids encoding Aspergillus oryzae pyruvate carboxylase (SEQ ID NO: 7) wherein said host cell is capable of producing L-aspartate and/or beta-alanine under aerobic conditions. In other embodiments, a recombinant host cell of the invention comprises one or more heterologous nucleic acids encoding PkPYC (SEQ ID NO: 58) wherein said host cell is capable of producing L-aspartate and/or beta-alanine under substantially anaerobic conditions. In yet still further embodiments, a recombinant host cell of the invention comprises one or more heterologous nucleic acids encoding PkPYC (SEQ ID NO: 58) wherein said host cell is capable of producing L-aspartate and/or beta-alanine under substantially anaerobic conditions.

Pyruvate carboxylase also useful in the compositions and methods provided herein include those enzymes that are said to be homologous to any of the pyruvate carboxylase enzymes described herein. Such homologs have the following characteristics: is capable of catalyzing the conversion of pyruvate to oxaloacetate and it shares substantial sequence identity with any pyruvate carboxylase described herein. A homolog is said to share substantial sequence identity to a pyruvate carboxylase if the amino acid sequence of the homolog is at least 60%, at least 70%, at least 80%, at least 90%, at least 95%, or at least 97% the same as that of a pyruvate carboxylase amino acid sequence set forth herein. In some embodiments, a recombinant host cell comprises heterologous nucleic acids encoding one or more pyruvate carboxylases with greater than 60% amino acid sequence identity to SEQ ID NOs: 7 and/or 58. In some embodiments, a recombinant host cell comprises heterologous nucleic acids encoding one or more pyruvate carboxylases with at least 70% amino acid sequence identity to SEQ ID NOs: 7 and/or 58. In some embodiments, a recombinant host cell comprises heterologous nucleic acids encoding one or more pyruvate carboxylases with at least 80% amino acid sequence identity to SEQ ID NOs: 7 and/or 58.

Highly conserved amino acids in Pseudomonas aeruginosa L-aspartate dehydrogenase (SEQ ID NO: 1) are G8, G10, A11, I12, G13, E69, C70, A71, A75, L84, V92, S94, G96, A97, G123, A124, I125, G126, D129, L131, A134, V142, K148, P149, F174, G176, A178, A181, L184, P186, N188, N190, V191, A192, A193, T194, L197, A198, G201, V207, A211, D212, P213, N218, G226, A227, F228, G229, P239, N243, P244, K245, T246, 5247, L249, T250, 5253, 8256, L258, and N260. In some embodiments, L-aspartate enzymes homologous to Pseudomonas aeruginosa L-aspartate dehydrogenase (SEQ ID NO: 1) comprise amino acids corresponding to at least a 50% of these highly conserved amino acids. In some embodiments, L-aspartate enzymes homologous to Pseudomonas aeruginosa L-aspartate dehydrogenase (SEQ ID NO: 1) comprise amino acids corresponding to at least 60%, at least 70%, at least 80%, at least 85%, at least 90%, at least 95%, or more than 95% of these highly conserved amino acids.

### 2.2.1.2 Phosphoenolpyruvate Carboxylase

Oxaloacetate can also be produced from phosphoenolpyruvate, which serves as the substrate for both phosphoenolpyruvate carboxylase and phosphoenolpyruvate carboxykinase enzymes. In some embodiments, a nucleic acid encoding phosphoenolpyruvate carboxylase is derived from a fungal source. A specific, non-limiting example of a phosphoenolpyruvate carboxylase enzyme derived from a fungal source suitable for use in accordance with the methods of the invention is Aspergillus niger phosphoenolpyruvate carboxylase (UniProt ID: A2QM99).

In other embodiments, a nucleic acid encoding phosphoenolpyruvate carboxylase is derived from a bacterial source. Non-limiting examples of phosphoenolpyruvate carboxylase enzymes derived from bacterial sources suitable for use in accordance with the methods of the invention include E. coli (UniProt ID: H9UZE7; SEQ ID NO: 8), Mycobacterium tuberculosis (UniProt ID: P9WIH3), and C. glutamicum (UniProt ID: P12880) phosphoenolpyruvate carboxylase enzymes. In a specific embodiment, said phosphoenolpyruvate carboxylase is E. coli phosphoenolpyruvate carboxylase (SEQ ID NO: 8).

In various embodiments, the recombinant host cell comprises one or more heterologous nucleic acids encoding a phosphoenolpyruvate carboxylase that results in increased production of L-aspartate and/or beta-alanine under substantially anaerobic conditions as compared to a parent cell not comprising said one or more heterologous nucleic acids. In a specific embodiment, said phosphoenolpyruvate carboxylase is E. coli phosphoenolpyruvate carboxylase (SEQ ID NO: 8).

### 2.2.1.3 Phosphoenolpyruvate Carboxylase

Non-limiting examples of phosphoenolpyruvate carboxykinase enzymes suitable for use in accordance with the methods of the invention include E. coli (UniProt ID: P22259), Anaerobiospirillum succiniciproducens (UniProt ID: O09460), Actinobacillus succinogenes (UniProt ID: A6VKV4), Mannheimia succiniciproducens (SEQ ID NO: 6), and Haemophilus influenzae (UniProt ID: A5UDR5) PEP carboxykinase enzymes. In yet another embodiment, the recombinant host cell comprises one or more heterologous nucleic acids encoding a phosphoenolpyruvate carboxykinase that results in increased production of L-aspartate and/or beta-alanine under substantially anaerobic conditions as compared to a parent cell not comprising said one or more heterologous nucleic acids. In a specific embodiment, said phosphoenolpyruvate carboxykinase is Mannheimia succiniciproducens phosphoenolpyruvate carboxykinase (SEQ ID NO: 6).

### 2.2.2 L-Aspartate Dehydrogenase Enzymes

Provided herein is a recombinant host cell capable of producing L-aspartate and/or beta-alanine, the cell comprising one or more heterologous nucleic acids encoding an L-aspartate dehydrogenase. An L-aspartate dehydrogenase as used herein refers to any protein with L-aspartate dehydrogenase activity, meaning the ability to catalyze the conversion of oxaloacetate to L-aspartate.

Proteins capable of catalyzing this reaction suitable for use in the compositions and methods provided herein include both NAD-dependent L-aspartate dehydrogenase and NADP-dependent L-aspartate dehydrogenase enzymes. NAD-dependent L-aspartate dehydrogenase enzymes catalyze the conversion of oxaloacetate and ammonia to L-aspartate using NADH as the electron donor. Likewise, NADP-dependent L-aspartate dehydrogenase enzymes catalyze the conversion of oxaloacetate and ammonia to L-aspartate using NADPH as the electron donor. Many L-aspartate dehydrogenase enzymes are capable of using both NADH and NADPH as electron acceptors; as such, an NAD-dependent L-aspartate dehydrogenase may also be an NADP-dependent L-aspartate dehydrogenase (and vice versa). In these cases, usage of either NADH or NADPH as the electron donor is dependent on both the relative concentration of, and affinity constant of the L-aspartate dehydrogenase exhibits for, NADH or NADPH, respectively.

In some embodiments, the recombinant host cell provided herein comprises a heterologous nucleic acid encoding an L-aspartate dehydrogenase, which is capable of producing L-aspartate and/or beta-alanine. L-aspartate dehydrogenases suitable for use in accordance with the methods of the invention include those selected from the non-limiting group consisting of Acinetobacter sp. SH024 (UniProt ID: D6JRV1; SEQ ID NO: 22), Arthrobacter aurescens (UniProt ID: A1R621), Burkholderia pseudomallei (UniProt ID: Q3JFK2; SEQ ID NO: 20), Burkholderia thailandensis (UniProt ID: Q2T559; SEQ ID NO: 19), Comamonas testosteroni (UniProt ID: D0IX49; SEQ ID NO: 26), Cupriavidus taiwanensis (UniProt ID: B3R8S4; SEQ ID NO: 2), Dinoroseobacter shibae (UniProt ID: A8LLH8; SEQ ID NO: 24), Klebsiella pneumoniae (UniProt ID: A6TDT8; SEQ ID NO: 23), Ochrobactrum anthropi (UniProt ID: A6X792; SEQ ID NO: 21), Polaromonas sp. (UniProt ID: Q126F5; SEQ ID NO: 18), Pseudomonas aeruginosa (UniProt ID: Q9HYA4; SEQ ID NO: 1), Ralstonia solanacearum (UniProt ID: Q8XRV9; SEQ ID NO: 17), Cupriavidus pinatubonensis (UniProt ID: Q46VA0; SEQ ID NO: 27), and Ruegeria pomeroyi (UniProt ID: Q5LPG8; SEQ ID NO: 25) L-aspartate dehydrogenase. In certain embodiments, the recombinant host cell provided herein comprises a heterologous nucleic acid encoding Pseudomonas aeruginosa L-aspartate dehydrogenase (SEQ ID NO: 1), which is capable of producing L-aspartate and/or beta-alanine. In other embodiments, the recombinant host cell provided herein comprises a heterologous nucleic acid encoding Cupriavidus taiwanensis L-aspartate dehydrogenase (SEQ ID NO: 2), which is capable of producing L-aspartate and/or beta-alanine. In some embodiments, a recombinant host cell of the present invention comprises a heterologous nucleic acid encoding an L-aspartate dehydrogenase selected from the group consisting of SEQ ID NOs: 17, 18, 19, 20, 21, 22, 23, 24, 25, 25, and 27, wherein the recombinant host cell is capable of producing L-aspartate and/or beta-alanine. In some embodiments, a recombinant host cell of the present invention comprises a plurality of heterologous nucleic acids, each encoding an L-aspartate dehydrogenase selected from the group consisting of SEQ ID NOs: 17, 18, 19, 20, 21, 22, 23, 24, 25, 25, and 27, wherein the recombinant host cell is capable of producing L-aspartate and/or beta-alanine.

### Homologs to L-Aspartate Dehydrogenase Enzymes

L-aspartate dehydrogenases also useful in the compositions and methods provided herein include those enzymes that are said to be “homologous” to any of the L-aspartate dehydrogenase enzymes described herein. Such homologs have the following characteristics: (1) is capable of catalyzing the conversion of oxaloacetate to L-aspartate; (2) it shares substantial sequence identity with any L-aspartate dehydrogenase described herein; (3) comprises a substantial number of amino acids corresponding to highly conserved amino acids in any L-aspartate dehydrogenase described herein; and (4) comprises one or more specific amino acids corresponding to strictly conserved amino acids in any L-aspartate dehydrogenase described herein.

A homolog is said to share substantial sequence identity to an L-aspartate dehydrogenase if the amino acid sequence of the homolog is at least 60%, at least 70%, at least 80%, at least 90%, at least 95%, or at least 97% the same as that of a L-aspartate dehydrogenase amino acid sequence set forth herein.

A number of amino acids in L-aspartate dehydrogenase enzymes provided by the invention are highly conserved, and proteins homologous to an L-aspartate dehydrogenase enzyme of the invention will generally comprise amino acids corresponding to a substantial number of highly conserved amino acids. A homolog is said to comprise a substantial number of amino acids corresponding to highly conserved amino acids in a reference sequence if at least 50%, at least 60%, at least 70%, at least 80%, at least 90%, at least 95%, or more than 95% of the highly conserved amino acids in the reference sequence are found in the homologous protein.

Highly conserved amino acids in Pseudomonas aeruginosa L-aspartate dehydrogenase (SEQ ID NO: 1) are G8, G10, A11, 112, G13, E69, C70, A71, A75, L84, V92, S94, G96, A97, G123, A124, 1125, G126, D129, L131, A134, V142, K148, P149, F174, G176, A178, A181, L184, P186, N188, N190, V191, A192, A193, T194, L197, A198, G201, V207, A211, D212, P213, N218, G226, A227, F228, G229, P239, N243, P244, K245, T246, 5247, L249, T250, 5253, 8256, L258, and N260. In some embodiments, L-aspartate enzymes homologous to Pseudomonas aeruginosa L-aspartate dehydrogenase (SEQ ID NO: 1) comprise amino acids corresponding to at least a 50% of these highly conserved amino acids. In some embodiments, L-aspartate enzymes homologous to Pseudomonas aeruginosa L-aspartate dehydrogenase (SEQ ID NO: 1) comprise amino acids corresponding to at least 60%, at least 70%, at least 80%, at least 85%, at least 90%, at least 95%, or more than 95% of these highly conserved amino acids.

Highly conserved amino acids in Cupriavidus taiwanensis L-aspartate dehydrogenase (SEQ ID NO: 2) are G8, G10, A11, 112, G13, C69, A70, A74, L83, V91, S93, G95, A96, 5121, G122, A123, 1124, G125, D128, L130, A133, V141, K147, P148, F173, E174, G175, A177, A180, L183, P185, N187, N189, V190, A191, A192, T193, L196, A197, G200, V206, A210, D211, P212, N217, G225, A226, F227, G228, P238, N242, P243, K244, T245, S246, L248, T249, 5252, S252, R255, A256, L257, L257, and N259. In some embodiments, L-aspartate enzymes homologous to Cupriavidus taiwanensis L-aspartate dehydrogenase (SEQ ID NO: 2) comprise amino acids corresponding to at least 50% of these highly conserved amino acids. In some embodiments, L-aspartate enzymes homologous to Cupriavidus taiwanensis L-aspartate dehydrogenase (SEQ ID NO: 2) comprise amino acids corresponding to at least 60%, at least 70%, at least 80%, at least 85%, at least 90%, at least 95%, or more than 95% of these highly conserved amino acids.

### Strictly Conserved Amino Acids in L-Aspartate Dehydrogenase Enzymes

Some amino acids in L-aspartate dehydrogenase enzymes provided by the invention are strictly conserved, and proteins homologous to an L-aspartate dehydrogenase enzyme of the invention must comprise amino acid(s) corresponding to these strictly conserved amino.

Amino acid H220 in SEQ ID NO: 1 functions as a general acid/base (although the invention is not to be limited by any theory of mechanism of action) and is necessary for enzyme activity; thus, an amino acid corresponding to H220 in SEQ ID NO: 1 is present in all enzymes homologous to SEQ ID NO: 1. Amino acid H220 in SEQ ID NO: 1 corresponds to amino acid H119 in SEQ ID NO: 2, and L-aspartate dehydrogenase enzymes homologous to SEQ ID NO: 2 must comprise an amino acid corresponding to H119 in SEQ ID NO: 2.

### Additional L-Aspartate Dehydrogenase Enzymes

In addition to L-aspartate dehydrogenase enzymes homologous to those described above, another class of L-aspartate dehydrogenase enzymes that can be expressed in recombinant P. kudriavzevii to produce L-aspartate from oxaloacetate are L-aspartate transaminase (EC 2.6.1.1) enzymes, which catalyzes reduction of oxaloacetate to L-aspartate along with concomitant oxidation of glutamate to alpha-ketoglutarate. Using this enzyme, it is important to recycle the alpha-ketoglutarate back to glutamate to provide the glutamate substrate necessary for additional rounds of L-aspartate transaminase catalysis. This can be accomplished by expressing a glutamate dehydrogenase (EC 1.4.1.2) that reduces alpha-ketoglutarate back to glutamate using NADH as the electron donor. This alternative metabolic pathway to L-aspartate from oxaloacetate is most useful in cases where L-aspartate dehydrogenase activity is insufficient to produce L-aspartate at the desired rate. In some embodiments of the present invention, the recombinant host cell comprises a heterologous nucleic acid encoding a L-aspartate dehydrogenase that is an L-aspartate transaminase.

Examples of suitable L-aspartate transaminase enzymes include those selected from the non-limiting group consisting of S. cerevisiae AAT2 (UnitProt ID: P23542), S. pombe L-aspartate transaminase (UniProt ID: O94320), E. coli AspC (UniProt ID: P00509), Pseudomonas aeruginosa AspC (UniProt ID: P72173), and Rhizobium meliloti AatB (UniProt ID: Q06191), among others.

### 2.2.3 L-Aspartate 1-Decarboxylase Enzymes

In various embodiments, the recombinant host cell further comprises a heterologous nucleic acid encoding a L-aspartate 1-decarboxylase. A L-aspartate 1-decarboxylase as used herein refers to any protein with L-aspartate decarboxylase activity, meaning the ability to catalyze the decarboxylation of L-aspartate to beta-alanine.

Proteins capable of catalyzing this reaction suitable for use in the compositions and methods provided herein include both bacterial L-aspartate 1-decarboxylases and eukaryotic L-aspartate decarboxylases. Bacterial L-aspartate 1-decarboxylases are pyruvoyl-dependent decarboxylases where the covalently bound pyruvoyl cofactor is produced by autocatalytic rearrangement of specific serine residues (e.g., S25 in SEQ IDs NO: 4 and 5). Eukaryotic L-aspartate decarboxylases, in contrast, do not possess a pyruvoyl cofactor and instead possess a pyridoxal 5′-phosphate cofactor. In some embodiments, the recombinant host cell comprises a heterologous nucleic acid encoding a bacterial L-aspartate 1-decarboxylase and is capable of producing beta-alanine. In other embodiments, the recombinant host cell comprises a heterologous nucleic acid encoding a eukaryotic L-aspartate 1-decarboxylase and is capable of producing beta-alanine.

Bacterial L-aspartate 1-decarboxylase enzymes suitable for use in accordance with the methods of the invention include those selected from the non-limiting group consisting of Arthrobacter aurescens (UniProt ID: A1RDH3), Bacillus cereus (UniProt ID: A7GN78), Bacillus subtilis (UniProt ID: P52999; SEQ ID NO: 5), Burkholderia xenovorans (UniProt ID: Q143J3), Clostridium acetobutylicum (UniProt ID: P58285), Clostridium beijerinckii (UniProt ID: A6LWN4), Corynebacterium efficiens (UniProt ID: Q8FU86), C. glutamicum (UniProt ID: Q9X4N0; SEQ ID NO: 4), Corynebacterium jeikeium (UniProt ID: Q4JXL3), Cupriavidus necator (UniProt ID: Q9ZHI5), Enterococcus faecalis (UniProt ID: Q833S7), E. coli (UniProt ID: Q0TLK2), Helicobacter pylori (UniProt ID: P56065), Lactobacillus plantarum (UniProt ID: Q88Z02), Mycobacterium smegmatis (UniProt ID: A0QNF3), Pseudomonas aeruginosa (UniProt ID: Q9HV68), Pseudomonas fluorescens (UniProt ID: Q84815), Staphylococcus aureus (UniProt ID: A6U4X7), and Streptomyces coelicolor (UniProt ID: P58286) L-aspartate 1-decarboxylase. In one embodiment, the recombinant host cell provided herein comprises a heterologous nucleic acid encoding Bacillus subtilis L-aspartate 1-decarboxylase (SEQ ID NO: 5) and is capable of producing beta-alanine. In another embodiment, the recombinant host cell provided herein comprises a heterologous nucleic acid encoding Corynebacterium L-aspartate 1-decarboxylase (SEQ ID NO: 4) and is capable of producing beta-alanine.

In addition to the bacterial L-aspartate 1-decarboxylase enzymes, the invention also provides eukaryotic L-aspartate 1-decarboxylases suitable for use in the compositions and methods of the invention. Eukaryotic L-aspartate 1-decarboxylase enzymes suitable for use in accordance with the methods of the invention include those selected from the non-limiting group consisting of Tribolium castaneum (UniProt ID: A9YVA8; SEQ ID NO: 3), Aedes aegypti (UniProt ID: Q17150), Drosophila mojavensis (UniProt ID: B4KIX9), and Dendroctonus ponderosae (UniProt ID: U4UTD4) L-aspartate 1-decarboxylase. In one embodiment, the recombinant host cell provided herein comprises a heterologous nucleic acid encoding Tribolium castaneum L-aspartate 1-decarboxylase (SEQ ID NO: 3) and is capable of producing beta-alanine.

L-aspartate 1-decarboxylase enzymes also useful in the compositions and methods provided herein include those enzymes which are said to be “homologous” to any of the L-aspartate 1-decarboxylase enzymes described herein. Such homologs have the following characteristics: (1) is capable of catalyzing the decarboxylation of L-aspartate to beta-alanine; (2) it shares substantial sequence identity with any L-aspartate 1-decarboxylase described herein; (3) comprises a substantial number of amino acids corresponding to highly conserved amino acids in any L-aspartate 1-decarboxylase described herein; and (4) comprises one or more specific amino acids corresponding to strictly conserved amino acids in any L-aspartate 1-decarboxylase described herein.

### Percent Sequence Identity

A homolog is said to share substantial sequence identity to an L-aspartate 1-decarboxylase if the amino acid sequence of the homolog is at least 60%, at least 70%, at least 80%, at least 90%, at least 95%, or at least 97% the same as that of a L-aspartate 1-decarboxylase amino acid sequence described herein.

### Highly Conserved Amino Acids in L-Aspartate 1-Decarboxylase Enzymes

A number of amino acids in both bacterial and eukaryotic L-aspartate 1-decarboxylase enzymes provided herein are highly conserved, and proteins homologous to either a bacterial or a eukaryotic L-aspartate dehydrogenase enzyme of the invention will generally comprise amino acids corresponding to a substantial number of highly conserved amino acids. As described above, a homolog is said to comprise a substantial number of amino acids corresponding to highly conserved amino acids in a reference sequence if at least 50%, at least 60%, at least 70%, at least 80%, at least 90%, at least 95%, or more than 95% of the highly conserved amino acids in the reference sequence are found in the homologous protein.

Highly conserved amino acids in C. glutamicum L-aspartate 1-decarboxylase (SEQ ID NO: 4) are K9, H11, R12, A13, V15, T16, A18, L20, Y22, G24, S25, D29, E42, N51, G52, R54, T57, Y58, 160, G62, G65, G67, N72, G73, A74, A75, A76, G82, D83, V85, 186, Y90, E97, P103, and N112. In some embodiments, L-aspartate 1-decarboxylase enzymes homologous to C. glutamicum L-aspartate 1-decarboxylase (SEQ ID NO: 4) comprise amino acids corresponding to at least a 50% of these highly conserved amino acids. In some embodiments, L-aspartate 1-decarboxylase enzymes homologous to C. glutamicum L-aspartate 1-decarboxylase (SEQ ID NO: 4) comprise amino acids corresponding to at least 60%, at least 70%, at least 80%, at least 85%, at least 90%, at least 95%, or more than 95% of these highly conserved amino acids.

Highly conserved amino acids in Bacillus subtilis L-aspartate 1-decarboxylase (SEQ ID NO: 5) are K9, H11, R12, A13, V15, T16, A18, L20, Y22, G24, S25, D29, E42, N51, G52, R54, T57, Y58, 160, G62, G65, G67, N72, G73, A74, A75, A76, G82, D83, V85, I86, Y90, E97, P103, and N112. In some embodiments, L-aspartate 1-decarboxylase enzymes homologous to Bacillus subtilis L-aspartate 1-decarboxylase (SEQ ID NO: 5) comprise amino acids corresponding to at least a 50% of these highly conserved amino acids. In some embodiments, L-aspartate 1-decarboxylase enzymes homologous to Bacillus subtilis L-aspartate 1-decarboxylase (SEQ ID NO: 5) comprise amino acids corresponding to at least 60%, at least 70%, at least 80%, at least 85%, at least 90%, at least 95%, or more than 95% of these highly conserved amino acids.

Highly conserved amino acids in Tribolium castaneum L-aspartate 1-decarboxylase (SEQ ID NO: 3) are V88, P94, D102, L115, 5126, V127, T129, H131, P132, F134, N136, Q137, L138, 5140, D143, Y145, Q150, T153, D154, L156, N157, P158, 5159, Y161, T162, E164, V165, P167, L171, M172, E173, E174, V176, L177, E179, M180, R181, 1183, G185, G191, G193, F195, P197, G198, G199, 5200, A202, N203, G204, Y205, 1207, A210, R211, P216, K219, G222, L229, F232, T233, 5234, E235, A237, H238, Y239, 5240, K243, A245, F247, G249, G251, G264, P285, V288, T291, G293, T294, T295, V296, G298, A299, F300, D301, C310, K312, W316, H318, D320, A321, A322, W323, G324, G325, G326, A327, L328, 5330, R334, L336, L337, G339, D344, 5345, V346, T347, W348, N349, P350, H351, K352, L353, L354, A356, Q358, Q359, C360, 5361, T362, L364, H367, L371, H375, A379, Y381, L382, F383, Q384, D386, K387, F388, Y389, D390, D394, G396, D397, H399, Q401, C402, G403, R404, A406, D407, V408, K410, F411, W412, M414, W415, A417, K418, G419, G422, H426, F431, R444, G446, P454, N458, F461, Y463, P465, R469, L481, A485, P486, K489, E490, M492, G496, M498, T501, Y502, Q503, N510, F511, F512, R513, V515, Q517, 5519, L521, D525, M526, E532, E534, L536. In some embodiments, L-aspartate 1-decarboxylase enzymes homologous to Tribolium castaneum L-aspartate 1-decarboxylase (SEQ ID NO: 3) comprise amino acids corresponding to at least a 50% of these highly conserved amino acids. In some embodiments, L-aspartate 1-decarboxylase enzymes homologous to Tribolium castaneum L-aspartate 1-decarboxylase (SEQ ID NO: 3) comprise amino acids corresponding to at least 60%, at least 70%, at least 80%, at least 85%, at least 90%, at least 95%, or more than 95% of these highly conserved amino acids.

### L-Aspartate 1-Decarboxylase Strictly Conserved Amino Acids

Some amino acids in L-aspartate 1-decarboxylase enzymes provided by the invention are strictly conserved, and proteins homologous to an L-aspartate 1-decarboxylase enzyme of the invention must comprise amino acid(s) corresponding to these strictly conserved amino acids.

Strictly conserved amino acids in both the Bacillus subtilis L-aspartate 1-decarboxylase (SEQ ID NO: 5) and C. glutamicum L-aspartate 1-decarobxylase (SEQ ID NO: 4) amino acid sequences are K9, G24, S25, R54, and Y58. The epsilon-amine group on K9 is believed to form an ion pair with alpha-carboxyl group on L-aspartate, R54 is believed to form an ion pair with the gamma-carboxyl group on L-aspartate, and Y58 is believed to donate a proton to an extended enolate reaction intermediate; thus, these three amino acids are important for L-aspartate binding and subsequent decarboxylation. Additionally, proteolytic cleavage between residues G24 and S25 produces an N-terminal pyruvoyl moiety also necessary for decarboxylase activity. Therefore, enzymes homologous to SEQ ID NO: 4 and/or SEQ ID 5 will comprise amino acids corresponding to K9, G24, S25, R54, and Y58 in SEQ ID NOs: 4 and/or 5.

Strictly conserved amino acids in the Tribolium castaneum L-aspartate 1-decarboxylase (SEQ ID NO: 3) amino acid sequence are Q137, H238, K352, and R513. Q137 and R513 form a salt bridge with the gamma-carboxyl group on L-aspartate, H238 is a base-stacking residue with the pyridine ring of the pyridoxal 5′-phosphate cofactor, and K352 forms a Schiff base linkage with the pyridoxal 5′-phosphate cofactor. Thus, these four amino acids are important for L-aspartate or cofactor binding and subsequent L-aspartate decarboxylation, and enzymes homologous to SEQ ID NO: 3 will comprise amino acids corresponding to Q137, H238, K352, and R513 in SEQ ID NO: 3.

### 2.2.4 Consensus Sequences

The present invention also provides consensus sequences useful in identifying and/or constructing L-aspartate dehydrogenases and L-aspartate 1-decarboxylases suitable for use in accordance with the methods of the invention. In various embodiments, these consensus sequences comprise active site amino acid residues believed to be necessary (although the invention is not to be limited by any theory of mechanism of action) for substrate recognition and reaction catalysis, as described below. Thus, an L-aspartate dehydrogenase encompassed by an L-aspartate dehydrogenase consensus sequence provided herein has an enzymatic activity that is identical, or essentially identical, or at least substantially similar with respect to ability to reduce oxaloacetate to L-aspartate to that of one of the enzymes exemplified herein. Likewise, an L-aspartate 1-decarboxylase encompassed by a L-aspartate 1-decarboxylase consensus sequence provided herein has an enzymatic activity that is identical, or essentially identical, or at least substantially similar with respect to ability to decarboxylate L-aspartate to beta-alanine to that of one of the enzymes exemplified herein.

Enzymes also useful in the compositions and methods provided herein include those that are homologous to consensus sequences provided by the invention. As noted above, any enzyme substantially homologous to an enzyme described herein can be used in a host cell of the invention.

The percent sequence identity of an enzyme relative to a consensus sequence is determined by aligning the enzyme sequence against the consensus sequence. Those skilled in the art will recognize that various sequence alignment algorithms are suitable for aligning an enzyme with a consensus sequence. See, for example, Needleman, S B, et al “A general method applicable to the search for similarities in the amino acid sequence of two proteins.” Journal of Molecular Biology 48 (3): 443-53 (1970). Following alignment of the enzyme sequence relative to the consensus sequence, the percentage of positions where the enzyme possesses an amino acid (or dash) described by the same position in the consensus sequence determines the percent sequence identity.

### 2.2.4.1 L-Aspartate Dehydrogenase Consensus Sequences

An L-aspartate dehydrogenase consensus sequence (SEQ ID NO: 14) provides the sequence of amino acids in which each position identifies the amino acid (if a specific amino acid is identified) or a subset of amino acids (if a position is identified as variable) most likely to be found at a specified position in an L-aspartate dehydrogenase. Those of skill in the art will recognize that fixed amino acids and conserved amino acids in these consensus sequences are identical to (in the case of fixed amino acids) or consistent with (in the case of conserved amino acids) with the wild-type sequence(s) on which the consensus sequence is based. Following alignment of a query protein with a consensus sequence provided herein, the occurrence of a dash in the aligned query protein sequence indicates an amino acid deletion in the query protein sequence relative to the consensus sequence at the indicated position. Likewise, the occurrence of a dash in the aligned consensus sequence indicates an amino acid addition in the query protein sequence relative to the consensus sequence at the indicated position. Amino acid additions and deletions are common to proteins encompassed by consensus sequences of the invention, and their occurrence is reflected as a lower percent sequence identity (i.e., amino acid addition or deletions are treated identically to amino acid mismatches when calculating percent sequence identity).

In various embodiments, L-aspartate dehydrogenase enzymes suitable for use in accordance with the methods of the invention have L-aspartate dehydrogenase activity and comprise an amino acid sequence with at least 60%, at least 70%, at least 80%, at least 90%, or at least 95% sequence identity to SEQ ID NO: 14. For example, the Pseudomonas aeruginosa L-aspartate dehydrogenase (SEQ ID NO: 1) and Cupriavidus taiwanensis L-aspartate dehydrogenase (SEQ ID NO: 2) sequences are 79% and 83% identical to consensus sequence SEQ ID NO: 14, and are therefore encompassed by consensus sequence SEQ ID NO: 14.

In enzymes homologous to SEQ ID NO: 14, amino acids that are highly conserved are G8, G10, A11, 112, G13, E69, A71, G72, H73, A75, H79, P82, L84, G87, S94, G96, A97, L98, A110, A111, G114, L120, G123, A124, 1125, G126, D129, A130, A133, A134, G137, G138, L139, V142, Y144, G146, R147, K148, P149, W153, T156, P157, E159, D163, L164, 1173, F174, G176, A178, A181, A182, P186, K187, N188, A189, N190, V191, A192, A193, T194, A198, G199, G201, L202, T205, V207, L209, A211, D212, P213, N218, H220, A224, G226, A227, F228, G229, L233, P239, L240, N243, P244, K245, T246, 5247, A248, L249, T250, 5253, R256, A257, N260, and 1267. In various embodiments, L-aspartate dehydrogenase enzymes homologous to SEQ ID NO: 14 comprise at least 50%, at least 60%, at least 70%, at least 80%, at least 85%, at least 90%, at least 95%, or sometimes all of these highly conserved amino acids at positions corresponding to the highly conserved amino acids identified in SEQ ID NO: 14. In some embodiments, each of these highly conserved amino acids are found in a desired L-aspartate dehydrogenase, as provided in SEQ ID NOs: 1 and 2.

Amino acid H220 in SEQ ID NO: 14 functions as a general acid/base (although the invention is not to be limited by any theory of mechanism of action) and is necessary for enzyme activity; thus, an amino acid corresponding to H220 in consensus sequence SEQ ID NO: 14 is found in enzymes homologous to SEQ ID NO: 14. For example, the strictly conserved amino acid corresponding to H220 in consensus sequence SEQ ID NO: 14 is found in L-aspartate dehydrogenases set forth in SEQ ID NOs: 1 and 2.

### 2.2.4.2 L-Aspartate 1-Decarboxylase Consensus Sequences

L-aspartate 1-decarboxylases also useful in the compositions and methods provided herein include those that are homologous to L-aspartate 1-decarboxylase consensus sequences described herein. Any L-aspartate 1-decarboxylase substantially homologous to an L-aspartate 1-decarboxylase consensus sequence described herein can be used in a host cell of the invention.

The invention provides two L-aspartate 1-decarboxylase consensus sequences: (i) L-aspartate 1-decarboxylase based on bacterial L-aspartate 1-decarboxylase enzymes (SEQ ID NO:15), and (ii) L-aspartate 1-decarboxylase based on eukaryotic L-aspartate 1-decarboxylase enzymes (SEQ ID NO:16). The consensus sequences provide a sequence of amino acids in which each position identifies the amino acid (if a specific amino acid is identified) or a subset of amino acids (if a position is identified as variable) most likely to be found at a specified position in an L-aspartate dehydrogenase of that class. Those of skill in the art will recognize that fixed amino acids and conserved amino acids in these consensus sequences are identical to (in the case of fixed amino acids) or consistent with (in the case of conserved amino acids) with the wild-type sequence(s) on which the consensus sequence is based. Following alignment of a query protein with a consensus sequence provided herein, the occurrence of a dash in the aligned query protein sequence indicates an amino acid deletion in the query protein sequence relative to the consensus sequence at the indicated position. Likewise, the occurrence of a dash in the aligned consensus sequence indicates an amino acid addition in the query protein sequence relative to the consensus sequence at the indicated position. Amino acid additions and deletions are common to proteins encompassed by consensus sequences of the invention, and their occurrence is reflected as a lower percent sequence identity (i.e., amino acid addition or deletions are treated identically to amino acid mismatches when calculating percent sequence identity).

### Bacterial L-Aspartate 1-Decarboxylase Consensus Sequences

The invention provides a L-aspartate 1-decarboxylase consensus sequence based on bacterial L-aspartate 1-decarboxylase enzymes (SEQ ID NO: 15), and in various embodiments, L-aspartate 1-decarboxylase enzymes suitable for use in accordance with the methods of the invention have L-aspartate 1-decarboxylase activity and comprise an amino acid sequence with at least 55%, at least 60%, at least 70%, at least 80%, at least 90%, or at least 95% sequence identity to SEQ ID NO: 15. The Bacillus subtilis L-aspartate 1-decarboxylase (SEQ ID NO: 5) and C. glutamicum L-aspartate 1-decarboxylase (SEQ ID NO: 4) amino acid sequences are 55% and 79% identical to consensus sequence SEQ ID NO: 15, and are therefore encompassed by consensus sequence SEQ ID NO: 15.

In enzymes homologous to SEQ ID NO: 15, amino acids that are highly conserved are K9, H11, R12, A13, V15, T16, A18, L20, Y22, G24, S25, D29, E42, N51, G52, R54, T57, Y58, 160, G62, G65, G67, N72, G73, A74, A75, A76, G82, D83, V85, 186, Y90, E97, P103, and N112. In various embodiments, L-aspartate 1-decarboxylase enzymes homologous to SEQ ID NO: 15 comprise at least 50%, at least 60%, at least 70%, at least 80%, at least 85%, at least 90%, at least 95%, or sometimes all of these highly conserved amino acids at positions corresponding to the highly conserved amino acids identified in SEQ ID NO: 15. For example, all of the highly conserved amino acids are found in the L-aspartate 1-decarboxylase sequences set forth in SEQ ID NOs: 4 and 5.

Five strictly conserved amino acids (K9, G24, S25, R54, and Y58) are present in consensus sequence SEQ ID NO: 15, and these residues are important for L-aspartate 1-decarboxylase activity. The function, although the invention is not to be limited by any theory of mechanism of action, of each strictly conserved amino acid is as follows. The epsilon-amine group on K9 forms an ion pair with alpha-carboxyl group on L-aspartate, R54 is forms an ion pair with the gamma-carboxyl group on L-aspartate, and Y58 donates a proton to an extended enolate reaction intermediate. Additional strictly conserved residues in SEQ ID NO: 15 are G24 and S25, and proteolytic cleavage between G24 and S25 results in production of an N-terminal pyruvoyl moiety required for decarboxylase activity. Enzymes homologous to consensus sequence SEQ ID NO: 15 comprise amino acids corresponding to all five of the strictly conserved amino acids identified in consensus sequence SEQ ID NO: 15.

### Eukaryotic L-Aspartate 1-Decarboxylase Consensus Sequences

The invention provides a second L-aspartate 1-decarboxylase consensus sequence based on eukaryotic L-aspartate 1-decarboxylase enzymes (SEQ ID NO: 16). In various embodiments, L-aspartate 1-decarboxylase enzymes suitable for use in accordance with the methods of the invention have L-aspartate 1-decarboxylase activity and comprise an amino acid sequence with at least 55%, at least 60%, at least 70%, at least 80%, at least 90%, or at least 95% sequence identity to SEQ ID NO: 16. The Tribolium castaneum L-aspartate 1-decarboxylase (SEQ ID NO: 3) amino acid sequence is 70% identical to consensus sequence SEQ ID NO: 16, and is therefore encompassed by consensus sequence SEQ ID NO: 16.

In enzymes homologous to SEQ ID NO: 16, highly conserved amino acids are V130, P136, D144, L157, 5168, V169, T171, H173, P174, F176, N178, Q179, L180, 5182, D185, Y187, Q192, T195, D196, L198, N199, P200, 5201, Y203, T204, E206, V207, P209, L213, M214, E215, E216, V218, L219, E221, M222, R223, 1225, G227, G234, G236, F238, P240, G241, G242, 5243, A245, N246, G247, Y248, 1250, A253, R254, P259, K262, G265, L272, F275, T276, 5277, E278, A280, H281, Y282, 5283, K286, A288, F290, G292, G294, G307, P328, V331, T334, G336, T337, T338, V339, G341, A342, F343, D344, C353, K355, W359, H361, D363, A364, A365, W366, G367, G368, G369, A370, L371, 5373, R377, L379, L380, G382, D387, 5388, V389, T390, W391, N392, P393, H394, K395, L396, L397, A399, Q401, Q402, C403, 5404, T405, L407, H410, L414, H418, A422, Y424, L425, F426, Q427, D429, K430, F431, Y432, D433, D437, G439, D440, H442, Q444, C445, G446, R447, A449, D450, V451, K453, F454, W455, M457, W458, A460, K461, G462, G465, H469, F474, R487, G489, P497, N501, F504, Y506, P508, R512, L525, A529, P530, K533, E534, M536, G540, M542, T545, Y546, Q547, N554, F555, F556, R557, V559, Q561, 5563, L565, D569, M570, E576, E578, and L580. In various embodiments, L-aspartate 1-decarboxylase enzymes homologous to SEQ ID NO: 16 comprise at least 50%, at least 60%, at least 70%, at least 80%, at least 85%, at least 90%, at least 95%, or sometimes all of these highly conserved amino acids at positions corresponding to the highly conserved amino acids identified in SEQ ID NO: 16. All of these highly conserved amino acids are found in the Tribolium castaneum L-aspartate 1-decarboxylases set forth in SEQ ID NO: 3.

Strictly conserved amino acids in the eukaryotic L-aspartate 1-decarboxylase consensus sequence (SEQ ID NO: 16) are Q179, H281, K395, and R557. The function, although the invention is not to be limited by any theory of mechanism of action, of each strictly conserved amino acid is as follows. Q179 and R557 form a salt bridge with the gamma-carboxyl group on L-aspartate, H281 is a base-stacking residue with the pyridine ring of the pyridoxal 5′-phosphate cofactor, and K395 forms a Schiff base linkage with the pyridoxal 5′-phosphate cofactor. Thus, these four amino acids are important for L-aspartate or cofactor binding and subsequent L-aspartate decarboxylation. Enzymes homologous to consensus sequence SEQ ID NO: 16 comprise amino acids corresponding to all four strictly conserved amino acids identified in consensus sequence SEQ ID NO: 16. All four of these strictly conserved amino acids are found in the Tribolium castaneum L-aspartate 1-decarboxylase set forth in SEQ ID NO: 3.

### Section 3: Deletions or Disruption of Endogenous Nucleic Acids

In another aspect, the invention provides host cells genetically modified to delete or otherwise reduce the activity of endogenous proteins. Specific nucleic acid sequences are partially, substantially, or completely deleted or disrupted, silenced, inactivated, or down-regulated in order to partially, substantially, or completely reduce or eliminate the activity for which they encode, as in, for example, expression or activity of an enzyme. As used herein, “deletion or disruption” with regard to a nucleic acid means that either all or part of a protein coding region, a promoter, a terminator, and/or other regulatory element is modified (such as by deletion, insertion, or mutation of nucleic acids) such that the nucleic acid no longer produces an protein, produces a reduced quantity of an protein, or produces a protein with reduced activity (e.g., reduced enzymatic activity).

As used herein, “deletion or disruption” with regard to an enzyme means deletion or disruption of at least one, and often more than one, and sometimes all copies of nucleic acid(s) encoding enzymes with the specified activity. Many host cells suitable for use in the compositions and methods of the invention comprise two or more endogenous nucleic acids encoding two or more enzymes with the same activity. For example, diploid, triploid, and tetraploid microbes comprise two, three, and four sets of chromosomes, respectively, and two nucleic acids encoding for two enzymes with the same enzyme activity are found on each chromosome pair. Likewise, gene duplication events can lead to the occurrence of two or more nucleic acids on the genome of a host cell encoding for two or more enzymes with the same activity. In some embodiments, the recombinant host cells comprise a deletion or disruption of one nucleic acid encoding an enzyme. In other embodiments, the recombinant host cells comprise a deletion or disruption of more than one nucleic acid encoding an enzyme, and sometimes all nucleic acids encoding an enzyme.

In certain embodiments, the recombinant host cells provided herein comprise a deletion or disruption of one or more metabolic pathways. As used herein, “deletion or disruption” with regard to a metabolic pathway means that the pathway produces a reduced quantity of one or more end-products of the metabolic pathway. In certain embodiments, deletion or disruption of a metabolic pathway is accomplished by deletion or disruption of one or more nucleic acids encoding metabolic pathway enzymes. In some of these embodiments, the recombinant host cell comprising said deleted or disrupted metabolic pathway no longer produces the end-product of the metabolic pathway, or produces at least 10%, at least 20%, at least 30%, at least 40%, at least 50%, at least 60%, at least 70%, at least 80%, at least 90%, at least 95%, or more than 95% less end-product of the metabolic pathway as compared to a parental cell. As used herein, parental cell refers to a cell that does not comprise the indicated genetic modification both is otherwise genetically identical to the cell comprising the indicated genetic modification.

In certain embodiments, the nucleic acids deleted or disrupted as described herein may be endogenous to the native strain of the microorganism, and may be understood to be “native nucleic acids” or “endogenous nucleic acids”. A nucleic acid is thus an endogenous nucleic acid if it has not been genetically modified or manipulated through human intervention in a manner that intentionally alters the genotype and/or phenotype of the microorganism. For example, a nucleic acid of a wild type organism may be considered to be an endogenous nucleic acid. In other embodiments, the nucleic acids targeted for deletion or disruption may be heterologous to the microorganism.

In certain embodiments, the recombinant host cells provided herein comprise a deletion or disruption of one or more nucleic acids encoding enzymes. In some of these embodiments, the host cells comprising the one or more deleted or disrupted nucleic acids no longer produce an enzyme, or produce less than 10%, less than 25%, less than 50%, less than 75%, less than 90%, less than 95%, or less than 97% of the amount of enzyme produced by parental cells. In other embodiments, the recombinant host cells comprising the deleted or disrupted nucleic acid(s) produces the same amount of enzyme as parental cells, but the enzyme exhibits reduced activity as compared to the enzyme encoded by the unmodified nucleic acid. In some of these embodiments, the deleted or disrupted nucleic acid no longer encodes for an active enzyme, or encodes for an enzyme with at least 10%, at least 20%, at least 30%, at least 40%, at least 50%, at least 60%, at least 70%, at least 80%, at least 90%, or more than 90% reduced activity as compared to the enzyme encoded by the endogenous nucleic acid. Those skilled in the art will recognize that deletion or disruption of a nucleic acid can simultaneously result in both a decrease in the quantity of an enzyme produced by a recombinant host cell as well as a decrease in the activity of an enzyme encoded by the deleted or disrupted nucleic acid.

### 3.1. Deletion or Disruption of Endogenous Anaerobic Pathways and Enzymes Encoding Endogenous Anaerobic Pathway Enzymes

The present invention describes the engineering of a recombinant host cell to convert various endogenous anaerobic fermentation pathways into anaerobic L-aspartate, and optionally beta-alanine, pathways. Microbes will not grow under anaerobic growth conditions unless the fermentation pathway is redox balanced (i.e., there is no net accumulation of NADH, NADPH, or other redox cofactor).

Reduction and oxidation (redox) reactions play a key role in anaerobic metabolism, allowing the transfer of electrons from one compound to another, and thereby creating free energy for use in cellular metabolism. Redox co-factors facilitate the transfer of electrons from one chemical to another within the host cell. Several compounds and proteins can function as redox co-factors. During anaerobic catabolism of carbohydrates the most relevant co-factors are nicotinamide adenine dinucleotides (NADH and NADPH), and the iron sulfur protein ferredoxin (Fd). Typically, NADH is the most relevant co-factor in yeast cells during anaerobic catabolism of carbohydrates.

In order for cellular growth, the redox co-factors must discharge the same number of electrons they accept; thus, the net electron accumulation in the host cell is zero. Electrons are placed onto redox co-factors during carbohydrate catabolism, and must be removed from redox co-factors during end-product formation. In order for an end-product to be produced at high yield under anaerobic conditions the type and number of redox co-factors used during carbohydrate catabolism must match the type and number of redox co-factors used during end-product formation.

Carbohydrate catabolism ends in the formation of pyruvate, and electrons are removed during the conversion of glyceraldehyde 3-phosphate to 1,3-biphosphoglycerate (providing two electrons). This reaction is catalyzed by glyceraldehyde phosphate dehydrogenase (GAPDH; EC 1.2.1.12), and in yeast the endogenous enzyme uses NAD+ is used as the electron acceptor. When using glucose as the carbohydrate, two mols glyceraldehyde 3-phosphate can be theoretically produced per mol glucose, and thus two mols NADH can theoretically be produced per mol glucose in host cells expressing an NAD-dependent GAPDH. GAPDH enzymes may use alternate co-factors, including NADPH; NADP-dependent GAPDH enzymes are categorized under enzyme commission number EC 1.2.1.13, and include those found in Chlamydomonas reinhardtii, Clostridium acetobutylicum, Spinacia oleracea, and Sulfolobus solfataricus, among others. Host cells comprising NAD-dependent GAPDH enzymes can be engineered using standard microbial engineering techniques to express NADP-dependent GAPDH enzymes and thus produce NADPH, or a combination of NADH and NADPH, during carbohydrate catabolism to pyruvate.

Redox co-factors accepting electrons during catabolism of carbohydrates to pyruvate must discharge those electrons during production of the fermentation end-product to enable anaerobic growth and/or production of the end-product at high yield. Microbes capable of growth under substantially anaerobic conditions comprise one or more endogenous anaerobic fermentation pathways whose activity results in the reconsumption of redox cofactors produced during carbohydrate catabolism. The activity of endogenous anaerobic fermentation pathway(s) reduces the availability of redox cofactors for use by the heterologous L-aspartate pathway enzymes of the invention, thereby decreasing L-aspartate and/or beta-alanine yields from carbohydrates. Therefore, deletion or disruption of endogenous anaerobic fermentation pathways and nucleic acids encoding endogenous anaerobic fermentation pathway enzymes is useful for increasing the yield of L-aspartate and/or beta-alanine produced by recombinant host cells of the invention grown under substantially anaerobic conditions.

An anaerobic fermentation pathway is any metabolic pathway that: (i) comprises enzymes that reconsume redox cofactors produced during carbohydrate catabolism, and (ii) whose activity results in a detectable level of end-product in host cells grown under substantially anaerobic conditions. Examples of anaerobic fermentation pathways include, but are not limited to, ethanol, glycerol, malate, lactate, 1-butanol, isobutanol, 1,3-propanediol, and 1,2-propanediol anaerobic fermentation pathways. For example, ethanol is the main fermentation end-product of most wild-type microbes, and especially yeast, grown anaerobically on carbohydrate, and the redox co-factors produced during catabolism of carbohydrates to pyruvate are reconsumed during conversion of pyruvate to ethanol. In the recombinant host cells of the present invention, the endogenous fermentation pathway, typically, but not limited to, an ethanol fermentation pathway, has been deleted or disrupted. Redox cofactors produced during pyruvate formation from glucose are reconsumed during production of L-aspartate through the activity of an L-aspartate dehydrogenase, and the net result is a redox balanced, and thus anaerobic, fermentation pathway capable of producing L-aspartate and/or beta-alanine at high yield.

### 3.1.1 Deletion or Disruption of Ethanol Fermentation Pathways and Nucleic Acids Encoding Ethanol Fermentation Pathway Enzymes

Deletion or disruption of ethanol fermentation pathway(s) and nucleic acids encoding ethanol fermentation pathway enzymes is important for engineering a recombinant host cell capable of efficient production of L-aspartate and/or beta-alanine under substantially anaerobic conditions.

In yeast host cells, an ethanol fermentation pathway comprises two enzymes: pyruvate decarboxylase and alcohol dehydrogenase. Pyruvate decarboxylase (EC 4.1.1.1) catalyzes the decarboxylation of pyruvate to acetaldehyde; alcohol dehydrogenase (EC 1.1.1.1) catalyzes the reduction of acetaldehyde to ethanol along with concomitant oxidation of NADH to NAD+ and/or NADPH to NADP+. In yeast cells of the invention, an ethanol fermentation pathway can be deleted or disrupted by deletion or disruption of one or more nucleic acids encoding pyruvate decarboxylase and/or alcohol dehydrogenase. In certain embodiments, the recombinant host cells provided herein comprise a deletion or disruption of one or more endogenous nucleic acids encoding an ethanol fermentation pathway enzyme. In some embodiments, the recombinant host cells provided herein comprise a deletion or disruption of one or more nucleic acids encoding pyruvate decarboxylase. In some embodiments, the recombinant host cells provided herein comprise a deletion or disruption of one or more nucleic acids encoding alcohol dehydrogenase. In some embodiments, the recombinant host cells provided herein comprise a deletion or disruption of one or more nucleic acids encoding pyruvate decarboxylase and alcohol dehydrogenase.

Deletion or disruption of nucleic acids encoding ethanol fermentation pathway enzymes decrease the ability of the recombinant host cell to produce ethanol and/or increases the ability of the recombinant host cell to produce L-aspartate and/or beta-alanine. In various embodiments, recombinant host cells comprising deletion or disruption of one or more nucleic acids encoding ethanol fermentation pathway enzymes decreases ethanol production by at least 10%, at least 25%, at least 50%, at least 60%, at least 70%, at least 90%, at least 95%, or at least 99% as compared to parental cells that do not comprise this genetic modification. In some embodiments, recombinant host cells comprising deletion or disruption of one or more nucleic acids encoding ethanol fermentation pathway enzymes increase L-aspartate and/or beta-alanine production by at least 10%, at least 25%, at least 50%, at least 75%, at least 100%, or more than 100% as compared to parental cells that do not comprise this genetic modification.

### Deletion or Disruption of Nucleic Acids Encoding Pyruvate Decarboxylase

In various embodiments, the recombinant host cells comprise a deletion or disruption of one or more nucleic acids encoding pyruvate decarboxylase. In some embodiments, one nucleic acid encoding pyruvate decarboxylase is deleted or disrupted. In other embodiments, two nucleic acids encoding pyruvate decarboxylase are deleted or disrupted. In other embodiments, more than two nucleic acids encoding pyruvate decarboxylase are deleted or disrupted. In still further embodiments, all nucleic acids encoding pyruvate decarboxylase are deleted or disrupted.

P. kudriavzevii has more than one nucleic acid encoding pyruvate decarboxylase, namely PDC1 (referred to herein as PkPDC1; SEQ ID NO: 9), PDC5 (referred to herein as PkPDC5; SEQ ID NO: 29), and PDC6 (referred to herein as PkPDC6; SEQ ID NO: 30). In various embodiments, the recombinant host cell comprises a deletion or disruption of one or more nucleic acids encoding pyruvate decarboxylases with the amino acid sequence set forth in SEQ ID NO: 9, or one or more nucleic acids encoding enzymes with an amino sequence with at least 50%, at least 60%, at least 70%, at least 80%, at least 95%, at least 97%, or at least 99% sequence identity to the amino acid sequence of SEQ ID NO: 9. In specific embodiments wherein the recombinant host cell of the invention is P. kudriavzevii, the recombinant host cell comprises deletion or disruption of two nucleic acids encoding pyruvate decarboxylases with the amino acid sequence set forth in SEQ ID NO: 9, or two nucleic acids encoding enzymes with amino sequences with at least 50%, at least 60%, at least 70%, at least 80%, at least 95%, at least 97%, or at least 99% sequence identity to the amino acid sequence of SEQ ID NO: 9.

In some embodiments, the recombinant host cell comprises a deletion or disruption of one or more nucleic acids encoding PkPDC5 (SEQ ID NO: 29), or one or more nucleic acids encoding enzymes with an amino sequence with at least 50%, at least 60%, at least 70%, at least 80%, at least 95%, at least 97%, or at least 99% sequence identity to the amino acid sequence of SEQ ID NO: 29. In specific embodiments wherein the recombinant host cell of the invention is P. kudriavzevii, the recombinant host cell comprises deletion or disruption of two nucleic acids encoding pyruvate decarboxylases with the amino acid sequence set forth in SEQ ID NO: 29, or two nucleic acids encoding enzymes with amino sequences with at least 50%, at least 60%, at least 70%, at least 80%, at least 95%, at least 97%, or at least 99% sequence identity to the amino acid sequence of SEQ ID NO: 29.

In some embodiments, the recombinant host cell comprises a deletion or disruption of one or more nucleic acids encoding PkPDC6 (SEQ ID NO: 30), or one or more nucleic acids encoding enzymes with an amino sequence with at least 50%, at least 60%, at least 70%, at least 80%, at least 95%, at least 97%, or at least 99% sequence identity to the amino acid sequence of SEQ ID NO: 30. In specific embodiments wherein the recombinant host cell of the invention is P. kudriavzevii, the recombinant host cell comprises deletion or disruption of two nucleic acids encoding pyruvate decarboxylases with the amino acid sequence set forth in SEQ ID NO: 30, or two nucleic acids encoding enzymes with amino sequences with at least 50%, at least 60%, at least 70%, at least 80%, at least 95%, at least 97%, or at least 99% sequence identity to the amino acid sequence of SEQ ID NO: 30.

In still further embodiments, the recombinant host cell comprises a deletion or disruption of one or more nucleic acids encoding PkPDC1 (SEQ ID NO: 9), PkPDC5 (SEQ ID NO: 29), and PkPDC6 (SEQ ID NO: 30); or, one or more nucleic acids encoding enzymes with an amino sequence with at least 50%, at least 60%, at least 70%, at least 80%, at least 95%, at least 97%, or at least 99% sequence identity to the amino acid sequence of SEQ ID NO: 9, SEQ ID NO: 29, and SEQ ID NO: 30. In specific embodiments wherein the recombinant host cell of the invention is P. kudriavzevii, the recombinant host cell comprises deletion or disruption of two nucleic acids encoding the pyruvate decarboxylase with amino acid sequence set forth in SEQ ID NO: 9, two nucleic acids encoding the pyruvate decarboxylase with amino acid sequence set forth in SEQ ID NO: 29, and two nucleic acids encoding the pyruvate decarboxylase with amino acid sequence set forth in SEQ ID NO: 30; or, six nucleic acids encoding enzymes with amino sequences with at least 50%, at least 60%, at least 70%, at least 80%, at least 95%, at least 97%, or at least 99% sequence identity to the amino acid sequences of SEQ ID NOs: 9, 29, and 30.

Similar to P. kudriavzevii, wild type S. cerevisiae has three endogenous pyruvate decarboxylases: PDC1 (SEQ ID NO: 10), PDC5, and PDC6. PDC1 is the major isoform (has the highest expression level and/or activity) in S. cerevisiae while PDC5 and PDC6 are minor isoforms. In certain embodiments wherein the recombinant host cell of the invention is S. cerevisiae, the recombinant host cell comprises a deletion or disruption of one or more nucleic acids encoding pyruvate decarboxylases with an amino acid sequence set forth in SEQ ID NO: 10, or one or more nucleic acids encoding enzymes with amino acid sequences with at least 50%, at least 60%, at least 70%, at least 80%, at least 95%, at least 97%, or at least 99% sequence identity to the amino acid sequence of SEQ ID NO: 10. For example, S. cerevisiae pyruvate decarboxylases PDC5 and PDC6 have 88% and 84% amino acid sequence identity, respectively, to the amino acid sequence set forth in SEQ ID NO: 10.

### Deletion or Disruption of Nucleic Acids Encoding Alcohol Dehydrogenase

In addition to deletion or disruption of nucleic acid encoding pyruvate decarboxylase, a yeast ethanol fermentation pathway can be deleted or disrupted by deletion or disruption of nucleic acids encoding alcohol dehydrogenase. In various embodiments, the recombinant host cells provided herein comprise a deletion or disruption of one or more nucleic acids encoding alcohol dehydrogenase. In some embodiments, one nucleic acid encoding alcohol dehydrogenase is deleted or disrupted. In other embodiments, two nucleic acids encoding alcohol dehydrogenase are deleted or disrupted. In other embodiments, more than two nucleic acids encoding alcohol dehydrogenase are deleted or disrupted. In still further embodiments, all nucleic acids encoding alcohol dehydrogenase are deleted or disrupted.

In certain embodiments, the recombinant host cell comprises a deletion or disruption of a nucleic acid encoding an alcohol dehydrogenase with an amino acid sequence set forth in SEQ ID NO: 11, or with at least 50%, at least 60%, at least 70%, at least 80%, at least 85%, at least 90%, at least 95%, at least 97%, or greater than 97% sequence identity to SEQ ID NO: 11. In specific embodiments wherein the recombinant host cell of the invention is Pichia kudriavzevii, the recombinant host cell comprises a deletion or disruption of two nucleic acids encoding alcohol dehydrogenase with an amino acid sequence set forth in SEQ ID NO: 11, or two nucleic acids encoding enzymes with amino sequences with at least 50%, at least 60%, at least 70%, at least 80%, at least 95%, at least 97%, or at least 99% sequence identity to the amino acid sequence of SEQ ID NO: 11.

### 3.1.2 Deletion or Disruption of Malate Fermentation Pathways and Nucleic Acids Encoding Malate Dehydrogenase

A malate fermentation pathway comprises one enzyme, malate dehydrogenase (EC 1.1.1.37), which catalyzes the formation of malate (the end-product of a malate fermentation pathway) from oxaloacetate along with concomitant oxidation of NADH to NAD+. Those skilled in the art will recognize that malate dehydrogenase and L-aspartate dehydrogenase use the same substrate (oxaloacetate) and will often use the same redox cofactor (NADH or NADPH) to produce their respective products. Thus, the expression of endogenous malate dehydrogenase, and particularly malate dehydrogenase located in the cytosol of yeast cells, can decrease anaerobic production of L-aspartate and/or beta-alanine. Thus, deletion or disruption of a malate fermentation pathway is useful for increasing L-aspartate and/or beta-alanine production in recombinant host cells of the invention grown under substantially anaerobic conditions. A malate fermentation pathway can be deleted or disrupted by deletion or disruption of nucleic acids encoding malate dehydrogenase.

In various embodiments, the recombinant host cells comprise a deletion or disruption of one or more nucleic acids encoding malate dehydrogenase. In some embodiments, one nucleic acid encoding malate dehydrogenase is deleted or disrupted. In other embodiments, two nucleic acids encoding malate dehydrogenase are deleted or disrupted. In other embodiments, more than two nucleic acids encoding malate dehydrogenase are deleted or disrupted. In still further embodiments, all nucleic acids encoding malate dehydrogenase are deleted or disrupted.

In various embodiments, the recombinant host cell comprises a deletion or disruption of one or more nucleic acids encoding malate dehydrogenase with an amino acid sequence set forth in SEQ ID NO: 13, or one or more nucleic acids encoding enzymes with an amino sequence with at least 50%, at least 60%, at least 70%, at least 80%, at least 95%, at least 97%, or at least 99% sequence identity to the amino acid sequence of SEQ ID NO: 13. In specific embodiments wherein the recombinant host cell of the invention is Pichia kudriavzevii, the recombinant host cell comprises a deletion or disruption of two nucleic acids encoding malate dehydrogenase with an amino acid sequence set forth in SEQ ID NO: 13, or two nucleic acids encoding enzymes with amino sequences with at least 50%, at least 60%, at least 70%, at least 80%, at least 95%, at least 97%, or at least 99% sequence identity to the amino acid sequence of SEQ ID NO: 13.

### 3.1.3 Deletion or Disruption of Glycerol Metabolic Pathways and Nucleic Acids Encoding Glycerol Metabolic Pathway Enzymes

In certain embodiments, recombinant host cells provided herein comprise a deletion or disruption of a glycerol fermentation pathway. A glycerol fermentation pathway comprises one enzyme, NAD-dependent glycerol-3-phosphate dehydrogenase (EC 1.1.1.8), which catalyzes the formation of glycerol (the end-product of a glycerol metabolic pathway) from glycerol-3-phosphate along with concomitant oxidation of NADH to NAD+. Glycerol fermentation pathway activity decreases the pool of NADH available for use by L-aspartate dehydrogenase in the production of L-aspartate from oxaloacetate in recombinant host cells of the invention grown under substantially anaerobic conditions. Thus, deletion or disruption of a glycerol fermentation pathway is useful for increasing L-aspartate and/or beta-alanine production in recombinant host cells of the invention. A glycerol metabolic pathway can be deleted or disrupted by deletion or disruption of nucleic acids encoding NAD-dependent glycerol-3-phosphate dehydrogenase.

In various embodiments, the recombinant host cells comprise a deletion or disruption of one or more nucleic acids encoding NAD-dependent glycerol-3-phosphate dehydrogenase. In some embodiments, one nucleic acid encoding NAD-dependent glycerol-3-phosphate dehydrogenase is deleted or disrupted. In other embodiments, two nucleic acids encoding NAD-dependent glycerol-3-phosphate dehydrogenase are deleted or disrupted. In other embodiments, more than two nucleic acids encoding NAD-dependent glycerol-3-phosphate dehydrogenase are deleted or disrupted. In still further embodiments, all nucleic acids encoding NAD-dependent glycerol-3-phosphate dehydrogenase are deleted or disrupted.

In various embodiments, the recombinant host cell comprises a deletion or disruption of one or more nucleic acids encoding NAD-dependent glycerol-3-phosphate dehydrogenase with amino acid sequences set forth in SEQ ID NOs: 12 and 31, or one or more nucleic acids encoding enzymes with an amino sequence with at least 50%, at least 60%, at least 70%, at least 80%, at least 95%, at least 97%, or at least 99% sequence identity to the amino acid sequences of SEQ ID NOs: 12 and 31. In some embodiments wherein the recombinant host cell of the invention is Pichia kudriavzevii, the recombinant host cell comprises a deletion or disruption of one or more nucleic acids encoding NAD-dependent glycerol-3-phosphate dehydrogenase with an amino acid sequence set forth in SEQ ID NO: 12, or one or more nucleic acids encoding enzymes with amino sequences with at least 50%, at least 60%, at least 70%, at least 80%, at least 95%, at least 97%, or at least 99% sequence identity to the amino acid sequence of SEQ ID NO: 12. In some embodiments wherein the recombinant host cell of the invention is Pichia kudriavzevii, the recombinant host cell comprises a deletion or disruption of one or more nucleic acids encoding NAD-dependent glycerol-3-phosphate dehydrogenase with an amino acid sequence set forth in SEQ ID NO: 31, or one or more nucleic acids encoding enzymes with amino sequences with at least 50%, at least 60%, at least 70%, at least 80%, at least 95%, at least 97%, or at least 99% sequence identity to the amino acid sequence of SEQ ID NO: 31.

### 3.2 Deletion or Disruption of Additional Byproduct Metabolic Pathways and Nucleic Acids Encoding Byproduct Metabolic Pathway Enzymes

Besides ethanol and malate, additional byproducts are formed by host cells of the invention, including glycerol, acetic acid, and various four-carbon dicarboxylic acids (e.g., fumarate and succinate). Additional byproducts formed by host cells of the invention can include 2-ketoacids (and amino acids other than aspartic acid derived from these 2-ketoacids) that are produced by transamination reactions with aspartic acid. Deletion or disruption of these byproduct metabolic pathways and nucleic acids encoding byproduct metabolic pathway enzymes are also useful for increasing L-aspartate and/or beta-alanine production by host cells of the invention.

### 3.2.1 Deletion or Disruption of Aspartate Aminotransferase Metabolic Pathways and Nucleic Acids Encoding Aspartate Aminotransferase Metabolic Pathway Enzymes

In certain embodiments, recombinant host cells provided herein comprise a deletion or disruption of an aspartate aminotransferase pathway. An aspartate aminotransferase pathway comprises one enzyme, aspartate aminotransferase (EC 2.6.1.1), which catalyzes the oxidation of L-aspartic acid to oxaloacetate along with concomitant reduction of L-glutamate to 2-oxoglutarate. Aspartate aminotransferase activity decreases the amount of L-aspartic acid produced and leads to formation of 2-oxoglutarate, an undesired byproduct. Thus, deletion or disruption of an aspartate aminotransferase pathway is useful for increasing L-aspartate and/or beta-alanine production in recombinant host cells of the invention. An aspartate aminotransferase metabolic pathway can be deleted or disrupted by deletion or disruption of nucleic acids encoding aspartate aminotransferase.

In various embodiments, the recombinant host cells comprise a deletion or disruption of one or more nucleic acids encoding aspartate aminotransferase. In some embodiments, one nucleic acid encoding aspartate aminotransferase is deleted or disrupted. In other embodiments, two nucleic acids encoding aspartate aminotransferase are deleted or disrupted. In other embodiments, more than two nucleic acids encoding aspartate aminotransferase are deleted or disrupted. In still further embodiments, all nucleic acids encoding aspartate aminotransferase are deleted or disrupted.

In various embodiments, the recombinant host cell comprises a deletion or disruption of one or more nucleic acids encoding an aspartate aminotransferase with an amino acid sequence set forth in SEQ ID NO: 32, or one or more nucleic acids encoding enzymes with an amino sequence with at least 50%, at least 60%, at least 70%, at least 80%, at least 95%, at least 97%, or at least 99% sequence identity to the amino acid sequence of SEQ ID NO: 32. In specific embodiments wherein the recombinant host cell of the invention is P. kudriavzevii, the recombinant host cell comprises a deletion or disruption of two nucleic acids encoding aspartate aminotransferase with an amino acid sequence set forth in SEQ ID NO: 32, or two nucleic acids encoding enzymes with amino sequences with at least 50%, at least 60%, at least 70%, at least 80%, at least 95%, at least 97%, or at least 99% sequence identity to the amino acid sequence of SEQ ID NO: 32.

### 3.2.2 Deletion or Disruption of Urea Carboxylase Metabolic Pathways and Nucleic Acids Encoding Urea Carboxylase Metabolic Pathway Enzymes

In certain embodiments, recombinant host cells provided herein comprise a deletion or disruption of a urea carboxylase pathway. A urea carboxylase pathway comprises two enzyme activities. The first enzymatic activity in the pathway is urea carboxylase (EC 6.3.4.6), which catalyzes the carboxylation of urea to urea-1-carboxylate with concomitant hydrolysis of ATP to ADP and orthophosphate. The second enzymatic activity in the pathway is allophanate hydrolyase (EC 3.5.1.54), which catalyzes the hydrolysis of one molecule urea-carboxylate to two molecules ammonium and two molecules bicarbonate. In some host cells, including P. kudriavzevii host cells, both the urea carboxylase and allophanate hydrolyase activities are performed by a single enzyme, namely urea amidolyase. In other host cells, the urea carboxylase and allophanate hydrolase activities are performed by different enzymes.

The catabolism of urea to ammonium through the urea carboxylase pathway requires expenditure of ATP, thereby increasing the ATP requirements for aspartic acid production. Specifically, one mol ATP is hydrolyzed to ADP for every two mols ammonium produced; stoichiometrically, this leads to a net loss of 0.5 mol-ATP/mol-aspartic acid. It is important to decrease the expenditure of ATP in order to increase aspartic acid yield and decrease the oxygen required for aerobic respiration as a source of ATP. Thus, deletion or disruption of a urea carboxylase pathway is useful for increasing L-aspartate and/or beta-alanine production in recombinant host cells of the invention. A urea carboxylase metabolic pathway can be deleted or disrupted by deletion or disruption of nucleic acids encoding urea carboxylase; or, in the case where a single enzyme performs both urea carboxylase pathway activities, by deletion or disruption of nucleic acids encoding urea amidolyase activity.

In various embodiments, the recombinant host cells comprise a deletion or disruption of one or more nucleic acids encoding urea carboxylase. In some embodiments, one nucleic acid encoding urea carboxylase is deleted or disrupted. In other embodiments, two nucleic acids encoding urea carboxylase are deleted or disrupted. In other embodiments, more than two nucleic acids encoding urea carboxylase are deleted or disrupted. In still further embodiments, all nucleic acids encoding urea carboxylase are deleted or disrupted.

In various embodiments, the recombinant host cells comprise a deletion or disruption of one or more nucleic acids encoding urea amidolyase. In some embodiments, one nucleic acid encoding urea amidolyase is deleted or disrupted. In other embodiments, two nucleic acids encoding urea amidolyase are deleted or disrupted. In other embodiments, more than two nucleic acids encoding urea amidolyase are deleted or disrupted. In still further embodiments, all nucleic acids encoding urea amidolyase are deleted or disrupted.

In various embodiments, the recombinant host cell comprises a deletion or disruption of one or more nucleic acids encoding a urea amidolyase with an amino acid sequence set forth in SEQ ID NO: 33, or one or more nucleic acids encoding enzymes with an amino sequence with at least 50%, at least 60%, at least 70%, at least 80%, at least 95%, at least 97%, or at least 99% sequence identity to the amino acid sequence of SEQ ID NO: 33. In specific embodiments wherein the recombinant host cell of the invention is P. kudriavzevii, the recombinant host cell comprises a deletion or disruption of two nucleic acids encoding urea amidolyase with an amino acid sequence set forth in SEQ ID NO: 33, or two nucleic acids encoding enzymes with amino sequences with at least 50%, at least 60%, at least 70%, at least 80%, at least 95%, at least 97%, or at least 99% sequence identity to the amino acid sequence of SEQ ID NO: 33.

### Section 4. Genetic Modifications to Increase L-Aspartic Acid Production

In another aspect, the invention provides host cells genetically modified to express heterologous nucleic acids encoding enzymes or proteins enabling energy efficient L-aspartic acid production. “Energy efficient”, as defined herein, refers to production of L-aspartic acid with a lower ATP requirement as compared to a parental, or control strain. Decreasing the expenditure of ATP is an important aspect of L-aspartate production under aerobic or substantially anaerobic conditions. If host cell ATP requirements become sufficiently high, additional oxygen must be provided to the culture to support L-aspartate production. Two processes useful for increasing the energy efficiency of L-aspartate production in genetically modified host cells of the invention are the urease pathway and L-aspartate export.

### 4.1 Urease Pathway

Urea is the preferred source of nitrogen as compared to ammonia for at least three reasons. First, urea is non-toxic and can be added at high concentrations to the fermentation broth; by comparison, ammonia, another commonly used nitrogen source in industry, is basic and high concentrations are toxic to many host cells. Second, urea is neutrally charged, can diffuse across the host cell plasma membrane (i.e., no energy is expended for transport), and the fermentation pH is unaffected by its addition to the fermentation broth; by comparison, ammonia is charged and must be transported into the cell enzymatically. Third, the breakdown of urea also releases ammonia and CO₂, both being co-substrates for enzymes in L-aspartate biosynthetic pathways; by comparison, no CO₂ is released during catabolism of ammonia. Therefore, in some embodiments, the recombinant host cells provided herein comprise at least one urease pathway comprising all the enzymes and proteins necessary for ATP-independent breakdown of urea to ammonia and carbon dioxide, and for growth of the engineered host cell on urea as the sole nitrogen source. Many host cells, including P. kudriavzevii host cells, do no naturally contain an active urease pathway. Therefore, a recombinant host cell having an active urease pathway may comprise one or more heterologous nucleic acids encoding one or more urease pathway enzymes or proteins. Non-limiting examples of urease pathway enzymes or proteins are urease enzymes, nickel transporters, and urease accessory proteins.

Urease enzymes (EC 3.5.1.5) catalyze the hydrolysis of one molecule urea to one molecule carbamate and one molecule ammonia; the one molecule carbamate then degrades into one molecule ammonia and one molecule carbonic acid. Thus, in sum, urease activity results in production of two molecules ammonia and one molecule carbon dioxide per molecule urea in each catalytic cycle. Importantly, urease performs this reaction without expenditure of ATP. In contrast to urease enzymes, alternative metabolic pathways capable of catalyzing conversion of urea to ammonia and carbon dioxide do require expenditure of ATP. For example, many host cells, including many yeast host cells, use a urea catabolic pathway comprising the enzymes urea carboxylase and allophanate hydrolase; using this pathway, one molecule ATP is expended per molecule urea catabolized.

Therefore, having a urease pathway is useful for increasing L-aspartate and/or beta-alanine production in recombinant host cells of the invention. In some embodiments, the recombinant host cells provided herein comprise a urease enzyme. In some embodiments, the urease is endogenous to the recombinant host cells. In other embodiments, the urease is heterologous to the recombinant host cells.

Urease enzymes require the presence of a nickel cofactor inside the host cell (i.e., in the cytosol) for activity. Nickel transporters can transport extracellular nickel ions across the cell membrane and into the cytosol. Therefore, in some embodiments, the recombinant host cells provided herein comprise a nickel transporter. In some embodiments, the nickel transporter is endogenous to the recombinant host cells. In other embodiments, the nickel transporter is heterologous to the recombinant host cells.

Urease enzymes require additional proteins (i.e., urease accessory proteins) for activity. Urease accessory proteins are believed to assemble the apoenzyme and load nickel cofactor into the urease enzyme active site (although the invention is not restricted by any specific mechanism of action). Therefore, in some embodiments, the recombinant host cells provided herein comprise one or more urease accessory proteins. In some embodiments, the recombinant host cells comprise one or more urease accessory proteins that are endogenous to the recombinant host cells. In other embodiments, the recombinant host cells comprise one or more urease accessory proteins that are heterologous to the recombinant host cells. In some embodiments, the recombinant host cells comprise one urease accessory protein. In other embodiments, the recombinant host cells comprise 2 urease accessory proteins. In yet other embodiments, the recombinant host cells comprise 3 ore more urease accessory proteins. In some embodiments, the recombinant host cells comprise 1 heterologous urease accessory protein. In other embodiments, the recombinant host cells comprise 2 heterologous urease accessory proteins. In yet other embodiments, the recombinant host cells comprise 3 or more heterologous urease accessory proteins.

In many embodiments, the recombinant host cells provided herein comprise one or more heterologous nucleic acids encoding a urease pathway enzyme or protein wherein the nucleic acid is expressed in sufficient amount to allow the host cell to grow on urea as the sole nitrogen source. In certain embodiments, the recombinant host cells comprise a single nucleic acid encoding a urease pathway enzyme or protein. In other embodiments, the recombinant host cells comprise multiple heterologous nucleic acids encoding urease pathway enzymes and/or proteins. In these embodiments, the recombinant host cells may comprise multiple copies of a single heterologous nucleic acid and/or multiple copies of two or more heterologous nucleic acids.

### Urease Enzymes

In some embodiments, the recombinant host cells of the invention comprise one or more heterologous nucleic acids encoding at least one urease enzyme (EC 3.5.1.5).

In some embodiments, the recombinant host cells provided herein comprise one or more heterologous nucleic acids encoding a urease enzyme derived from a fungal source. Non-limiting examples of urease enzymes derived from fungal sources include those selected from the group consisting of S. pombe urease (SpURE2; UniProt ID: O00084; SEQ ID NO: 34), Schizosaccharomyces cryophilus urease (UniProt ID: S9W2F7), Aspergillus oryzae urease (UniProt ID: Q2UKB4), and Neurospora crassa urease (UniProt ID: Q6MUT4).

In various embodiments, the recombinant host cells of the invention comprise one or more heterologous nucleic acids encoding SpURE2 urease (SEQ ID NO: 34), or one or more heterologous nucleic acids encoding urease enzymes with amino acid sequences with at least 50%, at least 60%, at least 70%, at least 80%, at least 90%, at least 95%, at least 97%, or at least 99% sequence identity to SpURE2 urease (SEQ ID NO: 34).

In some embodiments, the recombinant host cells further comprise a deletion or disruption of one or more nucleic acids encoding urea amidolyase.

In some embodiments in which the recombinant host cells comprise one or more heterologous nucleic acids encoding at least one urease enzyme, the recombinant host cells are capable of growing on urea as the sole nitrogen source and are capable of producing L-aspartate and/or beta-alanine. In some such embodiments, the recombinant host cells are capable of growing on urea as the sole nitrogen source and are capable of producing L-aspartate and/or beta-alanine under substantially anaerobic conditions.

In specific embodiments, the recombinant host cells of the invention comprise a heterologous nucleic acid encoding SpURE2 (SEQ ID NO: 34), and a deletion or disruption of a nucleic acid encoding urea amidolyase and/or a heterologous nucleic acid encoding an L-aspartate dehydrogenase. In some such embodiments, the recombinant host cells are capable of growing on urea as the sole nitrogen source and are capable of producing L-aspartate and/or beta-alanine under substantially anaerobic conditions. In many of these embodiments, the recombinant host cells are P. kudriavzevii host cells.

### Urease Accessory Proteins

In some embodiments, the recombinant host cells of the invention comprise one or more heterologous nucleic acids encoding at least one urease accessory protein.

In some embodiments, the recombinant host cells provided herein comprise one or more heterologous nucleic acids encoding at least one urease accessory protein derived from a fungal source. Non-limiting examples of urease accessory proteins derived from fungal sources include those selected from the group consisting of S. pombe urease accessory proteins URED (SpURED; UniProt ID: P87125; SEQ ID NO: 35), UREF (SpUREF; UniProt ID: O14016. SEQ ID NO: 36), and UREG (SpUREG; UniProt ID: Q96WV0, SEQ ID NO: 37).

In various embodiments, the recombinant host cells of the invention comprise one or more heterologous nucleic acids encoding urease accessory protein SpURED (SEQ ID NO: 35), or one or more heterologous nucleic acids encoding urease accessory proteins with amino acid sequences with at least 50%, at least 60%, at least 70%, at least 80%, at least 90%, at least 95%, at least 97%, or at least 99% sequence identity to SpUREF (SEQ ID NO: 35). In various embodiments, the recombinant host cells of the invention comprise one or more heterologous nucleic acids encoding urease accessory protein SpUREF (SEQ II) NO: 36), or one or more heterologous nucleic acids encoding urease accessory proteins with amino acid sequences with at least 50%, at least 60%, at least 70%, at least 80%, at least 90%, at least 95%, at least 97%, or at least 99% sequence identity to SpUREF (SEQ ID NO: 36). In various embodiments, the recombinant host cells of the invention comprise one or more heterologous nucleic acids encoding urease accessory protein SpUREG (SEQ ID NO: 37), or one or more heterologous nucleic acids encoding urease accessory proteins with amino acid sequences with at least 50%, at least 60%, at least 70%, at least 80%, at least 90%, at least 95%, at least 97%, or at least 99% sequence identity to SpUREG (SEQ ID NO: 37).

In some embodiments, the recombinant host cells further comprise a heterologous nucleic acid encoding a urease enzyme. In some embodiments, the recombinant host cells further comprise a deletion or disruption of one or more nucleic acids encoding urea amidolyase.

In some embodiments in which the recombinant host cells comprise one or more heterologous nucleic acids encoding at least one urease accessory protein, the recombinant host cells are capable of growing on urea as the sole nitrogen source. In some such embodiments, the recombinant host cells are further capable of producing L-aspartate and/or beta-alanine. In some such embodiments, the recombinant host cells are capable of growing on urea as the sole nitrogen source and are capable of producing L-aspartate and/or beta-alanine under substantially anaerobic conditions.

In specific embodiments, the recombinant host cells of the invention comprise a heterologous nucleic acid encoding SpURE2 (SEQ ID NO: 34) and a heterologous nucleic acid encoding SpURED (SEQ ID NO: 35) and/or a heterologous nucleic acid encoding SpUREF (SEQ ID NO: 36) and/or a heterologous nucleic acid encoding SpUREG (SEQ ID NO: 37). In some such embodiments, the recombinant host cells further comprise a deletion or disruption of a nucleic acid encoding urea amidolyase and/or a heterologous nucleic acid encoding an L-aspartate dehydrogenase. In some such embodiments, the recombinant host cells are capable of growing on urea as the sole nitrogen source and are capable of producing L-aspartate and/or beta-alanine under substantially anaerobic conditions. In many embodiments, the recombinant host cells are P. kudriavzevii host cells.

### Nickel Transport Protein

In some embodiments, the recombinant host cells of the invention comprise one or more heterologous nucleic acids encoding a nickel transporter.

In some embodiments, the recombinant host cells provided herein comprise one or more heterologous nucleic acids encoding a nickel transporter derived from a fungal source. Non-limiting examples of a nickel transporter derived from fungal sources include those selected from the group consisting of S. pombe NIC1 (SpNIC1; UniProt ID: O74869, SEQ ID NO: 38).

In various embodiments, the recombinant host cells of the invention comprise one or more heterologous nucleic acids encoding nickel transporter SpNIC1 (SEQ ID NO: 38), or one or more heterologous nucleic acids encoding a nickel transporter with an amino acid sequence with at least 50%, at least 60%, at least 70%, at least 80%, at least 90%, at least 95%, at least 97%, or at least 99% sequence identity to SpNIC1 (SEQ ID NO: 38).

In some embodiments, the recombinant host cells further comprise a heterologous nucleic acid encoding a urease enzyme. In some embodiments, the recombinant host cells further comprise a deletion or disruption of one or more nucleic acids encoding urea amidolyase. In some embodiments, the recombinant host cells further comprise one or more heterologous nucleic acids encoding at least one urease accessory protein.

In some embodiments in which the recombinant host cells comprise a heterologous nucleic acid encoding a nickel transporter, the recombinant host cells are capable of growing on urea as the sole nitrogen source. In some such embodiments, the recombinant host cells are further capable of producing L-aspartate and/or beta-alanine in some such embodiments, the recombinant host cells are capable of growing on urea as the sole nitrogen source and are capable of producing L-aspartate and/or beta-alanine under substantially anaerobic conditions.

In specific embodiments, the recombinant host cells of the invention comprise a heterologous nucleic acid encoding SpURE2 (SEQ II) NO: 34) and a heterologous nucleic acid encoding SpURED (SEQ ID NO: 35) and/or a heterologous nucleic acid encoding SpUREF (SEQ ID NO: 36) and/or a heterologous nucleic acid encoding SpUREG (SEQ ID NO: 37) and a heterologous nucleic acid encoding SpNIC1 (SEQ ID NO: 38). In some such embodiments, the recombinant host cells further comprise a deletion or disruption of a nucleic acid encoding urea amidolyase and/or a heterologous nucleic acid encoding an L-aspartate dehydrogenase. In some such embodiments, the recombinant host cells are capable of growing on urea as the sole nitrogen source and are capable of producing L-aspartate and/or beta-alanine under substantially anaerobic conditions. In many embodiments, the recombinant host cells are P. kudriavzevii host cells.

### 4.2 Aspartate Export

Low-cost L-aspartate production benefits from export of L-aspartate from the cytosol, across the host cell membrane, and into the surrounding culture medium. Likewise, it is desirable to export L-aspartate without ATP expenditure, thereby enabling more energy efficient L-aspartate production.

One L-aspartate transport protein suitable for L-aspartate export in engineered host cells of the invention is Arabidopsis thaliana SIAR1 (AtSIAR1; SEQ ID NO: 39) and its homologs. Another suitable L-aspartate transport protein is Arabidopsis thaliana bidirectional L-aspartate transport protein BAT1 (AtBAT1; SEQ ID NO: 40).

In many embodiments, a recombinant host cell capable of producing aspartic acid additionally comprises one or more nucleic acids encoding an aspartate permease and the host cell produces an increased amount of aspartic acid relative to the parental host cell that does not comprise the one or more nucleic acids encoding an aspartate permease. In some embodiments, the aspartate permease is AtSIAR1 (SEQ ID NO: 39). In other embodiments, the aspartate permease is AtBAT1 (SEQ ID NO: 40).

In addition to or instead of the Arabidopsis thaliana SIAR1 and BAT1 proteins provided herein, enzymes homologous to these proteins can be used. Any enzyme homologous to a Arabidopsis thaliana SIAR1 and BAT1 aspartate permease described herein is suitable for use in accordance with the methods of the invention so long as the engineered host cell is capable of exporting aspartic acid out of the host cell and into the fermentation broth.

### Section 5. Methods of Producing L-Aspartate or Beta-Alanine

In another aspect, methods are provided herein for producing L-aspartate or beta-alanine by recombinant host cells of the invention. In certain embodiments, these methods comprise the steps of: (a) culturing a recombinant host cell described herein in a medium containing at least one carbon source and one nitrogen source under substantially anaerobic conditions such that L-aspartate is produced; and (b) recovering said L-aspartate from the medium. In other embodiments, these methods comprise the steps of: (a) culturing a recombinant host cell described herein in a medium containing at least one carbon source and one nitrogen source under aerobic conditions such that L-aspartate is produced; and (b) recovering said L-aspartate from the medium. In other embodiments, these methods comprise the steps of: (a) culturing a recombinant host cell described herein in a medium containing at least one carbon source and one nitrogen source under substantially anaerobic conditions such that beta-alanine is produced; and (b) recovering said beta-alanine from the medium. The L-aspartate or beta-alanine can be secreted into the culture medium.

It is understood that, in the methods of the invention, any of the one or more heterologous nucleic acids provided herein can be introduced into a host cell to produce a recombinant host cell of the invention. For example, a heterologous nucleic acid can be introduced so as to confer a L-aspartate fermentation pathway onto the recombinant host cell. The recombinant host cell may further comprise heterologous nucleic acids encoding L-aspartate 1-decarboxylase so as to confer the ability for the recombinant host cell to produce beta-alanine. Alternatively, heterologous nucleic acids can be introduced to produce an intermediate host cell having the biosynthetic capability to catalyze some of the required metabolic reactions to confer L-aspartate or beta-alanine biosynthetic capability.

In some embodiments, the methods comprise the step of constructing nucleic acids for introduction into host cells. Methods for construction nucleic acids are well-known in the art (see, for example, Sambrook et al., Molecular Cloning: A Laboratory Manual, 2d ed., Cold Spring Harbor Laboratory Press, Cold Spring Harbor, N.Y., 1989; Ausubel et al., Current Protocols in Molecular Biology, Greene Publishing Associates, 1992, and Supplements to 2002).

In some embodiments, the methods comprise the step of transforming host cells with nucleic acids to obtain the recombinant host cells provided herein. Methods for transforming cells with nucleic acids are well-known in the art. Non-limiting examples of such methods include calcium phosphate transfection, dendrimer transfection, liposome transfection (e.g., cationic liposome transfection), cationic polymer transfection, electroporation, cell squeezing, sonoporation, optical transfection, protoplast fusion, impalefection, hyrodynamic delivery, gene gun, magnetofection, and viral transduction. One skilled in the art is able to select one or more suitable methods for transforming cells with vectors provided herein based on the knowledge in the art that certain techniques for introducing vectors work better for certain types of cells.

Any of the recombinant host cells described herein can be cultured to produce and/or secrete L-aspartate or beta-alanine. For example, recombinant host cells producing L-aspartate can be cultured for the biosynthetic production of L-aspartate. The L-aspartate can be isolated or treated as described below to produce beta-alanine or L-aspartate. Similarly, recombinant host cells producing beta-alanine can be cultured for the biosynthetic production of beta-alanine. The beta-alanine can be isolated and subjected to further treatments for the chemical synthesis of beta-alanine family of compounds, including, but not limited to, pantothenic acid, beta-alanine alkyl esters (e.g., beta-alanine methyl ester, beta-alanine ethyl ester, beta-alanine propyl ester, and the like), and poly(beta-alanine).

The methods of producing L-aspartate or beta-alanine provided herein may be performed in a suitable fermentation broth in a suitable fermentation vessel, including but not limited to a culture plate, a flask, or a fermentor. Further, the methods of the invention can be performed at any scale of fermentation known in the art to support industrial production of microbially produced small-molecules. Any suitable fermentor may be used including a stirred tank fermentor, an airlift fermentor, a bubble column fermentor, a fixed bed bioreactor, or any combination thereof.

In some embodiments, the fermentation broth is any fermentation broth in which a recombinant host cell capable of producing L-aspartate and/or beta-alanine can subsist (maintain growth and/or viability). In some embodiments, the fermentation broth is an aqueous medium comprising assimilable carbon, nitrogen, and phosphate sources. Such a medium can also include appropriate salts, minerals, metals, and other nutrients. In some embodiments, the carbon source and each of the essential cell nutrients are provided to the fermentation broth incrementally or continuously, and each essential cell nutrient is maintained at essentially the minimum level required for efficient assimilation by growing cells.

In some embodiments, culturing of the cells provided herein to produce L-aspartate and/or beta-alanine may be divided up into phases. For example, the cell culture process may be divided up into a growth phase, a production phase, and/or a recovery phase. The following paragraphs provide examples of specific conditions that may be used for these phases. One skilled in the art will recognize that these conditions may be varied based on the host cell used, the desired L-aspartate or beta-alanine yield, titer, and/or productivity, or other factors.

Carbon Source.

The carbon source provided to the fermentation can be any carbon source that can be fermented by the host cell. Suitable carbon sources include, but are not limited to, monosaccharides, disaccharides, polysaccharides, acetate, ethanol, methanol, methane, or one or more combinations thereof. Exemplary monosaccharides suitable for use in accordance to the methods of the invention include, but are not limited to, dextrose (glucose), fructose, galactose, xylose, arabinose, and combinations thereof. Exemplary disaccharides suitable for use in accordance to the methods of the invention include, but are not limited to, sucrose, lactose, maltose, trehalose, cellobiose, and combinations thereof. Exemplary polysaccharides suitable for use in accordance to the methods of the invention include, but are not limited to, starch, glycogen, cellulose, and combinations thereof. In some embodiments, the carbon source is dextrose. In other embodiments, the carbon source is sucrose.

Nitrogen.

Every molecule of L-aspartate or beta-alanine comprises nitrogen atom, and in order to produce L-aspartate and/or beta-alanine at a high yield, a suitable source of assimilable nitrogen must be provided to the fermentation during host cell cultivation. As used herein, assimilable nitrogen refers to nitrogen that is capable of being metabolized by the host cell of the invention and used in producing L-aspartate. The nitrogen source may be any assimilable nitrogen source that can be utilized by the host cell, including, but not limited to, anhydrous ammonia, ammonium sulfate, ammonium nitrate, diammonium phosphate, monoammonium phosphate, ammonium polyphosphate, sodium nitrate, urea, peptone, protein hydrolysates, and yeast extract. In one embodiment, the nitrogen source is anhydrous ammonia. In another embodiment, the nitrogen source is ammonium sulfate. In yet a further embodiment, the nitrogen source is urea. Those skilled in the art will recognize that the mols assimilable nitrogen is dependent on the nitrogen source, and, for example, one mol of anhydrous ammonia (NH₃) comprises 1 mol assimilable nitrogen while one mol of diammonium phosphate (NH₄)₂PO₄ comprises 2 mols assimilable nitrogen. A minimum amount of assimilable nitrogen must be provided to the fermentation during host cell cultivation to achieve high L-aspartate and/or beta-alanine yields. In certain embodiments of the methods provided herein wherein the carbon source is dextrose, the molar ratio of assimilable nitrogen to dextrose provided to the fermentation during host cell cultivation is at least 0.25:1, at least 0.5:1, at least 0.75:1, 1:1, at least 1.25:1, at least 1.5:1, at least 1.75:1, at least 2:1, or greater than 2:1. In certain embodiments of the methods provided herein the carbon source is sucrose, and the molar ratio of assimilable nitrogen to sucrose is at least 0.1:1, at least 0.2:1, at least 0.3:1, at least 0.4:1, at least 0.5:1, at least 0.6:1, at least 0.7:1, at least 0.8:1, at least 0.9:1, at least 1:1, or greater than 1:1.

pH.

The pH of the fermentation broth can be controlled by the addition of acid or base to the culture medium. Preferably, the pH is maintained from about 3.0 to about 8.0. Non-limiting examples of suitable acids include aspartic acid, acetic acid, hydrochloric acid, and sulfuric acid. Non-limiting examples of suitable bases include sodium hydroxide, potassium hydroxide, calcium hydroxide, calcium carbonate, ammonia, and diammonium phosphate. In some embodiments, a strong acid or strong base is used to limit dilution of the fermentation broth. Aspartic acid exhibits a relatively low solubility in water and will crystallize from solution (only about 6 g/L aspartic acid is soluble at 30° C.). Crystallization occurs when the concentration of the fully protonated, aspartic acid form of L-aspartate increases to above the solubility limit. It is advantageous to crystallize aspartic acid during the fermentation for several reasons. First, crystallization provides an aspartic acid sink, enabling a high concentration gradient to be maintained across the cell membrane and helping to increase the kinetics of product export outside the host cell. Second, the L-aspartic acid that has crystallized from solution in the fermentation can be more readily separated from the majority of the cells and fermentation broth, accomplishing a purification step. To facilitate efficient purification, in many cases, it is desirable for the majority of the L-aspartate to be in the insoluble, crystallized form (i.e. crystallized aspartic acid) prior to purification. Preferably, greater than about 50 g/L aspartic acid is in an insoluble, crystallized form prior to purification of the aspartic acid from the fermentation broth. More preferably, greater than about 75 g/L of aspartic acid produced is in an insoluble, crystallized form prior to purification of the aspartic acid from the fermentation broth. Aspartic acid can be crystallized from the fermentation broth by any method known in the art of obtaining crystallized compounds, including, for example, evaporation, decreasing temperature, or any other method that causes the concentration of the fully protonated aspartic acid form of L-aspartate in the fermentation broth to exceed its solubility limit. In some embodiments, aspartic acid is crystallized from the fermentation broth by decreasing the pH of the fermentation broth to below pH 3.86, the pKa of aspartic acid R-chain. In other embodiments, aspartic acid is crystallized from the fermentation broth by decreasing the pH of the fermentation broth to below the isoelectric point of aspartic acid (at a pH of about 2.5 to 3.5). The broth pH can be decreased during the fermentation (i.e., while the host cells are producing aspartic acid), and/or at the conclusion of the fermentation. The broth pH can be decreased due to endogenous production of aspartic acid, and/or due to supplementation of an acid to the fermentation. In some embodiments, at the end of the fermenting the fermentation broth comprises at least 50% by weight of crystallized aspartic acid. In some embodiments, at the end of the fermenting the fermentation broth comprises at least 80% by weight of crystallized aspartic acid.

Temperature.

The temperature of the fermentation broth can be any temperature suitable for growth of the recombinant host cells and/or production of L-aspartate or beta-alanine. Preferably, during production of L-aspartate or beta-alanine the fermentation broth is maintained at a temperature in the range of from about 20° C. to about 45° C., preferably in the range of from about 25° C. to about 37° C., and more preferably in the range from about 28° C. to about 32° C. The temperature of the fermentation broth can be decreased at the conclusion of the fermentation to aid crystallization of aspartic acid by decreasing solubility of aspartic acid in the fermentation broth. Alternatively, the temperature of the fermentation broth can be increased at the conclusion of the fermentation to aid crystallization of aspartic acid by evaporating solute and concentrating aspartic acid in the fermentation broth.

Oxygen.

During cultivation, aeration and agitation conditions are selected to produce a desired oxygen uptake rate. In various embodiments, conditions are selected to produce an oxygen uptake rate of around 0-25 mmol/l/hr. In some embodiments conditions are selected to produce an oxygen uptake rate of around 2.5-15 mmol/l/hr. Oxygen uptake rate as used herein refers to the volumetric rate at which oxygen is consumed during the fermentation. Inlet and outlet oxygen concentrations can be measured with exhaust gas analysis, for example by mass spectrometers. Oxygen uptake rate can be calculated by one of ordinary skill in the art using the Direct Method described in Bioreaction Engineering Principles 3^{rd} Edition, 2011, Spring Science+Business Media, p. 449. Although the L-aspartate pathways described herein are preferably used to produce L-aspartate and/or beta-alanine under substantially anaerobic conditions, they are capable of producing L-aspartate and/or beta-alanine under a range of oxygen concentrations. In some embodiments, the L-aspartate pathways produce L-aspartate and/or beta-alanine under aerobic conditions. In preferred embodiments, the L-aspartate pathways produce L-aspartate and/or beta-alanine under substantially anaerobic conditions.

A high yield of either L-aspartate or beta-alanine from the provided carbon and nitrogen source(s) is desirable to decrease the production cost. As used herein, yield is calculated as the percentage of the mass of carbon source catabolized by host cells of the invention and used to produce either L-aspartate or beta-alanine. In some cases, only a fraction of the carbon source provided to a fermentation is catabolized by host the cells, and the remainder is found unconsumed in the fermentation broth or is consumed by contaminating microbes in the fermentation. Thus, it is important to ensure that fermentation is both substantially pure of contaminating microbes and that the concentration of unconsumed carbon source at the completion of the fermentation is measured. For example, if 100 grams of glucose are provided to host cells, and at the end of the fermentation 25 grams of beta-alanine are produced and there remains 10 grams of glucose, the beta-alanine yield is 27.7% (i.e., 10 grams beta-alanine from 90 grams glucose). In certain embodiments of the methods provided herein, the final yield of L-aspartate on the carbon source is at least 10%, at least 20%, at least 30%, at least 40%, at least 50%, or greater than 50%. In certain embodiments, the host cells provided herein are capable of producing at least 80%, at least 85%, or at least 90% by weight of carbon source to L-aspartate. In certain embodiments of the methods provided herein, the final yield of beta-alanine on the carbon source is at least 10%, at least 20%, at least 30%, at least 40%, at least 50%, or greater than 50%. In certain embodiments, the host cells provided herein are capable of producing at least 80%, at least 85%, or at least 90% by weight of carbon source to beta-alanine.

In addition to yield, the titer, or concentration, of L-aspartate and/or beta-alanine produced in the fermentation is another important metric for decreasing production, and, assuming all other metrics are equal, a higher titer is preferred as compared to a lower titer. Generally speaking, titer is provided as grams product (e.g., L-aspartate or beta-alanine) produced per liter of fermentation broth (i.e., g/l). In some embodiments, the L-aspartate titer is at least 1 g/l, at least 5 g/l, at least 10 g/l, at least 15 g/l, at least 20 g/l, at least 25 g/l, at least 30 g/l, at least 40 g/l, at least 50 g/l, at least 60 g/l, at least 70 g/l, at least 80 g/l, at least 90 g/l, at least 100 g/l, or greater than 100 g/l at some point during the fermentation, and preferably at the conclusion of the fermentation. In other embodiments, the beta-alanine titer is at least 1 g/l, at least 5 g/l, at least 10 g/l, at least 15 g/l, at least 20 g/l, at least 25 g/l, at least 30 g/l, at least 40 g/l, at least 50 g/l, at least 60 g/l, at least 70 g/l, at least 80 g/l, at least 90 g/l, at least 100 g/l, or greater than 100 g/l at some point during the fermentation, and preferably at the conclusion of the fermentation.

Further, productivity, or the rate of product (i.e., L-aspartate or beta-alanine) formation, is important for decreasing production cost, and, assuming all other metrics are equal, a higher productivity is preferred over a lower productivity. Generally speaking, productivity is provided as grams product produced per liter of fermentation broth per hour (i.e., g/l/hr). In some embodiments, the L-aspartate productivity is at least 0.1 g/l, at least 0.25 g/l, at least 0.5 g/l, at least 0.75 g/l, at least 1.0 g/l, at least 1.25 g/l, at least 1.25 g/1, at least 1.5 g/l, or greater than 1.5 g/l over some time period during the fermentation. In other embodiments, the beta-alanine productivity is at least 0.1 g/l, at least 0.25 g/l, at least 0.5 g/l, at least 0.75 g/l, at least 1.0 g/l, at least 1.25 g/l, at least 1.25 g/1, at least 1.5 g/l, or greater than 1.5 g/l over some time period during the fermentation.

Decreasing byproduct formation is also important for decreasing production cost, and, generally speaking, the lower the byproduct concentration the lower the production cost. Byproducts that can occur during production of L-aspartate or beta-alanine producing host cells in accordance with the methods of the invention include ethanol, acetate, and pyruvate. In certain embodiments of the methods provided herein, the recombinant host cells produce ethanol at a low yield from the provided carbon source. In certain embodiments, ethanol may be produced at a yield of 10% or less, and preferably at a yield of 5% or less at the conclusion of the fermentation. In certain embodiments of the methods provided herein, the recombinant host cells produce acetate at a low yield from the provided carbon source. In certain embodiments, acetate may be produced at a yield of 10% or less, and preferably at a yield of 5% or less at the conclusion of the fermentation. In certain embodiments of the methods provided herein, the recombinant host cells produce pyruvate at a low yield from the provided carbon source. In certain embodiments, pyruvate may be produced at a yield of 10% or less, and preferably at a yield of 5% or less at the conclusion of the fermentation.

Fermentation procedures are particularly useful for the biosynthetic production of commercial quantities of L-aspartate and/or beta-alanine. Fermentation procedures can be scaled up for manufacturing of L-aspartate or beta-alanine. Exemplary fermentation procedures include, for example, fed-batch fermentation and batch product separation; fed-batch fermentation and continuous product separation; batch fermentation and batch product separation; and continuous fermentation and continuous product separation. All of these processes are well known in the art.

In addition to the biosynthesis of L-aspartate and beta-alanine as described herein, the recombinant host cells and methods of the invention can also be utilized in various combinations with each other and with other microbes and methods known in the art to achieve product biosynthesis by other routes. For example, one alternative to product beta-alanine other than the use of L-aspartate producing host cell of the invention and chemical conversion or other than the use of a beta-alanine producing host cell of the invention is through addition of a second microbe capable of converting L-aspartate to beta-alanine.

One such procedure includes, for example, the cultivation of a L-aspartate producing host cell of the invention to produce L-aspartate as described herein. The L-aspartate can then be used as a substrate for a second microbe that converts L-aspartate to beta-alanine. The L-aspartate can be added directly to another culture of the second microbe, or the L-aspartate producing microbes in the original culture can be removed by, for example, cell separation and the second microbe capable of producing beta-alanine from L-aspartate added to the culture in a sufficient amount to enable production of beta-alanine from the L-aspartate in the fermentation broth.

### Section 6. Methods of Purifying L-Aspartate

The methods provided herein comprise the step of purifying the L-aspartate produced by the recombinant host cells. Purification is greatly facilitated by crystallizing the fully protonated form of L-aspartate, aspartic acid, as described herein.

Crystallized aspartic acid can be isolated from the fermentation broth by any technique apparent to those of skill in the art. In some embodiments, crystallized aspartic acid is isolated based on size, weight, density, or combinations thereof. Isolating based on size can be accomplished, for example, via filtration, using, for example, a filter press, candlestick filter, or other industrially used filtration system with appropriate molecular weight cutoff. Isolating based on weight or density can be accomplished, for example, via gravitational settling or centrifugation, using, for example, a settler, low g-force decanter centrifuge, or hydrocyclone, wherein suitable g-forces and settling or centrifugation times can be determined using methods known in the art. In some embodiments, crystallized aspartic acid is isolated from the fermentation broth via settling for from 30 minutes to 2 hours at a g-force of 1. In other embodiments, crystallized aspartic acid is isolated from the fermentation broth via centrifugation for 20 seconds at a g-force of from 275 g to 325 g.

In some embodiments, cell or cell debris is removed from the fermentation broth prior to isolating crystallized aspartic acid from the fermentation broth. In some embodiments, cell or cell debris is removed from crystallized aspartic acid after isolating the crystallized aspartic acid from the fermentation broth. Such removing of cell and cell debris can be accomplished, for example, via filtration or centrifugation using molecular weight cutoffs, g-forces, and/or centrifugation or settling times that are suitable for separating cell and cell debris while leaving behind crystallized aspartic acid. In some embodiments, removal of biomass is repeated at least once at one or multiple steps in the methods provided herein.

Following isolation from the fermentation broth, the crystallized aspartic acid is wet with residual fermentation broth that coats the outside of the aspartic acid crystals. The residual fermentation broth contains impurities (for example, but not limited to, salts, proteins, cell and cell debris, and organic small-molecules) that adversely affect downstream aspartic acid purification. Thus, it is useful to wash the isolated aspartic acid crystals with water to remove these trace impurities. When washing the crystals it is important to minimize the dissolution of the isolated aspartic acid into the wash water; for this reason, cold wash (around 4° C.) water is generally used. Additionally, it is important to minimize the amount of wash water used to minimize the amount of aspartic acid that is lost to dissolution in the wash water. In many embodiments, less than 10% w/w wash water is used to wash the aspartic acid crystals separated from the fermentation broth.

In some embodiments, the methods further comprise the step of removing impurities from the isolated crystallized aspartic acid. Impurities may react with aspartic acid and reduce final yields, or contribute to the aspartic acid being of lower purity and having more limited industrial utility. Non-limiting examples of impurities include acetic acid, succinic acid, malic acid, ethanol, glycerol, citric acid, and propionic acid. In some embodiments, such removing of impurities is accomplished by re-suspending the isolated crystallized aspartic acid in aqueous solution, then re-crystallizing the aspartic acid (e.g., by acidifying or evaporating the aqueous solution and/or decreasing temperature), and finally re-isolating the crystallized aspartic acid by filtration or centrifugation.

### EXAMPLES

### Media Used in the Examples

Synthetic defined (SD) medium. SD medium comprises 2% (w/v) glucose, 6.7 g/l yeast nitrogen base (YNB) without amino acids, 20 mg/l histidine hydrochloride monohydrate, 100 mg/l leucine, 50 mg/l lysine hydrochloride, 50 mg/l arginine, 50 mg/l tryptophan, 100 mg/l threonine, 20 mg/l methionine, 50 mg/l phenylalanine, 80 mg/l aspartic acid, 50 mg/l isoleucine, 50 mg/l tyrosine, 140 mg/l valine, 10 mg/l adenine and 20 mg/l uracil. The YNB used in the SD medium comprised ammonium sulfate (5 g/l), Biotin (2 μg/l), calcium pantothenate (400 μg/l), folic acid (2 μg/l), inositol (2000 μg/l), niacin (400 μg/l), p-aminobenzoic acid (200 μg/l), pyridoxine hydrochloride (400 μg/l), riboflavin (200 μg/l), thiamine hydrochloride (400 μg/l), boric acid (500 μg/l), copper sulfate pentahydrate (40 μg/l), potassium iodide (100 μg/l), ferric chloride (200 μg/l), manganese sulfate monohydrate (400 μg/l), sodium molybdate (200 μg/l), zinc sulfate monohydrate (400 μg/l), monopotassium phosphate (1 g/l), magnesium sulfate (0.5 g/l), sodium chloride (0.1 g/l), and calcium chloride dihydrate (0.1 g/l).

Synthetic defined minus uracil (SD-U) medium. SD-U medium is identical to SD medium with the exception that uracil was not included in the medium. Engineered strains auxotrophic for uracil are unable to grown on SD-U medium while engineered strains containing a plasmid or integrated DNA cassette comprising a uracil selectable marker are capable of growth in SD-U medium.

PSA12 growth medium. PSA12 medium comprises 20 or 50 g/l glucose (as indicated), 2.86 g/l monopotassium phosphate, 1 g/l magnesium sulfate heptahydrate, 3.4 g/l urea, 2 mg/l myo-inositol, 0.4 mg/l thiamine HCl, 0.4 mg/l pyridoxal HCl, 0.4 mg/l niacin, 0.4 mg/l calcium pantothenate, 2 μg/l biotin, 2 μg/l folic acid, 200 μg/l p-aminobenzoic acid, 200 μg/l riboflavin, 0.13 g/l citric acid monohydrate, 0.5 mg/l boric acid, 574 μg/1 copper sulfate, 8 mg/l iron chloride hexahydrate, 0.333 mg/l manganese chloride, 200 m/1 sodium molybdate, and 4.67 mg/l zinc sulfate heptahydrate. When preparing solid medium plates, 2% agarose is additionally included.

### DNA Integration Cassettes Used in the Examples

Table 1 provides the name, a detailed description, and the SEQ ID NO for the DNA integration cassettes used to engineer the host strains in the Examples. Those skilled in the art will recognize that the genetic elements listed are nucleic acids that have specific functions useful when engineering a recombinant host cell. The genetic elements used herein include transcriptional promoters, transcriptional terminators, protein-coding sequences, sequences flanking the cassette used for homologous recombination of the cassette into the host cell genome at the specified loci, selectable markers, and non-coding DNA linkers. Abbreviations used herein include: 29-bp=29 bp non-coding DNA linkers included between the specified genetic elements, 59-bp=59 bp non-coding DNA linkers used to remove the URA3 selectable marker following successful integration of the DNA integration cassette, URA3(1/2)=first half of a coding sequence for the URA3 selectable marker, and URA3(2/2)=second half of a coding sequence for the URA3 selectable marker.

For protein coding sequences, the genus and species of the organism from which a sequence is derived are included as a two-letter abbreviation before the protein name. For example, Sc=S. cerevisiae, Pk=P. kudriavzevii, Sp=S. pombe, and At=Arabidopsis thaliana. Similarly, transcriptional promoters and transcriptional terminators are identified with a lower-case “p” (transcriptional promoter) or “t” (transcriptional terminator), followed by the genus and species abbreviation (described above), and then the name of the protein-coding gene the promoter or terminator is associated with on the genome of the indicated wild-type organism. For example, pPkTDH1 refers to the transcriptional promoter of the TDH1 gene in wild-type P. kudriavzevii. As a second example, tScGRE3 refers to the transcriptional terminator of the GRE3 gene in wild type S. cerevisiae.

Each DNA integration cassette described in Table 1 also contains 5′ and 3′ flanking genetic elements used for homologous recombination of each DNA cassette into the host cell genome. The abbreviation US refers to the genomic sequence upstream of the indicated gene on the genome of host cell being engineered. Likewise, DS refers to the genomic sequence downstream of the indicated gene. For example, when engineering P. kudriavzevii, ADH6C_US refers to a sequence that is homologous to the untranslated region immediately upstream (5′-) of the ADH6C coding sequence on the P. kudriavzevii genome. Likewise, ADH5C_DS refers to a sequences that is homologous to the untranslated region immediately downstream (3′-) of the ADH6C coding sequence on the P. kudriavzevii genome.

TABLE 1
DNA Integration Cassettes Used for Strain Engineering

| DNA Integration | | |
|---|---|---|
| Cassette | Genetic Elements (listed 5′ to 3′) | SEQ ID NO |
| s376 | PkURA3_2/2, tScTDH3, 59-bp, ADH6C_DS | 41 |
| s404 | ADH6C_US, pPkTDH1, D0IX49, tScGRE3, 59-bp, pPkTEF1, PkURA3_1/2 | 42 |
| s357 | GPD1_US, 59-bp, pPkTEF1, URA3, tScTDH3, 59-bp, GPD1_DS | 43 |
| s475 | ADH7_US, pPkTDH1, PkPYC, tPkPYC, 59-bp, pPkTEF1, PkURA3(1/2) | 44 |
| s422 | PkURA3(2/2), tScTDH3, 59-bp, ADH7_DS | 45 |
| s424 | PDC5_US, 59-bp, pPkTEF1, PkURA3, tScTDH3, 59-bp, PDC5_DS | 46 |
| s423 | PDC6_US, 59-bp, pPkTEF1, PkURA3, tScTDH3, 59-bp, PDC6_DS | 47 |
| s425 | PDC1_US, 59-bp, pPkTEF1, PkURA3, tScTDH3, 59-bp, PDC1_DS | 48 |
| s445 | DUR1,2A_US, 59-bp, pPkTEF1, PkURA3, tScTDH3, 59-bp, DUR1,2A_DS | 49 |
| s484/s485/s486 | ALD2A_US, 29-bp, pPkTDH1, SpURED, tScTDH3, pPkTEF1, SpUREF, tScGRE3, | 50 |
| | ScBUD9_US, 29-bp, pPkURA3, PkURA3, tPkURA3, 29-bp, ScBUD9_US, 59-bp, | |
| | pPkPGK1, SpUREG, ALD2A_DS | |
| s481 | DUR1,2A_US, pPkTDH1, SpURE2, tScGRE3, 59-bp, pPkTEF1, PkURA3(1/2) | 51 |
| s482 | PkURA3(2/2), tScTDH3, 59-bp, pPkPGK1, SpNIC1, DUR1,2A_DS | 52 |
| s483 | PkURA3(2/2), tScTDH3, 59-bp, DUR1,2_DS | 53 |
| s394 | ADH6c_US, pPkTDH1, B3R8S4, tScGRE3, 59-bp, pPkTEF1, PkURA3(1/2) | 54 |
| s396 | ADH6c_US, pPkTDH1, Q126F5, tScGRE3, 59-bp, pPkTEF1, PkURA3(1/2) | 55 |
| s408 | PkURA3(2/2), tScTDH3, 59 bp, pPkPGK1, AtSIAR1, tScTPI1, ADH6c_DS | 56 |
| s409 | PkURA3(2/2), tScTDH3, 59 bp, pPkPGK1, AtBAT1, tScTPI1, ADH6c_DS | 57 |

TABLE 2
Genotype of recombinant P. kudriavzevii strains

| | | | Heterologous nucleic acids encoding |
|---|---|---|---|
| Uracil | Uracil | Endogenous genes deleted for L-aspartate and/or | proteins expressed for L-aspartate and/or |
| Auxotroph | Prototroph | beta-alanine production | beta-alanine production |
| LPK15434, | LPK15419 | | |
| LPK15454 | | | |
| LPK15584 | LPK15490 | PkPDC5 | |
| LPK15588 | LPK15586 | PkPDC5, PkPDC6 | |
| LPK15620 | LPK15611 | PkPDC5, PkPDC6, PkPDC1 | |
| LPK15641 | LPK15613 | PkDUR1,2A | |
| LPK15719 | LPK15643 | PkGPD1 | |
| LPK15785 | LPK15756 | PkPDC5, PkPDC6, PkPDC1, PkGPD1 | PkPYC |
| LPK15786 | LPK15758 | PkGPD1 | PkPYC |
| LPK15783 | LPK15773 | PkDUR1,2A | SpURED, SpUREF, SpUREG |
| LPK15784 | LPK15774 | PkDUR1,2A | SpURED, SpUREF, SpUREG |
| | LPK15800, | PkDUR1,2A | SpURED, SpUREF, SpUREG, SpURE2, |
| | LPK15827 | | SpNIC1 |
| | LPK15801, | PkDUR1,2A | SpURED, SpUREF, SpUREG, SpURE2 |
| | LPK15831 | | |
| | LPK15785C | PkPDC5, PkPDC6, PkPDC1, PkGPD1 | PkPYC, Q126F5 |
| | LPK15785D | PkPDC5, PkPDC6, PkPDC1, PkGPD1 | PkPYC, D0IX49 |
| | LPK15786C | PkGPD1 | PkPYC, Q126F5 |
| | LPK15786D | PkGPD1 | PkPYC, D0IX49 |
| | LPK15786F | PkGPD1 | PkPYC, Q126F5, AtSIAR1 |
| | LPK15786G | PkGPD1 | PkPYC, D0IX49, AtSIAR1 |
| | LPK15786I | PkGPD1 | PkPYC, Q126F5, AtBAT1 |
| | LPK15786I | PkGPD1 | PkPYC, D0IX49, AtBAT1 |
| | LPK15785F | PkPDC5, PkPDC6, PkPDC1, PkGPD1 | PkPYC, Q126F5, AtSIAR1 |
| LPK15785G-1 | LPK15785G | PkPDC5, PkPDC6, PkPDC1, PkGPD1 | PkPYC, D0IX49, AtSIAR1 |
| LPK15785G-3 | LPK15785G-2 | PkPDC5, PkPDC6, PkPDC1, PkGPD1, PkDUR1,2A | PkPYC, D0IX49, AtSIAR1, SpURE2 |
| | LPK15785G-4 | PkPDC5, PkPDC6, PkPDC1, PkGPD1, PkDUR1,2A | PkPYC, D0IX49, AtSIAR1, SpURE2, |
| | | | SpURED, SpUREF, SpUREG |
| | LPK15785I | PkPDC5, PkPDC6, PkPDC1, PkGPD1 | PkPYC, Q126F5, AtBAT1 |
| | LPK15785J | PkPDC5, PkPDC6, PkPDC1, PkGPD1 | PkPYC, D0IX49, AtBAT1 |

LPK15343 and LPK15454 are identical with the exception that LPK15434 has a kanamycin resistance marker present (not listed in this table).
LPK15773 and LPK15774 are different isolates for the same transformation.
LPK15827 and LPK15831 are LPK15800 and LPK15801, respectively, adapted for growth on urea as the sole nitrogen source.

### Example 1: Construction of Recombinant P. Kudriavzevii Strains Expressing L-Aspartate Dehydrogenases, and their Use in the Production of L-Aspartate in Yeast

Nucleic acids encoding different L-aspartate dehydrogenases were codon-optimized for yeast, synthesized, and integrated into the Pichia kudriavzevii genome; in vivo expression of the L-aspartate dehydrogenases resulted in production of L-aspartate. Codon optimized DNA encoding for each L-aspartate dehydrogenase was first synthesized by a commercial DNA synthesis company (e.g., Gen9, Inc.). The synthetic DNA was then amplified by PCR using primers to add DNA sequences aiding molecular cloning of the DNA into expression constructs. The primers used were as follows (listed as UniProt ID for the protein encoded by the template DNA, forward primer name and sequence, reverse primer name and sequence): Q9HYA4 encoding template DNA, YO1504 forward primer (5′-CACAAACAAACACAATTACAAAAAATGTTGAATATCGTTATGATTGGTTG-3′) and YO1505 reverse primer (5′-GAGTATGGATTTTACTGGCTGGATTAAATAGAGATAGCGTGAGCATG); B3R8S4 encoding template DNA, YO1506 forward primer (5′-CACAAACAAACACAATTACAAAAAATGTTGCACGTTTCTATGGTTGG-3′) and YO1507 reverse primer (5′-GAGTATGGATTTTACTGGCTGGATTAGATAGAAACGGCGTGGG-3′); Q8XRV9 encoding template DNA, YO1508 forward primer (5′-CACAAACAAACACAATTACAAAAAATGTTACATGTTTCTATGGTCGG-3′) and YO1509 reverse primer (5′-GAGTATGGATTTTACTGGCTGGATTAGATAGAGACAGCATGAGCTC-3′); Q126F5 encoding template DNA, YO1510 forward primer (5′-CACAAACAAACACAATTACAAAAAATGTTGAAGATCGCTATGATTGG-3′) and YO1511 reverse primer (5′-GAGTATGGATTTTACTGGCTGGATTAAATAACCAAAGCTCTACCTCTG-3′); Q2T559 encoding template DNA, YO1512 forward primer (5′-CACAAACAAACACAATTACAAAAAATGAGAAACGCTCATGCC C-3′) and YO1513 reverse primer (5′-GAGTATGGATTTTACTGGCTGGATTAAATGACACAATGGGAAGCAC-3′); Q3JFK2 encoding template DNA, YO1514 forward primer (5′-CACAAACAAACACAATTACAAAAAATGCGTAACGCCCATGCTC-3′) and YO1515 reverse primer (5′-GAGTATGGATTTTACTGGCTGGATTAAATAACACAATGGGAGGCTC-3′); A6X792 encoding template DNA, YO1516 forward primer (5′-CACAAACAAACACAATTACAAAAAATGTCTGTCTCTGAAACTATCGTC-3′) and YO1517 reverse primer (5′-GAGTATGGATTTTACTGGCTGGATTAAATAACGGTGGTAGCAACTC-3′); D6JRV1 encoding template DNA, YO1518 forward primer (5′-CACAAACAAACACAATTACAAAAAATGAAGAAGTTGATGATGATCGG-3′) and YO1519 reverse primer (5′-GAGTATGGATTTTACTGGCTGGATTAAATTTGGATGGCCTCAACAG-3′); A6TDT8 encoding template DNA, YO1520 forward primer (5′-CACAAACAAACACAATTACAAAAAATGATGAAGAAGGTCATGTTAATTG-3′) and YO1521 reverse primer (5′-GAGTATGGATTTTACTGGCTGGATTAGGCCAATTCTCTACAAGC-3′); A8LLH8 encoding template DNA, YO1522 forward primer (5′-CACAAACAAACACAATTACAAAAAATGAGATTGGCTTTGATCGG-3′) and YO1523 reverse primer (5′-GAGTATGGATTTTACTGGCTGGATTAAACAACCCAGGCAGCG-3′); Q5LPG8 encoding template DNA, YO1524 forward primer (5′-CACAAACAAACACAATTACAAAAAATGTGGAAGTTGTGGGGTTC-3′) and YO1525 reverse primer (5′-GAGTATGGATTTTACTGGCTGGATTAGAAGGATGGTCTAATGGCAG-3′); DOIX49 encoding template DNA, YO1526 encoding forward primer (5′-CACAAACAAACACAATTACAAAAAATGAAAAACATCGCCTTAATTGG-3′) and YO1527 encoding reverse primer (5′-GAGTATGGATTTTACTGGCTGGATTAAATAGCCAATGGAGCGAC-3′). For DNA encoding L-aspartate dehydrogenase Q46VA0, 5′- and 3′-DNA sequences with homology to the adjacent parts needed for molecular cloning was included during synthesis and no PCR amplification step was used when cloning the Q46VA0 encoding DNA.

The resulting DNA fragments were purified and cloned downstream of the P. kudriavzevii TDH1 promoter and upstream of the S. cerevisiae GRE3 terminator, which are flanked in 5′ by 473 bp of sequence upstream of the P. kudriavzevii Adh6c gene and in 3′ by a non-functional portion of the Ura3 selection marker, in a plasmid vector containing the ampicillin resistance cassette and the pUC origin of replication using conventional molecular cloning methods. The resulting plasmids were transformed into E. coli competent host cells and selected on LB agar plates containing Amp¹⁰⁰. Following overnight incubation at 37° C., individual colonies were inoculated in 5 ml of LB-Amp′⁰⁰ grown overnight at 37° C. on a shaker before the plasmids were isolated and the identity and integrity of the constructs confirmed by sequencing, resulting in plasmids s393-405. The complementary construct for genomic integration containing the remaining part of the Ura3 marker and a region corresponding to 385 bp downstream of the P. kudriavzevii Adh6c gene was constructed similarly to produce plasmid s376.

P. kudriavzevii strain LPK15434 was used as the background strain for genomic integration of the L-aspartate dehydrogenase expression constructs. LPK15434 is a uracil auxotroph generated from wild type P. kudriavzevii through deletion of the URA3 gene. The plasmids encoding the various L-aspartate dehydrogenase expression cassettes (s393-405) were first digested with restriction enzyme MssI to release the linear integration cassette and co-transformed into the host strains with MssI-digested s376 using standard procedures and selected on defined agar medium lacking uracil. After 3 days incubation at 30° C., uracil prototroph transformants were re-streaked on selective medium lacking uracil, and correct integration of the L-aspartate dehydrogenase expression cassettes was confirmed by PCR.

PCR verified transformants (2-6 for each strain) were inoculated in a 96-well plate containing 0.5 ml of medium (YNB, 2% glucose, 100 mM citrate buffer pH 5.0) along with control strain LPK15419 and grown at 30° C. for 3 days, shaking at 300 rpm with 50 mm throw in an incubator maintained at 80% r.h. Control strain LPK15419 is identical to LPK15434 with the exception that the URA3 gene has not been deleted. After 3 days, the cultures were pelleted and the medium supernatant was filtered on a 0.2 micron PVDF membrane and stored at 4° C. until analysis.

For HPLC analysis, samples and L-aspartate standards were derivatized with one volume of phtaldialdehyde reagent according to standard procedures and immediately analyzed on a Shimadzu HPLC system configured as follows: Agilent C18 Plus (2.1×150 mm, 5 μm) column at 40° C., UV detector at 340 nm; 0.4 mL/min isocratic mobile phase (40 mM NaH₂PO₄, pH=7.8) flow; 5 μL injection volume; 18 min total run time.

The control strain LPK15419 did not produce a detectable amount of L-aspartate. In the LPK15434 background engineered for expression of L-aspartate dehydrogenase proteins, a detectable level of L-aspartate was measured. Expression of the following L-aspartate dehydrogenase proteins resulted in the indicate amount of L-aspartate (mean+/−standard deviation): Q9HYA4, 13±2 mg/L; B3R8S4, 9±0 mg/L; Q8XRV9, 13±3 mg/L; Q126F5, 13±1 mg/L; Q2T559, 11±1 mg/L; Q3JFK2, 15±2 mg/L; A6X792, 13±3 mg/L; D6JRV1, 13±4 mg/L; A6TDT8, 12±1 mg/L; A8LLH8, 11±2 mg/L; Q5LPG8, 14±1 mg/L; D0IX49, 12±2 mg/L; and Q46VA0, 10±2 mg/L. Thus, all engineered Pichia kudriavzevii strains expressing heterologous L-aspartate dehydrogenase proteins resulted in production of L-aspartate while no L-aspartate was observed in the parental, control strain. This example demonstrates, in accordance with the present invention, the expression of nucleic acids encoding L-aspartate dehydrogenase proteins in recombinant P. kudriavzevii for production of L-aspartate.

### Example 2: Construction of Engineered S. cerevisiae Strains Expressing Heterologous L-Aspartate Dehydrogenase and Demonstration of Functional L-Aspartate Dehydrogenase Activity

In this example, S. cerevisiae strains were engineered to express three different heterologous L-aspartate dehydrogenase enzymes, namely Cupriavidus taiwanensis L-aspartate dehydrogenase B3R8S4, Polaromonas sp. L-aspartate dehydrogenase Q126F5, and Comamonas testosteroni L-aspartate dehydrogenase D0IX49. Functional L-aspartate dehydrogenase activity was demonstrated in clarified whole-cell lysates obtained from the engineered strains. This example also provides a method for identifying nucleic acids encoding functional L-aspartate dehydrogenase enzymes suitable for expression in engineered host cells, including, but not limited to, engineered S. cerevisiae host cells.

Nucleic acids encoding Cupriavidus taiwanensis L-aspartate dehydrogenase B3R8S4 (SEQ ID NO: 02), Polaromonas sp. L-aspartate dehydrogenase Q126F5 (SEQ ID NO: 18), and Comamonas testosteroni L-aspartate dehydrogenase D0IX49 (SEQ ID NO: 26) were codon-optimized for expression in yeast and synthesized by a commercial DNA synthesis provider (e.g., IDT DNA, Coralville, Iowa). The nucleic acids were individually PCR amplified from the synthetic DNA using primers containing 25-50 bp overhangs with sequence homology to the 3′ and 5′ ends of Mss/restriction digested yeast expression vector pTL3 (SEQ ID NO: 28), and inserted via DNA sequence homology-based cloning between (5′ to 3′) the pPkTDH3 transcriptional promoter and the tScTPI1 transcriptional terminator of the linearized pTL3 vector backbone. Correct plasmid assembly was confirmed by PCR and DNA sequencing

Following assembly, plasmids were transformed into S. cerevisiae strain BY4742 using a lithium acetate transformation method. Transformants were selected on SD-U agarose plates, and individual colonies were isolated.

Replicate cultures of each engineered strain and the control strain harboring an empty pTL3 plasmid were grown in SD-U medium (5 ml growth volume; 30° C.; 250 rpm shaking). A 2-ml aliquot of each culture was pelleted (1 min; 13,000×-g), washed with DI water, and the two replicate culture samples were combined and pelleted a final time. The washed cell pellets were re-suspended in 150 μL of lysis reagent (CelLytic Y (Sigma Aldrich) with 5 μl/ml of 1M dithiothreitol and 10 μl/ml protease inhibitor cocktail (catalog#: P8215; Sigma Aldrich), and incubated for 30 minutes at room temperature with intermittent mixing. Cell debris was removed by centrifugation (5 min; 13,000×-g), and the clarified whole-cell lysates (i.e., supernatant) were transferred to new Eppendorf tubes.

L-aspartate dehydrogenase activity was measured by reduction of oxaloacetate to L-aspartic acid. To this end, 8 μl of each clarified whole-cell lysate was combined in an Eppendorf tube with 300 μl of an L-aspartate dehydrogenase assay mixture comprising 100 mM Tris HCl (pH 8.2), 20 mM oxaloacetate, 10 mM NADH, and 150 mM ammonium chloride. Each sample was incubated for 1 hour at room temperature and then frozen at −80° C. to inactivate the enzyme. After thawing, each sample was filtered through a 0.2 micron PVDF membrane prior to aspartic acid quantification by HPLC.

For HPLC analysis, 1.5 μl of sample (or L-aspartate standard) was derivatized at room temperature for 5 minutes immediately prior to injection in a reaction mixture containing 100 μl of water, 50 μl of 0.4M borate buffer (pH 10.2) and 50 μl of o-phthaldialdehyde (OPA) reagent (catalog # P0523, 1 mg/ml solution; Sigma-Aldrich). The derivatized samples were then analyzed on a Shimadzu HPLC system configured as follows: Agilent ZORBAX 80A Extend C-18 column (3.0 mm I.D.×150 mm L., 3.5 um P.S.) at 40° C., UV-VIS detector at 338 nm, 0.64 ml/min flow rate, and 1 μl injection volume. The mobile phase was a gradient of two solvents: A (40 mM NaH2PO4, pH 7.8 with 10N NaOH) and B (45% acetonitrile, 45% methanol, and 10% water; % v/v). The mobile phase composition over the sample run time was as follows (run time in minutes with % solvent B in parentheses): 0 (2%), 0.5 (2%), 1.5 (25%), 1.55 (4%), 9.0 (25%), 14.0 (41.5%), 14.1 (100%), 18.0 (100%), 18.5 (2%), 20.0—end or run. The retention time of L-aspartic acid using this protocol was ca. 2.67 minutes.

No L-aspartic acid activity was detected in the whole-cell lysate of S. cerevisiae control strain BY4742 harboring the empty plasmid. In contrast, whole-cell lysates of the engineered S. cerevisiae strains expressing L-aspartate dehydrogenase enzymes B3R8S4, Q126F5, and D0IX49 produced an average (n=2) of 11.60 mM, 12.27 mM, and 12.26 mM L-aspartic acid, respectively. Thus, this example demonstrated functional expression of three different L-aspartate dehydrogenase enzymes in engineered S. cerevisiae host cells.

### Example 3: Construction of P. Kudriavzevii Strains Lacking Endogenous NAD-Dependent Glycerol-3-Phosphate Dehydrogenase and Comprising Nucleic Acids Encoding Heterologous L-Aspartate Dehydrogenase and Heterologous Pyruvate Carboxylase

In this example, P. kudriavzevii strains were engineered to lack both alleles of the gene encoding endogenous NAD-dependent glycerol-3-phosphate dehydrogenase PkGPD1, and to comprise heterologous nucleic acids encoding Polaromonas sp. L-aspartate dehydrogenase Q126F5 (SEQ ID NO: 18) or Comamonas testosteroni L-aspartate dehydrogenase D0IX49 (SEQ ID NO: 26), and P. kudriavzevii pyruvate carboxylase PkPYC (SEQ ID NO: 58). Functional L-aspartate dehydrogenase activity was demonstrated in clarified whole-cell lysates obtained from the engineered strains. This example also provides a method for identifying nucleic acids encoding functional L-aspartate dehydrogenase enzymes suitable for expression in recombinant host cells, including, but not limited to, P. kudriavzevii host cells.

First, both alleles of the PkGPD1 gene were deleted from recombinant P. kudriavzevii strain LPK15454 comprising deletions of both alleles of the URA3 gene. To this end, the strain was transformed with DNA integration cassette s357. Deletion of both alleles of the PkGPD1 gene provided P. kudriavzevii strain LPK15643, and upon removal of the URA3 selectable marker between the 59-bp DNA linkers by Cre recombinase-mediated recombination P. kudriavzevii strain LPK15719.

Next, an expression construct encoding PkPYC was integrated at both alleles of the ADH7 locus in the genome of P. kudriavzevii strain LPK15719 by co-transforming the strain with DNA integration cassettes s475 and s422. Integration of the expression construct encoding PkPYC provided P. kudriavzevii strain LPK15758, and upon removal of the URA3 selectable marker between the 59-bp DNA linkers by Cre recombinase-mediated recombination P. kudriavzevii strain LPK15786.

Next, DNA integration cassettes for expression of L-aspartate dehydrogenases Q126F5 or D0IX49 were integrated at both alleles of the ADH6C locus of strain LPK15786 by co-transformation with the DNA integration cassettes s376 and s396 (for L-aspartate dehydrogenase Q126F5) or s376 and s404 (for L-aspartate dehydrogenase D0IX49). Integration s376 and s396 provided strain LPK15786C for expression of L-aspartate dehydrogenase Q126F5. Integration of s376 and s404 provided strain LPK15786D for expression of L-aspartate dehydrogenase D0IX49.

Methods for strain transformation, selection of uracil prototrophic strains, removal of the uracil selection cassettes to obtain uracil autotrophic strains, and confirmation of successful integrations were identical to those described above. The structures and sequences of the DNA integration cassettes used are given in Table 1. The recombinant P. kudriavzevii strains are summarized in Table 2.

L-aspartate dehydrogenase activity in clarified whole-cell lysates obtained from two colonies of each engineered strain was measured using a kinetic assay following the decrease in NADH absorbance at 340 nm over a 5-minute time period. A 150 ul reaction mixture was prepared in a 96-well plate comprising 5 mM oxaloacetate, 0.25 mM NADH, 100 mM Tris HCl pH 8.2, 100 mM NH4Cl, and 2.5 ul of clarified whole-cell lysate. Control reactions were prepared in which the NH4Cl was excluded from the reaction mixture as it was observed that ammonium was required to observe NADH oxidase activity. The linear portion of the curve was used to calculate the activity in each sample; one Unit of L-aspartate dehydrogenase activity was defined as the amount of enzyme required to oxidize 1 umol NADH per minute per mg of total protein in these conditions. Protein concentration in the extracts was measured with the Bradford method, and the results used to normalize the activity of the whole-cell lysates.

The whole-cell lysates derived from a control, parental P. kudriavzevii strain exhibited low NADH oxidase activity (6.2±0.5 U/mg total protein), and activity was independent of the presence of ammonium in the reaction mixture, indicating non-specific NADH oxidation. In comparison, whole-cell lysates derived from recombinant P. kudriavzevii strains LPK15786C and LPK15786D provided significantly higher NADH-oxidase activity (29±5 and 25.8±0.5 U/mg total protein, respectively); additionally, the activity was dependent on the presence of ammonium in the reaction mixture, confirming L-aspartate dehydrogenase activity in these samples.

### Example 4: Construction of P. kudriavzevii Strains Lacking Endogenous NAD-Dependent Glycerol-3-Phosphate Dehydrogenase and Endogenous Pyruvate Decarboxylase, and Comprising Nucleotide Sequences Encoding Heterologous Pyruvate Carboxylase and Heterologous L-Aspartate Dehydrogenase

In this example, P. kudriavzevii strains were engineered to lack both alleles of the gene encoding endogenous NAD-dependent glycerol-3-phosphate dehydrogenase PkGPD1 and both alleles of each of three genes encoding endogenous pyruvate decarboxylases PkPDC1 (SEQ ID NO: 9), PkPDC6 (SEQ ID NO: 29), and PkPDC5 (SEQ ID NO: 30), and to comprise heterologous nucleic acids encoding Polaromonas sp. L-aspartate dehydrogenase Q126F5 (SEQ ID NO: 18) or Comamonas testosteroni L-aspartate dehydrogenase D0IX49 (SEQ ID NO: 26) and P. kudriavzevii pyruvate carboxylase PkPYC (SEQ ID NO: 58).

First, both alleles of the PkPDC5 gene were deleted from recombinant P. kudriavzevii strain LPK15454 comprising deletions of both alleles of the URA3 gene. To this end, the strain was transformed with DNA integration cassette s424. Deletion of both alleles of the PDC5 gene provided P. kudriavzevii strain LPK15490, and upon removal of the URA3 selectable marker between the 59-bp DNA linkers by Cre recombinase-mediated recombination P. kudriavzevii strain LPK15584.

Next, both alleles of the PkPDC6 gene were deleted from P. kudriavzevii strain LPK15584 by transforming the strain with DNA integration cassette s423. Deletion of both alleles of the PDC6 gene provided P. kudriavzevii strain LPK15586, and upon removal of the URA3 selectable marker between the 59-bp DNA linkers by Cre recombinase-mediated recombination P. kudriavzevii strain LPK15588.

Next, both alleles of the PkPDC1 gene were deleted from P. kudriavzevii strain LPK15588 by transforming the strain with DNA integration cassette s425. Deletion of both alleles of the PDC6 gene provided P. kudriavzevii strain LPK15611, and upon removal of the URA3 selectable marker between the 59-bp DNA linkers by Cre recombinase-mediated recombination P. kudriavzevii strain LPK15620.

Next, both alleles of the PkGPD1 gene were deleted and an expression construct encoding PkPYC was integrated at both alleles of the ADH7 locus using identical DNA integration cassettes and methods as described in Example 3. Deletion of both alleles of the PkGPD1 gene and integration of the expression construct encoding PkPYC provided P. kudriavzevii strain LPK15756, and upon removal of the URA3 selectable marker between the 59-bp DNA linkers by Cre recombinase-mediated recombination P. kudriavzevii strain LPK15785.

Next, expression constructs for expression of L-aspartate dehydrogenase Q126F5 or DOIX49 were integrated at both alleles of the ADH6C locus of strain LPK15785 by co-transformation with DNA integration cassettes s376 and s396 (for L-aspartate dehydrogenase Q126F5) or s376 and s404 (for L-aspartate dehydrogenase DOIX49). Integration of s396 and s397 provided strain LPK15785C (for L-aspartate dehydrogenase Q126F5 expression). Integration of s376 and s404 provided strain LPK15785D (for L-aspartate dehydrogenase DOIX49 expression).

Methods for strain transformation, selection of uracil prototrophic strains, removal of the uracil selection cassettes to obtain uracil autotrophic strains, and confirmation of successful integrations were identical to those described above. The structures and sequences of the DNA integration cassettes used are given in Table 1. The recombinant P. kudriavzevii strains are summarized in Table 2.

### Example 5: Construction of P. kudriavzevii Strains Lacking Endogenous Urea Amidolyase

In this example, P. kudriavzevii strains were engineered to delete both alleles of the DUR1,2A gene encoding endogenous urea amidolyase DUR1,2A. The engineered strains were shown to be unable to grow on urea as the sole nitrogen source. This example demonstrates that deletion or disruption of genes encoding native ATP-dependent urea catabolic pathway enzymes reduces or eliminates a host cell's ability to catabolize urea through this pathway.

Both alleles of the DUR1,2A gene were deleted from the genome of a P. kudriavzevii strain LPK15454 comprising deletion of both alleles of the URA3 gene. To this end, the strain was transformed with DNA integration cassette s445. Deletion of both alleles of the DUR1,2A gene provided P. kudriavzevii strain LPK15613, and upon removal of the URA3 selectable marker between the 59-bp DNA linkers by Cre recombinase-mediated recombination, P. kudriavzevii strain LPK15641.

Methods for strain transformation, selection of uracil prototrophic strains, removal of the uracil selection cassettes to obtain uracil autotrophic strains, and confirmation of successful integrations were identical to those described above. The structures and sequences of the DNA integration cassettes used are given in Table 1. The genotype of the recombinant P. kudriavzevii strains are summarized in Table 2.

Duplicate single colony isolates of recombinant P. kudriavzevii strain LPK15613 and a control strain were inoculated into PSA12 (with 5% glucose), which contains urea as the sole nitrogen source, grown for a period of 20 hours (30° C.; 250 rpm shaking), and the OD600 of the cultures was measured. Strain LPK15613 reached cell densities of OD600 0.20±0.02 (mean+/−standard deviation; n=2) whereas the control strain reached an OD600 of 17.7±0.3. Thus, over 89-fold less biomass was observed for strain LPK15613 grown on urea. The low residual growth observed on urea was attributed to spontaneous degradation of urea in the liquid culture over time, resulting in the slow release of ammonia.

### Example 6: Construction of P. kudriavzevii Strains Lacking Endogenous Urea Amidolyase, and Comprising Nucleic Acids Encoding Heterologous Urease, Heterologous Urease Accessory Proteins, and Heterologous Nickel Transporter

In this example, P. kudriavzevii strains were engineered to lack both alleles of the gene encoding endogenous urea amidolyase DUR1,2A, and to comprise heterologous nucleic acids encoding S. pombe urease SpURE2 (SEQ ID NO: 34); S. pombe urease accessory proteins SpURED (SEQ ID NO: 35), SpUREF (SEQ ID NO: 36), and SpUREG (SEQ ID NO: 37); and S. pombe nickel transporter SpNIC1 (SEQ ID NO: 38).

First, an expression construct encoding S. pombe urease accessory proteins SpURED, SpUREF, and SpUREG was integrated at one allele of the ALD2A locus in the genome of P. kudriavzevii strain LPK15641 by transforming the strain with DNA integration cassette s484/s485/s486. Integration of s484/s485/s486 provided P. kudriavzevii strain LPK15773, and upon removal of the URA3 selectable marker between the 59-bp DNA linkers by Cre recombinase-mediated recombination P. kudriavzevii strain LPK15783.

Next, an expression construct encoding S. pombe urease SpURE2 and S. pombe nickel transporter SpNic1 was integrated at one allele of the DUR1,2A locus (both copies of the protein coding gene were previously deleted, see Example 5) in the genome of P. kudriavzevii strain LPK15783 by co-transforming the strain with DNA integration cassettes s481 and s482. Integration of s481 and s482 provided P. kudriavzevii strain LPK15800.

After verification of correct integration, strain LPK15800 was streaked on agarose plates of PSA12 (2% w/v glucose) supplemented with 20 nM NiCl₂ and incubated at 30° C. for 2 days, then at room temperature for 3 more days. A colony relatively larger than the median colony size was then isolated by restreaking on the same solid media. A single colony was then inoculated in PSA12 (5% w/v glucose)+20 nM NiCL₂ liquid media, cultured (2.5 ml in 15 ml tube, 30° C., 250 rpm), and then sub-cultured 3 times to confirm growth on urea as the sole nitrogen source. An aliquot of the final liquid growth culture was plated on PSA12 (2% w/v glucose)+20 nM NiCl₂, and a single colony isolated, which was labeled P. kudriavzevii strain LPK15827.

Methods for strain transformation, selection of uracil prototrophic strains, removal of the uracil selection cassettes to obtain uracil autotrophic strains, and confirmation of successful integrations were identical to those described above. The structures and sequences of the DNA integration cassettes used are given in Table 1. The recombinant P. kudriavzevii strains are summarized in Table 2.

### Example 7: Construction of P. kudriavzevii Strains Lacking Endogenous Urea Amidolyase, and Comprising Nucleic Acids Encoding Heterologous Urease and Heterologous Urease Accessory Proteins

In this example, P. kudriavzevii strains were engineered to lack both alleles of the gene encoding endogenous urea amidolyase DUR1,2A, and to comprise heterologous nucleic acids encoding S. pombe urease SpURE2 (SEQ ID NO: 34) and S. pombe urease accessory proteins SpURED (SEQ ID NO: 35), SpUREF (SEQ ID NO: 36), and SpUREG (SEQ ID NO: 37). This example differs from Example 6 in that the SpNIC1 transporter was not expressed.

First, an expression construct encoding S. pombe urease accessory proteins SpURED, SpUREF, and SpUREG was integrated at one allele of the ADL2A locus in recombinant P. kudriavzevii strain LPK15641 by transforming the strain with the DNA integration cassette s484/485/486 as described in Example 6, providing P. kudriavzevii strain LPK15774. This strain was a different clonal isolate than strain LPK15773, but was otherwise identical. Subsequent removal of the URA3 selectable marker as previously described generated P. kudriavzevii strain LPK15784.

Next, an expression construct encoding S. pombe urease SpURE2 was integrated at one allele of the DUR1,2A locus (both copies of the DUR1,2A gene were deleted in a previous strain engineering step, see Example 5) in the genome of P. kudriavzevii strain LPK15784 by co-transforming the strain with DNA integration cassettes s481 and s483. Integration of s481 and s483 provided P. kudriavzevii strain LPK15801. The strain was selected for growth on urea as described in Example 6, generating P. kudriavzevii strain LPK15831.

Methods for strain transformation, selection of uracil prototrophic strains, removal of the uracil selection cassettes to obtain uracil autotrophic strains, and confirmation of successful integrations were identical to those described above. The structures and sequences of the DNA integration cassettes used are given in Table 1. The recombinant P. kudriavzevii strains are summarized in Table 2.

### Example 8: Demonstration of Growth on Urea of Recombinant P. kudriavzevii Strains Lacking Endogenous Urea Amidolyase and Expressing Heterologous Urease

As demonstrated in Example 5, a recombinant P. kudriavzevii strain comprising deletion of both alleles of the DUR1,2A gene was unable to grow on urea as the sole nitrogen source. In this example, growth on urea as the sole nitrogen source was restored in this background strain through expression of a heterologous urease and heterologous urease accessory proteins, irrespective of whether the recombinant P. kudriavzevii strain further expressed a heterologous nickel transport (strain constructions are described in Examples 6 and 7).

Recombinant P. kudriavzevii strains LPK15827 and LPK15831 were grown on PSA12 (2% w/v glucose) agarose plates at 30° C. Individual colonies were then inoculated into 2.5 ml of PSA12 (5% w/v glucose) growth medium with or without 20 nM NiCl₂. The cultures were grown at 30° C. with shaking (250 rpm). Cell growth was assayed at 48 and 72 hours by measuring the optical density of the cultures. To this end, aliquots of the cultures were diluted 50-fold in DI water and the OD600 measured on a UV-VIS spectrophotometer (Spectramax Plus384; Molecular Devices). Adjusting for the dilution factor, the LPK15287 culture density at 48 hours in PSA12 and PSA12+20 nM NiCl₂ were 15.6 and 22.9 (OD600; arbitrary units), respectively. At 72 hours, the OD600 values increased to 21.4 and 23.0 for the PSA12 and PSA12+20 nM NiCl₂ samples, respectively. For LPK15831 cultures grown in PSA12 or PSA12+20 nM NiCl₂, the OD600 values at 48 hours were 7.6 and 20.4 and increased to 17.8 and 20.85 at 72 hours, respectively.

### Example 9: Construction of P. kudriavzevii Strains Lacking Endogenous NAD-Dependent Glycerol-3-Phosphate Dehydrogenase, and Comprising Nucleic Acids Encoding Heterologous L-Aspartate Dehydrogenase, Heterologous Pyruvate Carboxylase, and Heterologous L-Aspartate Transport Protein

In this example, P. kudriavzevii strains were engineered to lack both alleles of the GPD1 gene encoding endogenous NAD-dependent glycerol-3-phosphate dehydrogenase PkGPD1, and to comprise heterologous nucleic acids encoding Polaromonas sp. L-aspartate dehydrogenase Q126F5 (SEQ ID NO: 18) or Comamonas testosteroni L-aspartate dehydrogenase D0IX49 (SEQ ID NO: 26), P. kudriavzevii pyruvate carboxylase PkPYC (SEQ ID NO: 58), and Arabidopsis thaliana L-aspartate transport protein AtSIAR1 (SEQ ID NO: 39) or AtBAT1 (SEQ ID NO: 40).

An expression construct encoding a L-aspartate dehydrogenase and a L-aspartate transport protein (codon-optimized for expression in yeast) was integrated at one allele of the ADH6C locus in the genome of recombinant P. kudriavzevii strain LPK15786, which comprises deletions of both alleles of the GPD1 gene and overexpresses PkPYC. To this end, the strain was co-transformed with DNA integration cassettes s396 or s404 (for expression of L-aspartate dehydrogenases Q126F5 or D0IX49, respectively), and DNA integration cassettes s408 or s409 (for expression of L-aspartate transport protein AtSIAR1 or AtBAT1, respectively) using a lithium acetate transformation method. Integration of s396 and s408 provided P. kudriavzevii strain LPK15786F (for expression of L-aspartate dehydrogenase Q126F5 and L-aspartate transport protein AtSIAR1). Integration of s404 and s408 provided P. kudriavzevii strain LPK15786G (for expression of L-aspartate dehydrogenase D0IX49 and L-aspartate transport protein AtSIAR1). Integration of s396 and s409 provided P. kudriavzevii strain LPK157861 (for expression of L-aspartate dehydrogenase Q126F5 and L-aspartate transport protein AtBAT1). Integration of s404 and s409 provided P. kudriavzevii strain LPK15786J (for expression of L-aspartate dehydrogenase D0IX49 and L-aspartate transport protein AtBAT1).

Methods for strain transformation, selection of uracil prototrophic strains, removal of the uracil selection cassettes to obtain uracil autotrophic strains, and confirmation of successful integrations were identical to those described above. The structures and sequences of the DNA integration cassettes used are given in Table 1. The recombinant P. kudriavzevii strains are summarized in Table 2.

### Example 10: Construction of P. kudriavzevii Strains Lacking Endogenous Pyruvate Decarboxylase and Endogenous NAD-Dependent Glyceraldehyde-3-Phosphate Dehydrogenase, and Expressing Heterologous Pyruvate Carboxylase, Heterologous L-Aspartate Dehydrogenase, and Heterologous L-Aspartate Transport Protein

In this example, P. kudriavzevii strains were engineered to lack both alleles of each of three genes encoding endogenous pyruvate decarboxylases PkPDC1, PkPDC5, and PkPDC5; and both alleles of the gene encoding endogenous NAD-dependent glycerol-3-phosphate dehydrogenase PkGPD1, and to comprise heterologous nucleic acids encoding Polaromonas sp. L-aspartate dehydrogenase Q126F5 (SEQ ID NO: 18) or Comamonas testosteroni L-aspartate dehydrogenase D0IX49 (SEQ ID NO: 26), P. kudriavzevii pyruvate carboxylase PkPYC (SEQ ID NO: 58), and Arabidopsis thaliana L-aspartate transport protein AtSIAR1 (SEQ ID NO: 39) or AtBAT1 (SEQ ID NO: 40).

The integration cassettes used were identical to those described in Example 9. This example differs in that the background strain used for the strain engineering was LPK15785, which comprised deletions of both alleles of the genes encoding PkPDC5, PkPDC6, PkPDC1, and PkGPD1; comprised a heterologous nucleic acid encoding PkPYC; and was auxotrophic for uracil. P. kudriavzevii strain LPK15785 was co-transformed with DNA integration cassette s396 or s404 (for expression of L-aspartate dehydrogenases Q126F5 or D0IX49, respectively), and DNA integration cassette s408 or s409 (for expression of L-aspartate transport protein AtSIAR1 or AtBAT1, respectively) using a lithium acetate transformation method. Integration of s396 and s408 provided P. kudriavzevii strain LPK15785F. Integration of s404 and s408 provided P. kudriavzevii strain LPK15785G. Integration of s396 and s409 provided P. kudriavzevii strain LPK157851. Integration of s404 and s409 provided P. kudriavzevii strain LPK15785J.

Methods for strain transformation, selection of uracil prototrophic strains, removal of the uracil selection cassettes to obtain uracil autotrophic strains, and confirmation of successful integrations were identical to those described above. The structures and sequences of the DNA integration cassettes used are given in Table 1. The recombinant P. kudriavzevii strains are summarized in Table 2.

### Example 11: Construction of P. kudriavzevii Strains Lacking Endogenous Pyruvate Carboxylase, NAD-Dependent Glyceraldehyde 3-Phosphate Dehydrogenase, and Urea Amidolyase, and Comprising Nucleic Acids Encoding Heterologous L-Aspartate Dehydrogenase, Heterologous L-Aspartate Transport Protein, Heterologous Urease, and Heterologous Pyruvate Carboxylase

In this example, P. kudriavzevii strains are engineered to lack both alleles of each of three genes encoding endogenous pyruvate decarboxylases PkPDC1, PkPDC5, and PkPDC6; both alleles of the gene encoding endogenous glyceroladehyde-3-phosphate dehydrogenase PkGPD1; and both alleles of the gene encoding endogenous urea amidolyase, and to comprise heterologous nucleic acids encoding Comamonas testosteroni L-aspartate dehydrogenase D0IX49 (SEQ ID NO: 26); P. kudriavzevii pyruvate carboxylase PkPYC (SEQ ID NO: 58); Arabidopsis thaliana L-aspartate transport protein AtSIAR1 (SEQ ID NO: 39); S. pombe urease SpURE2 (SEQ ID NO: 34); and S. pombe urease accessory proteins SpURED, SpUREF, and SpUREG (SEQ ID NOs: 35, 36, and 37, respectively).

The DNA integration cassettes used (s481, s483, s484/s485/s486) are identical to those described in previous examples. The background strain used is recombinant P. kudriavzevii strain LPK15785G (for strain construction see Example 10), which comprises deletions of both alleles of the PkPDC5, PkPDC6, PkPDC1, and PkGPD1 genes, and which comprises heterologous nucleic acids encoding L-aspartate dehydrogenase D0IX49 and heterologous L-aspartate transport protein AtSIAR1. Prior to performing additional strain engineering, the URA3 selection marker in P. kudriavzevii strain LPK15785G is looped out, generating P. kudriavzevii strain LPK15785G-1. P. kudriavzevii strain LPK15785G-1 is co-transformed with DNA integration cassette s481 and s483 (for expression of urease SpURE2) using a lithium acetate transformation method. Integration of s481 and s483 provides P. kudriavzevii strain LPK15785G-2, and upon removal of the URA3 selectable marker between the 59-bp DNA linkers by Cre recombinase-mediated recombination P. kudriavzevii strain LPK15785G-3.

Next, P. kudriavzevii strain LPK15785G-3 was transformed with DNA integration cassette s484/s485/s486 (for expression of urease accessory proteins SpURED, SpUREF, and SpUREG). Integration of s484/s485/s486 provides P. kudriavzevii strain LPK15785G-4. The resulting strain comprises a heterologous URA3 selectable marker and is prototrophic for uracil.

Methods for strain transformation, selection of uracil prototrophic strains, removal of the uracil selection cassettes to obtain uracil autotrophic strains, and confirmation of successful integrations are identical to those described above. The structures and sequences of the DNA integration cassettes used are given in Table 1. The recombinant P. kudriavzevii strains are summarized in Table 2.

### Example 12: Fermentative Production of Aspartic Acid by Recombinant P. kudriavzevii Strain LPK15785G-4

In this example, recombinant P. kudriavzevii strain LPK15785G-4 is used to produce aspartic acid according to the methods of the invention.

An individual colony of LPK15785G-4 is inoculated into 50 ml of PSA12 growth medium (2% w/v glucose) in a 250 ml flask and grown at 30° C. overnight with shaking in a humidified incubator shaker. A culture of wild type P. kudriavzevii is also grown separately as a control strain.

Aliquots (5 ml) of the two overnight cultures are used to inoculate separate 1-liter fermenters containing 500 ml of PSA12 growth medium (10% w/v glucose). The fermentation is run for a period of 72 hours. The pH of the fermentation is controlled to pH 5 by addition of sodium hydroxide as base throughout the entire fermentation. The temperature is held at 30° C. for the entire fermentation. Sterile air is blown into the fermenter and an agitator is used to stir the fermenter for the entire fermentation. The airflow rate is controlled to achieve an oxygen transfer rate of about 20 mmol/l/hr for the first 16 hours of the fermentation, at which point the airflow is decreased to achieve a oxygen transfer rate of the about 5 mmol/l/hr for the remainder of the fermentation.

Samples (5 ml) of each fermentation are taken every 12 hours to measure the concentration of aspartic acid in the fermentation broth over time. Prior to analysis, the samples are pH-adjusted to about 7 to dissolve any aspartic acid that is found in insoluble form in the fermentation broth, and the samples are centrifuged to pellet out cells. Quantification of aspartic acid concentrations in the supernatants is performed using the method described in Example 3. Greater than 1 g/l aspartic acid is measured in the fermentation broth from the fermenter containing recombinant P. kudriavzevii strain LPK15785G-4. No aspartic acid is measured in the control fermentation containing wild type P. kudriavzevii.

### Example 13: Separation of Aspartic Acid Produced by Recombinant P. kudriavzevii from Cells and Fermentation Broth

In this example, a recombinant P. kudriavzevii strain capable of producing aspartic acid is fermented such that the majority of aspartic acid produced is insoluble in the fermenter. The insoluble aspartic acid is separated from the cells and majority of the fermentation broth by both settling and centrifugation at low g-force.

The recombinant P. kudriavzevii strain is fermented used identical methods as those described in Example 12 with the exception that 100 g/l glucose is used in the PSA12 growth medium and the fermentation is not buffered by addition of sodium hydroxide once the airflow rate is decreased to achieve a ca. 5 mmol/l/hr oxygen transfer rate. After 72 hours culture time the fermentation is ended, and ten 50 ml aliquots of well-mixed broth (i.e., cells and insoluble aspartic acid is suspended in the broth) are transferred into 50 ml conical centrifuge tubes.

One sample is allowed to sit upright, undisturbed for a period of 2-hours, and the insoluble aspartic acid is observed to settle at the bottom of the tube over time, separating itself from the cells and broth. By controlling for the amount of time the suspension is allowed to sit, aspartic acid yield can be increased while obtaining a minimum amount of cells in the settled aspartic acid pellet. The supernatant containing the majority of the cells and fermentation broth is decanted from the settled aspartic acid pellet.

Eight samples are centrifuged at different g-forces (50, 100, 150, 200, 250, 300, 350, and 400×-g) for a period of 20 seconds at room temperature. It is observed that the aspartic acid pellet is larger as the g-force is increased from 50 to 400×-g. It is also observed that a second, layer of cells (identified by their light brown color) also begins to form at higher g-forces. By adjusting the g-force and/or time, the insoluble aspartic acid can be separated from the majority of the cells and fermentation broth.

SEQUENCE LISTING

SEQ ID NO: 1. Pseudomonas aeruginosa L-aspartate dehydrogenase.

| | |
|---|---|
| 1- | MLNIVMIGCG AIGAGVLELL ENDPQLRVDA VIVPRDSETQ |
| 41- | VRHRLASLRR PPRVLSALPA GERPDLLVEC AGHRAIEQHV |
| 81- | LPALAQGIPC LVVSVGALSE PGLVERLEAA AQAGGSRIEL |
| 121- | LPGAIGAIDA LSAARVGGLE SVRYTGRKPA SAWLGTPGET |
| 161- | VCDLQRLEKA RVIFDGSARE AARLYPKNAN VAATLSLAGL |
| 201- | GLDRTQVRLI ADPESCENVH QVEASGAFGG FELTLRGKPL |
| 241- | AANPKTSALT VYSVVRALGN HAHAISI -267 |

SEQ ID NO: 2. Cupriavidus taiwanensis L-aspartate dehydrogenase.

| | |
|---|---|
| 1- | MLHVSMVGCG AIGRGVLELL KSDPDVVFDV VIVPEHTMDE |
| 41- | ARGAVSALAP RARVATHLDD QRPDLLVECA GHHALEEHIV |
| 81- | PALERGIPCM VVSVGALSEP GMAERLEAAA RRGGTQVQLL |
| 121- | SGAIGAIDAL AAARVGGLDE VIYTGRKPAR AWTGTPAEQL |
| 161- | FDLEALTEAT VIFEGTARDA ARLYPKNANV AATVSLAGLG |
| 201- | LDRTAVKLLA DPHAVENVHH VEARGAFGGF ELTMRGKPLA |
| 241- | ANPKTSALTV FSVVRALGNR AHAVSI -266 |

SEQ ID NO: 3. Tribolium castaneum L-aspartate 1-decarboxylase.

| | |
|---|---|
| 1- | MPATGEDQDL VQDLIEEPAT FSDAVLSSDE ELFHQKCPKP |
| 41- | APIYSPISKP VSFESLPNRR LHEEFLRSSV DVLLQEAVFE |
| 81- | GTNRKNRVLQ WREPEELRRL MDFGVRGAPS THEELLEVLK |
| 121- | KVVTYSVKTG HPYFVNQLFS AVDPYGLVAQ WATDALNPSV |
| 161- | YTYEVSPVFV LMEEVVLREM RAIVGFEGGK GDGIFCPGGS |
| 201- | IANGYAISCA RYRFMPDIKK KGLHSLPRLV LFTSEDAHYS |
| 241- | IKKLASFEGI GTDNVYLIRT DARGRMDVSH LVEEIERSLR |
| 281- | EGAAPFMVSA TAGTTVIGAF DPIEKIADVC QKYKLWLHVD |
| 321- | AAWGGGALVS AKHRHLLKGI ERADSVTWNP HKLLTAPQQC |
| 361- | STLLLRHEGV LAEAHSTNAA YLFQKDKFYD TKYDTGDKHI |
| 401- | QCGRRADVLK FWFMWKAKGT SGLEKHVDKV FENARFFTDC |
| 441- | IKNREGFEMV IAEPEYTNIC FWYVPKSLRG RKDEADYKDK |
| 481- | LHKVAPRIKE RMMKEGSMMV TYQAQKGHPN FFRIVFQNSG |
| 521- | LDKADMVHFV EEIERLGSDL -540 |

SEQ ID NO: 4. Corynebacterium glutamicum L-aspartate 1-decarboxylase.

| | |
|---|---|
| 1- | MLRTILGSKI HRATVTQADL DYVGSVTIDA DLVHAAGLIE |
| 41- | GEKVAIVDIT NGARLETYVI VGDAGTGNIC INGAAAHLIN |
| 81- | PGDLVIIMSY LQATDAEAKA YEPKIVHVDA DNRIVALGND |
| 121- | LAEALPGSGL LTSRSI -136 |

SEQ ID NO: 5. Bacillus subtilis L-aspartate 1-decarboxylase.

| | |
|---|---|
| 1- | MYRTMMSGKL HRATVTEANL NYVGSITIDE DLIDAVGMLP |
| 41- | NEKVQIVNNN NGARLETYII PGKRGSGVIC LNGAAARLVQ |
| 81- | EGDKVIIISY KMMSDQEAAS HEPKVAVLND QNKIEQMLGN |
| 121- | EPARTIL -127 |

SEQ ID NO: 6. Mannheimia succiniciproducens phosphoenolpyruvate carboxykinase.

| | |
|---|---|
| 1- | MTDLNQLTQE LGALGIHDVQ EVVYNPSYEL LFAEETKPGL |
| 41- | EGYEKGTVTN QGAVAVNTGI FTGRSPKDKY IVLDDKTKDT |
| 81- | VWWTSEKVKN DNKPMSQDTW NSLKGLVADQ LSGKRLFVVD |
| 121- | AFCGANKDTR LAVRVVTEVA WQAHFVTNMF IRPSAEELKG |
| 161- | FKPDFVVMNG AKCTNPNWKE QGLNSENFVA FNITEGVQLI |
| 201- | GGTWYGGEMK KGMFSMMNYF LPLRGIASMH CSANVGKDGD |
| 241- | TAIFFGLSGT GKTTLSTDPK RQLIGDDEHG WDDEGVFNFE |
| 281- | GGCYAKTINL SAENEPDIYG AIKRDALLEN VVVLDNGDVD |
| 321- | YADGSKTENT RVSYPIYHIQ NIVKPVSKAG PATKVIFLSA |
| 361- | DAFGVLPPVS KLTPEQTKYY FLSGFTAKLA GTERGITEPT |
| 401- | PTFSACFGAA FLSLHPTQYA EVLVKRMQES GAEAYLVNTG |
| 441- | WNGTGKRISI KDTRGIIDAI LDGSIDKAEM GSLPIFDFSI |
| 481- | PKALPGVNPA ILDPRDTYAD KAQWEEKAQD LAGRFVKNFE |
| 521- | KYTGTAEGQA LVAAGPKA -538 |

SEQ ID NO: 7. Aspergillus oryzae pyruvate carboxylase

| | |
|---|---|
| 1- | MAAPFRQPEE AVDDTEFIDD HHEHLRDTVH HRLRANSSIM |
| 41- | HFQKILVANR GEIPIRIFRT AHELSLQTVA IYSHEDRLSM |
| 81- | HRQKADEAYM IGHRGQYTPV GAYLAGDEII KIALEHGVQL |
| 121- | IHPGYGFLSE NADFARKVEN AGIVFVGPTP DTIDSLGDKV |
| 161- | SARRLAIKCE VPVVPGTEGP VERYEEVKAF TDTYGFPIII |
| 201- | KAAFGGGGRG MRVVRDQAEL RDSFERATSE ARSAFGNGTV |
| 241- | FVERFLDKPK HIEVQLLGDS HGNVVHLFER DCSVQRRHQK |
| 281- | VVEVAPAKDL PADVRDRILA DAVKLAKSVN YRNAGTAEFL |
| 321- | VDQQNRHYFI EINPRIQVEH TITEEITGID IVAAQIQIAA |
| 361- | GASLEQLGLT QDRISARGFA IQCRITTEDP AKGFSPDTGK |
| 401- | IEVYRSAGGN GVRLDGGNGF AGAIITPHYD SMLVKCTCRG |
| 441- | STYEIARRKV VRALVEFRIR GVKTNIPFLT SLLSHPTFVD |
| 481- | GNCWTTFIDD TPELFSLVGS QNRAQKLLAY LGDVAVNGSS |
| 521- | IKGQIGEPKL KGDVIKPKLF DAEGKPLDVS APCTKGWKQI |
| 561- | LDREGPAAFA KAVRANKGCL IMDTTWRDAH QSLLATRVRT |
| 601- | IDLLNIAHET SYAYSNAYSL ECWGGATFDV AMRFLYEDPW |
| 641- | DRLRKMRKAV PNIPFQMLLR GANGVAYSSL PDNAIYHFCK |
| 681- | QAKKCGVDIF RVFDALNDVD QLEVGIKAVH AAEGVVEATM |
| 721- | CYSGDMLNPH KKYNLEYYMA LVDKIVAMKP HILGIKDMAG |
| 761- | VLKPQAARLL VGSIRQRYPD LPIHVHTHDS AGTGVASMIA |
| 801- | CAQAGADAVD AATDSMSGMT SQPSIGAILA SLEGTEQDPG |
| 841- | LNLAHVRAID SYWAQLRLLY SPFEAGLTGP DPEVYEHEIP |
| 881- | GGQLTNLIFQ ASQLGLGQQW AETKKAYEAA NDLLGDIVKV |
| 921- | TPTSKVVGDL AQFMVSNKLT PEDVVERAGE LDFPGSVLEF |
| 961- | LEGLMGQPFG GFPEPLRSRA LRDRRKLEKR PGLYLEPLDL |
| 1001- | AKIKSQIREK FGAATEYDVA SYAMYPKVFE DYKKFVQKFG |
| 1041- | DLSVLPTRYF LAKPEIGEEF HVELEKGKVL ILKLLAIGPL |
| 1081 - | SEQTGQREVF YEVNGEVRQV AVDDNKASVD NTSRPKADVG |
| 1121- | DSSQVGAPMS GVVVEIRVHD GLEVKKGDPL AVLSAMKMEM |
| 1161- | VISAPHSGKV SSLLVKEGDS VDGQDLVCKI VKA -1193 |

SEQ ID NO: 8. Escherichia coli phosphoenolpyruvate carboxylase

| | |
|---|---|
| 1- | MNEQYSALRS NVSMLGKVLG ETIKDALGEH ILERVETIRK |
| 41- | LSKSSRAGND ANRQELLTTL QNLSNDELLP VARAFSQFLN |
| 81- | LANTAEQYHS ISPKGEAASN PEVIARTLRK LKNQPELSED |
| 121- | TIKKAVESLS LELVLTAHPT EITRRTLIHK MVEVNACLKQ |
| 161- | LDNKDIADYE HNQLMRRLRQ LIAQSWHTDE IRKLRPSPVD |
| 201- | EAKWGFAVVE NSLWQGVPNY LRELNEQLEE NLGYKLPVEF |
| 241- | VPVRFTSWMG GDRDGNPNVT ADITRHVLLL SRWKATDLFL |
| 281- | KDIQVLVSEL SMVEATPELL ALVGEEGAAE PYRYLMKNLR |
| 321- | SRLMATQAWL EARLKGEELP KPEGLLTQNE ELWEPLYACY |
| 361- | QSLQACGMGI IANGDLLDTL RRVKCFGVPL VRIDIRQEST |
| 401- | RHTEALGELT RYLGIGDYES WSEADKQAFL IRELNSKRPL |
| 441- | LPRNWQPSAE TREVLDTCQV IAEAPQGSIA AYVISMAKTP |
| 481- | SDVLAVHLLL KEAGIGFAMP VAPLFETLDD LNNANDVMTQ |
| 521- | LLNIDWYRGL IQGKQMVMIG YSDSAKDAGV MAASWAQYQA |
| 561- | QDALIKTCEK AGIELTLFHG RGGSIGRGGA PAHAALLSQP |
| 601- | PGSLKGGLRV TEQGEMIRFK YGLPEITVSS LSLYTGAILE |
| 641- | ANLLPPPEPK ESWRRIMDEL SVISCDVYRG YVRENKDFVP |
| 681- | YFRSATPEQE LGKLPLGSRP AKRRPTGGVE SLRAIPWIFA |
| 721- | WTQNRLMLPA WLGAGTALQK VVEDGKQSEL EAMCRDWPFF |
| 761- | STRLGMLEMV FAKADLWLAE YYDQRLVDKA LWPLGKELRN |
| 801- | LQEEDIKVVL AIANDSHLMA DLPWIAESIQ LRNIYTDPLN |
| 841- | VLQAELLHRS RQAEKEGQEP DPRVEQALMV TIAGIAAGMR |
| 881- | NTG -883 |

SEQ ID NO: 9. Pichia kudriavzevii pyruvate decarboxylase.

| | |
|---|---|
| 1- | MTDKISLGTY LFEKLKEAGS YSIFGVPGDF NLALLDHVKE |
| 41- | VEGIRWVGNA NELNAGYEAD GYARINGFAS LITTFGVGEL |
| 81- | SAVNAIAGSY AEHVPLIHIV GMPSLSAMKN NLLLHHTLGD |
| 121- | TRFDNFTEMS KKISAKVEIV YDLESAPKLI NNLIETAYHT |
| 161- | KRPVYLGLPS NFADELVPAA LVKENKLHLE EPLNNPVAEE |
| 201- | EFIHNVVEMV KKAEKPIILV DACAARHNIS KEVRELAKLT |
| 241- | KFPVFTTPMG KSTVDEDDEE FFGLYLGSLS APDVKDIVGP |
| 281- | TDCILSLGGL PSDFNTGSFS YGYTTKNVVE FHSNYCKFKS |
| 321- | ATYENLMMKG AVQRLISELK NIKYSNVSTL SPPKSKFAYE |
| 361- | SAKVAPEGII TQDYLWKRLS YFLKPRDIIV TETGTSSFGV |
| 401- | LATHLPRDSK SISQVLWGSI GFSLPAAVGA AFAAEDAHKQ |
| 441- | TGEQERRTVL FIGDGSLQLT VQSISDAARW NIKPYIFILN |
| 481- | NRGYTIEKLI HGRHEDYNQI QPWDHQLLLK LFADKTQYEN |
| 521- | HVVKSAKDLD ALMKDEAFNK EDKIRVIELF LDEFDAPEIL |
| 561- | VAQAKLSDEI NSKAA -575 |

SEQ ID NO: 10. Saccharomyces cerevisiae PDC1.

| | |
|---|---|
| 1- | MSEITLGKYL FERLKQVNVN TVFGLPGDFN LSLLDKIYEV |
| 41- | EGMRWAGNAN ELNAAYAADG YARIKGMSCI ITTFGVGELS |
| 81- | ALNGIAGSYA EHVGVLHVVG VPSISAQAKQ LLLHHTLGNG |
| 121- | DFTVFHRMSA NISETTAMIT DIATAPAEID RCIRTTYVTQ |
| 161- | RPVYLGLPAN LVDLNVPAKL LQTPIDMSLK PNDAESEKEV |
| 201- | IDTILALVKD AKNPVILADA CCSRHDVKAE TKKLIDLTQF |
| 241- | PAFVTPMGKG SIDEQHPRYG GVYVGTLSKP EVKEAVESAD |
| 281- | LILSVGALLS DFNTGSFSYS YKTKNIVEFH SDHMKIRNAT |
| 321- | FPGVQMKFVL QKLLTTIADA AKGYKPVAVP ARTPANAAVP |
| 361- | ASTPLKQEWM WNQLGNFLQE GDVVIAETGT SAFGINQTTF |
| 401- | PNNTYGISQV LWGSIGFTTG ATLGAAFAAE EIDPKKRVIL |
| 441- | FIGDGSLQLT VQEISTMIRW GLKPYLFVLN NDGYTIEKLI |
| 481- | HGPKAQYNEI QGWDHLSLLP TFGAKDYETH RVATTGEWDK |
| 521- | LTQDKSFNDN SKIRMIEIML PVFDAPQNLV EQAKLTAATN |
| 561- | AKQ -563 |

SEQ ID NO: 11. Pichia kudriavzevii alcohol dehydrogenase (ADH1).

| | |
|---|---|
| 1- | MFASTFRSQA VRAARFTRFQ STFAIPEKQM GVIFETHGGP |
| 41- | LQYKEIPVPK PKPTEILINV KYSGVCHTDL HAWKGDWPLP |
| 81- | AKLPLVGGHE GAGIVVAKGS AVTNFEIGDY AGIKWLNGSC |
| 121- | MSCEFCEQGD ESNCEHADLS GYTHDGSFQQ YATADAIQAA |
| 161- | KIPKGTDLSE VAPILCAGVT VYKALKTADL RAGQWVAISG |
| 201- | AAGGLGSLAV QYAKAMGLRV LGIDGGEGKK ELFEQCGGDV |
| 241- | FIDFTRYPRD APEKMVADIK AATNGLGPHG VINVSVSPAA |
| 281- | ISQSCDYVRA TGKVVLVGMP SGAVCKSDVF THVVKSLQIK |
| 321- | GSYVGNRADT REALEFFNEG KVRSPIKVVP LSTLPEIYEL |
| 361- | MEQGKILGRY VVDTSK -376 |

SEQ ID NO: 12. Pichia kudriavzevii glycerol 3-phosphate dehydrogenase.

| | |
|---|---|
| 1- | MVSPAERLST IASTIKPNRK DSTSLQPEDY PEHPFKVTVV |
| 41- | GSGNWGCTIA KVIAENTVER PRQFQRDVNM WVYEELIEGE |
| 81- | KLTEIINTKH ENVKYLPGIK LPVNVVAVPD IVEACAGSDL |
| 121- | IVFNIPHQFL PRILSQLKGK VNPKARAISC LKGLDVNPNG |
| 161- | CKLLSTVITE ELGIYCGALS GANLAPEVAQ CKWSETTVAY |
| 201- | TIPDDFRGKG KDIDHQILKS LFHRPYFHVR VISDVAGISI |
| 241- | AGALKNVVAM AAGFVEGLGW GDNAKAAVMR IGLVETIQFA |
| 281- | KTFFDGCHAA TFTHESAGVA DLITTCAGGR NVRVGRYMAQ |
| 321- | HSVSATEAEE KLLNGQSCQG IHTTREVYEF LSNMGRTDEF |
| 361- | PLFTTTYRII YENFPIEKLP ECLEPVED -388 |

SEQ ID NO: 13. Pichia kudriavzevii cytosolic malate dehydrogenase

| | |
|---|---|
| 1- | MSNVKVALLG AAGGIGQPLA LLLKLNPNIT HLALYDVVHV |
| 41- | PGVAADLHHI DTDVVITHHL KDEDGTALAN ALKDATFVIV |
| 81- | PAGVPRKPGM TRGDLFTINA GICAELANAI SLNAPNAFTL |
| 121- | VITNPVNSTV PIFKEIFAKN EAFNPRRLFG VTALDHVRSN |
| 161- | TFLSELIDGK NPQHFDVTVV GGHSGNSIVP LFSLVKAAEN |
| 201- | LDDEIIDALI HRVQYGGDEV VEAKSGAGSA TLSMAYAANK |
| 241- | FFNILLNGYL GLKKTMISSY VFLDDSINGV PQLKENLSKL |
| 281- | LKGSEVELPT YLAVPMTYGK EGIEQVFYDW VFEMSPKEKE |
| 321- | NFITAIEYID QNIEKGLNFM VR -342 |

SEQ ID NO: 14. L-aspartate dehydrogenase consensus sequence

| | |
|---|---|
| 1- | MLHIAMIGCG AIGAGVLELL KSDPDLRVDA VIVPEESMDA |
| 41- | VREAVAALAP VARVLTALPA DARPDLLVEC AGHRAIEEHV |
| 81- | VPALERGIPC AVASVGALSE PGLAERLEAA ARRGGTQVQL |
| 121- | LSGAIGAIDA LAAARVGGLD SVVYTGRKPP LAWKGTPAEQ |
| 161- | VCDLDALTEA TVIFEGSARE AARLYPKNAN VAATLSLAGL |
| 201- | GLDRTQVRLI ADPAVTENVH HVEARGAFGG FELTMRGKPL |
| 241- | AANPKTSALT VYSVVRALGN RAHALSI -267 |

SEQ ID NO: 15. Bacterial L-aspartate 1-decarboxylase consensus sequence

| | |
|---|---|
| 1- | MLRTMLKSKI HRATVTQADL HYVGSVTIDA DLLDAADILE |
| 41- | GEKVAIVDIT NGARLETYVI AGERGSGVIG INGAAAHLVH |
| 81- | PGDLVIIIAY AQMSDAEARA YEPRVVFVDA DNRIVEXLGN |
| 121- | DPAEALPGG -129 |

SEQ ID NO: 16. Eukaryotic L-aspartate 1-decarboxylase consensus sequence

| | |
|---|---|
| 1- | MPANGNFPVA LEVISIFKPY NSAVEDLASM AKTDTSASSS |
| 41- | GSDSAGSSED EDVQLFASKG NLLNSKLLKK SNNNNKNNNI |
| 81- | NENNNKNAAA GLKRFASLPN RAEHEEFLRD CVDEILKLAV |
| 121- | FEGTNRSSKV VEWHDPEELK KLFDFELRAE PDSHEKLLEL |
| 161- | LRATIRYSVK TGHPYFVNQL FSSVDPYGLV GQWLTDALNP |
| 201- | SVYTYEVAPV FTLMEEVVLR EMRRIVGFPN DGEGDGIFCP |
| 241- | GGSIANGYAI SCARYKYAPE VKKKGLHSLP RLVIFTSEDA |
| 281- | HYSVKKLASF MGIGSDNVYK IATDEVGKMR VSDLEQEILR |
| 321- | ALDEGAQPFM VSATAGTTVI GAFDPLEGIA DLCKKYNLWM |
| 361- | HVDAAWGGGA LMSKKYRHLL KGIERADSVT WNPHKLLAAP |
| 401- | QQCSTFLTRH EGILSECHST NATYLFQKDK FYDTSYDTGD |
| 441- | KHIQCGRRAD VLKFWFMWKA KGTSGFEAHV DKVFENAEYF |
| 481- | TDSIKARPGF ELVIEEPECT NICFWYVPPS LRGMERDNAE |
| 521- | FYEKLHKVAP KIKERMIKEG SMMITYQPLR DLPNFFRLVL |
| 561- | QNSGLDKSDM LYFINEIERL GSDLV -585 |

SEQ ID NO: 17. Ralstonia solanacearum L-aspartate dehydrogenase.

| | |
|---|---|
| 1- | MLHVSMVGCG AIGQGVLELL KSDPDLCFDT VIVPEHGMDR |
| 41- | ARAAIAPFAP RTRVMTRLPA QADRPDLLVE CAGHDALREH |
| 81- | VVPALEQGID CLVVSVGALS EPGLAERLEA AARRGHAQMQ |
| 121- | LLSGAIGAID ALAAARVGGL DAVVYTGRKP PRAWKGTPAE |
| 161- | RQFDLDALDR TTVIFEGKAS DAALLFPKNA NVAATLALAG |
| 201- | LGMERTHVRL LADPTIDENI HHVEARGAFG GFELIMRGKP |
| 241- | LAANPKTSAL TVFSVVRALG NRAHAVSI -268 |

SEQ ID NO: 18. Polaromonas sp. L-aspartate dehydrogenase.

| | |
|---|---|
| 1- | MLKIAMIGCG AIGASVLELL HGDSDVVVDR VITVPEARDR |
| 41- | TEIAVARWAP RARVLEVLAA DDAPDLVVEC AGHGAIAAHV |
| 81- | VPALERGIPC VVTSVGALSA PGMAQLLEQA ARRGKTQVQL |
| 121- | LSGAIGGIDA LAAARVGGLD SVVYTGRKPP MAWKGTPAEA |
| 161- | VCDLDSLTVA HCIFDGSAEQ AAQLYPKNAN VAATLSLAGL |
| 201- | GLKRTQVQLF ADPGVSENVH HVAAHGAFGS FELTMRGRPL |
| 241- | AANPKTSALT VYSVVRALLN RGRALVI -267 |

SEQ ID NO: 19. Burkholderia thailandensis L-aspartate dehydrogenase.

| | |
|---|---|
| 1- | MRNAHAPVDV AMIGFGAIGA AVYRAVEHDA ALRVAHVIVP |
| 41- | EHQCDAVRGA LGERVDVVSS VDALAYRPQF ALECAGHGAL |
| 81- | VDHVVPLLRA GTDCAVASIG ALSDLALLDA LSEAADEGGA |
| 121- | TLTLLSGAIG GVDALAAAKQ GGLDEVQYIG RKPPLGWLGT |
| 161- | PAEALCDLRA MTAEQTIFEG SARDAARLYP KNANVAATVA |
| 201- | LAGVGLDATK VRLIADPAVT RNVHRVVARG AFGEMSIEMS |
| 241- | GKPLPDNPKT SALTAFSAIR ALRNRASHCV I -271 |

SEQ ID NO: 20. Burkholderia pseudomallei L-aspartate dehydrogenase.

| | |
|---|---|
| 1- | MRNAHAPVDV AMIGFGAIGA AVYRAVEHDA ALRVAHVIVP |
| 41- | EHQCDAVRGA LGERVDVVSS VDALACRPQF ALECAGHGAL |
| 81- | VDHVVPLLKA GTDCAVASIG ALSDLALLDA LSNAADAGGA |
| 121- | TLTLLSGAIG GIDALAAARQ GGLDEVRYIG RKPPLGWLGT |
| 161- | PAEAICDLRA MAAEQTIFEG SARDAAQLYP RNANVAATIA |
| 201- | LAGVGLDATR VCLIADPAVT RNVHRIVARG AFGEMSIEMS |
| 241- | GKPLPDNPKT SALTAFSAIR ALRNRASHCV I -271 |

SEQ ID NO: 21. Ochrobactrum anthropi L-aspartate dehydrogenase.

| | |
|---|---|
| 1- | MSVSETIVLV GWGAIGKRVA DLLAERKSSV RIGAVAVRDR |
| 41- | SASRDRLPAG AVLIENPAEL AASGASLVVE AAGRPSVLPW |
| 81- | GEAALSTGMD FAVSSTSAFV DDALFQRLKD AAAASGAKLI |
| 121- | IPPGALGGID ALSAASRLSI ESVEHRIIKP AKAWAGTQAA |
| 161- | QLVPLDEISE ATVFFTDTAR KAADAFPQNA NVAVITSLAG |
| 201- | IGLDRTRVTL VADPAARLNT HEIIAEGDFG RMHLRFENGP |
| 241- | LATNPKSSEM TALNLVRAIE NRVATTVI -268 |

SEQ ID NO: 22. Acinetobacter sp. SH024 L-aspartate dehydrogenase.

| | |
|---|---|
| 1- | MKKLMMIGFG AMAAEVYAHL PQDLQLKWIV VPSRSIEKVQ |
| 41- | SQVSSEIQVI SDIEQCDGTP DYVIEVAGQA AVKEHAQKVL |
| 81- | AKGWTIGLIS VGTLADSEFL IQLKQTAEKN DAHLHLLAGA |
| 121- | IAGIDGISAA KEGGLQKVTY KGCKSPKSWK GSYAEQLVDL |
| 161- | DHVVEATVFF TGTAREAATK FPANANVAAT IALAGLGMDE |
| 201- | TMVELTVDPT INKNKHTIVA EGGFGQMTIE LVGVPLPSNP |
| 241- | KTSTLAALSV IRACRNSVEA IQI -263 |

SEQ ID NO: 23. Klebsiella pneumoniae L-aspartate dehydrogenase.

| | |
|---|---|
| 1- | MMKKVMLIGY GAMAQAVIER LPPQVRVEWI VARESHHAAI |
| 41- | CLQFGQAVTP LTDPLQCGGT PDLVLECASQ QAVAQYGEAV |
| 81- | LARGWHLAVI STGALADSEL EQRLRQAGGK LTLLAGAVAG |
| 121- | IDGLAAAKEG GLERVTYQSR KSPASWRGSY AEQLIDLSAV |
| 161- | NEAQIFFEGS AREAARLFPA NANVAATIAL GGIGLDATRV |
| 201- | QLMVDPATQR NTHTLHAEGL FGEFHLELSG LPLASNPKTS |
| 241- | TLAALSAVRA CRELA -255 |

SEQ ID NO: 24. Dinoroseobacter shibae L-aspartate dehydrogenase.

| | |
|---|---|
| 1- | MRLALIGLGA INRAVAAGMA GQAEMVALTR SGAEAPGVMA |
| 41- | VSDLSALRVF APDLVVEAAG HGAARAYLPG LLAAGIDVLM |
| 81- | ASVGVLADPE TEAAFRAAPA HGAQLTIPAG AIGGLDLLAA |
| 121- | LPKDSLRAVR YTGVKPPAAW AGSPAADGRD LSALDGPVTL |
| 161- | FEGTARQAAL RFPNNANVAA TLALAGAGFD RTEARLVADP |
| 201- | DAAGNGHAYD VISDTAEMTF SVRARPSDTP GTSATTAMSL |
| 241- | LRAIRNRDAA WVV -253 |

SEQ ID NO: 25. Ruegeria pomeroyi L-aspartate dehydrogenase.

| | |
|---|---|
| 1- | MWKLWGSWPE GDRVRIALIG HGPIAAHVAA HLPVGVQLTG |
| 41- | ALCRPGRDDA ARAALGVSVA QALEGLPQRP DLLVDCAGHS |
| 81- | GLRAHGLTAL GAGVEVLTVS VGALADAVFC AELEDAARAG |
| 121- | GTRLCLASGA IGALDALAAA AMGTGLQVTY TGRKPPQGWR |
| 161- | GSRAEKVLDL KALTGPVTHF TGTARAAAQA YPKNANVAAA |
| 201- | VALAGAGLDA TRAELIADPG AAANIHEIAA EGAFGRFRFQ |
| 241- | IEGLPLPGNP RSSALTALSL LAALRQRGAA IRPSF -275 |

SEQ ID NO: 26. Comamonas testosteroni L-aspartate dehydrogenase.

| | |
|---|---|
| 1- | MKNIALIGCG AIGSSVLELL SGDTQLQVGW VLVPEITPAV |
| 41- | RETAARLAPQ AQLLQALPGD AVPDLLVECA GHAAIEEHVL |
| 81- | PALARGIPAV IASIGALSAP GMAERVQAAA ETGKTQAQLL |
| 121- | SGAIGGIDAL AAARVGGLET VLYTGRKPPK AWSGTPAEQV |
| 161- | CDLDGLTEAF CIFEGSAREA AQLYPKNANV AATLSLAGLG |
| 201- | LDKTMVRLFA DPGVQENVHQ VEARGAFGAM ELTMRGKPLA |
| 241- | ANPKTSALTV YSVVRAVLNN VAPLAI -266 |

SEQ ID NO: 27. Cupriavidus pinatubonensis L-aspartate dehydrogenase.

| | |
|---|---|
| 1- | MSMLHVSMVG CGAIGRGVLE LLKADPDVAF DVVIVPEGQM |
| 41- | DEARSALSAL APNVRVATGL DGQRPDLLVE CAGHQALEEH |
| 81- | IVPALERGIP CMVVSVGALS EPGLVERLEA AARRGNTQVQ |
| 121- | LLSGAIGAID ALAAARVGGL DEVIYTGRKP ARAWTGTPAA |
| 161- | ELFDLEALTE PTVIFEGTAR DAARLYPKNA NVAATVSLAG |
| 201- | LGLDRTSVRL LADPNAVENV HHIEARGAFG GFELTMRGKP |
| 241- | LAANPKTSAL TVFSVVRALG NRAHAVSI -268 |

SEQ ID NO: 28. Plasmid vector pTL3.
gacgaaagggcctcgtgatacgcctatttttataggttaatgtcatgataataatggtttcttaggacggatcgcttgcctgtaacttaca
cgcgcctcgtatcttttaatgatggaataatttgggaatttactctgtgtttatttatttttatgttttgtatttggattttagaaagtaa
ataaagaaggtagaagagttacggaatgaagaaaaaaaaataaacaaaggtttaaaaaatttcaacaaaaagcgtactttacatatatatt
tattagacaagaaaagcagattaaatagatatacattcgattaacgataagtaaaatgtaaaatcacaggattttcgtgtgtggtcttcta
cacagacaagatgaaacaattcggcattaatacctgagagcaggaagagcaagataaaaggtagtatttgttggcgatccccctagagtct
tttacatcttcggaaaacaaaaactattttttctttaatttctttttttactttctatttttaatttatatatttatattaaaaaatttaa
attataattatttttatagcacgtgatgaaaaggacccaggtggcacttttcggggaaatgtgcgcggaacccctatttgtttatttttct
aaatacattcaaatatgtatccgctcatgagacaataaccctgataaatgcttcaataatattgaaaaaggaagagtatgagtattcaaca
tttccgtgtcgcccttattcccttttttgcggcattttgccttcctgtttttgctcacccagaaacgctggtgaaagtaaaagatgctgaa
gatcagttgggtgcacgagtgggttacatcgaactggatctcaacagcggtaagatccttgagagttttcgccccgaagaacgttttccaa
tgatgagcacttttaaagttctgctatgtggcgcggtattatcccgtattgacgccgggcaagagcaactcggtcgccgcatacactattc
tcagaatgacttggttgagtactcaccagtcacagaaaagcatcttacggatggcatgacagtaagagaattatgcagtgctgccataacc
atgagtgataacactgcggccaacttacttctgacaacgatcggaggaccgaaggagctaaccgcttttttgcacaacatgggggatcatg
taactcgccttgatcgttgggaaccggagctgaatgaagccataccaaacgacgagcgtgacaccacgatgcctgtagcaatggcaacaac
gttgcgcaaactattaactggcgaactacttactctagcttcccggcaacaattaatagactggatggaggcggataaagttgcaggacca
cttctgcgctcggcccttccggctggctggtttattgctgataaatctggagccggtgagcgtgggtctcgcggtatcattgcagcactgg
ggccagatggtaagccctcccgtatcgtagttatctacacgacggggagtcaggcaactatggatgaacgaaatagacagatcgctgagat
aggtgcctcactgattaagcattggtaactgtcagaccaagtttactcatatatactttagattgatttaaaacttcatttttaatttaaa
aggatctaggtgaagatcctttttgataatctcatgaccaaaatcccttaacgtgagttttcgttccactgagcgtcagaccccgtagaaa
agatcaaaggatcttcttgagatcctttttttctgcgcgtaatctgctgcttgcaaacaaaaaaaccaccgctaccagcggtggtttgttt
gccggatcaagagctaccaactctttttccgaaggtaactggcttcagcagagcgcagataccaaatactgttcttctagtgtagccgtag
ttaggccaccacttcaagaactctgtagcaccgcctacatacctcgctctgctaatcctgttaccagtggctgctgccagtggcgataagt
cgtgtcttaccgggttggactcaagacgatagttaccggataaggcgcagcggtcgggctgaacggggggttcgtgcacacagcccagctt
ggagcgaacgacctacaccgaactgagatacctacagcgtgagctatgagaaagcgccacgcttcccgaagggagaaaggcggacaggtat
ccggtaagcggcagggtcggaacaggagagcgcacgagggagcttccagggggaaacgcctggtatctttatagtcctgtcgggtttcgcc
acctctgacttgagcgtcgatttttgtgatgctcgtcaggggggcggagcctatggaaaaacgccagcaacgcggcctttttacggttcct
ggccttttgctggccttttgctcacatgttctttcctgcgttatcccctgattctgtggataaccgtattaccgcctttgagtgagctgat
accgctcgccgcagccgaacgaccgagcgcagcgagtcagtgagcgaggaagcggaagagcgcccaatacgcaaaccgcctctccccgcgc
gttggccgattcattaatgcagctgacagtttattcctggcatccactaaatataatggagcccgctttttaagctggcatccagaaaaaa
aaagaatcccagcaccaaaatattgttttcttcaccaaccatcagttcataggtccattctcttagcgcaactacagagaacaggggcaca
aacaggcaaaaaacgggcacaacctcaatggagtgatgcaacctgcctggagtaaatgatgacacaaggcaattgacccacgcatgtatct
atctcattttcttacaccttctattaccttctgctctctctgatttggaaaaagctgaaaaaaaaggttgaaaccagttccctgaaattat
tcccctacttgactaataagtatataaagacggtaggtattgattgtaattctgtaaatctatttcttaaacttcttaaattctactttta
tagttagtcttttttttagttttaaaacaccaagaacttagtttcgaataaacacacataaacaaacaaaagtttaaacgattaatataat
tatataaaaatattatcttcttttctttatatctagtgttatgtaaaataaattgatgactacggaaagcttttttatattgtttcttttt
cattctgagccacttaaatttcgtgaatgttcttgtaagggacggtagatttacaagtgatacaacaaaaagcaaggcgctttttctaata
aaaagaagaaaagcatttaacaattgaacacctctatatcaacgaagaatattactttgtctctaaatccttgtaaaatgtgtacgatctc
tatatgggttactcacagctggcgtaatagcgaagaggcccgcaccgatcgcccttcccaacagttgcgcagcctgaatggcgaatggacg
cgccctgtagcggcgcattaagcgcggcgggtgtggtggttacgcgcagcgtgaccgctacacttgccagcgccctagcgcccgctccttt
cgctttcttcccttcctttctcgccacgttcgccggctttccccgtcaagctctaaatcgggggctccctttagggttccgatttagtgct
ttacggcacctcgaccccaaaaaacttgattagggtgatggttcacgtagtgggccatcgccctgatagacggtttttcgccctttgacgt
tggagtccacgttctttaatagtggactcttgttccaaactggaacaacactcaaccctatctcggtctattcttttgatttataagggat
tttgccgatttcggcctattggttaaaaaatgagctgatttaacaaaaatttaacgcgaattttaacaaaatattaacgcttacaatttcc
tgatgcggtattttctccttacgcatctgtgcggtatttcacaccgcatagggtaataactgatataattaaattgaagctctaatttgtg
agtttagtatacatgcatttacttataatacagttttttagttttgctggccgcatcttctcaaatatgcttcccagcctgcttttctgta
acgttcaccctctaccttagcatcccttccctttgcaaatagtcctcttccaacaataataatgtcagatcctgtagagaccacatcatcc
acggttctatactgttgacccaatgcgtctcccttgtcatctaaacccacaccgggtgtcataatcaaccaatcgtaaccttcatctcttc
cacccatgtctctttgagcaataaagccgataacaaaatctttgtcgctcttcgcaatgtcaacagtacccttagtatattctccagtaga
tagggagcccttgcatgacaattctgctaacatcaaaaggcctctaggttcctttgttacttcttctgccgcctgcttcaaaccgctaaca
atacctgggcccaccacaccgtgtgcattcgtaatgtctgcccattctgctattctgtatacacccgcagagtactgcaatttgactgtat
taccaatgtcagcaaattttctgtcttcgaagagtaaaaaattgtacttggcggataatgcctttagcggcttaactgtgccctccatgga
aaaatcagtcaagatatccacatgtgtttttagtaaacaaattttgggacctaatgcttcaactaactccagtaattccttggtggtacga
acatccaatgaagcacacaagtttgtttgcttttcgtgcatgatattaaatagcttggcagcaacaggactaggatgagtagcagcacgtt
ccttatatgtagctttcgacatgatttatcttcgtttcctgcaggtttttgttctgtgcagttgggttaagaatactgggcaatttcatgt
ttcttcaacactacatatgcgtatatataccaatctaagtctgtgctccttccttcgttcttccttctgttcggagattaccgaatcaaaa
aaatttcaaagaaaccgaaatcaaaaaaaagaataaaaaaaaaatgatgaattgaattgaaaagctgtggtatggtgcactctcagtacaa
tctgctctgatgccgcatagttaagccagccccgacacccgccaacacccgctgacgcgccctgacgggcttgtctgctcccggcatccgc
ttacagacaagctgtgaccgtctccgggagctgcatgtgtcagaggttttcaccgtcatcaccgaaacgcgcga -5261
SEQ ID NO: 29. Disrupted Pichia kudriazevii pyruvate decarboxylase PDC5

| | |
|---|---|
| 1- | MLQTANSEVP NASQITIDAA SGLPADRVLP NITNTEITIS |
| 41- | EYIFYRILQL GVRSVFGVPG DFNLRFLEHI YDVHGLNWIG |
| 81- | CCNELNAAYA ADAYAKASKK MGVLLTTYGV GELSALNGVA |
| 121- | GAYTEFAPVL HLVGTSALKF KRNPRTLNLH HLAGDKKTFK |
| 161- | KSDHYKYERI ASEFSVDSAS IEDDPIEACE MIDRVIYSTW |
| 181- | RESRPGYIFL PCDLSEMKVD AQRLASPIEL TYRFNSPVSR |
| 221- | VEGVADQILQ LIYQNKNVSI IVDGFIRKFR MESEFYDIME |
| 261- | KFGDKVNIFS TMYGKGLIGE EHPRFVGTYF GKYEKAVGNL |
| 301- | LEASDLIIHF GNFDHELNMG GFTFNIPQEK YIDLSAQYVD |
| 341- | ITGNLDESIT MMEVLPVLAS KLDSSRVNVA DKFEKFDKYY |
| 381- | ETPDYQREAS LQETDIMQSL NENLTGDDIL IVETCSFLFA |
| 421- | VPDLKVKQHT NIILQAYWAS IGYALPATLG ASLAIRDFNL |
| 461- | SGKVYTIEGD GSAQMSLQEL SSMLRYNIDA TMILLNNSGY |
| 501- | TIERVIVGPH SSYNDINTNW QWTDLLRAFG DVANEKSVSY |
| 541- | TIKEREQLLN ILSDPSFKHN GKFRLLECVL PMFDVPKKLG 600 |

SEQ ID NO: 30. Disrupted Pichia kudriazevii pyruvate decarboxylase PDC6

| | |
|---|---|
| 1- | MAPVSLETCT LEFSCKLPLS EYIFRRIASL GIHNIFGVPG |
| 41- | DYNLSFLEHL YSVPELSWVG CCNELNSAYA TDGYSRTIGH |
| 81- | DKFGVLLTTQ GVGELSAANA IAGSFAEHVP ILHIVGTTPY |
| 121- | SLKHKGSHHH HLINGVSTRE PTNHYAYEEM SKNISCKILS |
| 161- | LSDDLTNAAN EIDDLFRTIL MLKKPGYLYI PCDLVNVEID |
| 201- | ASNLQSVPAN KLRERVPSTD SQTIAKITST IVDKLLSSSN |
| 241- | PVVLCDILTD RYGMTAYAQD LVDSLKVPCC NSFMGKALLN |
| 281- | ESKEHYIGDF NGEESNKMVH SYISNTDCFL HIGDYYNEIN |
| 321- | SGHWSLYNGI NKESIVILNP EYVKIGSQTY QNVSFEDILP |
| 361- | AILSSIKANP NLPCFHIPKI MSTIEQIPSN TPISQTLMLE |
| 401- | KLQSFLKPND VLVTETCSLM FGLPDIRMPE NSKVIGQHFY |
| 441- | LSIGMALPCS FGVSVALNEL KKDSRLILIE GDGSAQMTVQ |
| 481- | ELSNFNRENV VKPLIILLNN SGYTVERVIK GPKREYNDIR |
| 521- | PDWKWTQLLQ TFGMDDAKSM KVTTPEELDD ALDEYGNNLS |
| 561- | TPRLLEVVLD KLDVPWRFNK MVGN -584 |

SEQ ID NO: 31. Disrupted Pichia kudriazevii glyceraldehyde 3-phosphate dehydrogenase

| | |
|---|---|
| 1- | MVSPAERLST IASTIKPNRK DSTSLQPEDY PEHPFKVTVV |
| 41- | GSGNWGCTIA KVIAENTVER PRQFQRDVNM WVYEELIEGE |
| 81- | KLTEIINTKH ENVKYLPGIK LPVNVVAVPD IVEACAGSDL |
| 121- | IVFNIPHQFL PRILSQLKGK VNPKARAISC LKGLDVNPNG |
| 161- | CKLLSTVITE ELGIYCGALS GANLAPEVAQ CKWSETTVAY |
| 201- | TIPDDFRGKG KDIDHQILKS LFHRPYFHVR VISDVAGISI |
| 241- | AGALKNVVAM AAGFVEGLGW GDNAKAAVMR IGLVETIQFA |
| 281- | KTFFDGCHAA TFTHESAGVA DLITTCAGGR NVRVGRYMAQ |
| 321- | HSVSATEAEE KLLNGQSCQG IHTTREVYEF LSNMGRTDEF |
| 361- | PLFTTTYRII YENFPIEKLP ECLEPVED -388 |

SEQ ID NO: 32. Disrupted Pichia kudriazevii aspartate aminotransferase

| | |
|---|---|
| 1- | MSRGFFTENI TQLPPDPLFG LKARFSNDSR ENKVDLGIGA |
| 41- | YRDDNGKPWI LPSVRLAENL IQNSPDYNHE YLPIGGLADF |
| 81- | TSAAARVVFG GDSKAISQNR LVSIQSLSGT GALHVAGLFI |
| 121- | KRQYKSLDGT SEDPLIYLSE PTWANHVQIF EVIGLKPVFY |
| 161- | PYWHAASKTL DLKGYLKAIN DAPEGSVFVL HATAHNPTGL |
| 201- | DPTQEQWMEI LAAISAKKHL PLFDCAYQGF TSGSLDRDAW |
| 241- | AVREAVNNDK YEFPGIIVCQ SFAKNVGMYG ERIGAVHIVL |
| 281- | PESDASLNSA IFSQLQKTIR SEISNPPGYG AKIVSKVLNT |
| 321- | PELYKQWEQD LITMSSRITA MRKELVNELE RLGTPGTWRH |
| 361- | ITEQQGMFSF TGLNPEQVAK LEKEHGVYLV RSGRASIAGL |
| 401- | NMGNVKYVAK AIDSVVRDL -419 |

SEQ ID NO: 33. Disrupted Pichia kudriazevii urea amidolyase

| | |
|---|---|
| 1- | MNTIGWSVSD WVSFNRETTP DESFNTLKAL VDYIKSTPND |
| 41- | PAWISIISEE NLNHQWNILQ SKSNKPSLKL YGVPIAVKDN |
| 81- | IDALGFPTTA ACPSFSYMPT SDSTIVSLLR DQGAIIIGKT |
| 121- | NLDQFATGLV GTRSPYGITP CVFSDKHVSG GSSAGSASVV |
| 161- | ARGLVPIALG TDTAGSGRVP AALNNIIGLK PTVGAFSTNG |
| 201- | VVPACKSLDC PSIFSLNLND AQLVFNICAK PDLTNCEYSR |
| 241- | EGPQNYKRKF TGKVKIAIPI DFNGLWFNDE ENPKIFNDAI |
| 281- | ENFKKLNVEI VPIDFNPLLE LAKCLYEGPW VSERYSAVKS |
| 321- | FYKSNPKKED LDPIVTKIIE NGANYDASTA FEYEYKRRGI |
| 361- | LNKVKLLIKD IDALLVPTCP LNPTIEQVLK EPIKVNSIQG |
| 401- | TWTNFCNLAD FAALALPNGF RNDGLPNGFT LLGRAFEDYA |
| 441- | LLSLAKDYFN AKYPKHDRSI GNIKDKTSGV EDLLDNSLPQ |
| 481- | PNLNSSIKLA VVGAHLEGLP LYWQLEKVQA YKLETTKTSS |
| 521- | NYKLYALPNS NKNSIMKPGL RRISSSNEVG GSQIEVEVYS |
| 561- | IPLENFGDFI SMVPQPLGIG SVELESGEWV KSFICEECGY |
| 601- | KENGSIEITH FGGWRNYLKH LNLNSRLEKS KKPFNKVLVA |
| 641- | NRGEIAVRII KTLKKLNIIS VAVYSDPDKY SDHVLLADEA |
| 681- | YPLNGISASE TYINIEKMLK VIKLSKAEAV IPGYGFLSEN |
| 721- | ADFADKLIEE GIVWVGPSGD TIRKLGLKHS AREIAKNAGV |
| 761- | PLVPGSNLIN DSLEAKEIAQ KLEYPIMIKS TAGGGGIGLQ |
| 801- | KVDSEDDIER VFETVQHQGK SYFGDSGVFL ERFVENSRHV |
| 841- | EIQIFGDGNG NAIAIGERDC SLQRRNQKVI EETPAPNLPE |
| 881- | ITRKKMRKAA EQLASSMNYK CAGTVEFIYD EKRDEFYFLE |
| 921- | VNTRLQVEHP ITEMVTGLDL VEWMLFIAAD MPPDFNQVIP |
| 961- | VEGASMEARL YAENPVKDFK PSPGQLIEVK FPEFARVDTW |
| 1001- | VKTGTIISSE YDPTLAKIIV HGKDRIDALN KLRKALNETV |
| 1041- | IYGCITNIDY LRSIANSKMF EDAKMHTKIL DTFDYKPNAF |
| 1081- | EILSPGAYTT VQDYPGRVGY WRIGVPPSGP MDSYSFRLAN |
| 1121- | RIVGNHYKSP AIEITLNGPS ILFHHETVIA ITGGEVPVTL |
| 1161- | NDERVNMYEP INIKRGDKLV IGKLTTGCRS YLSIRGGIDV |
| 1201- | TEYLGSRSTF ALGNLGGYNG RVLKMGDVLF LSQPGLSSNK |
| 1241- | LPEPISKPQI APTSVIPQIS TTKEWTVGVT CGPHGSPDFF |
| 1281- | TAESIKDFFS NPWKVHYNSN RFGVRLIGPK PKWARNDGGE |
| 1321- | GGLHPSNAHD YVYSLGAINF TGDEPVILTC DGPSLGGFVC |
| 1361- | QAVVADAEMW KIGQVKPGDS INFVPISFDQ AIELKQQQNS |
| 1401- | LIESLSGEYN SIAIAKPLSE PEDPVLAVYQ ANDHSPKITY |
| 1441- | RQAGDRYVLV EYGENIMDLN YSYRVHKLIE MVESHKTIGI |
| 1481- | IEMSQGVRSV LIEYDGFEIH QKVLVKTLLS YEAEVAFTN |
| 1521- | WSVPSRVIRL PMAFEDRQTL DAVKRYQETI RSDAPWLPNN |
| 1561- | VDFIANINGI ERSEVKDMLY SARFLVLGLG DVFLGAPCAV |
| 1601- | PLDPRQRFLG TKYNPSRTFT PNGTVGIGGM YMCIYTMESP |
| 1641- | GGYQLVGRTI PIWDKLSLGE YTKKYNNGKP WLLTPFDQVS |
| 1681- | FYPVTEEELE VMVEDSKHGR FEVDIIESVF DHTKYLSWIT |
| 1721- | ENSDSIEEFQ RQQDGEKLQE FKRLIQVANE DLAKSGTKIV |
| 1761- | ETEEKFPENA ELIYSEYSGR FWKSLVNVGD EVKKGQGLVV |
| 1801- | IEAMKTEMVV NATKDGKVLK IVHGNGDMVD AGDLVVVIA -1839 |

SEQ ID NO: 34. Schizosaccharomyces pombe urease

| | |
|---|---|
| 1- | MQPRELHKLT LHQLGSLAQK RLCRGVKLNK LEATSLIASQ |
| 41- | IQEYVRDGNH SVADLMSLGK DMLGKRHVQP NVVHLLHEIM |
| 81- | IEATFPDGTY LITIHDPICT TDGNLEHALY GSFLPTPSQE |
| 121- | LFPLEEEKLY APENSPGFVE VLEGEIELLP NLPRTPIEVR |
| 161- | NMGDRPIQVG SHYHFIETNE KLCFDRSKAY GKRLDIPSGT |
| 201- | AIRFEPGVMK IVNLIPIGGA KLIQGGNSLS KGVFDDSRTR |
| 241- | EIVDNLMKQG FMHQPESPLN MPLQSARPFV VPRKLYAVMY |
| 281- | GPTTNDKIRL GDTNLIVRVE KDFTEYGNES VFGGGKVIRD |
| 321- | GTGQSSSKSM DECLDTVITN AVIIDHTGIY KADIGIKNGY |
| 361- | IVGIGKAGNP DTMDNIGENM VIGSSTDVIS AENKIVTYGG |
| 401- | MDSHVHFICP QQIEEALASG ITTMYGGGTG PSTGTNATTC |
| 441- | TPNKDLIRSM LRSTDSYPMN IGLTGKGNDS GSSSLKEQIE |
| 481- | AGCSGLKLHE DWGSTPAAID SCLSVCDEYD VQCLIHTDTL |
| 521- | NESSFVEGTF KAFKNRTIHT YHVEGAGGGH APDIISLVQN |
| 561- | PNILPSSTNP TRPFTTNTLD EELDMLMVCH HLSRNVPEDV |
| 601- | AFAESRIRAE TIAAEDILQD LGAISMISSD SQAMGRCGEV |
| 641- | ISRTWKTAHK NKLQRGALPE DEGSGVDNFR VKRYVSKYTI |
| 681- | NPAITHGISH IVGSVEIGKF ADLVLWDFAD FGARPSMVLK |
| 721- | GGMIALASMG DPNGSIPTVS PLMSWQMFGA HDPERSIAFV |
| 761- | SKASITSGVI ESYGLHKRVE AVKSTRNIGK KDMVYNSYMP |
| 801- | KMTVDPEAYT VTADGKVMEC EPVDKLPLSQ SYFIF -835 |

SEQ ID NO: 35. Schizosaccharomyces pombe urease accessory protein D

| | |
|---|---|
| 1- | MEDKEGRFRV ECIENVHYVT DMFCKYPLKL IAPKTKLDFS |
| 41- | ILYIMSYGGG LVSGDRVALD IIVGKNATLC IQSQGNTKLY |
| 81- | KQIPGKPATQ QKLDVEVGTN ALCLLLQDPV QPFGDSNYIQ |
| 121- | TQNFVLEDET SSLALLDWTL HGRSHINEQW SMRSYVSKNC |
| 161- | IQMKIPASNQ RKTLLRDVLK IFDEPNLHIG LKAERMHHFE |
| 201- | CIGNLYLIGP KFLKTKEAVL NQYRNKEKRI SKTTDSSQMK |
| 241- | KIIWTACEIR SVTIIKFAAY NTETARNFLL KLFSDYASFL |
| 281- | DHETLRAFWY -290 |

SEQ ID NO: 36. Schizosaccharomyces pombe urease accessory protein F

| | |
|---|---|
| 1- | MTDSQTETHL SLILSDTAFP LSSFSYSYGL ESYLSHQQVR |
| 41- | DVNAFFNFLP LSLNSVLHTN LPTVKAAWES PQQYSEIEDF |
| 81- | FESTQTCTIA QKVSTMQGKS LLNIWTKSLS FFVTSTDVFK |
| 121- | YLDEYERRVR SKKALGHFPV VWGVVCRALG LSLERTCYLF |
| 161- | LLGHAKSICS AAVRLDVLTS FQYVSTLAHP QTESLLRDSS |
| 201- | QLALNMQLED TAQSWYTLDL WQGRHSLLYS RIFNS -235 |

SEQ ID NO: 37. Schizosaccharomyces pombe urease accessory protein G

| | |
|---|---|
| 1- | MAIPFLHKGG SDDSTHHHTH DYDHHNHDHH GHDHHSHDSS |
| 41- | SNSSSEAARL QFIQEHGHSH DAMETPGSYL KRELPQFNHR |
| 81- | DFSRRAFTIG VGGPVGSGKT ALLLQLCRLL GEKYSIGVVT |
| 121- | NDIFTREDQE FLIRNKALPE ERIRAIETGG CPHAAIREDV |
| 161- | SGNLVALEEL QSEFNTELLL VESGGDNLAA NYSRDLADFI |
| 201- | IYVIDVSGGD KIPRKGGPGI TESDLLIINK TDLAKLVGAD |
| 241- | LSVMDRDAKK IRENGPIVFA QVKNQVGMDE ITELILGAAK |
| 281- | SAGALK -286 |

SEQ ID NO: 38. Schizosaccharomyces pombe nickel transporter

| | |
|---|---|
| 1- | MNSMSEYVKP RKNEFLRKFE NFYFEIPFLS KLPPKVSVPI |
| 41- | FSLISVNIVV WIVAAIVISL VNRSLFLSVL LSWTLGLRHA |
| 81- | LDADHITAID NLTRRLLSTD KPMSTVGTWF SIGHSTVVLI |
| 121- | TCIVVAATSS KFADRWNNFQ TIGGIIGTSV SMGLLLLLAI |
| 161- | GNTVLLVRLS YWLWMYRKSG VTKDEGVTGF LARKMQRLFR |
| 201- | LVDSPWKIYV LGFVFGLGFD TSTEVSLLGI ATLQALKGTS |
| 241- | IWAILLFPIV FLVGMCLVDT TDGALMYYAY SYSSGETNPY |
| 281- | FSRLYYSIIL TFVSVIAAFT IGIIQMLMLI ISVHPMESTF |
| 321- | WNGLNRLSDN YEIVGGCICG AFVLAGLFGI SMHNYFKKKF |
| 361- | TPPVQVGNDR EDEVLEKNKE LENVSKNSIS VQISESEKVS |
| 401- | YDTVDSKV -408 |

SEQ ID NO: 39. Arabidopsis thaliana aspartic acid transporter AtSIAR1

| | |
|---|---|
| 1- | MKGGSMEKIK PILAIISLQF GYAGMYIITM VSFKHGMDHW |
| 41- | VLATYRHVVA TVVMAPFALM FERKIRPKMT LAIFWRLLAL |
| 81- | GILEPLMDQN LYYIGLKNTS ASYTSAFTNA LPAVTFILAL |
| 121- | IFRLETVNFR KVHSVAKVVG TVITVGGAMI MTLYKGPAIE |
| 161- | IVKAAHNSFH GGSSSTPTGQ HWVLGTIAIM GSISTWAAFF |
| 201- | ILQSYTLKVY PAELSLVTLI CGIGTILNAI ASLIMVRDPS |
| 241- | AWKIGMDSGT LAAVYSGVVC SGIAYYIQSI VIKQRGPVFT |
| 281- | TSFSPMCMII TAFLGALVLA EKIHLGSIIG AVFIVLGLYS |
| 321- | VVWGKSKDEV NPLDEKIVAK SQELPITNVV KQTNGHDVSG |
| 361- | APTNGVVTST -370 |

SEQ ID NO: 40. Arabidopsis thaliana aspartic acid transporter AtBAT1

| | |
|---|---|
| 1- | MGLGGDQSFV PVMDSGQVRL KELGYKQELK RDLSVFSNFA |
| 41- | ISFSIISVLT GITTTYNTGL RFGGTVTLVY GWFLAGSFTM |
| 81- | CVGLSMAEIC SSYPTSGGLY YWSAMLAGPR WAPLASWMTG |
| 121- | WFNIVGQWAV TASVDFSLAQ LIQVIVLLST GGRNGGGYKG |
| 161- | SDFVVIGIHG GILFIHALLN SLPISVLSFI GQLAALWNLL |
| 201- | GVLVLMILIP LVSTERATTK FVFTNFNTDN GLGITSYAYI |
| 241- | FVLGLLMSQY TITGYDASAH MTEETVDADK NGPRGIISAI |
| 281- | GISILFGWGY ILGISYAVTD IPSLLSETNN SGGYAIAEIF |
| 321- | YLAFKNRFGS GTGGIVCLGV VAVAVFFCGM SSVTSNSRMA |
| 361- | YAFSRDGAMP MSPLWHKVNS REVPINAVWL SALISFCMAL |
| 401- | TSLGSIVAFQ AMVSIATIGL YIAYAIPIIL RVTLARNTFV |
| 441- | PGPFSLGKYG MVVGWVAVLW VVTISVLFSL PVAYPITAET |
| 481- | LNYTPVAVAG LVAITLSYWL FSARHWFTGP ISNILS -516 |

SEQ ID NO: 41. DNA integration cassette s376

| | |
|---|---|
| aatcaatata aatctggtgt cttccgtatt gccgaatggg ctgacatcac taatgcacat | 60 |
| ggtgtaacgg gtgcaggtat tgtttctggc ttgaaggagg cagcccaaga aacaaccagt | 120 |
| gaacctagag gtttgctaat gcttgctgag ttatcatcaa agggttcttt agcatatggt | 180 |
| gaatatacag aaaaaacagt agaaattgct aaatctgata aagagtttgt cattggtttt | 240 |
| attgcgcaac acgatatggg cggtagagaa gaaggttttg actggatcat tatgactcca | 300 |
| ggggttggtt tagatgacaa aggtgatgca cttggtcaac aatatagaac tgttgatgaa | 360 |
| gttgtaaaga ctggaacgga tatcataatt gttggtagag gtttgtacgg tcaaggaaga | 420 |
| gatcctatag agcaagctaa aagataccaa caagctggtt ggaatgctta tttaaacaga | 480 |
| tttaaatgag tgaatttact ttaaatcttg catttaaata aattttcttt ttatagcttt | 540 |
| atgacttagt ttcaatttat atactatttt aatgacattt tcgattcatt gattgaaagc | 600 |
| tttgtgtttt ttcttgatgc gctattgcat tgttcttgtc tttttcgcca catttaatat | 660 |
| ctgtagtaga tacctgatac attgtggatc gcctggcagc agggcgataa cctcataact | 720 |
| tcgtataatg tatgctatac gaacggtaga tagacatctg agtgagcgat agatagatag | 780 |
| atagatagat agatgtatgg gtagatagat gcatatatag atgcatggaa tgaaaggaag | 840 |
| atagatagag agaaatgcag aaataagcgt atgaggttta attttaatgt acatacatgt | 900 |
| atagataaac gatgtcgata taatttattt agtaaacaga ttccctgata tgtgttttta | 960 |
| gttttatttt tttttgtttt ttctatgttg aaaaacttga tgacatgatc gagtaaaatt | 1020 |
| ggagcttgat ttcattcatc ttgttgattc ctttatcata atgcaaagct gggggggggg | 1080 |
| agggtaaaaa aaagtgaaga aaaagaaagt atgatacaac tgtggaagtg gag | 1133 |

SEQ ID NO: 42. DNA integration cassette s404

| | |
|---|---|
| gcaggcttat ggcagacagg tacttttttt ttgtctctgt ataatgagtc aaattgtcaa | 60 |
| tattgaaggg ttgtatccaa actgcagttc ttgacagtca gacacactca tctttcataa | 120 |
| ccttccctaa atagatgtgc tcctatttca gccaagtatc tttattgtcg gtgaaaataa | 180 |
| tggaaacggt ctaaatgcgc ttgttactaa ggctgttact ttgataaacg catttgactt | 240 |
| tgagatatat aacttcaact ctaacgacct aatttcaaac ggaagagcta cttagaccat | 300 |
| agattaaaag tgaattctct ctaacacact ttgaggagca ttaatttcac accaaaacgt | 360 |
| ctatagatgc tgactttagc ggtttcaatg ggaattgatc ttgcaacacc aaggaattgc | 420 |
| cattgaagag aaacttactg atacatcatt caaccactcc gatgatatac accgggctag | 480 |
| atttcgatat ggatatggat atggatatgg atatggagat gaatttgaat ttagatttgg | 540 |
| gtcttgattt ggggttggaa ttaaaagggg ataacaatga gggttttcct gttgatttaa | 600 |
| acaatggacg tgggaggtga ttgatttaac ctgatccaaa aggggtatgt ctatttttta | 660 |
| gagtgtgtct ttgtgtcaaa ttatagtaga atgtgtaaag tagtataaac tttcctctca | 720 |
| aatgacgagg tttaaaacac cccccgggtg agccgagccg agaatggggc aattgttcaa | 780 |
| tgtgaaatag aagtatcgag tgagaaactt gggtgttggc cagccaaggg ggaaggaaaa | 840 |
| tggcgcgaat gctcaggtga gattgttttg gaattgggtg aagcgaggaa atgagcgacc | 900 |
| cggaggttgt gactttagtg gcggaggagg acggaggaaa agccaagagg gaagtgtata | 960 |
| taaggggagc aatttgccac caggatagaa ttggatgagt tataattcta ctgtatttat | 1020 |
| tgtataattt atttctcctt ttgtatcaaa cacattacaa aacacacaaa acacacaaac | 1080 |
| aaacacaatt acaaaaaatg aaaaacatcg ccttaattgg ttgtggtgct attggttcct | 1140 |
| ctgtcttgga attattgtcc ggtgataccc aattgcaagt tggttgggtt ttggtcccag | 1200 |
| aaattactcc agctgttaga gaaactgctg ccagattggc tccacaagct caattgttgc | 1260 |
| aagctttgcc aggtgatgct gttccagact tgttggttga atgtgctggt cacgctgcta | 1320 |
| ttgaagaaca cgtcttgcca gccttggcta gaggtatccc agctgtcatt gcctccatcg | 1380 |
| gtgctttatc tgccccaggt atggctgaaa gagtccaagc tgccgctgaa accggtaaaa | 1440 |
| ctcaagctca attgttgtcc ggtgccatcg gtggtatcga tgctttagct gctgctagag | 1500 |
| ttggtggttt agaaactgtc ttgtacaccg gtagaaagcc accaaaagcc tggtctggta | 1560 |
| ctccagctga gcaagtttgt gacttagacg gtttgaccga agctttttgt attttcgagg | 1620 |
| gttctgctag agaagctgcc caattgtacc caaagaacgc taatgttgct gctaccttgt | 1680 |
| ccttggccgg tttgggtttg gacaagacca tggttagatt attcgccgat cctggtgtcc | 1740 |
| aagaaaatgt ccaccaagtt gaagctagag gtgctttcgg tgccatggaa ttgactatga | 1800 |
| gaggtaagcc attagctgct aacccaaaaa cttctgcctt aaccgtttac tctgttgtta | 1860 |
| gagctgtttt gaataacgtc gctccattgg ctatttaatc cagccagtaa aatccatact | 1920 |
| caacgacgat atgaacaaat ttccctcatt ccgatgctgt atatgtgtat aaatttttac | 1980 |
| atgctcttct gtttagacac agaacagctt taaataaaat gttggatata ctttttctgc | 2040 |
| ctgtggtgta ccgttcgtat aatgtatgct atacgaagtt ataaccggcg ttgccagcga | 2100 |
| taaacgggaa acatcatgaa aactgtttca ccctctggga agcataaaca ctagaaagcc | 2160 |
| aatgaagagc tctacaagcc tcttatgggt tcaatgggtc tgcaatgacc gcatacgggc | 2220 |
| ttggacaatt accttctatt gaatttctga gaagagatac atctcaccag caatgtaagc | 2280 |
| agacaatccc aattctgtaa acaacctctt tgtccataat tccccatcag aagagtgaaa | 2340 |
| aatgccctca aaatgcatgc gccacaccca cctctcaact gcactgcgcc acctctgagg | 2400 |
| gtcttttcag gggtcgacta ccccggacac ctcgcagagg agcgaggtca cgtactttta | 2460 |
| aaatggcaga gacgcgcagt ttcttgaaga aaggataaaa atgaaatggt gcggaaatgc | 2520 |
| gaaaatgatg aaaaattttc ttggtggcga ggaaattgag tgcaataatt ggcacgaggt | 2580 |
| tgttgccacc cgagtgtgag tatatatcct agtttctgca cttttcttct tcttttcttt | 2640 |
| accttttctt ttcaactttt ttttactttt tccttcaaca gacaaatcta acttatatat | 2700 |
| cacaatggcg tcatacaaag aaagatcaga atcacacact tcccctgttg ctaggagact | 2760 |
| tttctccatc atggaggaaa agaagtctaa cctttgtgca tcattggata ttactgaaac | 2820 |
| tgaaaagctt ctctctattt tggacactat tggtccttac atctgtctag ttaaaacaca | 2880 |
| catcgatatt gtttctgatt ttacgtatga aggaactgtg ttgcctttga aggagcttgc | 2940 |
| caagaaacat aattttatga tttttgaaga tagaaaattt gctgatattg gtaacaccgt | 3000 |
| taaaaatcaa tataaatctg gtgtcttccg tattgccgaa tgggctgaca tcactaatgc | 3060 |
| acatggtgta acgggtgcag gtattgtttc tggcttgaag gaggcagccc aagaaacaac | 3120 |
| cagtgaacct agaggtttgc taatgcttgc tgagttatca tcaaagggtt ctttagcata | 3180 |
| tggtgaatat acagaaaaaa cagtagaaat tgctaaatct gataaagagt ttgtcattgg | 3240 |
| ttttattgcg caacacgata tgggcggtag agaagaaggt tttgactgga tcattatgac | 3300 |
| tcca | 3304 |

SEQ ID NO: 43. DNA integration cassette s357

| | |
|---|---|
| tagacgttgt atttccagct ccaacatggt taaactattg ctatggtgat ggtattacag | 60 |
| atagtaaaag aaggaagggg gggggtggca atctcaccct aacagttact aagaacgtct | 120 |
| acttcatcta ctgtcaatat acattggcca catgccgaga aattacgtcg acgccaaaga | 180 |
| agggcccagc cgaaaaaaga aatggaaaac ttggccgaaa agggaaacaa acaaaaaggt | 240 |
| gatgtaaaat tagcggaaag gggaattggc aaattgaggg agaaaaaaaa aaaggcagaa | 300 |
| aaggaggcgg aaagtcagta cgttttgaag gcgtcattgg ttttcccttt tgcagagtgt | 360 |
| ttcatttctt ttgtttcatg acgtagtggc gtttcttttc ctgcacttta gaaatctatc | 420 |
| ttttccttat caagtaacaa gcggttggca aaggtgtata taaatcaagg aattcccact | 480 |
| ttgaaccctt tgaattttga tatcggttat tttaaattta ttttatgttt ctaatctcaa | 540 |
| agagtttaca ctttacaagg agtttctcta ccgttcgtat aatgtatgct atacgaagtt | 600 |
| ataaccggcg ttgccagcga taaacgggaa acatcatgaa aactgtttca ccctctggga | 660 |
| agcataaaca ctagaaagcc aatgaagagc tctacaagcc tcttatgggt tcaatgggtc | 720 |
| tgcaatgacc gcatacgggc ttggacaatt accttctatt gaatttctga gaagagatac | 780 |
| atctcaccag caatgtaagc agacaatccc aattctgtaa acaacctctt tgtccataat | 840 |
| tccccatcag aagagtgaaa aatgccctca aaatgcatgc gccacaccca cctctcaact | 900 |
| gcactgcgcc acctctgagg gtcttttcag gggtcgacta ccccggacac ctcgcagagg | 960 |
| agcgaggtca cgtactttta aaatggcaga gacgcgcagt ttcttgaaga aaggataaaa | 1020 |
| atgaaatggt gcggaaatgc gaaaatgatg aaaaattttc ttggtggcga ggaaattgag | 1080 |
| tgcaataatt ggcacgaggt tgttgccacc cgagtgtgag tatatatcct agtttctgca | 1140 |
| cttttcttct tcttttcttt accttttctt ttcaactttt ttttactttt tccttcaaca | 1200 |
| gacaaatcta acttatatat cacaatggcg tcatacaaag aaagatcaga atcacacact | 1260 |
| tcccctgttg ctaggagact tttctccatc atggaggaaa agaagtctaa cctttgtgca | 1320 |
| tcattggata ttactgaaac tgaaaagctt ctctctattt tggacactat tggtccttac | 1380 |
| atctgtctag ttaaaacaca catcgatatt gtttctgatt ttacgtatga aggaactgtg | 1440 |
| ttgcctttga aggagcttgc caagaaacat aattttatga tttttgaaga tagaaaattt | 1500 |
| gctgatattg gtaacaccgt taaaaatcaa tataaatctg gtgtcttccg tattgccgaa | 1560 |
| tgggctgaca tcactaatgc acatggtgta acgggtgcag gtattgtttc tggcttgaag | 1620 |
| gaggcagccc aagaaacaac cagtgaacct agaggtttgc taatgcttgc tgagttatca | 1680 |
| tcaaagggtt ctttagcata tggtgaatat acagaaaaaa cagtagaaat tgctaaatct | 1740 |
| gataaagagt ttgtcattgg ttttattgcg caacacgata tgggcggtag agaagaaggt | 1800 |
| tttgactgga tcattatgac tccaggggtt ggtttagatg acaaaggtga tgcacttggt | 1860 |
| caacaatata gaactgttga tgaagttgta aagactggaa cggatatcat aattgttggt | 1920 |
| agaggtttgt acggtcaagg aagagatcct atagagcaag ctaaaagata ccaacaagct | 1980 |
| ggttggaatg cttatttaaa cagatttaaa tgagtgaatt tactttaaat cttgcattta | 2040 |
| aataaatttt ctttttatag ctttatgact tagtttcaat ttatatacta ttttaatgac | 2100 |
| attttcgatt cattgattga aagctttgtg ttttttcttg atgcgctatt gcattgttct | 2160 |
| tgtctttttc gccacattta atatctgtag tagatacctg atacattgtg gatcgcctgg | 2220 |
| cagcagggcg ataacctcat aacttcgtat aatgtatgct atacgaacgg taataacctc | 2280 |
| aaggagaact ttggcattgt actctccatt gacgagtccg ccaacccatt cttgttaaac | 2340 |
| ccaaccttgc attatcacat tccctttgac cccctttagc tgcatttcca cttgtctaca | 2400 |
| ttaagattca ttacacattc tttttcgtat ttctcttacc tccctccccc ctccatggat | 2460 |
| cttatatata aatcttttct ataacaataa tatctactag agttaaacaa caattccact | 2520 |
| tggcatggct gtctcagcaa atctgcttct acctactgca cgggtttgca tgtcattgtt | 2580 |
| tctagcaggg aatcgtccat gtacgttgtc ctccatgatg gtcttcccgc tgccactttc | 2640 |
| tttagtatct taaatagagc agatcttacg tccacagtgc atccgtgcac cccgaaaatc | 2700 |
| gtatggtttt ccttgccacc tctcaca | 2727 |

SEQ ID NO: 44. DNA integration cassette s475

| | |
|---|---|
| agttgccatt gtgggtttgt gttgcaatcc ttgcaaatgt ttatattgac tatacaagtg | 60 |
| taggtcttta cgtttcatgg atttccttca tctttataag attgaatcat cagccatatt | 120 |
| tgagctctac ataattcata atggtctgat ttctacagga ctgttttgac aagaaagaat | 180 |
| ctcatgccgt gtttccaaca gtgtggcacc tggtgtcttt gataaacggc tcagaaactc | 240 |
| ctgtacctcg tgaaaaacaa aattgctgtt tcaactcctt ttcaatattt ttcgagcttt | 300 |
| ggcaactacc taaaaaggca attcctatcc tgaaaagtat cttgggcatt tctgtggctt | 360 |
| ttgctcctcc taagatgatt atcttttgtg gctctctcac tgagttggac cactttttca | 420 |
| gagcaaatgc agctgttaca taatagagaa gattcgatat aaaaaaaatt gcaccataat | 480 |
| caacttagtt tcgtggaggt accaaagcca agggcaaaac taacaactac agggctagat | 540 |
| ttcgatatgg atatggatat ggatatggat atggagatga atttgaattt agatttgggt | 600 |
| cttgatttgg ggttggaatt aaaaggggat aacaatgagg gttttcctgt tgatttaaac | 660 |
| aatggacgtg ggaggtgatt gatttaacct gatccaaaag gggtatgtct attttttaga | 720 |
| gtgtgtcttt gtgtcaaatt atagtagaat gtgtaaagta gtataaactt tcctctcaaa | 780 |
| tgacgaggtt taaaacaccc cccgggtgag ccgagccgag aatggggcaa ttgttcaatg | 840 |
| tgaaatagaa gtatcgagtg agaaacttgg gtgttggcca gccaaggggg gggggaagga | 900 |
| aaatggcgcg aatgctcagg tgagattgtt ttggaattgg gtgaagcgag gaaatgagcg | 960 |
| acccggaggt tgtgacttta gtggcggagg aggacggagg aaaagccaag agggaagtgt | 1020 |
| atataagggg agcaatttgc caccaggata gaattggatg agttataatt ctactgtatt | 1080 |
| tattgtataa tttatttctc cttttatatc aaacacatta caaaacacac aaaacacaca | 1140 |
| aacaaacaca attacaaaaa atgtcaactg tggaagatca ctcctcctta cataaattga | 1200 |
| gaaaggaatc tgagattctt tccaatgcaa acaaaatctt agtggctaat agaggtgaaa | 1260 |
| ttccaattag aattttcagg tcagcccatg aattgtcaat gcatactgtg gcgatctatt | 1320 |
| cccatgaaga tcggttgtcc atgcataggt tgaaggccga cgaggcttat gcaatcggta | 1380 |
| agacgggtca atattcgcca gttcaagctt atctacaaat tgacgaaatt atcaaaatag | 1440 |
| caaaggaaca tgatgtttcc atgatccatc caggttatgg tttcttatct gaaaactccg | 1500 |
| aattcgcaaa gaaggttgaa gaatccggta tgatttgggt tgggcctcct gctgaagtta | 1560 |
| ttgattctgt tggtgacaag gtttctgcaa gaaatttggc aattaaatgt gacgttcctg | 1620 |
| ttgttcctgg taccgatggt ccaattgaag acattgaaca ggctaaacag tttgtggaac | 1680 |
| aatatggtta tcctgtcatt ataaaggctg catttggtgg tggtggtaga ggtatgagag | 1740 |
| ttgttagaga aggtgatgat atagttgatg ctttccaaag agcgtcatct gaagcaaagt | 1800 |
| ctgcctttgg taatggtact tgttttattg aaagattttt ggataagcca aaacatattg | 1860 |
| aggttcaatt attggctgat aattatggta acacaatcca tctctttgaa agagattgtt | 1920 |
| ctgttcaaag aagacatcaa aaggttgttg aaattgcacc tgccaaaact ttacctgttg | 1980 |
| aagttagaaa tgctatatta aaggatgctg taacgttagc taaaaccgct aactatagaa | 2040 |
| atgctggtac tgcagaattt ttagttgatt cccaaaacag acattatttt attgaaatta | 2100 |
| atccaagaat tcaagttgaa catacaatta ctgaagaaat cacaggtgtt gatattgttg | 2160 |
| ccgctcaaat tcaaattgct gcaggtgcat cattggaaca attgggtcta ttacaaaaca | 2220 |
| aaattacaac tagaggtttt gcaattcaat gtagaattac aaccgaggat cctgctaaga | 2280 |
| attttgcccc agatacaggt aaaattgagg tttatagatc tgcaggtggt aatggtgtca | 2340 |
| gattagatgg tggtaatggg tttgccggtg ctgttatatc tcctcattat gactcgatgt | 2400 |
| tggttaaatg ttcaacatct ggttctaact atgaaattgc cagaagaaag atgattagag | 2460 |
| ctttagttga atttagaatc agaggtgtca agaccaatat tcctttctta ttggcattgc | 2520 |
| taactcatcc agtcttcatt tcgggtgatt gttggacaac ttttattgat gatacccctt | 2580 |
| cgttattcga aatggtttct tcaaagaata gagcccaaaa attattggca tatattggtg | 2640 |
| acttgtgtgt caatggttct tcaattaaag gtcaaattgg tttccctaaa ttgaacaagg | 2700 |
| aagcagaaat cccagatttg ttggatccaa atgatgaggt tattgatgtt tctaaacctt | 2760 |
| ctaccaatgg tctaagaccg tatctattaa agtatggacc agatgcattt tccaaaaaag | 2820 |
| ttcgtgaatt cgatggttgt atgattatgg ataccacctg gagagatgca catcaatcat | 2880 |
| tattggctac aagagttaga actattgatt tactgagaat tgctccaacg actagtcatg | 2940 |
| ccttacaaaa tgcatttgca ttagaatgtt ggggtggcgc aacatttgat gttgcgatga | 3000 |
| ggttcctcta tgaagatcct tgggagagat taagacaact tagaaaggca gttccaaata | 3060 |
| ttcctttcca aatgttattg agaggtgcta atggtgttgc ttattcgtca ttacctgata | 3120 |
| atgcaattga tcattttgtt aagcaagcaa aggataatgg tgttgatatt ttcagagtct | 3180 |
| ttgatgcttt gaacgatttg gaacaattga aggttggtgt tgatgctgtc aagaaagccg | 3240 |
| gaggtgttgt tgaagctaca gtttgttact caggtgatat gttaattcca ggtaaaaagt | 3300 |
| ataacttgga ttattattta gagactgttg gaaagattgt ggaaatgggt acccatattt | 3360 |
| taggtattaa ggatatggct ggcacgttaa agccaaaggc tgctaagttg ttgattggct | 3420 |
| cgatcagatc aaaataccct gacttggtta tccatgtcca tacccatgac tctgctggta | 3480 |
| ccggtatttc aacttatgtt gcatgcgcat tggcaggtgc cgacattgtc gattgtgcaa | 3540 |
| tcaattcgat gtctggttta acttctcaac cttcaatgag tgcttttatt gctgctttag | 3600 |
| atggtgatat cgaaactggt gttccagaac attttgcaag acaattagat gcatattggg | 3660 |
| cagaaatgag attgttatac tcatgtttcg aagccgactt gaagggacca gacccagaag | 3720 |
| tttataaaca tgaaattcca ggtggacagt tgactaacct aatcttccaa gcccaacaag | 3780 |
| ttggtttggg tgaacaatgg gaagaaacta agaagaagta tgaagatgct aacatgttgt | 3840 |
| tgggtgatat tgtcaaggtt accccaacct ccaaggttgt tggtgattta gcccaattta | 3900 |
| tggtttctaa taaattagaa aaagaagatg ttgaaaaact tgctaatgaa ttagatttcc | 3960 |
| cagattcagt tcttgatttc tttgaaggat taatgggtac accatatggt ggattcccag | 4020 |
| agcctttgag aacaaatgtc atttccggca agagaagaaa attaaagggt agaccaggtt | 4080 |
| tagaattaga acctttcaac ctcgaggaaa tcagagaaaa tttggtttcc agatttggtc | 4140 |
| caggtattac tgaatgtgat gttgcatctt ataacatgta tccaaaggtt tacgagcaat | 4200 |
| atcgtaaggt ggttgaaaaa tatggtgatt tatctgtttt accaacaaaa gcatttttgg | 4260 |
| cccctccaac tattggtgaa gaagttcatg tggaaattga gcaaggtaag actttgatta | 4320 |
| ttaagttgtt agccatttct gacttgtcta aatctcatgg tacaagagaa gtatactttg | 4380 |
| aattgaatgg tgaaatgaga aaggttacaa ttgaagataa aacagctgca attgagactg | 4440 |
| ttacaagagc aaaggctgac ggacacaatc caaatgaagt tggtgcgcca atggctggtg | 4500 |
| tcgttgttga agttagagtg aagcatggaa cagaagttaa gaagggtgat ccattagccg | 4560 |
| ttttgagtgc aatgaaaatg gaaatggtta tttctgctcc tgttagtggt agggtcggtg | 4620 |
| aagtttttgt caacgaaggc gattccgttg atatgggtga tttgcttgtg aaaattgcca | 4680 |
| aagatgaagc gccagcagct taatcttgat tcatgtaact catgtatttg ttttgtattc | 4740 |
| aattatgtta taccttggta tacatataac gatttgtatt tacatattta tttattagtg | 4800 |
| gtagtttttt ttttcagaga gtactgtatt tcctcccaaa caaccgtgaa ggctttaagg | 4860 |
| tccacttatc accagtataa gtttccttag tgacgacgcc tatttgctta attgtgattt | 4920 |
| caaagactca atttgttgct ccaagtcttt gatgtcttcg tctagttttc tttcatcaaa | 4980 |
| acatatacct atgttattaa tgttttgttg taacctgcga tcatggtcat aaatgtcggt | 5040 |
| gtaaatgtta gacagtaccg ttcgtataat gtatgctata cgaagttata accggcgttg | 5100 |
| ccagcgataa acgggaaaca tcatgaaaac tgtttcaccc tctgggaagc ataaacacta | 5160 |
| gaaagccaat gaagagctct acaagcctct tatgggttca atgggtctgc aatgaccgca | 5220 |
| tacgggcttg gacaattacc ttctattgaa tttctgagaa gagatacatc tcaccagcaa | 5280 |
| tgtaagcaga caatcccaat tctgtaaaca acctctttgt ccataattcc ccatcagaag | 5340 |
| agtgaaaaat gccctcaaaa tgcatgcgcc acacccacct ctcaactgca ctgcgccacc | 5400 |
| tctgagggtc ttttcagggg tcgactaccc cggacacctc gcagaggagc gaggtcacgt | 5460 |
| acttttaaaa tggcagagac gcgcagtttc ttgaagaaag gataaaaatg aaatggtgcg | 5520 |
| gaaatgcgaa aatgatgaaa aattttcttg gtggcgagga aattgagtgc aataattggc | 5580 |
| acgaggttgt tgccacccga gtgtgagtat atatcctagt ttctgcactt ttcttcttct | 5640 |
| tttctttacc ttttcttttc aacttttttt tactttttcc ttcaacagac aaatctaact | 5700 |
| tatatatcac aatggcgtca tacaaagaaa gatcagaatc acacacttcc cctgttgcta | 5760 |
| ggagactttt ctccatcatg gaggaaaaga agtctaacct ttgtgcatca ttggatatta | 5820 |
| ctgaaactga aaagcttctc tctattttgg acactattgg tccttacatc tgtctagtta | 5880 |
| aaacacacat cgatattgtt tctgatttta cgtatgaagg aactgtgttg cctttgaagg | 5940 |
| agcttgccaa gaaacataat tttatgattt ttgaagatag aaaatttgct gatattggta | 6000 |
| acaccgttaa aaatcaatat aaatctggtg tcttccgtat tgccgaatgg gctgacatca | 6060 |
| ctaatgcaca tggtgtaacg ggtgcaggta ttgtttctgg cttgaaggag gcagcccaag | 6120 |
| aaacaaccag tgaacctaga ggtttgctaa tgcttgctga gttatcatca aagggttctt | 6180 |
| tagcatatgg tgaatataca gaaaaaacag tagaaattgc taaatctgat aaagagtttg | 6240 |
| tcattggttt tattgcgcaa cacgatatgg gcggtagaga agaaggtttt gactggatca | 6300 |
| ttatgactcc a | 6311 |

SEQ ID NO: 45. DNA integration cassette s422

| | |
|---|---|
| aatcaatata aatctggtgt cttccgtatt gccgaatggg ctgacatcac taatgcacat | 60 |
| ggtgtaacgg gtgcaggtat tgtttctggc ttgaaggagg cagcccaaga aacaaccagt | 120 |
| gaacctagag gtttgctaat gcttgctgag ttatcatcaa agggttcttt agcatatggt | 180 |
| gaatatacag aaaaaacagt agaaattgct aaatctgata aagagtttgt cattggtttt | 240 |
| attgcgcaac acgatatggg cggtagagaa gaaggttttg actggatcat tatgactcca | 300 |
| ggggttggtt tagatgacaa aggtgatgca cttggtcaac aatatagaac tgttgatgaa | 360 |
| gttgtaaaga ctggaacgga tatcataatt gttggtagag gtttgtacgg tcaaggaaga | 420 |
| gatcctatag agcaagctaa aagataccaa caagctggtt ggaatgctta tttaaacaga | 480 |
| tttaaatgag tgaatttact ttaaatcttg catttaaata aattttcttt ttatagcttt | 540 |
| atgacttagt ttcaatttat atactatttt aatgacattt tcgattcatt gattgaaagc | 600 |
| tttgtgtttt ttcttgatgc gctattgcat tgttcttgtc tttttcgcca catttaatat | 660 |
| ctgtagtaga tacctgatac attgtggatc gcctggcagc agggcgataa cctcataact | 720 |
| tcgtataatg tatgctatac gaacggtaaa gcatgttttt tctttgaaaa ctatctttgg | 780 |
| atgttccaaa tacgaattag gttaggaatt gtatttatct tgtatatgac ccaaaaacac | 840 |
| ctaaaagttc attcaccgaa ctttaatgcg atttgcgatt ctgaaactga ttcatataaa | 900 |
| tcgtcaccag tagtattata caaggctctt atacctcttc tttttccacc ctacagatca | 960 |
| gtgcgcaaac atgcagcact gtgctttgta tagttttagt tggacctttt tataactaga | 1020 |
| agtccagctc gtcattttct ctcttcgttg gaccttcaca tttcaagagt ttgtcaacat | 1080 |
| agtttctaaa aagtaatata ctatccttca aaggtgtatt tttccactca aattcgtcag | 1140 |
| cagaaaaaat ttgttgtaga tttggggcat ccgtaaacgg attgaattct ctttcattcg | 1200 |
| gatcaaatac aacacaaac | 1219 |

SEQ ID NO: 46. DNA integration cassette s424

| | |
|---|---|
| gggggatatg gagggctcgg aatacagatg gatgcaactg tggcagcaat ttgagctgct | 60 |
| aatttttgct cctctttaac gcaatcattt cctcctccca acaacaaaat acacttccat | 120 |
| ggtcctacaa atgtaggcgg ctgtgaaaaa gactgtatta tgtattttaa tcaactgtgg | 180 |
| ctttttgaaa tagtctctta acattgccga aaaatagatg agctactccg tttaaacggg | 240 |
| cccaagatac aaaaaaaaag ttgcggctac tcacggatat taaaggttag aaagggcaat | 300 |
| atgttagtag aaacaaggtt taacttaagc atgatcaccg aaattgctgc ctttaagttg | 360 |
| taaatcaaga agtgcaaaaa ggagtatata aggaccatga ttctcccagc aagtcctttt | 420 |
| tttaataacg ccatctattt gtacccactt aatctagctt tacagtttat tatatagcaa | 480 |
| gtacatagat tttaattacc gttcgtataa tgtatgctat acgaagttat aaccggcgtt | 540 |
| gccagcgata aacgggaaac atcatgaaaa ctgtttcacc ctctgggaag cataaacact | 600 |
| agaaagccaa tgaagagctc tacaagcctc ttatgggttc aatgggtctg caatgaccgc | 660 |
| atacgggctt ggacaattac cttctattga atttctgaga agagatacat ctcaccagca | 720 |
| atgtaagcag acaatcccaa ttctgtaaac aacctctttg tccataattc cccatcagaa | 780 |
| gagtgaaaaa tgccctcaaa atgcatgcgc cacacccacc tctcaactgc actgcgccac | 840 |
| ctctgagggt cttttcaggg gtcgactacc ccggacacct cgcagaggag cgaggtcacg | 900 |
| tacttttaaa atggcagaga cgcgcagttt cttgaagaaa ggataaaaat gaaatggtgc | 960 |
| ggaaatgcga aaatgatgaa aaattttctt ggtggcgagg aaattgagtg caataattgg | 1020 |
| cacgaggttg ttgccacccg agtgtgagta tatatcctag tttctgcact tttcttcttc | 1080 |
| ttttctttac cttttctttt caactttttt ttactttttc cttcaacaga caaatctaac | 1140 |
| ttatatatca caatggcgtc atacaaagaa agatcagaat cacacacttc ccctgttgct | 1200 |
| aggagacttt tctccatcat ggaggaaaag aagtctaacc tttgtgcatc attggatatt | 1260 |
| actgaaactg aaaagcttct ctctattttg gacactattg gtccttacat ctgtctagtt | 1320 |
| aaaacacaca tcgatattgt ttctgatttt acgtatgaag gaactgtgtt gcctttgaag | 1380 |
| gagcttgcca agaaacataa ttttatgatt tttgaagata gaaaatttgc tgatattggt | 1440 |
| aacaccgtta aaaatcaata taaatctggt gtcttccgta ttgccgaatg ggctgacatc | 1500 |
| actaatgcac atggtgtaac gggtgcaggt attgtttctg gcttgaagga ggcagcccaa | 1560 |
| gaaacaacca gtgaacctag aggtttgcta atgcttgctg agttatcatc aaagggttct | 1620 |
| ttagcatatg gtgaatatac agaaaaaaca gtagaaattg ctaaatctga taaagagttt | 1680 |
| gtcattggtt ttattgcgca acacgatatg ggcggtagag aagaaggttt tgactggatc | 1740 |
| attatgactc caggggttgg tttagatgac aaaggtgatg cacttggtca acaatataga | 1800 |
| actgttgatg aagttgtaaa gactggaacg gatatcataa ttgttggtag aggtttgtac | 1860 |
| ggtcaaggaa gagatcctat agagcaagct aaaagatacc aacaagctgg ttggaatgct | 1920 |
| tatttaaaca gatttaaatg agtgaattta ctttaaatct tgcatttaaa taaattttct | 1980 |
| ttttatagct ttatgactta gtttcaattt atatactatt ttaatgacat tttcgattca | 2040 |
| ttgattgaaa gctttgtgtt ttttcttgat gcgctattgc attgttcttg tctttttcgc | 2100 |
| cacatttaat atctgtagta gatacctgat acattgtgga tcgcctggca gcagggcgat | 2160 |
| aacctcataa cttcgtataa tgtatgctat acgaacggta tggtattgct tgagcaaaaa | 2220 |
| aaaaagagag ggaaatacat ttgccacatt ataattatgt aatccatgga gtttatagag | 2280 |
| ataatcatat tagttacatg taatttttgg cacttgctat tgtagtatgc agtcgttcac | 2340 |
| gtgcaaacat gcatctgata atttttaagc atgcgaattt tctagatttt tcggttagtg | 2400 |
| cttaggggat actttttggg ttatagatac atgccttcat aaaaaacaga caagatgtgc | 2460 |
| tctttaccaa catagagaga tagatagaaa tttctaaaaa caattccctc actgacagaa | 2520 |
| acaagtagaa ttgaacatga aatggatatc catattttca ttagtgtcgg ctgttactgg | 2580 |
| gataagttcc ttgaaatcga tcgaggagga gatatcgaga atagattcaa aatttagaaa | 2640 |
| cgtaggaccg actcttgaaa ttctaaatga atacgattca gtgatcagcc t | 2691 |

SEQ ID NO: 47. DNA integration cassette s423

| | |
|---|---|
| atcgcaacag aagaggtatc aaatcatgtc ggcctgtgag ttagattgcc tgtccagcgt | 60 |
| gtcgcagatg gcatactacc cagctacagg cgccgtccca gatgcaattt ctgcacctcc | 120 |
| ccctacttat gaacgaagcg gcaatgacaa agttgttgtt tgatcagttg ttggctccgt | 180 |
| ccagttaaac aaaagctggg tcaacccctt acccgagtag attcgatgaa aattccccta | 240 |
| gcgacttctc cggttagcat cttcaacggt gaccggttat agccgccggt acccgtcctc | 300 |
| cccatgcgcg gacttcgctg ggaacttttg cggtgtatgc tacctcttta actgtagaca | 360 |
| ttctgtttta tttatgtaca aaagagtccc tcttggtgct cccattttct gattttcaac | 420 |
| tgctcaacat ctcttagacc aagtcctttc tttgataaag aatctagata acagagacaa | 480 |
| ggtatcttca tacagaaaat taccgttcgt ataatgtatg ctatacgaag ttataaccgg | 540 |
| cgttgccagc gataaacggg aaacatcatg aaaactgttt caccctctgg gaagcataaa | 600 |
| cactagaaag ccaatgaaga gctctacaag cctcttatgg gttcaatggg tctgcaatga | 660 |
| ccgcatacgg gcttggacaa ttaccttcta ttgaatttct gagaagagat acatctcacc | 720 |
| agcaatgtaa gcagacaatc ccaattctgt aaacaacctc tttgtccata attccccatc | 780 |
| agaagagtga aaaatgccct caaaatgcat gcgccacacc cacctctcaa ctgcactgcg | 840 |
| ccacctctga gggtcttttc aggggtcgac taccccggac acctcgcaga ggagcgaggt | 900 |
| cacgtacttt taaaatggca gagacgcgca gtttcttgaa gaaaggataa aaatgaaatg | 960 |
| gtgcggaaat gcgaaaatga tgaaaaattt tcttggtggc gaggaaattg agtgcaataa | 1020 |
| ttggcacgag gttgttgcca cccgagtgtg agtatatatc ctagtttctg cacttttctt | 1080 |
| cttcttttct ttaccttttc ttttcaactt ttttttactt tttccttcaa cagacaaatc | 1140 |
| taacttatat atcacaatgg cgtcatacaa agaaagatca gaatcacaca cttcccctgt | 1200 |
| tgctaggaga cttttctcca tcatggagga aaagaagtct aacctttgtg catcattgga | 1260 |
| tattactgaa actgaaaagc ttctctctat tttggacact attggtcctt acatctgtct | 1320 |
| agttaaaaca cacatcgata ttgtttctga ttttacgtat gaaggaactg tgttgccttt | 1380 |
| gaaggagctt gccaagaaac ataattttat gatttttgaa gatagaaaat ttgctgatat | 1440 |
| tggtaacacc gttaaaaatc aatataaatc tggtgtcttc cgtattgccg aatgggctga | 1500 |
| catcactaat gcacatggtg taacgggtgc aggtattgtt tctggcttga aggaggcagc | 1560 |
| ccaagaaaca accagtgaac ctagaggttt gctaatgctt gctgagttat catcaaaggg | 1620 |
| ttctttagca tatggtgaat atacagaaaa aacagtagaa attgctaaat ctgataaaga | 1680 |
| gtttgtcatt ggttttattg cgcaacacga tatgggcggt agagaagaag gttttgactg | 1740 |
| gatcattatg actccagggg ttggtttaga tgacaaaggt gatgcacttg gtcaacaata | 1800 |
| tagaactgtt gatgaagttg taaagactgg aacggatatc ataattgttg gtagaggttt | 1860 |
| gtacggtcaa ggaagagatc ctatagagca agctaaaaga taccaacaag ctggttggaa | 1920 |
| tgcttattta aacagattta aatgagtgaa tttactttaa atcttgcatt taaataaatt | 1980 |
| ttctttttat agctttatga cttagtttca atttatatac tattttaatg acattttcga | 2040 |
| ttcattgatt gaaagctttg tgttttttct tgatgcgcta ttgcattgtt cttgtctttt | 2100 |
| tcgccacatt taatatctgt agtagatacc tgatacattg tggatcgcct ggcagcaggg | 2160 |
| cgataacctc ataacttcgt ataatgtatg ctatacgaac ggtatctatc actagtctta | 2220 |
| tcgagatcga gcgaacaaac taaacctttt tcatcgcgga gtatattcca tcacactttg | 2280 |
| caatattata tagaaaaaag taaaaaaaaa actctgtata actaggaaat acgatcaata | 2340 |
| aagtcattga tacacagttt aacgaaatca tcaatattgg ggagaatata tgctttgaaa | 2400 |
| aagggatcgt tcagaacata cccaaaaaat ttcttgaatt cagcagtaac tagatttttc | 2460 |
| ggtttcttac cttgcctatt tttaatgata ctcgactttt cagagggtaa aaacaaagag | 2520 |
| gcaatcagca atagctttat aaacctcgaa tttgccaagt ttgagagaat aaacgatatg | 2580 |
| tcatctttaa ccttaggcat attttcgtga atgctagaat tgctacaacg ggcttttgaa | 2640 |
| tgtttcatgt ccaaattttc tgctacgttt tcttcggcag tttccctgat tgcgtctttg | 2700 |
| acaa | 2704 |

SEQ ID NO: 48. DNA integration cassette s425

| | |
|---|---|
| tgtgcaccat tttaatttct attgctataa tgtccttatt agttgccact gtgaggtgac | 60 |
| caatggacga gggcgagccg ttcagaagcc gcgaagggtg ttcttcccat gaatttctta | 120 |
| aggagggcgg ctcagctccg agagtgaggc gagacgtctc ggttagcgta tcccccttcc | 180 |
| tcggctttta caaatgatgc gctcttaata gtgtgtcgtt atccttttgg cattgacggg | 240 |
| ggagggaaat tgattgagcg catccatatt ttggcggact gctgaggaca atggtggttt | 300 |
| ttccgggtgg cgtgggctac aaatgatacg atggtttttt tcttttcgga gaaggcgtat | 360 |
| aaaaaggaca cggagaaccc atttattcta ataacagttg agcttcttta attatttgtt | 420 |
| aatataatat tctattatta tatattttct tcccaataaa acaaaataaa acaaaacaca | 480 |
| gcaaaacaca aaaattaccg ttcgtataat gtatgctata cgaagttata accggcgttg | 540 |
| ccagcgataa acgggaaaca tcatgaaaac tgtttcaccc tctgggaagc ataaacacta | 600 |
| gaaagccaat gaagagctct acaagcctct tatgggttca atgggtctgc aatgaccgca | 660 |
| tacgggcttg gacaattacc ttctattgaa tttctgagaa gagatacatc tcaccagcaa | 720 |
| tgtaagcaga caatcccaat tctgtaaaca acctctttgt ccataattcc ccatcagaag | 780 |
| agtgaaaaat gccctcaaaa tgcatgcgcc acacccacct ctcaactgca ctgcgccacc | 840 |
| tctgagggtc ttttcagggg tcgactaccc cggacacctc gcagaggagc gaggtcacgt | 900 |
| acttttaaaa tggcagagac gcgcagtttc ttgaagaaag gataaaaatg aaatggtgcg | 960 |
| gaaatgcgaa aatgatgaaa aattttcttg gtggcgagga aattgagtgc aataattggc | 1020 |
| acgaggttgt tgccacccga gtgtgagtat atatcctagt ttctgcactt ttcttcttct | 1080 |
| tttctttacc ttttcttttc aacttttttt tactttttcc ttcaacagac aaatctaact | 1140 |
| tatatatcac aatggcgtca tacaaagaaa gatcagaatc acacacttcc cctgttgcta | 1200 |
| ggagactttt ctccatcatg gaggaaaaga agtctaacct ttgtgcatca ttggatatta | 1260 |
| ctgaaactga aaagcttctc tctattttgg acactattgg tccttacatc tgtctagtta | 1320 |
| aaacacacat cgatattgtt tctgatttta cgtatgaagg aactgtgttg cctttgaagg | 1380 |
| agcttgccaa gaaacataat tttatgattt ttgaagatag aaaatttgct gatattggta | 1440 |
| acaccgttaa aaatcaatat aaatctggtg tcttccgtat tgccgaatgg gctgacatca | 1500 |
| ctaatgcaca tggtgtaacg ggtgcaggta ttgtttctgg cttgaaggag gcagcccaag | 1560 |
| aaacaaccag tgaacctaga ggtttgctaa tgcttgctga gttatcatca aagggttctt | 1620 |
| tagcatatgg tgaatataca gaaaaaacag tagaaattgc taaatctgat aaagagtttg | 1680 |
| tcattggttt tattgcgcaa cacgatatgg gcggtagaga agaaggtttt gactggatca | 1740 |
| ttatgactcc aggggttggt ttagatgaca aaggtgatgc acttggtcaa caatatagaa | 1800 |
| ctgttgatga agttgtaaag actggaacgg atatcataat tgttggtaga ggtttgtacg | 1860 |
| gtcaaggaag agatcctata gagcaagcta aaagatacca acaagctggt tggaatgctt | 1920 |
| atttaaacag atttaaatga gtgaatttac tttaaatctt gcatttaaat aaattttctt | 1980 |
| tttatagctt tatgacttag tttcaattta tatactattt taatgacatt ttcgattcat | 2040 |
| tgattgaaag ctttgtgttt tttcttgatg cgctattgac atttaatatc tgtagtagat | 2100 |
| acctgataca ttgtggatcg cctggcagca gggcgataac ctcataactt cgtataatgt | 2160 |
| atgctatacg aacggtatga catctgaatg taaaatgaac attaaaatga attactaaac | 2220 |
| tttacgtcta ctttacaatc tataaacttt gtttaatcat ataacgaaat acactaatac | 2280 |
| acaatcctgt acgtatgtaa tacttttatc catcaaggat tgagaaaaaa aagtaatgat | 2340 |
| tccctgggcc attaaaactt agacccccaa gcttggatag gtcactctct attttcgttt | 2400 |
| ctcccttccc tgatagaagg gtgatatgta attaagaata atatataatt ttataataaa | 2460 |
| aactaaaaca atccatcaat ctcaccatct tcgttgactt caacattcat aaatccggca | 2520 |
| taagttgata gacctggaat tgtcatgatc tttgcagcta gtgcatataa atatcctgct | 2580 |
| cctgcactta ttctaacttc tctgattggg aagatgaaat cctttggaac acctttcaat | 2640 |
| gttggatcat gggagagaga atattgcgtc t | 2671 |

SEQ ID NO: 49. DNA integration cassette s445

| | |
|---|---|
| acttggagaa attattaccg tttattgcct tctcagtgtc tgagttcctc attcgggcct | 60 |
| ttcctatcaa gtttctcaac aatcgactgc cttgtcttat cctcttatca gcttcatgcc | 120 |
| ttcctatttg ggacacggcg ctttgtttct tgtaaggtag gtgaaagaga gggacaaaaa | 180 |
| aaagggggca atatttcaac caaagtgttg tatataaaga caatgttctc ccctccctcc | 240 |
| ctctcccact cttctctttg ctgttgtgtt gttttctttt gttttctaat tacatatcct | 300 |
| ctctcttgtc tgtacactac ctctagtgtt tcttcttcaa catcaagtag ttttttgttt | 360 |
| ggccgcatcc ttgcgctttc cagcttaatt gaagagaaaa tataaacatc cccacacaca | 420 |
| tctataaaca tacaaacaga tacaaattga aagacacatt gaaagacaca ttgaaacacc | 480 |
| cattgatata cacataaatt tcaattaatc aaaagtacgt atctacagct aacccgagtg | 540 |
| tttttttttt ttttgttttt cttggtttcc agattctttc tttttttgtt ttttttgaga | 600 |
| agtgcttgtc tactaacata cttgcaaaaa catcctgcct atttaccgtt cgtataatgt | 660 |
| atgctatacg aagttataac cggcgttgcc agcgataaac gggaaacatc atgaaaactg | 720 |
| tttcaccctc tgggaagcat aaacactaga aagccaatga agagctctac aagcctctta | 780 |
| tgggttcaat gggtctgcaa tgaccgcata cgggcttgga caattacctt ctattgaatt | 840 |
| tctgagaaga gatacatctc accagcaatg taagcagaca atcccaattc tgtaaacaac | 900 |
| ctctttgtcc ataattcccc atcagaagag tgaaaaatgc cctcaaaatg catgcgccac | 960 |
| acccacctct caactgcact gcgccacctc tgagggtctt ttcaggggtc gactaccccg | 1020 |
| gacacctcgc agaggagcga ggtcacgtac ttttaaaatg gcagagacgc gcagtttctt | 1080 |
| gaagaaagga taaaaatgaa atggtgcgga aatgcgaaaa tgatgaaaaa ttttcttggt | 1140 |
| ggcgaggaaa ttgagtgcaa taattggcac gaggttgttg ccacccgagt gtgagtatat | 1200 |
| atcctagttt ctgcactttt cttcttcttt tctttacctt ttcttttcaa ctttttttta | 1260 |
| ctttttcctt caacagacaa atctaactta tatatcacaa tggcgtcata caaagaaaga | 1320 |
| tcagaatcac acacttcccc tgttgctagg agacttttct ccatcatgga ggaaaagaag | 1380 |
| tctaaccttt gtgcatcatt ggatattact gaaactgaaa agcttctctc tattttggac | 1440 |
| actattggtc cttacatctg tctagttaaa acacacatcg atattgtttc tgattttacg | 1500 |
| tatgaaggaa ctgtgttgcc tttgaaggag cttgccaaga aacataattt tatgattttt | 1560 |
| gaagatagaa aatttgctga tattggtaac accgttaaaa atcaatataa atctggtgtc | 1620 |
| ttccgtattg ccgaatgggc tgacatcact aatgcacatg gtgtaacggg tgcaggtatt | 1680 |
| gtttctggct tgaaggaggc agcccaagaa acaaccagtg aacctagagg tttgctaatg | 1740 |
| cttgctgagt tatcatcaaa gggttcttta gcatatggtg aatatacaga aaaaacagta | 1800 |
| gaaattgcta aatctgataa agagtttgtc attggtttta ttgcgcaaca cgatatgggc | 1860 |
| ggtagagaag aaggttttga ctggatcatt atgactccag gggttggttt agatgacaaa | 1920 |
| ggtgatgcac ttggtcaaca atatagaact gttgatgaag ttgtaaagac tggaacggat | 1980 |
| atcataattg ttggtagagg tttgtacggt caaggaagag atcctataga gcaagctaaa | 2040 |
| agataccaac aagctggttg gaatgcttat ttaaacagat ttaaatgagt gaatttactt | 2100 |
| taaatcttgc atttaaataa attttctttt tatagcttta tgacttagtt tcaatttata | 2160 |
| tactatttta atgacatttt cgattcattg attgaaagct ttgtgttttt tcttgatgcg | 2220 |
| ctattgcatt gttcttgtct ttttcgccac atttaatatc tgtagtagat acctgataca | 2280 |
| ttgtggatcg cctggcagca gggcgataac ctcataactt cgtataatgt atgctatacg | 2340 |
| aacggtattt aggtgtcaga catttgcact tgaaggatag gagccccaac ctgttgtaat | 2400 |
| ttatgtttga tgttttgtaa cgtttatctt tatctttatc ttgatctttg ttttcgtttt | 2460 |
| tgtttatgtt tttgatttta tacagttata cttatgctaa gatctatatc tttgtttggt | 2520 |
| cttacatata aatgtaccaa tatgctttgc ttccaagtta tcccactttg aatgcgagct | 2580 |
| gacagtatga ctccaaaaag cgtataaacg tgggtggtac aaattgaagc ggttactgaa | 2640 |
| tgtcagattg tcaatttttt tcccttgtat tatttttttt tttcactcct gtttccttct | 2700 |
| gtattttgtc gttctctgtg cattactcga cagatctgtc gaaatcccca cctagtcagt | 2760 |
| gcatttctta tttgaaacca tgcatatcct ccatagtaca ttaggtctca actcaaacaa | 2820 |
| aacgctgact gacgtatggt tccaatacgt tctccgaaat tacaaatctc cgagattcat | 2880 |
| aatcacaact tttggtgtgt tattgacatc atatattttt ttcccgtcat cgttacttgc | 2940 |
| agtctctcac aaaccttcta aaaggccaga taagtacaca tgtgggttca aaaacagcgg | 3000 |
| gaatgactgt tttgccaatt ctacactaca gtcactgtct tcgctagata cactttattt | 3060 |
| gtatctagcc gagatgctga gtttccaaat gccaccagga tacaccatct acccattacc | 3120 |
| attacatacg tctctatatc atatgc | 3146 |

SEQ ID NO: 50. DNA integration cassette s484/s485/s486

| | |
|---|---|
| gtatgatagg tgtttccatg ataaacaaca tgattgggtg tatctttaca ttcacttgct | 60 |
| ccccatggtt aaatgcaatg ggtaacacaa acacatatgc aattttgact gccttccaag | 120 |
| tcattgcatg tttatctgct gttccatttc tcatttgggg taaaaagatg cgtttatgga | 180 |
| ccagaaaata ctaccttgat tttgtggaaa agagagatgg agtcgaaaaa tcaagctgac | 240 |
| atatgcactg tcctatatac ctcatcgaag ctactttttt agtttcgttt tctaagcact | 300 |
| attctcttta attaatccga taattgtaca aaaaaaaaca tgcttctttc aaaatcatga | 360 |
| atgggatact acagaactta gccaccaata ttagtggtta ttttgtaatt tttggagtaa | 420 |
| acattataac gtaaagtagg tcagctctcc tcctctgtgt tgtctaaatg aaacaaatct | 480 |
| gtatacatca tgctcatggc tcgttgtgtg gataaacacg taatacattc catttttata | 540 |
| aagggcgtca cgctgctcct aattgagaaa acactacttg cataaaggtg agatccatga | 600 |
| tagcaaaatg tagggtaatg tacaaataga caagcacatg ggtcgataga ttgtttatat | 660 |
| taatctctac cagcctatca ttggctttgg ttagagacaa atcaaattat ccctccctcc | 720 |
| cttaattgta atcatatcct tttgtacagg attggaatct aaggcgggga acaaattcta | 780 |
| aaatgcgaac aattctccgc cacacttgcc ttatcaagga ataatttcca ccacctgtta | 840 |
| cggtacgttg tcaaattgat gatggcctgg tataaatgtt tgttcattct atttgaaact | 900 |
| ctacctgtta ctggacctct agcatttccc attggttttt gatatatcaa ccacatttcc | 960 |
| ctaattgcgc ggcgcgactt cgacagaacc agggctagat ttcgatatgg atatggatat | 1020 |
| ggatatggat atggagatga atttgaattt agatttgggt cttgatttgg ggttggaatt | 1080 |
| aaaaggggat aacaatgagg gttttcctgt tgatttaaac aatggacgtg ggaggtgatt | 1140 |
| gatttaacct gatccaaaag gggtatgtct attttttaga gtgtgtcttt gtgtcaaatt | 1200 |
| atagtagaat gtgtaaagta gtataaactt tcctctcaaa tgacgaggtt taaaacaccc | 1260 |
| cccgggtgag ccgagccgag aatggggcaa ttgttcaatg tgaaatagaa gtatcgagtg | 1320 |
| agaaacttgg gtgttggcca gccaaggggg gggggaagga aaatggcgcg aatgctcagg | 1380 |
| tgagattgtt ttggaattgg gtgaagcgag gaaatgagcg acccggaggt tgtgacttta | 1440 |
| gtggcggagg aggacggagg aaaagccaag agggaagtgt atataagggg agcaatttgc | 1500 |
| caccaggata gaattggatg agttataatt ctactgtatt tattgtataa tttatttctc | 1560 |
| cttttgtatc aaacacatta caaaacacac aaaacacaca aacaaacaca attacaaaaa | 1620 |
| atggaagata aagaaggacg atttcgagtg gaatgcattg aaaatgtaca ttatgtaaca | 1680 |
| gatatgtttt gtaaatatcc attaaaactt atcgctccta aaacaaaact tgatttttct | 1740 |
| attctgtaca tcatgagcta tggaggtggc ctggtatcag gggatcgtgt agcgctggat | 1800 |
| attatagttg gaaaaaatgc tacattgtgc atacagagtc aaggaaatac aaaattatat | 1860 |
| aaacaaatac caggaaagcc tgcaacacag caaaagttgg atgtagaagt tggaacgaat | 1920 |
| gcattgtgct tgttattaca agatccagtg caaccttttg gagatagtaa ttacattcag | 1980 |
| actcaaaact ttgtattaga agacgaaact tcttctcttg cattactgga ttggacatta | 2040 |
| catggtcgaa gccatatcaa tgaacaatgg agtatgcgat cttatgtgtc caaaaattgt | 2100 |
| atccagatga agattccagc ttcaaaccag agaaaaacgc ttttgagaga tgtgttaaaa | 2160 |
| atattcgatg agcctaacct acatattggt ttaaaagccg aacgaatgca tcactttgaa | 2220 |
| tgtataggca atttgtatct tataggacca aaatttctta aaactaaaga agcagttttg | 2280 |
| aaccaatata ggaacaagga gaagaggata tcaaaaacaa cggattcatc tcaaatgaag | 2340 |
| aagattatct ggactgcttg tgaaattcgg tcggttacaa taattaaatt cgctgcttac | 2400 |
| aacactgaaa ctgcacgaaa ttttcttctg aaattatttt cggactacgc aagctttcta | 2460 |
| gatcatgaaa ctcttcgcgc tttttggtac tgagtgaatt tactttaaat cttgcattta | 2520 |
| aataaatttt ctttttatag ctttatgact tagtttcaat ttatatacta ttttaatgac | 2580 |
| attttcgatt cattgattga aagctttgtg ttttttcttg atgcgctatt gcattgttct | 2640 |
| tgtctttttc gccacatgta atatctgtag tagatacctg atacattgtg gatgaaacat | 2700 |
| catgaaaact gtttcaccct ctgtgaagca taaacactag aaagccaatg aagagctcta | 2760 |
| caagcctctt atgggttcaa tgggtctgca atgaccgcat acgggcttgg acaattacct | 2820 |
| tctattgaat ttctgagaag agatacatct caccagcaat gtaagcagac aatcccaatt | 2880 |
| ctgtaaacaa cctctttgtc cataattccc catcagaaga gtgaaaaatg ccctcaaaat | 2940 |
| gcatgcgcca cacccatctt tcaactgcac tgcgccacct ctgagggtct tttcaggggt | 3000 |
| cgactacccc ggacacctcg cagaggagcg aggtcacgta cttttaaaat ggcagagacg | 3060 |
| cgcagtttct tgaagaaagg ataaaaatga aatggtgcgg aaatgcgaaa atgatgaaaa | 3120 |
| attttcttgg tggcgaggaa attgagtgca ataattggca cgaggttgtt gccacccgag | 3180 |
| tgtgagtata tatcctagtt tctgcacttt tcttcttctt ttctttacct tttcttttca | 3240 |
| actttttttt actttttcct tcaacagaca aatctaactt atatatcaca atgactgatt | 3300 |
| cgcaaacgga aacacacttg tcgctaattc tttcagacac tgcgtttcct ctgtcatctt | 3360 |
| tttcttattc gtatgggtta gagtcgtatt tgtctcatca gcaggtgaga gacgtcaatg | 3420 |
| catttttcaa ctttttacca ttgtccctca attcagtgct acataccaat ttgccaactg | 3480 |
| tcaaagcagc ttgggagtca ccgcaacaat attccgaaat cgaagacttt tttgaaagca | 3540 |
| cacagacatg cacaattgcc caaaaggtct ccaccatgca gggtaaatct ttgttaaata | 3600 |
| tttggacaaa atcactctcc tttttcgtta catcaaccga tgtcttcaaa tacttggatg | 3660 |
| agtacgaaag aagagttcgt agtaaaaagg cactcggtca tttcccagtg gtttggggtg | 3720 |
| tggtatgtag agccttggga ttatcgttag aaaggacatg ttatctgttc ttattggggc | 3780 |
| atgcaaaatc gatttgctca gcagctgttc gcttagatgt tttgacctcc ttccagtacg | 3840 |
| tttccacttt ggctcatcct caaaccgaaa gtttacttag agattcgtcg caactagctt | 3900 |
| tgaacatgca actagaggac actgctcagt catggtatac gctggacctt tggcagggta | 3960 |
| gacacagttt gttatatagt agaatattta atagttaatc cagccagtaa aatccatact | 4020 |
| caacgacgat atgaacaaat ttccctcatt ccgatgctgt atatgtgtat aaatttttac | 4080 |
| atgctcttct gtttagacac agaacagctt taaataaaat gttggatata ctttttctgc | 4140 |
| ctgtggtgta ccgttcgtat aatgtatgct atacgaagtt ataaccggcg ttgccagcga | 4200 |
| taaacggctc catgctggac ttactcgtcg aagatttcct gctactctct atataattag | 4260 |
| acacccatgt tatagatttc agaaaacaat gtaataatat atggtagcct cctgaaacta | 4320 |
| ccaagggaaa aatctcaaca ccaagagctc atattcgttg gaatagcgat aatatctctt | 4380 |
| tacctcaatc ttatatgcat gttatttcgc ctggcagcag ggcgataacc tcatttggtt | 4440 |
| cattaacttt tggttctgtt cttggaaacg ggtaccaact ctctcagagt gcttcaaaaa | 4500 |
| tttttcagca catttggtta gacatgaact ttctctgctg gttaaggatt cagaggtgaa | 4560 |
| gtcttgaaca caatcgttga aacatctgtc cacaagagat gtgtatagcc tcatgaaatc | 4620 |
| agccatttgc ttttgttcaa cgatcttttg aaattgttgt tgttcttggt agttaagttg | 4680 |
| atccatcttg gcttatgttg tgtgtatgtt gtagttattc ttagtatatt cctgtcctga | 4740 |
| gtttagtgaa acataatatc gccttgaaat gaaaatgctg aaattcgtcg acatacaatt | 4800 |
| tttcaaactt ttttttttgt tggtgcacgg acatgttttt aaaggaagta ctctatacca | 4860 |
| gttattcttc acaaatttaa ttgctggaga atagatcttc aacgctttaa taaagtagtt | 4920 |
| tgtttgttaa ggatggcgtc atacaaagaa agatcagaat cacacacttc ccctgttgct | 4980 |
| aggagacttt tctccatcat ggaggaaaag aagtctaacc tttgtgcatc attggatatt | 5040 |
| actgaaactg aaaagcttct ctctattttg gacactattg gtccttacat ctgtctagtt | 5100 |
| aaaacacaca tcgatattgt ttctgatttt acgtatgaag gaactgtgtt gcctttgaag | 5160 |
| gagcttgcca agaaacataa ttttatgatt tttgaagata gaaaatttgc tgatattggt | 5220 |
| aacactgtta aaaatcaata taaatctggt gtcttccgta ttgccgaatg ggctgacatc | 5280 |
| actaatgcac atggtgtaac gggtgcaggt attgtttctg gcttgaagga ggccgcccaa | 5340 |
| gaaacaacca gtgaacctag aggtttgcta atgcttgctg agttatcatc aaagggttct | 5400 |
| ttagcatatg gtgaatatac agaaaaaaca gtagaaattg ctaaatctga taaagagttt | 5460 |
| gtcattggtt ttattgcgca acacgatatg ggcggtagag aagaaggttt tgactggatc | 5520 |
| attatgactc caggggttgg tttagatgac aaaggtgatg cacttggtca acaatataga | 5580 |
| actgttgatg aagttgtaaa gactggaacg gatatcataa ttgttggtag aggtttgtat | 5640 |
| ggtcaaggaa gagatcctgt agagcaagct aaaagatacc aacaagctgg ttggaatgct | 5700 |
| tatttaaaca gatttaaatg attcttacac aaagatttga tacatgtaca ctagtttaaa | 5760 |
| taagcatgaa aagaattaca caagcaaaaa aaaaattaaa tgaggtactt tgagtaaaat | 5820 |
| cttatgattt agaaaaagtt gtttaacaaa ggctttagta tgtgaatttt taatgtagca | 5880 |
| aagcgataac taataaacat aaacaaaagt atggttttct taaccggcgt tgccagcgat | 5940 |
| aaacggctcc atgctggact tactcgtcga agatttcctg ctactctcta tataattaga | 6000 |
| cacccatgtt atagatttca gaaaacaatg taataatata tggtagcctc ctgaaactac | 6060 |
| caagggaaaa atctcaacac caagagctca tattcgttgg aatagcgata atatctcttt | 6120 |
| acctcaatct tatatgcatg ttatttcgcc tggcagcagg gcgataacct cataacttcg | 6180 |
| tataatgtat gctatacgaa cggtagctac ttagcttcta tagttagtta atgcactcac | 6240 |
| gatattcaaa attgacaccc ttcaactact ccctactatt gtctactact gtctactact | 6300 |
| cctctttact atagctgctc ccaataggct ccaccaatag gctctgccaa tacattttgc | 6360 |
| gccgccacct ttcaggttgt gtcactcctg aaggaccata ttgggtaatc gtgcaatttc | 6420 |
| tggaagagag tccgcgagaa gtgaggcccc cactgtaaat cctcgagggg gcatggagta | 6480 |
| tggggcatgg aggatggagg atgggggggg ggcgaaaaat aggtagcaaa aggacccgct | 6540 |
| atcaccccac ccggagaact cgttgccggg aagtcatatt tcgacactcc ggggagtcta | 6600 |
| taaaaggcgg gttttgtctt ttgccagttg atgttgctga aaggacttgt ttgccgtttc | 6660 |
| ttccgattta acagtataga aatcaaccac tgttaattat acacgttata ctaacacaac | 6720 |
| aaaaacaaaa acaacgacaa caacaacaac aatggcgatt ccttttcttc acaagggagg | 6780 |
| ttctgatgac tcgactcatc accatacaca cgattacgac catcataacc atgatcatca | 6840 |
| tggtcacgat catcacagcc atgattcatc ttccaactct tccagcgaag ctgccagatt | 6900 |
| gcagttcatc caagagcatg gccattctca cgatgctatg gaaacgcctg gcagctactt | 6960 |
| gaagcgtgaa cttcctcagt tcaatcatag agacttctct cgtcgtgcct ttaccattgg | 7020 |
| cgtcggagga ccggtcggtt ctggtaaaac tgcacttttg cttcagcttt gcaggctctt | 7080 |
| gggtgaaaaa tatagcatcg gagttgttac caacgacata tttactcgtg aagatcaaga | 7140 |
| atttttaatt cgtaacaagg cacttcccga agagagaatt cgcgcaatcg aaacaggcgg | 7200 |
| ttgtccacac gctgctattc gtgaagacgt ctccggtaat ttggtcgcat tggaggagtt | 7260 |
| gcaatccgag ttcaacacag aattactact cgtggagtca ggaggtgata acttagctgc | 7320 |
| aaattactct cgtgatctcg ctgatttcat tatctatgta attgatgtat ctggaggcga | 7380 |
| caagattcca cgtaagggtg gacctggtat cacggagtca gatctgttga ttatcaacaa | 7440 |
| aacagatcta gctaagttgg tcggtgctga tttgtcggtc atggatcgtg atgcaaaaaa | 7500 |
| gattcgtgag aatggaccca ttgtttttgc acaagtcaaa aatcaagttg ggatggatga | 7560 |
| gatcaccgaa cttattctag gcgccgctaa gagtgctggt gctctcaagt aaatgagcta | 7620 |
| tacaggcaat ttatatcgaa gtatgtaaca tttggtaatc cgccgaactg cagtaataac | 7680 |
| aagtactggc cctaattact tgagcaatac attatccttt ttcttctgcc ataacacaga | 7740 |
| ttgctttgtt tttttgtgtc ttggcactta aacagtctgg tagcatcagc tttttccaaa | 7800 |
| atcacgaaat ttcaaatttt ttaggctcca tttagagcat caataattaa aacaacttca | 7860 |
| tgttacaagt ctataataaa ccgtaaaatt tacgtatccc tagattacac acaaaaaaaa | 7920 |
| ctacataggt cccaattagc gggatttatt aaagataagt tccaacgtca gacatggcat | 7980 |
| actaactact atggtcgccc aagttaaaga cgactcgctc cacagctgtg cttaccgaag | 8040 |
| gggcaatcgg ttttgtttct tgcaagatgc caaatcagcg agtgatattc tggctttttt | 8100 |
| tttttttgca caaacgaaca ccatgaattc catgatgccg tagttgcagc tttgcaggat | 8160 |
| atataactgc cgactattga ccttctgata agcagaccgt taacatgttg ttttctaaaa | 8220 |
| aggaagaaac gagtgaaccg ccatctcgtt cgaaacgtga gcaatgctgg gcatcaagag | 8280 |
| atgcatactt tgcttgcctt gacaagcaca atatcgagaa tccactagac ccagaaaagg | 8340 |
| cgaagattgc atcaaaaaat tgtgctgctg aagacaagca attttctaaa gattgtgttg | 8400 |
| caagttgggt gaagtacttc aaagagaaaa ggccattcga cattaaaaag gaaaggatgt | 8460 |
| tgaaagaagc tgcagaaaat gggcaagaaa tcgttcaaat ggaaggatat agaaagtagc | 8520 |
| tggaatttcc aataaaaaat accctttaca gaaaaatata ttcatgtaaa tacaaatga | 8579 |

SEQ ID NO: 51. DNA integration cassette s481

| | |
|---|---|
| acttggagaa attattaccg tttattgcct tctcagtgtc tgagttcctc attcgggcct | 60 |
| ttcctatcaa gtttctcaac aatcgactgc cttgtcttat cctcttatca gcttcatgcc | 120 |
| ttcctatttg ggacacggcg ctttgtttct tgtaaggtag gtgaaagaga gggacaaaaa | 180 |
| aaagggggca atatttcaac caaagtgttg tatataaaga caatgttctc ccctccctcc | 240 |
| ctctcccact cttctctttg ctgttgtgtt gttttctttt gttttctaat tacatatcct | 300 |
| ctctcttgtc tgtacactac ctctagtgtt tcttcttcaa catcaagtag ttttttgttt | 360 |
| ggccgcatcc ttgcgctttc cagcttaatt gaagagaaaa tataaacatc cccacacaca | 420 |
| tctataaaca tacaaacaga tacaaattga aagacacatt gaaagacaca ttgaaacacc | 480 |
| cattgatata cacataaatt tcaattaatc aaaagtacgt atctacagct aacccgagtg | 540 |
| tttttttttt ttttgttttt cttggtttcc agattctttc tttttttgtt ttttttgaga | 600 |
| agtgcttgtc tactaacata cttgcaaaaa catcctgcct attgggctag atttcgatat | 660 |
| ggatatggat atggatatgg atatggagat gaatttgaat ttagatttgg gtcttgattt | 720 |
| ggggttggaa ttaaaagggg ataacaatga gggttttcct gttgatttaa acaatggacg | 780 |
| tgggaggtga ttgatttaac ctgatccaaa aggggtatgt ctatttttta gagtgtgtct | 840 |
| ttgtgtcaaa ttatagtaga atgtgtaaag tagtataaac tttcctctca aatgacgagg | 900 |
| tttaaaacac cccccgggtg agccgagccg agaatggggc aattgttcaa tgtgaaatag | 960 |
| aagtatcgag tgagaaactt gggtgttggc cagccaaggg ggaaggaaaa tggcgcgaat | 1020 |
| gctcaggtga gattgttttg gaattgggtg aagcgaggaa atgagcgacc cggaggttgt | 1080 |
| gactttagtg gcggaggagg acggaggaaa agccaagagg gaagtgtata taaggggagc | 1140 |
| aatttgccac caggatagaa ttggatgagt tataattcta ctgtatttat tgtataattt | 1200 |
| atttctcctt ttgtatcaaa cacattacaa aacacacaaa acacacaaac aaacacaatt | 1260 |
| acaaaaaatg caacccagag agctacacaa attaacgctt caccagctgg gatctttagc | 1320 |
| ccaaaaaagg ctgtgtagag gggtaaagct taacaagtta gaggctactt cacttattgc | 1380 |
| atctcaaatt caagaatatg ttcgcgacgg taatcattcc gtagcagatt tgatgagtct | 1440 |
| tggtaaagat atgctgggta aacgccatgt tcagcccaat gtcgttcatt tgttacatga | 1500 |
| aattatgatt gaagcgactt tccctgatgg aacctatcta attaccattc atgatcccat | 1560 |
| ttgcactaca gatggtaatc tcgaacatgc tttatatgga agcttcctgc ctacgccaag | 1620 |
| ccaagaactg ttccctctgg aagaggaaaa gttatatgct ccggaaaata gccctggttt | 1680 |
| tgttgaagtc ttggagggcg agattgaact attgcctaat ttacctcgta ctcccatcga | 1740 |
| ggtacgaaac atgggtgaca ggccaattca agttggatca cactatcatt ttattgaaac | 1800 |
| taatgaaaaa ctatgcttcg atcgctcaaa ggcttatgga aagcgcttgg acattccgtc | 1860 |
| aggtactgct attcgatttg aacctggcgt aatgaaaatt gtcaatttaa tccctatcgg | 1920 |
| tggtgcaaaa ctaattcaag gaggtaattc actttcgaag ggtgtcttcg atgattctag | 1980 |
| gactcgggaa attgttgaca atttgatgaa acagggattc atgcatcaac ctgaatctcc | 2040 |
| gttgaatatg ccattacaat ctgcacgccc ttttgttgtt cctcgtaaat tatacgctgt | 2100 |
| aatgtatggt ccaacaacga atgataaaat tcgtctggga gatacaaatt tgattgtgcg | 2160 |
| cgtggaaaag gactttactg aatatggaaa tgaatctgtt ttcggcggcg gaaaggttat | 2220 |
| acgtgatggt acgggacagt ctagctcaaa atcgatggac gaatgcttgg acactgtaat | 2280 |
| tacaaatgct gtaatcattg atcataccgg tatctacaag gctgacattg gcattaaaaa | 2340 |
| cggatatatc gtaggtatag gtaaagcagg aaacccggat acaatggata acattggaga | 2400 |
| aaacatggtc attggatctt ctacagatgt tatttcagct gagaataaaa ttgttactta | 2460 |
| tggtggtatg gacagccacg ttcatttcat ctgtcctcaa caaattgaag aggcattggc | 2520 |
| ttccggtata actactatgt atggtggagg aactggccct agtacgggaa ctaatgctac | 2580 |
| tacctgcacc ccaaataaag acttaatccg ttctatgctt cgttctactg attcttatcc | 2640 |
| catgaacatt ggtctcaccg gaaaaggaaa tgatagcggt tcaagttctt tgaaggagca | 2700 |
| aatagaagca ggctgcagtg gacttaagct tcacgaagat tggggatcta ctcccgcagc | 2760 |
| aattgacagt tgtttgtctg tttgtgatga gtatgacgtt cagtgcctaa ttcataccga | 2820 |
| caccctcaat gaatcctctt ttgtagaagg tacatttaaa gcttttaaaa ataggaccat | 2880 |
| tcacacgtat cacgttgaag gagccggtgg tgggcatgcc cccgatatta tttctttagt | 2940 |
| ccaaaatcca aatattcttc cctctagcac caatcccaca cgaccattta ctacaaatac | 3000 |
| gcttgatgag gaactggaca tgttaatggt atgccatcat ctttctagga atgttcctga | 3060 |
| agacgttgca tttgcagaat cccgtattcg tgctgaaaca attgctgctg aagatatttt | 3120 |
| acaggatttg ggagctatta gtatgattag ttcagactct caagccatgg gtcgttgtgg | 3180 |
| tgaagtaatt tcaagaactt ggaaaaccgc ccataaaaat aagctacaac gaggagcact | 3240 |
| tcctgaggac gagggttcag gtgttgataa tttccgtgtg aaacgttatg tatccaaata | 3300 |
| cactataaac cctgcaatta ctcatggaat ttctcatatt gttggttctg tggagatagg | 3360 |
| caagtttgct gatcttgtct tatgggactt tgctgacttt ggggcaagac ccagtatggt | 3420 |
| gctgaaagga ggaatgattg cattggcctc tatgggtgat ccaaatggat cgattccaac | 3480 |
| ggtttctccc ctcatgtcct ggcaaatgtt tggtgcacat gaccccgaga ggagcattgc | 3540 |
| atttgtttcc aaggcctcta taacatccgg tgttattgaa agctatggac ttcataagag | 3600 |
| agttgaagcc gtaaaatata cgagaaacat tgggaagaaa gacatggttt acaattcata | 3660 |
| tatgccaaaa atgactgttg atccagaagc ttacacagtt actgcagatg gtaaagttat | 3720 |
| ggaatgtgag cctgtagaca aacttccact ttcccagtct tattttatct tttaatccag | 3780 |
| ccagtaaaat ccatactcaa cgacgatatg aacaaatttc cctcattccg atgctgtata | 3840 |
| tgtgtataaa tttttacatg ctcttctgtt tagacacaga acagctttaa ataaaatgtt | 3900 |
| ggatatactt tttctgcctg tggtgtaccg ttcgtataat gtatgctata cgaagttata | 3960 |
| accggcgttg ccagcgataa acgggaaaca tcatgaaaac tgtttcaccc tctgggaagc | 4020 |
| ataaacacta gaaagccaat gaagagctct acaagcctct tatgggttca atgggtctgc | 4080 |
| aatgaccgca tacgggcttg gacaattacc ttctattgaa tttctgagaa gagatacatc | 4140 |
| tcaccagcaa tgtaagcaga caatcccaat tctgtaaaca acctctttgt ccataattcc | 4200 |
| ccatcagaag agtgaaaaat gccctcaaaa tgcatgcgcc acacccacct ctcaactgca | 4260 |
| ctgcgccacc tctgagggtc ttttcagggg tcgactaccc cggacacctc gcagaggagc | 4320 |
| gaggtcacgt acttttaaaa tggcagagac gcgcagtttc ttgaagaaag gataaaaatg | 4380 |
| aaatggtgcg gaaatgcgaa aatgatgaaa aattttcttg gtggcgagga aattgagtgc | 4440 |
| aataattggc acgaggttgt tgccacccga gtgtgagtat atatcctagt ttctgcactt | 4500 |
| ttcttcttct tttctttacc ttttcttttc aacttttttt tactttttcc ttcaacagac | 4560 |
| aaatctaact tatatatcac aatggcgtca tacaaagaaa gatcagaatc acacacttcc | 4620 |
| cctgttgcta ggagactttt ctccatcatg gaggaaaaga agtctaacct ttgtgcatca | 4680 |
| ttggatatta ctgaaactga aaagcttctc tctattttgg acactattgg tccttacatc | 4740 |
| tgtctagtta aaacacacat cgatattgtt tctgatttta cgtatgaagg aactgtgttg | 4800 |
| cctttgaagg agcttgccaa gaaacataat tttatgattt ttgaagatag aaaatttgct | 4860 |
| gatattggta acaccgttaa aaatcaatat aaatctggtg tcttccgtat tgccgaatgg | 4920 |
| gctgacatca ctaatgcaca tggtgtaacg ggtgcaggta ttgtttctgg cttgaaggag | 4980 |
| gcagcccaag aaacaaccag tgaacctaga ggtttgctaa tgcttgctga gttatcatca | 5040 |
| aagggttctt tagcatatgg tgaatataca gaaaaaacag tagaaattgc taaatctgat | 5100 |
| aaagagtttg tcattggttt tattgcgcaa cacgatatgg gcggtagaga agaaggtttt | 5160 |
| gactggatca ttatgactcc a | 5181 |

SEQ ID NO: 52. DNA integration cassette s482

| | |
|---|---|
| aatcaatata aatctggtgt cttccgtatt gccgaatggg ctgacatcac taatgcacat | 60 |
| ggtgtaacgg gtgcaggtat tgtttctggc ttgaaggagg cagcccaaga aacaaccagt | 120 |
| gaacctagag gtttgctaat gcttgctgag ttatcatcaa agggttcttt agcatatggt | 180 |
| gaatatacag aaaaaacagt agaaattgct aaatctgata aagagtttgt cattggtttt | 240 |
| attgcgcaac acgatatggg cggtagagaa gaaggttttg actggatcat tatgactcca | 300 |
| ggggttggtt tagatgacaa aggtgatgca cttggtcaac aatatagaac tgttgatgaa | 360 |
| gttgtaaaga ctggaacgga tatcataatt gttggtagag gtttgtacgg tcaaggaaga | 420 |
| gatcctatag agcaagctaa aagataccaa caagctggtt ggaatgctta tttaaacaga | 480 |
| tttaaatgag tgaatttact ttaaatcttg catttaaata aattttcttt ttatagcttt | 540 |
| atgacttagt ttcaatttat atactatttt aatgacattt tcgattcatt gattgaaagc | 600 |
| tttgtgtttt ttcttgatgc gctattgcat tgttcttgtc tttttcgcca catttaatat | 660 |
| ctgtagtaga tacctgatac attgtggatc gcctggcagc agggcgataa cctcataact | 720 |
| tcgtataatg tatgctatac gaacggtagc tacttagctt ctatagttag ttaatgcact | 780 |
| cacgatattc aaaattgaca cccttcaact actccctact attgtctact actgtctact | 840 |
| actcctcttt actatagctg ctcccaatag gctccaccaa taggctctgc caatacattt | 900 |
| tgcgccgcca cctttcaggt tgtgtcactc ctgaaggacc atattgggta atcgtgcaat | 960 |
| ttctggaaga gagtccgcga gaagtgaggc ccccactgta aatcctcgag ggggcatgga | 1020 |
| gtatggggca tggaggatgg aggatggggg gggggcgaaa aataggtagc aaaaggaccc | 1080 |
| gctatcaccc cacccggaga actcgttgcc gggaagtcat atttcgacac tccggggagt | 1140 |
| ctataaaagg cgggttttgt cttttgccag ttgatgttgc tgaaaggact tgtttgccgt | 1200 |
| ttcttccgat ttaacagtat agaaatcaac cactgttaat tatacacgtt atactaacac | 1260 |
| aacaaaaaca aaaacaacga caacaacaac aacaatgaac agtatgtctg aatatgttaa | 1320 |
| acctagaaaa aatgaattta taaggaagtt tgagaatttt tatttcgaaa taccctttct | 1380 |
| atcaaagctt ccaccaaagg ttagcgtgcc tatcttttct ttgatatcgg taaatatcgt | 1440 |
| agtttggata attgcggcaa tagtcatcag tttagttaac agatcgttat ttctctcagt | 1500 |
| tttattatct tggacacttg gtttaagaca cgctctcgat gctgatcata ttactgcaat | 1560 |
| tgacaactta acgcgccgtt tattatcaac agacaaacca atgtcaacag ttggaacctg | 1620 |
| gttcagcatt ggtcattcaa ctgtagtcct tataacttgc atcgtagtag cagctacttc | 1680 |
| cagtaagttt gcagatcgat gggataactt tcaaaccata ggaggaataa ttggaacttc | 1740 |
| agttagcatg ggactattac ttttgttggc aattggaaat accgttttac tagtccggtt | 1800 |
| atcgtattgg ctttggatgt atcgcaaatc tggtgtcact aaagatgaag gggtcaccgg | 1860 |
| attcttagct cgaaaaatgc agagattgtt tagattggtt gactctccgt ggaagattta | 1920 |
| tgtacttggt tttgttttcg gtttgggatt tgataccagt actgaggttt ccttgctggg | 1980 |
| tatcgcaacc ttgcaagcct taaaaggaac ttctatatgg gcaatcttac ttttccccat | 2040 |
| tgtatttctt gttggaatgt gcttagttga taccacagat ggagcattaa tgtattatgc | 2100 |
| ttactcatat tcttcgggtg aaaccaatcc ttatttctct aggctttatt actccataat | 2160 |
| tttaacattt gtttcggtta tagcagcatt tacaatcggt atcattcaaa tgcttatgct | 2220 |
| aatcataagt gtccacccaa tggaaagtac attttggaat ggcctcaata gattatctga | 2280 |
| taattacgaa atagtcggtg gatgtatatg cggtgccttt gttctagcag gtttgtttgg | 2340 |
| tatttccatg cataattact ttaagaaaaa attcacacct ctagtgcaag taggaaatga | 2400 |
| cagagaggac gaagttctag agaaaaataa agaattagaa aacgtatcaa aaaactcgat | 2460 |
| ttctgttcaa atttccgaaa gtgaaaaggt gagttacgat acagtggatt ctaaggtttg | 2520 |
| atttaggtgt cagacatttg cacttgaagg ataggagccc caacctgttg taatttatgt | 2580 |
| ttgatgtttt gtaacgttta tctttatctt tatcttgatc tttgttttcg tttttgttta | 2640 |
| tgtttttgat tttatacagt tatacttatg ctaagatcta tatctttgtt tggtcttaca | 2700 |
| tataaatgta ccaatatgct ttgcttccaa gttatcccac tttgaatgcg agctgacagt | 2760 |
| atgactccaa aaagcgtata aacgtgggtg gtacaaattg aagcggttac tgaatgtcag | 2820 |
| attgtcaatt tttttccctt gtattatttt tttttttcac tcctgtttcc ttctgtattt | 2880 |
| tgtcgttctc tgtgcattac tcgacagatc tgtcgaaatc cccacctagt cagtgcattt | 2940 |
| cttatttgaa accatgcata tcctccatag tacattaggt ctcaactcaa acaaaacgct | 3000 |
| gactgacgta tggttccaat acgttctccg aaattacaaa tctccgagat tcataatcac | 3060 |
| aacttttggt gtgttattga catcatatat ttttttcccg tcatcgttac ttgcagtctc | 3120 |
| tcacaaacct tctaaaaggc cagataagta cacatgtggg ttcaaaaaca gcgggaatga | 3180 |
| ctgttttgcc aattctacac tacagtcact gtcttcgcta gatacacttt atttgtatct | 3240 |
| agccgagatg ctgagtttcc aaatgccacc aggatacacc atctacccat taccattaca | 3300 |
| tacgtctcta tatcatatgc | 3320 |

SEQ ID NO: 53. DNA integration cassette s483

| | |
|---|---|
| aatcaatata aatctggtgt cttccgtatt gccgaatggg ctgacatcac taatgcacat | 60 |
| ggtgtaacgg gtgcaggtat tgtttctggc ttgaaggagg cagcccaaga aacaaccagt | 120 |
| gaacctagag gtttgctaat gcttgctgag ttatcatcaa agggttcttt agcatatggt | 180 |
| gaatatacag aaaaaacagt agaaattgct aaatctgata aagagtttgt cattggtttt | 240 |
| attgcgcaac acgatatggg cggtagagaa gaaggttttg actggatcat tatgactcca | 300 |
| ggggttggtt tagatgacaa aggtgatgca cttggtcaac aatatagaac tgttgatgaa | 360 |
| gttgtaaaga ctggaacgga tatcataatt gttggtagag gtttgtacgg tcaaggaaga | 420 |
| gatcctatag agcaagctaa aagataccaa caagctggtt ggaatgctta tttaaacaga | 480 |
| tttaaatgag tgaatttact ttaaatcttg catttaaata aattttcttt ttatagcttt | 540 |
| atgacttagt ttcaatttat atactatttt aatgacattt tcgattcatt gattgaaagc | 600 |
| tttgtgtttt ttcttgatgc gctattgcat tgttcttgtc tttttcgcca catttaatat | 660 |
| ctgtagtaga tacctgatac attgtggatc gcctggcagc agggcgataa cctcataact | 720 |
| tcgtataatg tatgctatac gaacggtatt taggtgtcag acatttgcac ttgaaggata | 780 |
| ggagccccaa cctgttgtaa tttatgtttg atgttttgta acgtttatct ttatctttat | 840 |
| cttgatcttt gttttcgttt ttgtttatgt ttttgatttt atacagttat acttatgcta | 900 |
| agatctatat ctttgtttgg tcttacatat aaatgtacca atatgctttg cttccaagtt | 960 |
| atcccacttt gaatgcgagc tgacagtatg actccaaaaa gcgtataaac gtgggtggta | 1020 |
| caaattgaag cggttactga atgtcagatt gtcaattttt ttcccttgta ttattttttt | 1080 |
| ttttcactcc tgtttccttc tgtattttgt cgttctctgt gcattactcg acagatctgt | 1140 |
| cgaaatcccc acctagtcag tgcatttctt atttgaaacc atgcatatcc tccatagtac | 1200 |
| attaggtctc aactcaaaca aaacgctgac tgacgtatgg ttccaatacg ttctccgaaa | 1260 |
| ttacaaatct ccgagattca taatcacaac ttttggtgtg ttattgacat catatatttt | 1320 |
| tttcccgtca tcgttacttg cagtctctca caaaccttct aaaaggccag ataagtacac | 1380 |
| atgtgggttc aaaaacagcg ggaatgactg ttttgccaat tctacactac agtcactgtc | 1440 |
| ttcgctagat acactttatt tgtatctagc cgagatgctg agtttccaaa tgccaccagg | 1500 |
| atacaccatc tacccattac cattacatac gtctctatat catatgc | 1547 |

SEQ ID NO: 54. DNA integration cassette s394

| | |
|---|---|
| gcaggcttat ggcagacagg tacttttttt ttgtctctgt ataatgagtc aaattgtcaa | 60 |
| tattgaaggg ttgtatccaa actgcagttc ttgacagtca gacacactca tctttcataa | 120 |
| ccttccctaa atagatgtgc tcctatttca gccaagtatc tttattgtcg gtgaaaataa | 180 |
| tggaaacggt ctaaatgcgc ttgttactaa ggctgttact ttgataaacg catttgactt | 240 |
| tgagatatat aacttcaact ctaacgacct aatttcaaac ggaagagcta cttagaccat | 300 |
| agattaaaag tgaattctct ctaacacact ttgaggagca ttaatttcac accaaaacgt | 360 |
| ctatagatgc tgactttagc ggtttcaatg ggaattgatc ttgcaacacc aaggaattgc | 420 |
| cattgaagag aaacttactg atacatcatt caaccactcc gatgatatac accgggctag | 480 |
| atttcgatat ggatatggat atggatatgg atatggagat gaatttgaat ttagatttgg | 540 |
| gtcttgattt ggggttggaa ttaaaagggg ataacaatga gggttttcct gttgatttaa | 600 |
| acaatggacg tgggaggtga ttgatttaac ctgatccaaa aggggtatgt ctatttttta | 660 |
| gagtgtgtct ttgtgtcaaa ttatagtaga atgtgtaaag tagtataaac tttcctctca | 720 |
| aatgacgagg tttaaaacac cccccgggtg agccgagccg agaatggggc aattgttcaa | 780 |
| tgtgaaatag aagtatcgag tgagaaactt gggtgttggc cagccaaggg ggaaggaaaa | 840 |
| tggcgcgaat gctcaggtga gattgttttg gaattgggtg aagcgaggaa atgagcgacc | 900 |
| cggaggttgt gactttagtg gcggaggagg acggaggaaa agccaagagg gaagtgtata | 960 |
| taaggggagc aatttgccac caggatagaa ttggatgagt tataattcta ctgtatttat | 1020 |
| tgtataattt atttctcctt ttgtatcaaa cacattacaa aacacacaaa acacacaaac | 1080 |
| aaacacaatt acaaaaaatg ttgcacgttt ctatggttgg ttgtggtgct atcggtcgtg | 1140 |
| gtgtcttaga attgttgaag tccgatccag acgttgtttt cgatgttgtt attgttccag | 1200 |
| aacatactat ggatgaagct cgtggtgctg tctccgcttt agccccaaga gctagagttg | 1260 |
| ccacccactt ggatgatcaa cgtccagatt tgttagttga atgcgccggt catcacgctt | 1320 |
| tagaagaaca cattgtccca gccttagaaa gaggtatccc ttgtatggtt gtctctgttg | 1380 |
| gtgctttgtc tgagcctggt atggctgaac gtttggaagc cgctgctcgt agaggtggta | 1440 |
| cccaagtcca attgttgtcc ggtgctatcg gtgccatcga tgctttagcc gctgctcgtg | 1500 |
| tcggtggttt ggacgaagtt atctacaccg gtagaaaacc agctagagct tggaccggta | 1560 |
| ctccagctga gcaattgttc gacttggaag ctttaactga agccactgtc attttcgaag | 1620 |
| gtactgctag agatgccgct agattatacc ctaagaacgc taacgttgcc gctaccgttt | 1680 |
| ctttagctgg tttgggtttg gatagaaccg ctgttaagtt attggctgat cctcacgctg | 1740 |
| ttgaaaacgt ccaccatgtc gaagccagag gtgccttcgg tggtttcgaa ttgaccatga | 1800 |
| gaggtaagcc attggctgcc aacccaaaga cctctgcttt aactgtcttt tccgttgtta | 1860 |
| gagctttggg taatagagcc cacgccgttt ctatctaatc cagccagtaa aatccatact | 1920 |
| caacgacgat atgaacaaat ttccctcatt ccgatgctgt atatgtgtat aaatttttac | 1980 |
| atgctcttct gtttagacac agaacagctt taaataaaat gttggatata ctttttctgc | 2040 |
| ctgtggtgta ccgttcgtat aatgtatgct atacgaagtt ataaccggcg ttgccagcga | 2100 |
| taaacgggaa acatcatgaa aactgtttca ccctctggga agcataaaca ctagaaagcc | 2160 |
| aatgaagagc tctacaagcc tcttatgggt tcaatgggtc tgcaatgacc gcatacgggc | 2220 |
| ttggacaatt accttctatt gaatttctga gaagagatac atctcaccag caatgtaagc | 2280 |
| agacaatccc aattctgtaa acaacctctt tgtccataat tccccatcag aagagtgaaa | 2340 |
| aatgccctca aaatgcatgc gccacaccca cctctcaact gcactgcgcc acctctgagg | 2400 |
| gtcttttcag gggtcgacta ccccggacac ctcgcagagg agcgaggtca cgtactttta | 2460 |
| aaatggcaga gacgcgcagt ttcttgaaga aaggataaaa atgaaatggt gcggaaatgc | 2520 |
| gaaaatgatg aaaaattttc ttggtggcga ggaaattgag tgcaataatt ggcacgaggt | 2580 |
| tgttgccacc cgagtgtgag tatatatcct agtttctgca cttttcttct tcttttcttt | 2640 |
| accttttctt ttcaactttt ttttactttt tccttcaaca gacaaatcta acttatatat | 2700 |
| cacaatggcg tcatacaaag aaagatcaga atcacacact tcccctgttg ctaggagact | 2760 |
| tttctccatc atggaggaaa agaagtctaa cctttgtgca tcattggata ttactgaaac | 2820 |
| tgaaaagctt ctctctattt tggacactat tggtccttac atctgtctag ttaaaacaca | 2880 |
| catcgatatt gtttctgatt ttacgtatga aggaactgtg ttgcctttga aggagcttgc | 2940 |
| caagaaacat aattttatga tttttgaaga tagaaaattt gctgatattg gtaacaccgt | 3000 |
| taaaaatcaa tataaatctg gtgtcttccg tattgccgaa tgggctgaca tcactaatgc | 3060 |
| acatggtgta acgggtgcag gtattgtttc tggcttgaag gaggcagccc aagaaacaac | 3120 |
| cagtgaacct agaggtttgc taatgcttgc tgagttatca tcaaagggtt ctttagcata | 3180 |
| tggtgaatat acagaaaaaa cagtagaaat tgctaaatct gataaagagt ttgtcattgg | 3240 |
| ttttattgcg caacacgata tgggcggtag agaagaaggt tttgactgga tcattatgac | 3300 |
| tcca | 3304 |

SEQ ID NO: 55. DNA integration cassette s396

| | |
|---|---|
| gcaggcttat ggcagacagg tacttttttt ttgtctctgt ataatgagtc aaattgtcaa | 60 |
| tattgaaggg ttgtatccaa actgcagttc ttgacagtca gacacactca tctttcataa | 120 |
| ccttccctaa atagatgtgc tcctatttca gccaagtatc tttattgtcg gtgaaaataa | 180 |
| tggaaacggt ctaaatgcgc ttgttactaa ggctgttact ttgataaacg catttgactt | 240 |
| tgagatatat aacttcaact ctaacgacct aatttcaaac ggaagagcta cttagaccat | 300 |
| agattaaaag tgaattctct ctaacacact ttgaggagca ttaatttcac accaaaacgt | 360 |
| ctatagatgc tgactttagc ggtttcaatg ggaattgatc ttgcaacacc aaggaattgc | 420 |
| cattgaagag aaacttactg atacatcatt caaccactcc gatgatatac accgggctag | 480 |
| atttcgatat ggatatggat atggatatgg atatggagat gaatttgaat ttagatttgg | 540 |
| gtcttgattt ggggttggaa ttaaaagggg ataacaatga gggttttcct gttgatttaa | 600 |
| acaatggacg tgggaggtga ttgatttaac ctgatccaaa aggggtatgt ctatttttta | 660 |
| gagtgtgtct ttgtgtcaaa ttatagtaga atgtgtaaag tagtataaac tttcctctca | 720 |
| aatgacgagg tttaaaacac cccccgggtg agccgagccg agaatggggc aattgttcaa | 780 |
| tgtgaaatag aagtatcgag tgagaaactt gggtgttggc cagccaaggg ggaaggaaaa | 840 |
| tggcgcgaat gctcaggtga gattgttttg gaattgggtg aagcgaggaa atgagcgacc | 900 |
| cggaggttgt gactttagtg gcggaggagg acggaggaaa agccaagagg gaagtgtata | 960 |
| taaggggagc aatttgccac caggatagaa ttggatgagt tataattcta ctgtatttat | 1020 |
| tgtataattt atttctcctt ttgtatcaaa cacattacaa aacacacaaa acacacaaac | 1080 |
| aaacacaatt acaaaaaatg ttgaagatcg ctatgattgg ttgtggtgct atcggtgcct | 1140 |
| ccgtcttgga attgttgcat ggtgactctg acgttgttgt tgatagagtt atcaccgttc | 1200 |
| cagaagctag agacagaact gaaatcgctg ttgccagatg ggctccaaga gccagagttt | 1260 |
| tggaagtttt ggctgctgac gatgccccag acttggttgt tgaatgtgcc ggtcacggtg | 1320 |
| ctatcgctgc tcatgttgtc ccagccttgg aaagaggtat tccatgtgtt gttacctccg | 1380 |
| ttggtgcttt gtctgctcca ggtatggctc aattattgga gcaagccgcc agaagaggta | 1440 |
| agacccaagt ccaattgttg tccggtgcta tcggtggtat cgacgcttta gctgccgcta | 1500 |
| gagtcggtgg tttggattcc gtcgtttaca ctggtagaaa gccaccaatg gcctggaagg | 1560 |
| gtactcctgc tgaagctgtc tgtgatttgg actctttgac cgttgcccac tgtattttcg | 1620 |
| acggttctgc tgaacaagcc gcccaattat acccaaagaa cgctaacgtt gctgctactt | 1680 |
| tgtctttagc cggtttgggt ttgaagagaa ctcaagtcca attgttcgct gacccaggtg | 1740 |
| tttctgagaa tgttcaccac gtcgctgctc atggtgcttt cggttctttc gaattgacta | 1800 |
| tgagaggtag accattggct gccaacccta agacctctgc tttgaccgtc tattctgttg | 1860 |
| tcagagcttt gttaaacaga ggtagagctt tggttattta atccagccag taaaatccat | 1920 |
| actcaacgac gatatgaaca aatttccctc attccgatgc tgtatatgtg tataaatttt | 1980 |
| tacatgctct tctgtttaga cacagaacag ctttaaataa aatgttggat atactttttc | 2040 |
| tgcctgtggt gtaccgttcg tataatgtat gctatacgaa gttataaccg gcgttgccag | 2100 |
| cgataaacgg gaaacatcat gaaaactgtt tcaccctctg ggaagcataa acactagaaa | 2160 |
| gccaatgaag agctctacaa gcctcttatg ggttcaatgg gtctgcaatg accgcatacg | 2220 |
| ggcttggaca attaccttct attgaatttc tgagaagaga tacatctcac cagcaatgta | 2280 |
| agcagacaat cccaattctg taaacaacct ctttgtccat aattccccat cagaagagtg | 2340 |
| aaaaatgccc tcaaaatgca tgcgccacac ccacctctca actgcactgc gccacctctg | 2400 |
| agggtctttt caggggtcga ctaccccgga cacctcgcag aggagcgagg tcacgtactt | 2460 |
| ttaaaatggc agagacgcgc agtttcttga agaaaggata aaaatgaaat ggtgcggaaa | 2520 |
| tgcgaaaatg atgaaaaatt ttcttggtgg cgaggaaatt gagtgcaata attggcacga | 2580 |
| ggttgttgcc acccgagtgt gagtatatat cctagtttct gcacttttct tcttcttttc | 2640 |
| tttacctttt cttttcaact tttttttact ttttccttca acagacaaat ctaacttata | 2700 |
| tatcacaatg gcgtcataca aagaaagatc agaatcacac acttcccctg ttgctaggag | 2760 |
| acttttctcc atcatggagg aaaagaagtc taacctttgt gcatcattgg atattactga | 2820 |
| aactgaaaag cttctctcta ttttggacac tattggtcct tacatctgtc tagttaaaac | 2880 |
| acacatcgat attgtttctg attttacgta tgaaggaact gtgttgcctt tgaaggagct | 2940 |
| tgccaagaaa cataatttta tgatttttga agatagaaaa tttgctgata ttggtaacac | 3000 |
| cgttaaaaat caatataaat ctggtgtctt ccgtattgcc gaatgggctg acatcactaa | 3060 |
| tgcacatggt gtaacgggtg caggtattgt ttctggcttg aaggaggcag cccaagaaac | 3120 |
| aaccagtgaa cctagaggtt tgctaatgct tgctgagtta tcatcaaagg gttctttagc | 3180 |
| atatggtgaa tatacagaaa aaacagtaga aattgctaaa tctgataaag agtttgtcat | 3240 |
| tggttttatt gcgcaacacg atatgggcgg tagagaagaa ggttttgact ggatcattat | 3300 |
| gactcca | 3307 |

SEQ ID NO: 56. DNA integration cassette s408

| | |
|---|---|
| aatcaatata aatctggtgt cttccgtatt gccgaatggg ctgacatcac taatgcacat | 60 |
| ggtgtaacgg gtgcaggtat tgtttctggc ttgaaggagg cagcccaaga aacaaccagt | 120 |
| gaacctagag gtttgctaat gcttgctgag ttatcatcaa agggttcttt agcatatggt | 180 |
| gaatatacag aaaaaacagt agaaattgct aaatctgata aagagtttgt cattggtttt | 240 |
| attgcgcaac acgatatggg cggtagagaa gaaggttttg actggatcat tatgactcca | 300 |
| ggggttggtt tagatgacaa aggtgatgca cttggtcaac aatatagaac tgttgatgaa | 360 |
| gttgtaaaga ctggaacgga tatcataatt gttggtagag gtttgtacgg tcaaggaaga | 420 |
| gatcctatag agcaagctaa aagataccaa caagctggtt ggaatgctta tttaaacaga | 480 |
| tttaaatgag tgaatttact ttaaatcttg catttaaata aattttcttt ttatagcttt | 540 |
| atgacttagt ttcaatttat atactatttt aatgacattt tcgattcatt gattgaaagc | 600 |
| tttgtgtttt ttcttgatgc gctattgcat tgttcttgtc tttttcgcca catttaatat | 660 |
| ctgtagtaga tacctgatac attgtggatc gcctggcagc agggcgataa cctcataact | 720 |
| tcgtataatg tatgctatac gaacggtagc tacttagctt ctatagttag ttaatgcact | 780 |
| cacgatattc aaaattgaca cccttcaact actccctact attgtctact actgtctact | 840 |
| actcctcttt actatagctg ctcccaatag gctccaccaa taggctctgc caatacattt | 900 |
| tgcgccgcca cctttcaggt tgtgtcactc ctgaaggacc atattgggta atcgtgcaat | 960 |
| ttctggaaga gagtccgcga gaagtgaggc ccccactgta aatcctcgag ggggcatgga | 1020 |
| gtatggggca tggaggatgg aggatggggg gggggcgaaa aataggtagc aaaaggaccc | 1080 |
| gctatcaccc cacccggaga actcgttgcc gggaagtcat atttcgacac tccggggagt | 1140 |
| ctataaaagg cgggttttgt cttttgccag ttgatgttgc tgaaaggact tgtttgccgt | 1200 |
| ttcttccgat ttaacagtat agaaatcaac cactgttaat tatacacgtt atactaacac | 1260 |
| aacaaaaaca aaaacaacga caacaacaac aacaatgaag ggcggctcta tggagaaaat | 1320 |
| aaagcccatc ttagcaatta tttctttgca attcggctac gcagggatgt acatcattac | 1380 |
| aatggtgagt ttcaagcacg gtatggacca ttgggtgctt gcaacctata gacacgttgt | 1440 |
| ggccaccgta gtcatggccc cgtttgccct gatgtttgag cgtaaaatca gaccgaagat | 1500 |
| gacgttggct atcttctgga gacttctggc cctagggatc ctagagccct tgatggatca | 1560 |
| gaatctgtat tacatcggtt tgaagaatac ctctgcttca tacacgtccg cattcacaaa | 1620 |
| cgccttgcct gctgtcacat tcattctggc cctgatcttc cgtttggaaa cggtcaattt | 1680 |
| caggaaagtc catagtgtcg ccaaggtagt cggtacagtg attacagtgg gcggtgcaat | 1740 |
| gattatgacg ctatacaaag gccccgcgat agagattgtc aaggcagcac acaactcctt | 1800 |
| tcacgggggc tcctcctcca cgcctacagg tcagcactgg gtgctaggca caatcgccat | 1860 |
| tatgggtagc attagcactt gggcagcgtt ttttatactt caatcctata cattaaaagt | 1920 |
| ctacccagct gagctgagct tggtaactct tatctgcggt attggaacga tcctaaacgc | 1980 |
| tatagccagt ttaatcatgg ttagggatcc atccgcttgg aaaataggca tggattctgg | 2040 |
| gactttagct gctgtttatt ccggagtggt atgtagtgga atcgcgtatt acatccagag | 2100 |
| catcgtcatt aagcaacgtg gtcccgtatt cacgacctcc ttctctccaa tgtgtatgat | 2160 |
| aataaccgcc ttcctgggcg ccctggtact agctgagaag attcatcttg gttcaatcat | 2220 |
| tggagcggtg tttatcgtat tgggcctgta cagtgttgtg tggggaaaaa gtaaggatga | 2280 |
| ggttaatcca ttggacgaaa aaatagtagc aaagtctcag gagctgccca tcacaaacgt | 2340 |
| tgtaaagcag acgaacggtc acgatgtaag cggtgcccca acaaatggag tagtgaccag | 2400 |
| tacctaagat taatataatt atataaaaat attatcttct tttctttata tctagtgtta | 2460 |
| tgtaaaataa attgatgact acggaaagct tttttatatt gtttcttttt cattctgagc | 2520 |
| cacttaaatt tcgtgaatgt tcttgtaagg gacggtagat ttacaagtga tacaacaaaa | 2580 |
| agcaaggcgc tttttctaat aaaaagaaga aaagcattta acaattgaac acctctatat | 2640 |
| caacgaagaa tattactttg tctctaaatc cttgtaaaat gtgtacgatc tctatatggg | 2700 |
| ttactcagat agacatctga gtgagcgata gatagataga tagatagata gatgtatggg | 2760 |
| tagatagatg catatataga tgcatggaat gaaaggaaga tagatagaga gaaatgcaga | 2820 |
| aataagcgta tgaggtttaa ttttaatgta catacatgta tagataaacg atgtcgatat | 2880 |
| aatttattta gtaaacagat tccctgatat gtgtttttag ttttattttt ttttgttttt | 2940 |
| tctatgttga aaaacttgat gacatgatcg agtaaaattg gagcttgatt tcattcatct | 3000 |
| tgttgattcc tttatcataa tgcaaagctg ggggggggga gggtaaaaaa aagtgaagaa | 3060 |
| aaagaaagta tgatacaact gtggaagtgg ag | 3092 |

SEQ ID NO: 57. DNA integration cassette s409

| | |
|---|---|
| aatcaatata aatctggtgt cttccgtatt gccgaatggg ctgacatcac taatgcacat | 60 |
| ggtgtaacgg gtgcaggtat tgtttctggc ttgaaggagg cagcccaaga aacaaccagt | 120 |
| gaacctagag gtttgctaat gcttgctgag ttatcatcaa agggttcttt agcatatggt | 180 |
| gaatatacag aaaaaacagt agaaattgct aaatctgata aagagtttgt cattggtttt | 240 |
| attgcgcaac acgatatggg cggtagagaa gaaggttttg actggatcat tatgactcca | 300 |
| ggggttggtt tagatgacaa aggtgatgca cttggtcaac aatatagaac tgttgatgaa | 360 |
| gttgtaaaga ctggaacgga tatcataatt gttggtagag gtttgtacgg tcaaggaaga | 420 |
| gatcctatag agcaagctaa aagataccaa caagctggtt ggaatgctta tttaaacaga | 480 |
| tttaaatgag tgaatttact ttaaatcttg catttaaata aattttcttt ttatagcttt | 540 |
| atgacttagt ttcaatttat atactatttt aatgacattt tcgattcatt gattgaaagc | 600 |
| tttgtgtttt ttcttgatgc gctattgcat tgttcttgtc tttttcgcca catttaatat | 660 |
| ctgtagtaga tacctgatac attgtggatc gcctggcagc agggcgataa cctcataact | 720 |
| tcgtataatg tatgctatac gaacggtagc tacttagctt ctatagttag ttaatgcact | 780 |
| cacgatattc aaaattgaca cccttcaact actccctact attgtctact actgtctact | 840 |
| actcctcttt actatagctg ctcccaatag gctccaccaa taggctctgc caatacattt | 900 |
| tgcgccgcca cctttcaggt tgtgtcactc ctgaaggacc atattgggta atcgtgcaat | 960 |
| ttctggaaga gagtccgcga gaagtgaggc ccccactgta aatcctcgag ggggcatgga | 1020 |
| gtatggggca tggaggatgg aggatggggg gggggcgaaa aataggtagc aaaaggaccc | 1080 |
| gctatcaccc cacccggaga actcgttgcc gggaagtcat atttcgacac tccggggagt | 1140 |
| ctataaaagg cgggttttgt cttttgccag ttgatgttgc tgaaaggact tgtttgccgt | 1200 |
| ttcttccgat ttaacagtat agaaatcaac cactgttaat tatacacgtt atactaacac | 1260 |
| aacaaaaaca aaaacaacga caacaacaac aacaatgggg ctgggcgggg atcagtcctt | 1320 |
| cgtaccggta atggatagcg gacaggtaag attgaaggaa ctgggctata agcaggaact | 1380 |
| gaaaagggac ttgtcagtgt tctcaaactt cgcgatatct tttagcataa taagcgtctt | 1440 |
| aacaggcatt accaccacgt acaatacagg cttgagattc ggaggaactg tcaccctagt | 1500 |
| ctacggttgg tttttagccg ggagtttcac tatgtgcgta ggtcttagca tggctgaaat | 1560 |
| atgcagcagc tatcctacca gcggcggtct ttattactgg agcgcaatgc ttgctggacc | 1620 |
| gcgttgggct ccattggcaa gttggatgac cggttggttt aatatagtgg gtcagtgggc | 1680 |
| cgtaacagcc tcagtggact ttagtcttgc ccaattgatc caggtcatcg tgcttttgtc | 1740 |
| tacgggcggg aggaacgggg gcggatataa ggggagcgac ttcgtcgtaa tagggattca | 1800 |
| cgggggtatc ttatttatcc acgcccttct aaattccctt cctatcagcg tattgtcctt | 1860 |
| catcgggcaa ttggccgctc tatggaatct tctaggggtc ctagttctta tgatattgat | 1920 |
| ccctctggtg agcacagaaa gagctaccac aaaatttgtc tttaccaatt tcaataccga | 1980 |
| taatggactt gggattactt cttatgctta tatcttcgtt cttggcctgc tgatgagtca | 2040 |
| atacacaata accggctatg atgctagcgc tcacatgacg gaggaaactg tcgacgcgga | 2100 |
| taaaaatggg cctaggggta ttatcagtgc cattgggatc tccatattgt tcggttgggg | 2160 |
| gtacatcttg ggtatatcct atgcagtcac agacattcct tcccttcttt ccgaaactaa | 2220 |
| taacagtggc ggatacgcga tcgcagaaat tttttatctt gcgtttaaga atcgtttcgg | 2280 |
| ttctgggact ggtggtattg tctgtctggg ggtagtagcg gttgcggtgt ttttctgtgg | 2340 |
| gatgagtagc gtcacatcaa attccagaat ggcatacgcc ttttctagag acggagcaat | 2400 |
| gcctatgtcc cccctatggc ataaggttaa ctcaagagag gtgcctataa acgcggtgtg | 2460 |
| gctttctgct ctgatttctt tttgcatggc gttaacgtcc ttaggatcaa tagtcgcgtt | 2520 |
| ccaggcgatg gtcagtattg ctaccatcgg gttgtacata gcctatgcaa tacccattat | 2580 |
| actaagggta actttggcac gtaatacctt tgttcccggt ccattcagcc ttggcaaata | 2640 |
| tggtatggtt gttggctggg tagcggttct gtgggtagtt acaatttccg ttttgttttc | 2700 |
| tttacccgtg gcctacccca taactgcgga aacgcttaat tatacaccgg tcgccgtagc | 2760 |
| agggctggtt gccattacat taagttactg gctgttttca gcgcgtcatt ggtttacagg | 2820 |
| tccaatatct aatattttgt cataagatta atataattat ataaaaatat tatcttcttt | 2880 |
| tctttatatc tagtgttatg taaaataaat tgatgactac ggaaagcttt tttatattgt | 2940 |
| ttctttttca ttctgagcca cttaaatttc gtgaatgttc ttgtaaggga cggtagattt | 3000 |
| acaagtgata caacaaaaag caaggcgctt tttctaataa aaagaagaaa agcatttaac | 3060 |
| aattgaacac ctctatatca acgaagaata ttactttgtc tctaaatcct tgtaaaatgt | 3120 |
| gtacgatctc tatatgggtt actcagatag acatctgagt gagcgataga tagatagata | 3180 |
| gatagataga tgtatgggta gatagatgca tatatagatg catggaatga aaggaagata | 3240 |
| gatagagaga aatgcagaaa taagcgtatg aggtttaatt ttaatgtaca tacatgtata | 3300 |
| gataaacgat gtcgatataa tttatttagt aaacagattc cctgatatgt gtttttagtt | 3360 |
| ttattttttt ttgttttttc tatgttgaaa aacttgatga catgatcgag taaaattgga | 3420 |
| gcttgatttc attcatcttg ttgattcctt tatcataatg caaagctggg gggggggagg | 3480 |
| gtaaaaaaaa gtgaagaaaa agaaagtatg atacaactgt ggaagtggag | 3530 |

SEQ ID NO: 58. Pichia kudrizaevii pyruvate carboxylase

| | |
|---|---|
| 1- | MSTVEDHSSL HKLRKESEIL SNANKILVAN RGEIPIRIFR |
| 41- | SAHELSMHTV AIYSHEDRLS MHRLKADEAY AIGKTGQYSP |
| 81- | VQAYLQIDEI IKIAKEHDVS MIHPGYGFLS ENSEFAKKVE |
| 120- | ESGMIWVGPP AEVIDSVGDK VSARNLAIKC DVPVVPGTDG |
| 161- | PIEDIEQAKQ FVEQYGYPVI IKAAFGGGGR GMRVVREGDD |
| 201- | IVDAFQRASS EAKSAFGNGT CFIERFLDKP KHIEVQLLAD |
| 241- | NYGNTIHLFE RDCSVQRRHQ KVVEIAPAKT LPVEVRNAIL |
| 281- | KDAVTLAKTA NYRNAGTAEF LVDSQNRHYF IEINPRIQVE |
| 321- | HTITEEITGV DIVAAQIQIA AGASLEQLGL LQNKITTRGF |
| 361- | AIQCRITTED PAKNFAPDTG KIEVYRSAGG NGVRLDGGNG |
| 401- | FAGAVISPHY DSMLVKCSTS GSNYEIARRK MIRALVEFRI |
| 441- | RGVKTNIPFL LALLTHPVFI SGDCWTTFID DTPSLFEMVS |
| 481- | SKNRAQKLLA YIGDLCVNGS SIKGQIGFPK LNKEAEIPDL |
| 521- | LDPNDEVIDV SKPSTNGLRP YLLKYGPDAF SKKVREFDGC |
| 561- | MIMDTTWRDA HQSLLATRVR TIDLLRIAPT TSHALQNAFA |
| 601- | LECWGGATFD VAMRFLYEDP WERLRQLRKA VPNIPFQMLL |
| 641- | RGANGVAYSS LPDNAIDHFV KQAKDNGVDI FRVFDALNDL |
| 681- | EQLKVGVDAV KKAGGVVEAT VCYSGDMLIP GKKYNLDYYL |
| 721- | ETVGKIVEMG THILGIKDMA GTLKPKAAKL LIGSIRSKYP |
| 761- | DLVIHVHTHD SAGTGISTYV ACALAGADIV DCAINSMSGL |
| 801- | TSQPSMSAFI AALDGDIETG VPEHFARQLD AYWAEMRLLY |
| 841- | SCFEADLKGP DPEVYKHEIP GGQLTNLIFQ AQQVGLGEQW |
| 881- | EETKKKYEDA NMLLGDIVKV TPTSKVVGDL AQFMVSNKLE |
| 921- | KEDVEKLANE LDFPDSVLDF FEGLMGTPYG GFPEPLRTNV |
| 961- | ISGKRRKLKG RPGLELEPFN LEEIRENLVS RFGPGITECD |
| 1001- | VASYNMYPKV YEQYRKVVEK YGDLSVLPTK AFLAPPTIGE |
| 1041- | EVHVEIEQGK TLIIKLLAIS DLSKSHGTRE VYFELNGEMR |
| 1081- | KVTIEDKTAA IETVTRAKAD GHNPNEVGAP MAGVVVEVRV |
| 1121- | KHGTEVKKGD PLAVLSAMKM EMVISAPVSG RVGEVFVNEG |
| 1161- | DSVDMGDLLV KIAKDEAPAA -1180 |

1. A recombinant yeast cell comprising:
(a) a heterologous nucleic acid encoding an L-aspartate dehydrogenase; and
(b) a heterologous nucleic acid encoding an oxaloacetate-forming enzyme selected from the group consisting of pyruvate carboxylase, phosphoenolpyruvate carboxylase, and phosphoenolpyruvate carboxykinase.

2. The recombinant yeast cell of claim 1, wherein the heterologous nucleic acid encoding an oxaloacetate-forming enzyme is pyruvate carboxylase.

3. A recombinant yeast cell comprising:
(a) a heterologous nucleic acid encoding an L-aspartate dehydrogenase;
(b) a heterologous nucleic acid encoding an oxaloacetate-forming enzyme selected from the group consisting of pyruvate carboxylase, phosphoenolpyruvate carboxylase, and phosphoenolpyruvate carboxykinase; and
(c) a deletion or disruption of a nucleic acid encoding pyruvate decarboxylase.

4. The recombinant yeast cell of claim 2 wherein the recombinant host cell is capable of producing L-aspartate and/or beta-alanine under substantially anaerobic conditions.

5. The recombinant yeast cell of claim 2 wherein the recombinant host cell is capable of producing L-aspartate and/or beta-alanine under aerobic conditions.

6. The recombinant yeast cell of claim 3 wherein the heterologous nucleic acid encoding an oxaloacetate-forming enzyme is pyruvate carboxylase.

7. The recombinant host cell of claim 1, further comprising a heterologous nucleic acid encoding a L-aspartate 1-decarboxylase wherein the recombinant host cell is capable of producing beta-alanine under substantially anaerobic conditions.


SEQUENCE LISTING (58 sequences)

SEQ ID NO: 1 - protein, 267 aa, Pseudomonas aeruginosa
MLNIVMIGCGAIGAGVLELLENDPQLRVDAVIVPRDSETQVRHRLASLRRPPRVLSALPAGERPDLLVECAGHRAIEQHVLPALAQGIPCLVVSVGALSEPGLVERLEAAAQAGGSRIELLPGAIGAIDALSAARVGGLESVRYTGRKPASAWLGTPGETVCDLQRLEKARVIFDGSAREAARLYPKNANVAATLSLAGLGLDRTQVRLIADPESCENVHQVEASGAFGGFELTLRGKPLAANPKTSALTVYSVVRALGNHAHAISI

SEQ ID NO: 2 - protein, 266 aa, Cupriavidus taiwanensis
MLHVSMVGCGAIGRGVLELLKSDPDVVFDVVIVPEHTMDEARGAVSALAPRARVATHLDDQRPDLLVECAGHHALEEHIVPALERGIPCMVVSVGALSEPGMAERLEAAARRGGTQVQLLSGAIGAIDALAAARVGGLDEVIYTGRKPARAWTGTPAEQLFDLEALTEATVIFEGTARDAARLYPKNANVAATVSLAGLGLDRTAVKLLADPHAVENVHHVEARGAFGGFELTMRGKPLAANPKTSALTVFSVVRALGNRAHAVSI

SEQ ID NO: 3 - protein, 540 aa, Tribolium castaneum
MPATGEDQDLVQDLIEEPATFSDAVLSSDEELFHQKCPKPAPIYSPISKPVSFESLPNRRLHEEFLRSSVDVLLQEAVFEGTNRKNRVLQWREPEELRRLMDFGVRGAPSTHEELLEVLKKVVTYSVKTGHPYFVNQLFSAVDPYGLVAQWATDALNPSVYTYEVSPVFVLMEEVVLREMRAIVGFEGGKGDGIFCPGGSIANGYAISCARYRFMPDIKKKGLHSLPRLVLFTSEDAHYSIKKLASFEGIGTDNVYLIRTDARGRMDVSHLVEEIERSLREGAAPFMVSATAGTTVIGAFDPIEKIADVCQKYKLWLHVDAAWGGGALVSAKHRHLLKGIERADSVTWNPHKLLTAPQQCSTLLLRHEGVLAEAHSTNAAYLFQKDKFYDTKYDTGDKHIQCGRRADVLKFWFMWKAKGTSGLEKHVDKVFENARFFTDCIKNREGFEMVIAEPEYTNICFWYVPKSLRGRKDEADYKDKLHKVAPRIKERMMKEGSMMVTYQAQKGHPNFFRIVFQNSGLDKADMVHFVEEIERLGSDL

SEQ ID NO: 4 - protein, 136 aa, Corynebacterium glutamicum
MLRTILGSKIHRATVTQADLDYVGSVTIDADLVHAAGLIEGEKVAIVDITNGARLETYVIVGDAGTGNICINGAAAHLINPGDLVIIMSYLQATDAEAKAYEPKIVHVDADNRIVALGNDLAEALPGSGLLTSRSI

SEQ ID NO: 5 - protein, 127 aa, Bacillus subtilis
MYRTMMSGKLHRATVTEANLNYVGSITIDEDLIDAVGMLPNEKVQIVNNNNGARLETYIIPGKRGSGVICLNGAAARLVQEGDKVIIISYKMMSDQEAASHEPKVAVLNDQNKIEQMLGNEPARTIL

SEQ ID NO: 6 - protein, 538 aa, Mannheimia succiniciproducens
MTDLNQLTQELGALGIHDVQEVVYNPSYELLFAEETKPGLEGYEKGTVTNQGAVAVNTGIFTGRSPKDKYIVLDDKTKDTVWWTSEKVKNDNKPMSQDTWNSLKGLVADQLSGKRLFVVDAFCGANKDTRLAVRVVTEVAWQAHFVTNMFIRPSAEELKGFKPDFVVMNGAKCTNPNWKEQGLNSENFVAFNITEGVQLIGGTWYGGEMKKGMFSMMNYFLPLRGIASMHCSANVGKDGDTAIFFGLSGTGKTTLSTDPKRQLIGDDEHGWDDEGVFNFEGGCYAKTINLSAENEPDIYGAIKRDALLENVVVLDNGDVDYADGSKTENTRVSYPIYHIQNIVKPVSKAGPATKVIFLSADAFGVLPPVSKLTPEQTKYYFLSGFTAKLAGTERGITEPTPTFSACFGAAFLSLHPTQYAEVLVKRMQESGAEAYLVNTGWNGTGKRISIKDTRGIIDAILDGSIDKAEMGSLPIFDFSIPKALPGVNPAILDPRDTYADKAQWEEKAQDLAGRFVKNFEKYTGTAEGQALVAAGPKA

SEQ ID NO: 7 - protein, 1193 aa, Aspergillus oryzae
MAAPFRQPEEAVDDTEFIDDHHEHLRDTVHHRLRANSSIMHFQKILVANRGEIPIRIFRTAHELSLQTVAIYSHEDRLSMHRQKADEAYMIGHRGQYTPVGAYLAGDEIIKIALEHGVQLIHPGYGFLSENADFARKVENAGIVFVGPTPDTIDSLGDKVSARRLAIKCEVPVVPGTEGPVERYEEVKAFTDTYGFPIIIKAAFGGGGRGMRVVRDQAELRDSFERATSEARSAFGNGTVFVERFLDKPKHIEVQLLGDSHGNVVHLFERDCSVQRRHQKVVEVAPAKDLPADVRDRILADAVKLAKSVNYRNAGTAEFLVDQQNRHYFIEINPRIQVEHTITEEITGIDIVAAQIQIAAGASLEQLGLTQDRISARGFAIQCRITTEDPAKGFSPDTGKIEVYRSAGGNGVRLDGGNGFAGAIITPHYDSMLVKCTCRGSTYEIARRKVVRALVEFRIRGVKTNIPFLTSLLSHPTFVDGNCWTTFIDDTPELFSLVGSQNRAQKLLAYLGDVAVNGSSIKGQIGEPKLKGDVIKPKLFDAEGKPLDVSAPCTKGWKQILDREGPAAFAKAVRANKGCLIMDTTWRDAHQSLLATRVRTIDLLNIAHETSYAYSNAYSLECWGGATFDVAMRFLYEDPWDRLRKMRKAVPNIPFQMLLRGANGVAYSSLPDNAIYHFCKQAKKCGVDIFRVFDALNDVDQLEVGIKAVHAAEGVVEATMCYSGDMLNPHKKYNLEYYMALVDKIVAMKPHILGIKDMAGVLKPQAARLLVGSIRQRYPDLPIHVHTHDSAGTGVASMIACAQAGADAVDAATDSMSGMTSQPSIGAILASLEGTEQDPGLNLAHVRAIDSYWAQLRLLYSPFEAGLTGPDPEVYEHEIPGGQLTNLIFQASQLGLGQQWAETKKAYEAANDLLGDIVKVTPTSKVVGDLAQFMVSNKLTPEDVVERAGELDFPGSVLEFLEGLMGQPFGGFPEPLRSRALRDRRKLEKRPGLYLEPLDLAKIKSQIREKFGAATEYDVASYAMYPKVFEDYKKFVQKFGDLSVLPTRYFLAKPEIGEEFHVELEKGKVLILKLLAIGPLSEQTGQREVFYEVNGEVRQVAVDDNKASVDNTSRPKADVGDSSQVGAPMSGVVVEIRVHDGLEVKKGDPLAVLSAMKMEMVISAPHSGKVSSLLVKEGDSVDGQDLVCKIVKA

SEQ ID NO: 8 - protein, 883 aa, Escherichia coli
MNEQYSALRSNVSMLGKVLGETIKDALGEHILERVETIRKLSKSSRAGNDANRQELLTTLQNLSNDELLPVARAFSQFLNLANTAEQYHSISPKGEAASNPEVIARTLRKLKNQPELSEDTIKKAVESLSLELVLTAHPTEITRRTLIHKMVEVNACLKQLDNKDIADYEHNQLMRRLRQLIAQSWHTDEIRKLRPSPVDEAKWGFAVVENSLWQGVPNYLRELNEQLEENLGYKLPVEFVPVRFTSWMGGDRDGNPNVTADITRHVLLLSRWKATDLFLKDIQVLVSELSMVEATPELLALVGEEGAAEPYRYLMKNLRSRLMATQAWLEARLKGEELPKPEGLLTQNEELWEPLYACYQSLQACGMGIIANGDLLDTLRRVKCFGVPLVRIDIRQESTRHTEALGELTRYLGIGDYESWSEADKQAFLIRELNSKRPLLPRNWQPSAETREVLDTCQVIAEAPQGSIAAYVISMAKTPSDVLAVHLLLKEAGIGFAMPVAPLFETLDDLNNANDVMTQLLNIDWYRGLIQGKQMVMIGYSDSAKDAGVMAASWAQYQAQDALIKTCEKAGIELTLFHGRGGSIGRGGAPAHAALLSQPPGSLKGGLRVTEQGEMIRFKYGLPEITVSSLSLYTGAILEANLLPPPEPKESWRRIMDELSVISCDVYRGYVRENKDFVPYFRSATPEQELGKLPLGSRPAKRRPTGGVESLRAIPWIFAWTQNRLMLPAWLGAGTALQKVVEDGKQSELEAMCRDWPFFSTRLGMLEMVFAKADLWLAEYYDQRLVDKALWPLGKELRNLQEEDIKVVLAIANDSHLMADLPWIAESIQLRNIYTDPLNVLQAELLHRSRQAEKEGQEPDPRVEQALMVTIAGIAAGMRNTG

SEQ ID NO: 9 - protein, 575 aa, Pichia kudriavzevii
MTDKISLGTYLFEKLKEAGSYSIFGVPGDFNLALLDHVKEVEGIRWVGNANELNAGYEADGYARINGFASLITTFGVGELSAVNAIAGSYAEHVPLIHIVGMPSLSAMKNNLLLHHTLGDTRFDNFTEMSKKISAKVEIVYDLESAPKLINNLIETAYHTKRPVYLGLPSNFADELVPAALVKENKLHLEEPLNNPVAEEEFIHNVVEMVKKAEKPIILVDACAARHNISKEVRELAKLTKFPVFTTPMGKSTVDEDDEEFFGLYLGSLSAPDVKDIVGPTDCILSLGGLPSDFNTGSFSYGYTTKNVVEFHSNYCKFKSATYENLMMKGAVQRLISELKNIKYSNVSTLSPPKSKFAYESAKVAPEGIITQDYLWKRLSYFLKPRDIIVTETGTSSFGVLATHLPRDSKSISQVLWGSIGFSLPAAVGAAFAAEDAHKQTGEQERRTVLFIGDGSLQLTVQSISDAARWNIKPYIFILNNRGYTIEKLIHGRHEDYNQIQPWDHQLLLKLFADKTQYENHVVKSAKDLDALMKDEAFNKEDKIRVIELFLDEFDAPEILVAQAKLSDEINSKAA

SEQ ID NO: 10 - protein, 563 aa, Saccharomyces cerevisiae
MSEITLGKYLFERLKQVNVNTVFGLPGDFNLSLLDKIYEVEGMRWAGNANELNAAYAADGYARIKGMSCIITTFGVGELSALNGIAGSYAEHVGVLHVVGVPSISAQAKQLLLHHTLGNGDFTVFHRMSANISETTAMITDIATAPAEIDRCIRTTYVTQRPVYLGLPANLVDLNVPAKLLQTPIDMSLKPNDAESEKEVIDTILALVKDAKNPVILADACCSRHDVKAETKKLIDLTQFPAFVTPMGKGSIDEQHPRYGGVYVGTLSKPEVKEAVESADLILSVGALLSDFNTGSFSYSYKTKNIVEFHSDHMKIRNATFPGVQMKFVLQKLLTTIADAAKGYKPVAVPARTPANAAVPASTPLKQEWMWNQLGNFLQEGDVVIAETGTSAFGINQTTFPNNTYGISQVLWGSIGFTTGATLGAAFAAEEIDPKKRVILFIGDGSLQLTVQEISTMIRWGLKPYLFVLNNDGYTIEKLIHGPKAQYNEIQGWDHLSLLPTFGAKDYETHRVATTGEWDKLTQDKSFNDNSKIRMIEIMLPVFDAPQNLVEQAKLTAATNAKQ

SEQ ID NO: 11 - protein, 376 aa, Pichia kudriavzevii
MFASTFRSQAVRAARFTRFQSTFAIPEKQMGVIFETHGGPLQYKEIPVPKPKPTEILINVKYSGVCHTDLHAWKGDWPLPAKLPLVGGHEGAGIVVAKGSAVTNFEIGDYAGIKWLNGSCMSCEFCEQGDESNCEHADLSGYTHDGSFQQYATADAIQAAKIPKGTDLSEVAPILCAGVTVYKALKTADLRAGQWVAISGAAGGLGSLAVQYAKAMGLRVLGIDGGEGKKELFEQCGGDVFIDFTRYPRDAPEKMVADIKAATNGLGPHGVINVSVSPAAISQSCDYVRATGKVVLVGMPSGAVCKSDVFTHVVKSLQIKGSYVGNRADTREALEFFNEGKVRSPIKVVPLSTLPEIYELMEQGKILGRYVVDTSK

SEQ ID NO: 12 - protein, 388 aa, Pichia kudriavzevii
MVSPAERLSTIASTIKPNRKDSTSLQPEDYPEHPFKVTVVGSGNWGCTIAKVIAENTVERPRQFQRDVNMWVYEELIEGEKLTEIINTKHENVKYLPGIKLPVNVVAVPDIVEACAGSDLIVFNIPHQFLPRILSQLKGKVNPKARAISCLKGLDVNPNGCKLLSTVITEELGIYCGALSGANLAPEVAQCKWSETTVAYTIPDDFRGKGKDIDHQILKSLFHRPYFHVRVISDVAGISIAGALKNVVAMAAGFVEGLGWGDNAKAAVMRIGLVETIQFAKTFFDGCHAATFTHESAGVADLITTCAGGRNVRVGRYMAQHSVSATEAEEKLLNGQSCQGIHTTREVYEFLSNMGRTDEFPLFTTTYRIIYENFPIEKLPECLEPVED

SEQ ID NO: 13 - protein, 342 aa, Pichia kudriavzevii
MSNVKVALLGAAGGIGQPLALLLKLNPNITHLALYDVVHVPGVAADLHHIDTDVVITHHLKDEDGTALANALKDATFVIVPAGVPRKPGMTRGDLFTINAGICAELANAISLNAPNAFTLVITNPVNSTVPIFKEIFAKNEAFNPRRLFGVTALDHVRSNTFLSELIDGKNPQHFDVTVVGGHSGNSIVPLFSLVKAAENLDDEIIDALIHRVQYGGDEVVEAKSGAGSATLSMAYAANKFFNILLNGYLGLKKTMISSYVFLDDSINGVPQLKENLSKLLKGSEVELPTYLAVPMTYGKEGIEQVFYDWVFEMSPKEKENFITAIEYIDQNIEKGLNFMVR

SEQ ID NO: 14 - protein, 267 aa, Artificial Sequence
  Bacterial consensus sequence
MLHIAMIGCGAIGAGVLELLKSDPDLRVDAVIVPEESMDAVREAVAALAPVARVLTALPADARPDLLVECAGHRAIEEHVVPALERGIPCAVASVGALSEPGLAERLEAAARRGGTQVQLLSGAIGAIDALAAARVGGLDSVVYTGRKPPLAWKGTPAEQVCDLDALTEATVIFEGSAREAARLYPKNANVAATLSLAGLGLDRTQVRLIADPAVTENVHHVEARGAFGGFELTMRGKPLAANPKTSALTVYSVVRALGNRAHALSI

SEQ ID NO: 15 - protein, 128 aa, Artificial Sequence
  Bacterial consensus sequence
MLRTMLKSKIHRATVTQADLHYVGSVTIDADLLDAADILEGEKVAIVDITNGARLETYVIAGERGSGVIGINGAAAHLVHPGDLVIIIAYAQMSDAEARAYEPRVVFVDADNRIVELGNDPAEALPGG

SEQ ID NO: 16 - protein, 585 aa, Artificial Sequence
  Eukaryotic consensus sequence
MPANGNFPVALEVISIFKPYNSAVEDLASMAKTDTSASSSGSDSAGSSEDEDVQLFASKGNLLNSKLLKKSNNNNKNNNINENNNKNAAAGLKRFASLPNRAEHEEFLRDCVDEILKLAVFEGTNRSSKVVEWHDPEELKKLFDFELRAEPDSHEKLLELLRATIRYSVKTGHPYFVNQLFSSVDPYGLVGQWLTDALNPSVYTYEVAPVFTLMEEVVLREMRRIVGFPNDGEGDGIFCPGGSIANGYAISCARYKYAPEVKKKGLHSLPRLVIFTSEDAHYSVKKLASFMGIGSDNVYKIATDEVGKMRVSDLEQEILRALDEGAQPFMVSATAGTTVIGAFDPLEGIADLCKKYNLWMHVDAAWGGGALMSKKYRHLLKGIERADSVTWNPHKLLAAPQQCSTFLTRHEGILSECHSTNATYLFQKDKFYDTSYDTGDKHIQCGRRADVLKFWFMWKAKGTSGFEAHVDKVFENAEYFTDSIKARPGFELVIEEPECTNICFWYVPPSLRGMERDNAEFYEKLHKVAPKIKERMIKEGSMMITYQPLRDLPNFFRLVLQNSGLDKSDMLYFINEIERLGSDLV

SEQ ID NO: 17 - protein, 268 aa, Ralstonia solanacearum
MLHVSMVGCGAIGQGVLELLKSDPDLCFDTVIVPEHGMDRARAAIAPFAPRTRVMTRLPAQADRPDLLVECAGHDALREHVVPALEQGIDCLVVSVGALSEPGLAERLEAAARRGHAQMQLLSGAIGAIDALAAARVGGLDAVVYTGRKPPRAWKGTPAERQFDLDALDRTTVIFEGKASDAALLFPKNANVAATLALAGLGMERTHVRLLADPTIDENIHHVEARGAFGGFELIMRGKPLAANPKTSALTVFSVVRALGNRAHAVSI

SEQ ID NO: 18 - protein, 267 aa, Polaromonas species
MLKIAMIGCGAIGASVLELLHGDSDVVVDRVITVPEARDRTEIAVARWAPRARVLEVLAADDAPDLVVECAGHGAIAAHVVPALERGIPCVVTSVGALSAPGMAQLLEQAARRGKTQVQLLSGAIGGIDALAAARVGGLDSVVYTGRKPPMAWKGTPAEAVCDLDSLTVAHCIFDGSAEQAAQLYPKNANVAATLSLAGLGLKRTQVQLFADPGVSENVHHVAAHGAFGSFELTMRGRPLAANPKTSALTVYSVVRALLNRGRALVI

SEQ ID NO: 19 - protein, 271 aa, Burkholder thailandensis
MRNAHAPVDVAMIGFGAIGAAVYRAVEHDAALRVAHVIVPEHQCDAVRGALGERVDVVSSVDALAYRPQFALECAGHGALVDHVVPLLRAGTDCAVASIGALSDLALLDALSEAADEGGATLTLLSGAIGGVDALAAAKQGGLDEVQYIGRKPPLGWLGTPAEALCDLRAMTAEQTIFEGSARDAARLYPKNANVAATVALAGVGLDATKVRLIADPAVTRNVHRVVARGAFGEMSIEMSGKPLPDNPKTSALTAFSAIRALRNRASHCVI

SEQ ID NO: 20 - protein, 271 aa, Burkholderia pseudomallei
MRNAHAPVDVAMIGFGAIGAAVYRAVEHDAALRVAHVIVPEHQCDAVRGALGERVDVVSSVDALACRPQFALECAGHGALVDHVVPLLKAGTDCAVASIGALSDLALLDALSNAADAGGATLTLLSGAIGGIDALAAARQGGLDEVRYIGRKPPLGWLGTPAEAICDLRAMAAEQTIFEGSARDAAQLYPRNANVAATIALAGVGLDATRVCLIADPAVTRNVHRIVARGAFGEMSIEMSGKPLPDNPKTSALTAFSAIRALRNRASHCVI

SEQ ID NO: 21 - protein, 268 aa, Ochrobactrum anthropi
MSVSETIVLVGWGAIGKRVADLLAERKSSVRIGAVAVRDRSASRDRLPAGAVLIENPAELAASGASLVVEAAGRPSVLPWGEAALSTGMDFAVSSTSAFVDDALFQRLKDAAAASGAKLIIPPGALGGIDALSAASRLSIESVEHRIIKPAKAWAGTQAAQLVPLDEISEATVFFTDTARKAADAFPQNANVAVITSLAGIGLDRTRVTLVADPAARLNTHEIIAEGDFGRMHLRFENGPLATNPKSSEMTALNLVRAIENRVATTVI

SEQ ID NO: 22 - protein, 263 aa, Acinetobacter species
MKKLMMIGFGAMAAEVYAHLPQDLQLKWIVVPSRSIEKVQSQVSSEIQVISDIEQCDGTPDYVIEVAGQAAVKEHAQKVLAKGWTIGLISVGTLADSEFLIQLKQTAEKNDAHLHLLAGAIAGIDGISAAKEGGLQKVTYKGCKSPKSWKGSYAEQLVDLDHVVEATVFFTGTAREAATKFPANANVAATIALAGLGMDETMVELTVDPTINKNKHTIVAEGGFGQMTIELVGVPLPSNPKTSTLAALSVIRACRNSVEAIQI

SEQ ID NO: 23 - protein, 255 aa, Klebsiella pneumoniae
MMKKVMLIGYGAMAQAVIERLPPQVRVEWIVARESHHAAICLQFGQAVTPLTDPLQCGGTPDLVLECASQQAVAQYGEAVLARGWHLAVISTGALADSELEQRLRQAGGKLTLLAGAVAGIDGLAAAKEGGLERVTYQSRKSPASWRGSYAEQLIDLSAVNEAQIFFEGSAREAARLFPANANVAATIALGGIGLDATRVQLMVDPATQRNTHTLHAEGLFGEFHLELSGLPLASNPKTSTLAALSAVRACRELA

SEQ ID NO: 24 - protein, 253 aa, Dinoroseobacter shibae
MRLALIGLGAINRAVAAGMAGQAEMVALTRSGAEAPGVMAVSDLSALRVFAPDLVVEAAGHGAARAYLPGLLAAGIDVLMASVGVLADPETEAAFRAAPAHGAQLTIPAGAIGGLDLLAALPKDSLRAVRYTGVKPPAAWAGSPAADGRDLSALDGPVTLFEGTARQAALRFPNNANVAATLALAGAGFDRTEARLVADPDAAGNGHAYDVISDTAEMTFSVRARPSDTPGTSATTAMSLLRAIRNRDAAWVV

SEQ ID NO: 25 - protein, 275 aa, Ruegeria pomeroyi
MWKLWGSWPEGDRVRIALIGHGPIAAHVAAHLPVGVQLTGALCRPGRDDAARAALGVSVAQALEGLPQRPDLLVDCAGHSGLRAHGLTALGAGVEVLTVSVGALADAVFCAELEDAARAGGTRLCLASGAIGALDALAAAAMGTGLQVTYTGRKPPQGWRGSRAEKVLDLKALTGPVTHFTGTARAAAQAYPKNANVAAAVALAGAGLDATRAELIADPGAAANIHEIAAEGAFGRFRFQIEGLPLPGNPRSSALTALSLLAALRQRGAAIRPSF

SEQ ID NO: 26 - protein, 266 aa, Comamonas testosteroni
MKNIALIGCGAIGSSVLELLSGDTQLQVGWVLVPEITPAVRETAARLAPQAQLLQALPGDAVPDLLVECAGHAAIEEHVLPALARGIPAVIASIGALSAPGMAERVQAAAETGKTQAQLLSGAIGGIDALAAARVGGLETVLYTGRKPPKAWSGTPAEQVCDLDGLTEAFCIFEGSAREAAQLYPKNANVAATLSLAGLGLDKTMVRLFADPGVQENVHQVEARGAFGAMELTMRGKPLAANPKTSALTVYSVVRAVLNNVAPLAI

SEQ ID NO: 27 - protein, 268 aa, Cupriavidus pinatubonensis
MSMLHVSMVGCGAIGRGVLELLKADPDVAFDVVIVPEGQMDEARSALSALAPNVRVATGLDGQRPDLLVECAGHQALEEHIVPALERGIPCMVVSVGALSEPGLVERLEAAARRGNTQVQLLSGAIGAIDALAAARVGGLDEVIYTGRKPARAWTGTPAAELFDLEALTEPTVIFEGTARDAARLYPKNANVAATVSLAGLGLDRTSVRLLADPNAVENVHHIEARGAFGGFELTMRGKPLAANPKTSALTVFSVVRALGNRAHAVSI

SEQ ID NO: 28 - DNA, 5261 bp, Artificial Sequence
  This sequence is the complete sequence of plasmid pTL3, for use in yeast.
gacgaaagggcctcgtgatacgcctatttttataggttaatgtcatgataataatggtttcttaggacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaaggacccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaagtttaaacgattaatataattatataaaaatattatcttcttttctttatatctagtgttatgtaaaataaattgatgactacggaaagcttttttatattgtttctttttcattctgagccacttaaatttcgtgaatgttcttgtaagggacggtagatttacaagtgatacaacaaaaagcaaggcgctttttctaataaaaagaagaaaagcatttaacaattgaacacctctatatcaacgaagaatattactttgtctctaaatccttgtaaaatgtgtacgatctctatatgggttactcacagctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatagggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctgtggtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 29 - protein, 608 aa, Pichia kudriavzevii
MLQTANSEVPNASQITIDAASGLPADRVLPNITNTEITISEYIFYRILQLGVRSVFGVPGDFNLRFLEHIYDVHGLNWIGCCNELNAAYAADAYAKASKKMGVLLTTYGVGELSALNGVAGAYTEFAPVLHLVGTSALKFKRNPRTLNLHHLAGDKKTFKKSDHYKYERIASEFSVDSASIEDDPIEACEMIDRVIYSTWRESRPGYIFLPCDLSEMKVDAQRLASPIELTYRFNSPVSRVEGVADQILQLIYQNKNVSIIVDGFIRKFRMESEFYDIMEKFGDKVNIFSTMYGKGLIGEEHPRFVGTYFGKYEKAVGNLLEASDLIIHFGNFDHELNMGGFTFNIPQEKYIDLSAQYVDITGNLDESITMMEVLPVLASKLDSSRVNVADKFEKFDKYYETPDYQREASLQETDIMQSLNENLTGDDILIVETCSFLFAVPDLKVKQHTNIILQAYWASIGYALPATLGASLAIRDFNLSGKVYTIEGDGSAQMSLQELSSMLRYNIDATMILLNNSGYTIERVIVGPHSSYNDINTNWQWTDLLRAFGDVANEKSVSYTIKEREQLLNILSDPSFKHNGKFRLLECVLPMFDVPKKLGQFTGKIPA

SEQ ID NO: 30 - protein, 584 aa, Pichia kudriavzevii
MAPVSLETCTLEFSCKLPLSEYIFRRIASLGIHNIFGVPGDYNLSFLEHLYSVPELSWVGCCNELNSAYATDGYSRTIGHDKFGVLLTTQGVGELSAANAIAGSFAEHVPILHIVGTTPYSLKHKGSHHHHLINGVSTREPTNHYAYEEMSKNISCKILSLSDDLTNAANEIDDLFRTILMLKKPGYLYIPCDLVNVEIDASNLQSVPANKLRERVPSTDSQTIAKITSTIVDKLLSSSNPVVLCDILTDRYGMTAYAQDLVDSLKVPCCNSFMGKALLNESKEHYIGDFNGEESNKMVHSYISNTDCFLHIGDYYNEINSGHWSLYNGINKESIVILNPEYVKIGSQTYQNVSFEDILPAILSSIKANPNLPCFHIPKIMSTIEQIPSNTPISQTLMLEKLQSFLKPNDVLVTETCSLMFGLPDIRMPENSKVIGQHFYLSIGMALPCSFGVSVALNELKKDSRLILIEGDGSAQMTVQELSNFNRENVVKPLIILLNNSGYTVERVIKGPKREYNDIRPDWKWTQLLQTFGMDDAKSMKVTTPEELDDALDEYGNNLSTPRLLEVVLDKLDVPWRFNKMVGN

SEQ ID NO: 31 - protein, 388 aa, Pichia kudriavzevii; identical to SEQ ID NO: 12

SEQ ID NO: 32 - protein, 419 aa, Pichia kudriavzevii
MSRGFFTENITQLPPDPLFGLKARFSNDSRENKVDLGIGAYRDDNGKPWILPSVRLAENLIQNSPDYNHEYLPIGGLADFTSAAARVVFGGDSKAISQNRLVSIQSLSGTGALHVAGLFIKRQYKSLDGTSEDPLIYLSEPTWANHVQIFEVIGLKPVFYPYWHAASKTLDLKGYLKAINDAPEGSVFVLHATAHNPTGLDPTQEQWMEILAAISAKKHLPLFDCAYQGFTSGSLDRDAWAVREAVNNDKYEFPGIIVCQSFAKNVGMYGERIGAVHIVLPESDASLNSAIFSQLQKTIRSEISNPPGYGAKIVSKVLNTPELYKQWEQDLITMSSRITAMRKELVNELERLGTPGTWRHITEQQGMFSFTGLNPEQVAKLEKEHGVYLVRSGRASIAGLNMGNVKYVAKAIDSVVRDL

SEQ ID NO: 33 - protein, 1839 aa, Pichia kudriavzevii
MNTIGWSVSDWVSFNRETTPDESFNTLKALVDYIKSTPNDPAWISIISEENLNHQWNILQSKSNKPSLKLYGVPIAVKDNIDALGFPTTAACPSFSYMPTSDSTIVSLLRDQGAIIIGKTNLDQFATGLVGTRSPYGITPCVFSDKHVSGGSSAGSASVVARGLVPIALGTDTAGSGRVPAALNNIIGLKPTVGAFSTNGVVPACKSLDCPSIFSLNLNDAQLVFNICAKPDLTNCEYSREGPQNYKRKFTGKVKIAIPIDFNGLWFNDEENPKIFNDAIENFKKLNVEIVPIDFNPLLELAKCLYEGPWVSERYSAVKSFYKSNPKKEDLDPIVTKIIENGANYDASTAFEYEYKRRGILNKVKLLIKDIDALLVPTCPLNPTIEQVLKEPIKVNSIQGTWTNFCNLADFAALALPNGFRNDGLPNGFTLLGRAFEDYALLSLAKDYFNAKYPKHDRSIGNIKDKTSGVEDLLDNSLPQPNLNSSIKLAVVGAHLEGLPLYWQLEKVQAYKLETTKTSSNYKLYALPNSNKNSIMKPGLRRISSSNEVGGSQIEVEVYSIPLENFGDFISMVPQPLGIGSVELESGEWVKSFICEECGYKENGSIEITHFGGWRNYLKHLNLNSRLEKSKKPFNKVLVANRGEIAVRIIKTLKKLNIISVAVYSDPDKYSDHVLLADEAYPLNGISASETYINIEKMLKVIKLSKAEAVIPGYGFLSENADFADKLIEEGIVWVGPSGDTIRKLGLKHSAREIAKNAGVPLVPGSNLINDSLEAKEIAQKLEYPIMIKSTAGGGGIGLQKVDSEDDIERVFETVQHQGKSYFGDSGVFLERFVENSRHVEIQIFGDGNGNAIAIGERDCSLQRRNQKVIEETPAPNLPEITRKKMRKAAEQLASSMNYKCAGTVEFIYDEKRDEFYFLEVNTRLQVEHPITEMVTGLDLVEWMLFIAADMPPDFNQVIPVEGASMEARLYAENPVKDFKPSPGQLIEVKFPEFARVDTWVKTGTIISSEYDPTLAKIIVHGKDRIDALNKLRKALNETVIYGCITNIDYLRSIANSKMFEDAKMHTKILDTFDYKPNAFEILSPGAYTTVQDYPGRVGYWRIGVPPSGPMDSYSFRLANRIVGNHYKSPAIEITLNGPSILFHHETVIAITGGEVPVTLNDERVNMYEPINIKRGDKLVIGKLTTGCRSYLSIRGGIDVTEYLGSRSTFALGNLGGYNGRVLKMGDVLFLSQPGLSSNKLPEPISKPQIAPTSVIPQISTTKEWTVGVTCGPHGSPDFFTAESIKDFFSNPWKVHYNSNRFGVRLIGPKPKWARNDGGEGGLHPSNAHDYVYSLGAINFTGDEPVILTCDGPSLGGFVCQAVVADAEMWKIGQVKPGDSINFVPISFDQAIELKQQQNSLIESLSGEYNSIAIAKPLSEPEDPVLAVYQANDHSPKITYRQAGDRYVLVEYGENIMDLNYSYRVHKLIEMVESHKTIGIIEMSQGVRSVLIEYDGFEIHQKVLVKTLLSYEAEVAFTNKWSVPSRVIRLPMAFEDRQTLDAVKRYQETIRSDAPWLPNNVDFIANINGIERSEVKDMLYSARFLVLGLGDVFLGAPCAVPLDPRQRFLGTKYNPSRTFTPNGTVGIGGMYMCIYTMESPGGYQLVGRTIPIWDKLSLGEYTKKYNNGKPWLLTPFDQVSFYPVTEEELEVMVEDSKHGRFEVDIIESVFDHTKYLSWITENSDSIEEFQRQQDGEKLQEFKRLIQVANEDLAKSGTKIVETEEKFPENAELIYSEYSGRFWKSLVNVGDEVKKGQGLVVIEAMKTEMVVNATKDGKVLKIVHGNGDMVDAGDLVVVIA

SEQ ID NO: 34 - protein, 835 aa, Schizosaccharomyces pombe
MQPRELHKLTLHQLGSLAQKRLCRGVKLNKLEATSLIASQIQEYVRDGNHSVADLMSLGKDMLGKRHVQPNVVHLLHEIMIEATFPDGTYLITIHDPICTTDGNLEHALYGSFLPTPSQELFPLEEEKLYAPENSPGFVEVLEGEIELLPNLPRTPIEVRNMGDRPIQVGSHYHFIETNEKLCFDRSKAYGKRLDIPSGTAIRFEPGVMKIVNLIPIGGAKLIQGGNSLSKGVFDDSRTREIVDNLMKQGFMHQPESPLNMPLQSARPFVVPRKLYAVMYGPTTNDKIRLGDTNLIVRVEKDFTEYGNESVFGGGKVIRDGTGQSSSKSMDECLDTVITNAVIIDHTGIYKADIGIKNGYIVGIGKAGNPDTMDNIGENMVIGSSTDVISAENKIVTYGGMDSHVHFICPQQIEEALASGITTMYGGGTGPSTGTNATTCTPNKDLIRSMLRSTDSYPMNIGLTGKGNDSGSSSLKEQIEAGCSGLKLHEDWGSTPAAIDSCLSVCDEYDVQCLIHTDTLNESSFVEGTFKAFKNRTIHTYHVEGAGGGHAPDIISLVQNPNILPSSTNPTRPFTTNTLDEELDMLMVCHHLSRNVPEDVAFAESRIRAETIAAEDILQDLGAISMISSDSQAMGRCGEVISRTWKTAHKNKLQRGALPEDEGSGVDNFRVKRYVSKYTINPAITHGISHIVGSVEIGKFADLVLWDFADFGARPSMVLKGGMIALASMGDPNGSIPTVSPLMSWQMFGAHDPERSIAFVSKASITSGVIESYGLHKRVEAVKSTRNIGKKDMVYNSYMPKMTVDPEAYTVTADGKVMECEPVDKLPLSQSYFIF

SEQ ID NO: 35 - protein, 290 aa, Schizosaccharomyces pombe
MEDKEGRFRVECIENVHYVTDMFCKYPLKLIAPKTKLDFSILYIMSYGGGLVSGDRVALDIIVGKNATLCIQSQGNTKLYKQIPGKPATQQKLDVEVGTNALCLLLQDPVQPFGDSNYIQTQNFVLEDETSSLALLDWTLHGRSHINEQWSMRSYVSKNCIQMKIPASNQRKTLLRDVLKIFDEPNLHIGLKAERMHHFECIGNLYLIGPKFLKTKEAVLNQYRNKEKRISKTTDSSQMKKIIWTACEIRSVTIIKFAAYNTETARNFLLKLFSDYASFLDHETLRAFWY

SEQ ID NO: 36 - protein, 235 aa, Schizosaccharomyces pombe
MTDSQTETHLSLILSDTAFPLSSFSYSYGLESYLSHQQVRDVNAFFNFLPLSLNSVLHTNLPTVKAAWESPQQYSEIEDFFESTQTCTIAQKVSTMQGKSLLNIWTKSLSFFVTSTDVFKYLDEYERRVRSKKALGHFPVVWGVVCRALGLSLERTCYLFLLGHAKSICSAAVRLDVLTSFQYVSTLAHPQTESLLRDSSQLALNMQLEDTAQSWYTLDLWQGRHSLLYSRIFNS

SEQ ID NO: 37 - protein, 286 aa, Schizosaccharomyces pombe
MAIPFLHKGGSDDSTHHHTHDYDHHNHDHHGHDHHSHDSSSNSSSEAARLQFIQEHGHSHDAMETPGSYLKRELPQFNHRDFSRRAFTIGVGGPVGSGKTALLLQLCRLLGEKYSIGVVTNDIFTREDQEFLIRNKALPEERIRAIETGGCPHAAIREDVSGNLVALEELQSEFNTELLLVESGGDNLAANYSRDLADFIIYVIDVSGGDKIPRKGGPGITESDLLIINKTDLAKLVGADLSVMDRDAKKIRENGPIVFAQVKNQVGMDEITELILGAAKSAGALK

SEQ ID NO: 38 - protein, 408 aa, Schizosaccharomyces pombe
MNSMSEYVKPRKNEFLRKFENFYFEIPFLSKLPPKVSVPIFSLISVNIVVWIVAAIVISLVNRSLFLSVLLSWTLGLRHALDADHITAIDNLTRRLLSTDKPMSTVGTWFSIGHSTVVLITCIVVAATSSKFADRWNNFQTIGGIIGTSVSMGLLLLLAIGNTVLLVRLSYWLWMYRKSGVTKDEGVTGFLARKMQRLFRLVDSPWKIYVLGFVFGLGFDTSTEVSLLGIATLQALKGTSIWAILLFPIVFLVGMCLVDTTDGALMYYAYSYSSGETNPYFSRLYYSIILTFVSVIAAFTIGIIQMLMLIISVHPMESTFWNGLNRLSDNYEIVGGCICGAFVLAGLFGISMHNYFKKKFTPPVQVGNDREDEVLEKNKELENVSKNSISVQISESEKVSYDTVDSKV

SEQ ID NO: 39 - protein, 370 aa, Arabidopsis thaliana
MKGGSMEKIKPILAIISLQFGYAGMYIITMVSFKHGMDHWVLATYRHVVATVVMAPFALMFERKIRPKMTLAIFWRLLALGILEPLMDQNLYYIGLKNTSASYTSAFTNALPAVTFILALIFRLETVNFRKVHSVAKVVGTVITVGGAMIMTLYKGPAIEIVKAAHNSFHGGSSSTPTGQHWVLGTIAIMGSISTWAAFFILQSYTLKVYPAELSLVTLICGIGTILNAIASLIMVRDPSAWKIGMDSGTLAAVYSGVVCSGIAYYIQSIVIKQRGPVFTTSFSPMCMIITAFLGALVLAEKIHLGSIIGAVFIVLGLYSVVWGKSKDEVNPLDEKIVAKSQELPITNVVKQTNGHDVSGAPTNGVVTST

SEQ ID NO: 40 - protein, 516 aa, Arabidopsis thaliana
MGLGGDQSFVPVMDSGQVRLKELGYKQELKRDLSVFSNFAISFSIISVLTGITTTYNTGLRFGGTVTLVYGWFLAGSFTMCVGLSMAEICSSYPTSGGLYYWSAMLAGPRWAPLASWMTGWFNIVGQWAVTASVDFSLAQLIQVIVLLSTGGRNGGGYKGSDFVVIGIHGGILFIHALLNSLPISVLSFIGQLAALWNLLGVLVLMILIPLVSTERATTKFVFTNFNTDNGLGITSYAYIFVLGLLMSQYTITGYDASAHMTEETVDADKNGPRGIISAIGISILFGWGYILGISYAVTDIPSLLSETNNSGGYAIAEIFYLAFKNRFGSGTGGIVCLGVVAVAVFFCGMSSVTSNSRMAYAFSRDGAMPMSPLWHKVNSREVPINAVWLSALISFCMALTSLGSIVAFQAMVSIATIGLYIAYAIPIILRVTLARNTFVPGPFSLGKYGMVVGWVAVLWVVTISVLFSLPVAYPITAETLNYTPVAVAGLVAITLSYWLFSARHWFTGPISNILS

SEQ ID NO: 41 - DNA, 1133 bp, Artificial Sequence
  Synthetic DNA integration cassette s376
aatcaatataaatctggtgtcttccgtattgccgaatgggctgacatcactaatgcacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgtcattggttttattgcgcaacacgatatgggcggtagagaagaaggttttgactggatcattatgactccaggggttggtttagatgacaaaggtgatgcacttggtcaacaatatagaactgttgatgaagttgtaaagactggaacggatatcataattgttggtagaggtttgtacggtcaaggaagagatcctatagagcaagctaaaagataccaacaagctggttggaatgcttatttaaacagatttaaatgagtgaatttactttaaatcttgcatttaaataaattttctttttatagctttatgacttagtttcaatttatatactattttaatgacattttcgattcattgattgaaagctttgtgttttttcttgatgcgctattgcattgttcttgtctttttcgccacatttaatatctgtagtagatacctgatacattgtggatcgcctggcagcagggcgataacctcataacttcgtataatgtatgctatacgaacggtagatagacatctgagtgagcgatagatagatagatagatagatagatgtatgggtagatagatgcatatatagatgcatggaatgaaaggaagatagatagagagaaatgcagaaataagcgtatgaggtttaattttaatgtacatacatgtatagataaacgatgtcgatataatttatttagtaaacagattccctgatatgtgtttttagttttatttttttttgttttttctatgttgaaaaacttgatgacatgatcgagtaaaattggagcttgatttcattcatcttgttgattcctttatcataatgcaaagctggggggggggagggtaaaaaaaagtgaagaaaaagaaagtatgatacaactgtggaagtggag

SEQ ID NO: 42 - DNA, 3304 bp, Artificial Sequence
  DNA integration cassette s404
gcaggcttatggcagacaggtactttttttttgtctctgtataatgagtcaaattgtcaatattgaagggttgtatccaaactgcagttcttgacagtcagacacactcatctttcataaccttccctaaatagatgtgctcctatttcagccaagtatctttattgtcggtgaaaataatggaaacggtctaaatgcgcttgttactaaggctgttactttgataaacgcatttgactttgagatatataacttcaactctaacgacctaatttcaaacggaagagctacttagaccatagattaaaagtgaattctctctaacacactttgaggagcattaatttcacaccaaaacgtctatagatgctgactttagcggtttcaatgggaattgatcttgcaacaccaaggaattgccattgaagagaaacttactgatacatcattcaaccactccgatgatatacaccgggctagatttcgatatggatatggatatggatatggatatggagatgaatttgaatttagatttgggtcttgatttggggttggaattaaaaggggataacaatgagggttttcctgttgatttaaacaatggacgtgggaggtgattgatttaacctgatccaaaaggggtatgtctattttttagagtgtgtctttgtgtcaaattatagtagaatgtgtaaagtagtataaactttcctctcaaatgacgaggtttaaaacaccccccgggtgagccgagccgagaatggggcaattgttcaatgtgaaatagaagtatcgagtgagaaacttgggtgttggccagccaagggggaaggaaaatggcgcgaatgctcaggtgagattgttttggaattgggtgaagcgaggaaatgagcgacccggaggttgtgactttagtggcggaggaggacggaggaaaagccaagagggaagtgtatataaggggagcaatttgccaccaggatagaattggatgagttataattctactgtatttattgtataatttatttctccttttgtatcaaacacattacaaaacacacaaaacacacaaacaaacacaattacaaaaaatgaaaaacatcgccttaattggttgtggtgctattggttcctctgtcttggaattattgtccggtgatacccaattgcaagttggttgggttttggtcccagaaattactccagctgttagagaaactgctgccagattggctccacaagctcaattgttgcaagctttgccaggtgatgctgttccagacttgttggttgaatgtgctggtcacgctgctattgaagaacacgtcttgccagccttggctagaggtatcccagctgtcattgcctccatcggtgctttatctgccccaggtatggctgaaagagtccaagctgccgctgaaaccggtaaaactcaagctcaattgttgtccggtgccatcggtggtatcgatgctttagctgctgctagagttggtggtttagaaactgtcttgtacaccggtagaaagccaccaaaagcctggtctggtactccagctgagcaagtttgtgacttagacggtttgaccgaagctttttgtattttcgagggttctgctagagaagctgcccaattgtacccaaagaacgctaatgttgctgctaccttgtccttggccggtttgggtttggacaagaccatggttagattattcgccgatcctggtgtccaagaaaatgtccaccaagttgaagctagaggtgctttcggtgccatggaattgactatgagaggtaagccattagctgctaacccaaaaacttctgccttaaccgtttactctgttgttagagctgttttgaataacgtcgctccattggctatttaatccagccagtaaaatccatactcaacgacgatatgaacaaatttccctcattccgatgctgtatatgtgtataaatttttacatgctcttctgtttagacacagaacagctttaaataaaatgttggatatactttttctgcctgtggtgtaccgttcgtataatgtatgctatacgaagttataaccggcgttgccagcgataaacgggaaacatcatgaaaactgtttcaccctctgggaagcataaacactagaaagccaatgaagagctctacaagcctcttatgggttcaatgggtctgcaatgaccgcatacgggcttggacaattaccttctattgaatttctgagaagagatacatctcaccagcaatgtaagcagacaatcccaattctgtaaacaacctctttgtccataattccccatcagaagagtgaaaaatgccctcaaaatgcatgcgccacacccacctctcaactgcactgcgccacctctgagggtcttttcaggggtcgactaccccggacacctcgcagaggagcgaggtcacgtacttttaaaatggcagagacgcgcagtttcttgaagaaaggataaaaatgaaatggtgcggaaatgcgaaaatgatgaaaaattttcttggtggcgaggaaattgagtgcaataattggcacgaggttgttgccacccgagtgtgagtatatatcctagtttctgcacttttcttcttcttttctttaccttttcttttcaacttttttttactttttccttcaacagacaaatctaacttatatatcacaatggcgtcatacaaagaaagatcagaatcacacacttcccctgttgctaggagacttttctccatcatggaggaaaagaagtctaacctttgtgcatcattggatattactgaaactgaaaagcttctctctattttggacactattggtccttacatctgtctagttaaaacacacatcgatattgtttctgattttacgtatgaaggaactgtgttgcctttgaaggagcttgccaagaaacataattttatgatttttgaagatagaaaatttgctgatattggtaacaccgttaaaaatcaatataaatctggtgtcttccgtattgccgaatgggctgacatcactaatgcacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgtcattggttttattgcgcaacacgatatgggcggtagagaagaaggttttgactggatcattatgactcca

SEQ ID NO: 43 - DNA, 2727 bp, Artificial Sequence
  DNA integration cassette s357
tagacgttgtatttccagctccaacatggttaaactattgctatggtgatggtattacagatagtaaaagaaggaagggggggggtggcaatctcaccctaacagttactaagaacgtctacttcatctactgtcaatatacattggccacatgccgagaaattacgtcgacgccaaagaagggcccagccgaaaaaagaaatggaaaacttggccgaaaagggaaacaaacaaaaaggtgatgtaaaattagcggaaaggggaattggcaaattgagggagaaaaaaaaaaaggcagaaaaggaggcggaaagtcagtacgttttgaaggcgtcattggttttcccttttgcagagtgtttcatttcttttgtttcatgacgtagtggcgtttcttttcctgcactttagaaatctatcttttccttatcaagtaacaagcggttggcaaaggtgtatataaatcaaggaattcccactttgaaccctttgaattttgatatcggttattttaaatttattttatgtttctaatctcaaagagtttacactttacaaggagtttctctaccgttcgtataatgtatgctatacgaagttataaccggcgttgccagcgataaacgggaaacatcatgaaaactgtttcaccctctgggaagcataaacactagaaagccaatgaagagctctacaagcctcttatgggttcaatgggtctgcaatgaccgcatacgggcttggacaattaccttctattgaatttctgagaagagatacatctcaccagcaatgtaagcagacaatcccaattctgtaaacaacctctttgtccataattccccatcagaagagtgaaaaatgccctcaaaatgcatgcgccacacccacctctcaactgcactgcgccacctctgagggtcttttcaggggtcgactaccccggacacctcgcagaggagcgaggtcacgtacttttaaaatggcagagacgcgcagtttcttgaagaaaggataaaaatgaaatggtgcggaaatgcgaaaatgatgaaaaattttcttggtggcgaggaaattgagtgcaataattggcacgaggttgttgccacccgagtgtgagtatatatcctagtttctgcacttttcttcttcttttctttaccttttcttttcaacttttttttactttttccttcaacagacaaatctaacttatatatcacaatggcgtcatacaaagaaagatcagaatcacacacttcccctgttgctaggagacttttctccatcatggaggaaaagaagtctaacctttgtgcatcattggatattactgaaactgaaaagcttctctctattttggacactattggtccttacatctgtctagttaaaacacacatcgatattgtttctgattttacgtatgaaggaactgtgttgcctttgaaggagcttgccaagaaacataattttatgatttttgaagatagaaaatttgctgatattggtaacaccgttaaaaatcaatataaatctggtgtcttccgtattgccgaatgggctgacatcactaatgcacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgtcattggttttattgcgcaacacgatatgggcggtagagaagaaggttttgactggatcattatgactccaggggttggtttagatgacaaaggtgatgcacttggtcaacaatatagaactgttgatgaagttgtaaagactggaacggatatcataattgttggtagaggtttgtacggtcaaggaagagatcctatagagcaagctaaaagataccaacaagctggttggaatgcttatttaaacagatttaaatgagtgaatttactttaaatcttgcatttaaataaattttctttttatagctttatgacttagtttcaatttatatactattttaatgacattttcgattcattgattgaaagctttgtgttttttcttgatgcgctattgcattgttcttgtctttttcgccacatttaatatctgtagtagatacctgatacattgtggatcgcctggcagcagggcgataacctcataacttcgtataatgtatgctatacgaacggtaataacctcaaggagaactttggcattgtactctccattgacgagtccgccaacccattcttgttaaacccaaccttgcattatcacattccctttgaccccctttagctgcatttccacttgtctacattaagattcattacacattctttttcgtatttctcttacctccctcccccctccatggatcttatatataaatcttttctataacaataatatctactagagttaaacaacaattccacttggcatggctgtctcagcaaatctgcttctacctactgcacgggtttgcatgtcattgtttctagcagggaatcgtccatgtacgttgtcctccatgatggtcttcccgctgccactttctttagtatcttaaatagagcagatcttacgtccacagtgcatccgtgcaccccgaaaatcgtatggttttccttgccacctctcaca

SEQ ID NO: 44 - DNA, 6311 bp, Artificial Sequence
  DNA integration cassette s$&%
agttgccattgtgggtttgtgttgcaatccttgcaaatgtttatattgactatacaagtgtaggtctttacgtttcatggatttccttcatctttataagattgaatcatcagccatatttgagctctacataattcataatggtctgatttctacaggactgttttgacaagaaagaatctcatgccgtgtttccaacagtgtggcacctggtgtctttgataaacggctcagaaactcctgtacctcgtgaaaaacaaaattgctgtttcaactccttttcaatatttttcgagctttggcaactacctaaaaaggcaattcctatcctgaaaagtatcttgggcatttctgtggcttttgctcctcctaagatgattatcttttgtggctctctcactgagttggaccactttttcagagcaaatgcagctgttacataatagagaagattcgatataaaaaaaattgcaccataatcaacttagtttcgtggaggtaccaaagccaagggcaaaactaacaactacagggctagatttcgatatggatatggatatggatatggatatggagatgaatttgaatttagatttgggtcttgatttggggttggaattaaaaggggataacaatgagggttttcctgttgatttaaacaatggacgtgggaggtgattgatttaacctgatccaaaaggggtatgtctattttttagagtgtgtctttgtgtcaaattatagtagaatgtgtaaagtagtataaactttcctctcaaatgacgaggtttaaaacaccccccgggtgagccgagccgagaatggggcaattgttcaatgtgaaatagaagtatcgagtgagaaacttgggtgttggccagccaaggggggggggaaggaaaatggcgcgaatgctcaggtgagattgttttggaattgggtgaagcgaggaaatgagcgacccggaggttgtgactttagtggcggaggaggacggaggaaaagccaagagggaagtgtatataaggggagcaatttgccaccaggatagaattggatgagttataattctactgtatttattgtataatttatttctccttttatatcaaacacattacaaaacacacaaaacacacaaacaaacacaattacaaaaaatgtcaactgtggaagatcactcctccttacataaattgagaaaggaatctgagattctttccaatgcaaacaaaatcttagtggctaatagaggtgaaattccaattagaattttcaggtcagcccatgaattgtcaatgcatactgtggcgatctattcccatgaagatcggttgtccatgcataggttgaaggccgacgaggcttatgcaatcggtaagacgggtcaatattcgccagttcaagcttatctacaaattgacgaaattatcaaaatagcaaaggaacatgatgtttccatgatccatccaggttatggtttcttatctgaaaactccgaattcgcaaagaaggttgaagaatccggtatgatttgggttgggcctcctgctgaagttattgattctgttggtgacaaggtttctgcaagaaatttggcaattaaatgtgacgttcctgttgttcctggtaccgatggtccaattgaagacattgaacaggctaaacagtttgtggaacaatatggttatcctgtcattataaaggctgcatttggtggtggtggtagaggtatgagagttgttagagaaggtgatgatatagttgatgctttccaaagagcgtcatctgaagcaaagtctgcctttggtaatggtacttgttttattgaaagatttttggataagccaaaacatattgaggttcaattattggctgataattatggtaacacaatccatctctttgaaagagattgttctgttcaaagaagacatcaaaaggttgttgaaattgcacctgccaaaactttacctgttgaagttagaaatgctatattaaaggatgctgtaacgttagctaaaaccgctaactatagaaatgctggtactgcagaatttttagttgattcccaaaacagacattattttattgaaattaatccaagaattcaagttgaacatacaattactgaagaaatcacaggtgttgatattgttgccgctcaaattcaaattgctgcaggtgcatcattggaacaattgggtctattacaaaacaaaattacaactagaggttttgcaattcaatgtagaattacaaccgaggatcctgctaagaattttgccccagatacaggtaaaattgaggtttatagatctgcaggtggtaatggtgtcagattagatggtggtaatgggtttgccggtgctgttatatctcctcattatgactcgatgttggttaaatgttcaacatctggttctaactatgaaattgccagaagaaagatgattagagctttagttgaatttagaatcagaggtgtcaagaccaatattcctttcttattggcattgctaactcatccagtcttcatttcgggtgattgttggacaacttttattgatgataccccttcgttattcgaaatggtttcttcaaagaatagagcccaaaaattattggcatatattggtgacttgtgtgtcaatggttcttcaattaaaggtcaaattggtttccctaaattgaacaaggaagcagaaatcccagatttgttggatccaaatgatgaggttattgatgtttctaaaccttctaccaatggtctaagaccgtatctattaaagtatggaccagatgcattttccaaaaaagttcgtgaattcgatggttgtatgattatggataccacctggagagatgcacatcaatcattattggctacaagagttagaactattgatttactgagaattgctccaacgactagtcatgccttacaaaatgcatttgcattagaatgttggggtggcgcaacatttgatgttgcgatgaggttcctctatgaagatccttgggagagattaagacaacttagaaaggcagttccaaatattcctttccaaatgttattgagaggtgctaatggtgttgcttattcgtcattacctgataatgcaattgatcattttgttaagcaagcaaaggataatggtgttgatattttcagagtctttgatgctttgaacgatttggaacaattgaaggttggtgttgatgctgtcaagaaagccggaggtgttgttgaagctacagtttgttactcaggtgatatgttaattccaggtaaaaagtataacttggattattatttagagactgttggaaagattgtggaaatgggtacccatattttaggtattaaggatatggctggcacgttaaagccaaaggctgctaagttgttgattggctcgatcagatcaaaataccctgacttggttatccatgtccatacccatgactctgctggtaccggtatttcaacttatgttgcatgcgcattggcaggtgccgacattgtcgattgtgcaatcaattcgatgtctggtttaacttctcaaccttcaatgagtgcttttattgctgctttagatggtgatatcgaaactggtgttccagaacattttgcaagacaattagatgcatattgggcagaaatgagattgttatactcatgtttcgaagccgacttgaagggaccagacccagaagtttataaacatgaaattccaggtggacagttgactaacctaatcttccaagcccaacaagttggtttgggtgaacaatgggaagaaactaagaagaagtatgaagatgctaacatgttgttgggtgatattgtcaaggttaccccaacctccaaggttgttggtgatttagcccaatttatggtttctaataaattagaaaaagaagatgttgaaaaacttgctaatgaattagatttcccagattcagttcttgatttctttgaaggattaatgggtacaccatatggtggattcccagagcctttgagaacaaatgtcatttccggcaagagaagaaaattaaagggtagaccaggtttagaattagaacctttcaacctcgaggaaatcagagaaaatttggtttccagatttggtccaggtattactgaatgtgatgttgcatcttataacatgtatccaaaggtttacgagcaatatcgtaaggtggttgaaaaatatggtgatttatctgttttaccaacaaaagcatttttggcccctccaactattggtgaagaagttcatgtggaaattgagcaaggtaagactttgattattaagttgttagccatttctgacttgtctaaatctcatggtacaagagaagtatactttgaattgaatggtgaaatgagaaaggttacaattgaagataaaacagctgcaattgagactgttacaagagcaaaggctgacggacacaatccaaatgaagttggtgcgccaatggctggtgtcgttgttgaagttagagtgaagcatggaacagaagttaagaagggtgatccattagccgttttgagtgcaatgaaaatggaaatggttatttctgctcctgttagtggtagggtcggtgaagtttttgtcaacgaaggcgattccgttgatatgggtgatttgcttgtgaaaattgccaaagatgaagcgccagcagcttaatcttgattcatgtaactcatgtatttgttttgtattcaattatgttataccttggtatacatataacgatttgtatttacatatttatttattagtggtagttttttttttcagagagtactgtatttcctcccaaacaaccgtgaaggctttaaggtccacttatcaccagtataagtttccttagtgacgacgcctatttgcttaattgtgatttcaaagactcaatttgttgctccaagtctttgatgtcttcgtctagttttctttcatcaaaacatatacctatgttattaatgttttgttgtaacctgcgatcatggtcataaatgtcggtgtaaatgttagacagtaccgttcgtataatgtatgctatacgaagttataaccggcgttgccagcgataaacgggaaacatcatgaaaactgtttcaccctctgggaagcataaacactagaaagccaatgaagagctctacaagcctcttatgggttcaatgggtctgcaatgaccgcatacgggcttggacaattaccttctattgaatttctgagaagagatacatctcaccagcaatgtaagcagacaatcccaattctgtaaacaacctctttgtccataattccccatcagaagagtgaaaaatgccctcaaaatgcatgcgccacacccacctctcaactgcactgcgccacctctgagggtcttttcaggggtcgactaccccggacacctcgcagaggagcgaggtcacgtacttttaaaatggcagagacgcgcagtttcttgaagaaaggataaaaatgaaatggtgcggaaatgcgaaaatgatgaaaaattttcttggtggcgaggaaattgagtgcaataattggcacgaggttgttgccacccgagtgtgagtatatatcctagtttctgcacttttcttcttcttttctttaccttttcttttcaacttttttttactttttccttcaacagacaaatctaacttatatatcacaatggcgtcatacaaagaaagatcagaatcacacacttcccctgttgctaggagacttttctccatcatggaggaaaagaagtctaacctttgtgcatcattggatattactgaaactgaaaagcttctctctattttggacactattggtccttacatctgtctagttaaaacacacatcgatattgtttctgattttacgtatgaaggaactgtgttgcctttgaaggagcttgccaagaaacataattttatgatttttgaagatagaaaatttgctgatattggtaacaccgttaaaaatcaatataaatctggtgtcttccgtattgccgaatgggctgacatcactaatgcacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgtcattggttttattgcgcaacacgatatgggcggtagagaagaaggttttgactggatcattatgactcca

SEQ ID NO: 45 - DNA, 1219 bp, Artificial Sequence
  DNA integration cassette s422
aatcaatataaatctggtgtcttccgtattgccgaatgggctgacatcactaatgcacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgtcattggttttattgcgcaacacgatatgggcggtagagaagaaggttttgactggatcattatgactccaggggttggtttagatgacaaaggtgatgcacttggtcaacaatatagaactgttgatgaagttgtaaagactggaacggatatcataattgttggtagaggtttgtacggtcaaggaagagatcctatagagcaagctaaaagataccaacaagctggttggaatgcttatttaaacagatttaaatgagtgaatttactttaaatcttgcatttaaataaattttctttttatagctttatgacttagtttcaatttatatactattttaatgacattttcgattcattgattgaaagctttgtgttttttcttgatgcgctattgcattgttcttgtctttttcgccacatttaatatctgtagtagatacctgatacattgtggatcgcctggcagcagggcgataacctcataacttcgtataatgtatgctatacgaacggtaaagcatgttttttctttgaaaactatctttggatgttccaaatacgaattaggttaggaattgtatttatcttgtatatgacccaaaaacacctaaaagttcattcaccgaactttaatgcgatttgcgattctgaaactgattcatataaatcgtcaccagtagtattatacaaggctcttatacctcttctttttccaccctacagatcagtgcgcaaacatgcagcactgtgctttgtatagttttagttggacctttttataactagaagtccagctcgtcattttctctcttcgttggaccttcacatttcaagagtttgtcaacatagtttctaaaaagtaatatactatccttcaaaggtgtatttttccactcaaattcgtcagcagaaaaaatttgttgtagatttggggcatccgtaaacggattgaattctctttcattcggatcaaatacaacacaaac

SEQ ID NO: 46 - DNA, 2691 bp, Artificial Sequence
  DNA integration cassette s424
gggggatatggagggctcggaatacagatggatgcaactgtggcagcaatttgagctgctaatttttgctcctctttaacgcaatcatttcctcctcccaacaacaaaatacacttccatggtcctacaaatgtaggcggctgtgaaaaagactgtattatgtattttaatcaactgtggctttttgaaatagtctcttaacattgccgaaaaatagatgagctactccgtttaaacgggcccaagatacaaaaaaaaagttgcggctactcacggatattaaaggttagaaagggcaatatgttagtagaaacaaggtttaacttaagcatgatcaccgaaattgctgcctttaagttgtaaatcaagaagtgcaaaaaggagtatataaggaccatgattctcccagcaagtcctttttttaataacgccatctatttgtacccacttaatctagctttacagtttattatatagcaagtacatagattttaattaccgttcgtataatgtatgctatacgaagttataaccggcgttgccagcgataaacgggaaacatcatgaaaactgtttcaccctctgggaagcataaacactagaaagccaatgaagagctctacaagcctcttatgggttcaatgggtctgcaatgaccgcatacgggcttggacaattaccttctattgaatttctgagaagagatacatctcaccagcaatgtaagcagacaatcccaattctgtaaacaacctctttgtccataattccccatcagaagagtgaaaaatgccctcaaaatgcatgcgccacacccacctctcaactgcactgcgccacctctgagggtcttttcaggggtcgactaccccggacacctcgcagaggagcgaggtcacgtacttttaaaatggcagagacgcgcagtttcttgaagaaaggataaaaatgaaatggtgcggaaatgcgaaaatgatgaaaaattttcttggtggcgaggaaattgagtgcaataattggcacgaggttgttgccacccgagtgtgagtatatatcctagtttctgcacttttcttcttcttttctttaccttttcttttcaacttttttttactttttccttcaacagacaaatctaacttatatatcacaatggcgtcatacaaagaaagatcagaatcacacacttcccctgttgctaggagacttttctccatcatggaggaaaagaagtctaacctttgtgcatcattggatattactgaaactgaaaagcttctctctattttggacactattggtccttacatctgtctagttaaaacacacatcgatattgtttctgattttacgtatgaaggaactgtgttgcctttgaaggagcttgccaagaaacataattttatgatttttgaagatagaaaatttgctgatattggtaacaccgttaaaaatcaatataaatctggtgtcttccgtattgccgaatgggctgacatcactaatgcacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgtcattggttttattgcgcaacacgatatgggcggtagagaagaaggttttgactggatcattatgactccaggggttggtttagatgacaaaggtgatgcacttggtcaacaatatagaactgttgatgaagttgtaaagactggaacggatatcataattgttggtagaggtttgtacggtcaaggaagagatcctatagagcaagctaaaagataccaacaagctggttggaatgcttatttaaacagatttaaatgagtgaatttactttaaatcttgcatttaaataaattttctttttatagctttatgacttagtttcaatttatatactattttaatgacattttcgattcattgattgaaagctttgtgttttttcttgatgcgctattgcattgttcttgtctttttcgccacatttaatatctgtagtagatacctgatacattgtggatcgcctggcagcagggcgataacctcataacttcgtataatgtatgctatacgaacggtatggtattgcttgagcaaaaaaaaaagagagggaaatacatttgccacattataattatgtaatccatggagtttatagagataatcatattagttacatgtaatttttggcacttgctattgtagtatgcagtcgttcacgtgcaaacatgcatctgataatttttaagcatgcgaattttctagatttttcggttagtgcttaggggatactttttgggttatagatacatgccttcataaaaaacagacaagatgtgctctttaccaacatagagagatagatagaaatttctaaaaacaattccctcactgacagaaacaagtagaattgaacatgaaatggatatccatattttcattagtgtcggctgttactgggataagttccttgaaatcgatcgaggaggagatatcgagaatagattcaaaatttagaaacgtaggaccgactcttgaaattctaaatgaatacgattcagtgatcagcct

SEQ ID NO: 47 - DNA, 2704 bp, Artificial Sequence
  DNA integration cassette s423
atcgcaacagaagaggtatcaaatcatgtcggcctgtgagttagattgcctgtccagcgtgtcgcagatggcatactacccagctacaggcgccgtcccagatgcaatttctgcacctccccctacttatgaacgaagcggcaatgacaaagttgttgtttgatcagttgttggctccgtccagttaaacaaaagctgggtcaaccccttacccgagtagattcgatgaaaattcccctagcgacttctccggttagcatcttcaacggtgaccggttatagccgccggtacccgtcctccccatgcgcggacttcgctgggaacttttgcggtgtatgctacctctttaactgtagacattctgttttatttatgtacaaaagagtccctcttggtgctcccattttctgattttcaactgctcaacatctcttagaccaagtcctttctttgataaagaatctagataacagagacaaggtatcttcatacagaaaattaccgttcgtataatgtatgctatacgaagttataaccggcgttgccagcgataaacgggaaacatcatgaaaactgtttcaccctctgggaagcataaacactagaaagccaatgaagagctctacaagcctcttatgggttcaatgggtctgcaatgaccgcatacgggcttggacaattaccttctattgaatttctgagaagagatacatctcaccagcaatgtaagcagacaatcccaattctgtaaacaacctctttgtccataattccccatcagaagagtgaaaaatgccctcaaaatgcatgcgccacacccacctctcaactgcactgcgccacctctgagggtcttttcaggggtcgactaccccggacacctcgcagaggagcgaggtcacgtacttttaaaatggcagagacgcgcagtttcttgaagaaaggataaaaatgaaatggtgcggaaatgcgaaaatgatgaaaaattttcttggtggcgaggaaattgagtgcaataattggcacgaggttgttgccacccgagtgtgagtatatatcctagtttctgcacttttcttcttcttttctttaccttttcttttcaacttttttttactttttccttcaacagacaaatctaacttatatatcacaatggcgtcatacaaagaaagatcagaatcacacacttcccctgttgctaggagacttttctccatcatggaggaaaagaagtctaacctttgtgcatcattggatattactgaaactgaaaagcttctctctattttggacactattggtccttacatctgtctagttaaaacacacatcgatattgtttctgattttacgtatgaaggaactgtgttgcctttgaaggagcttgccaagaaacataattttatgatttttgaagatagaaaatttgctgatattggtaacaccgttaaaaatcaatataaatctggtgtcttccgtattgccgaatgggctgacatcactaatgcacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgtcattggttttattgcgcaacacgatatgggcggtagagaagaaggttttgactggatcattatgactccaggggttggtttagatgacaaaggtgatgcacttggtcaacaatatagaactgttgatgaagttgtaaagactggaacggatatcataattgttggtagaggtttgtacggtcaaggaagagatcctatagagcaagctaaaagataccaacaagctggttggaatgcttatttaaacagatttaaatgagtgaatttactttaaatcttgcatttaaataaattttctttttatagctttatgacttagtttcaatttatatactattttaatgacattttcgattcattgattgaaagctttgtgttttttcttgatgcgctattgcattgttcttgtctttttcgccacatttaatatctgtagtagatacctgatacattgtggatcgcctggcagcagggcgataacctcataacttcgtataatgtatgctatacgaacggtatctatcactagtcttatcgagatcgagcgaacaaactaaacctttttcatcgcggagtatattccatcacactttgcaatattatatagaaaaaagtaaaaaaaaaactctgtataactaggaaatacgatcaataaagtcattgatacacagtttaacgaaatcatcaatattggggagaatatatgctttgaaaaagggatcgttcagaacatacccaaaaaatttcttgaattcagcagtaactagatttttcggtttcttaccttgcctatttttaatgatactcgacttttcagagggtaaaaacaaagaggcaatcagcaatagctttataaacctcgaatttgccaagtttgagagaataaacgatatgtcatctttaaccttaggcatattttcgtgaatgctagaattgctacaacgggcttttgaatgtttcatgtccaaattttctgctacgttttcttcggcagtttccctgattgcgtctttgacaa

SEQ ID NO: 48 - DNA, 2671 bp, Artificial Sequence
  DNA integration cassette s425
tgtgcaccattttaatttctattgctataatgtccttattagttgccactgtgaggtgaccaatggacgagggcgagccgttcagaagccgcgaagggtgttcttcccatgaatttcttaaggagggcggctcagctccgagagtgaggcgagacgtctcggttagcgtatcccccttcctcggcttttacaaatgatgcgctcttaatagtgtgtcgttatccttttggcattgacgggggagggaaattgattgagcgcatccatattttggcggactgctgaggacaatggtggtttttccgggtggcgtgggctacaaatgatacgatggtttttttcttttcggagaaggcgtataaaaaggacacggagaacccatttattctaataacagttgagcttctttaattatttgttaatataatattctattattatatattttcttcccaataaaacaaaataaaacaaaacacagcaaaacacaaaaattaccgttcgtataatgtatgctatacgaagttataaccggcgttgccagcgataaacgggaaacatcatgaaaactgtttcaccctctgggaagcataaacactagaaagccaatgaagagctctacaagcctcttatgggttcaatgggtctgcaatgaccgcatacgggcttggacaattaccttctattgaatttctgagaagagatacatctcaccagcaatgtaagcagacaatcccaattctgtaaacaacctctttgtccataattccccatcagaagagtgaaaaatgccctcaaaatgcatgcgccacacccacctctcaactgcactgcgccacctctgagggtcttttcaggggtcgactaccccggacacctcgcagaggagcgaggtcacgtacttttaaaatggcagagacgcgcagtttcttgaagaaaggataaaaatgaaatggtgcggaaatgcgaaaatgatgaaaaattttcttggtggcgaggaaattgagtgcaataattggcacgaggttgttgccacccgagtgtgagtatatatcctagtttctgcacttttcttcttcttttctttaccttttcttttcaacttttttttactttttccttcaacagacaaatctaacttatatatcacaatggcgtcatacaaagaaagatcagaatcacacacttcccctgttgctaggagacttttctccatcatggaggaaaagaagtctaacctttgtgcatcattggatattactgaaactgaaaagcttctctctattttggacactattggtccttacatctgtctagttaaaacacacatcgatattgtttctgattttacgtatgaaggaactgtgttgcctttgaaggagcttgccaagaaacataattttatgatttttgaagatagaaaatttgctgatattggtaacaccgttaaaaatcaatataaatctggtgtcttccgtattgccgaatgggctgacatcactaatgcacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgtcattggttttattgcgcaacacgatatgggcggtagagaagaaggttttgactggatcattatgactccaggggttggtttagatgacaaaggtgatgcacttggtcaacaatatagaactgttgatgaagttgtaaagactggaacggatatcataattgttggtagaggtttgtacggtcaaggaagagatcctatagagcaagctaaaagataccaacaagctggttggaatgcttatttaaacagatttaaatgagtgaatttactttaaatcttgcatttaaataaattttctttttatagctttatgacttagtttcaatttatatactattttaatgacattttcgattcattgattgaaagctttgtgttttttcttgatgcgctattgacatttaatatctgtagtagatacctgatacattgtggatcgcctggcagcagggcgataacctcataacttcgtataatgtatgctatacgaacggtatgacatctgaatgtaaaatgaacattaaaatgaattactaaactttacgtctactttacaatctataaactttgtttaatcatataacgaaatacactaatacacaatcctgtacgtatgtaatacttttatccatcaaggattgagaaaaaaaagtaatgattccctgggccattaaaacttagacccccaagcttggataggtcactctctattttcgtttctcccttccctgatagaagggtgatatgtaattaagaataatatataattttataataaaaactaaaacaatccatcaatctcaccatcttcgttgacttcaacattcataaatccggcataagttgatagacctggaattgtcatgatctttgcagctagtgcatataaatatcctgctcctgcacttattctaacttctctgattgggaagatgaaatcctttggaacacctttcaatgttggatcatgggagagagaatattgcgtct

SEQ ID NO: 49 - DNA, 3146 bp, Artificial Sequence
  DNA integration cassette s445
acttggagaaattattaccgtttattgccttctcagtgtctgagttcctcattcgggcctttcctatcaagtttctcaacaatcgactgccttgtcttatcctcttatcagcttcatgccttcctatttgggacacggcgctttgtttcttgtaaggtaggtgaaagagagggacaaaaaaaagggggcaatatttcaaccaaagtgttgtatataaagacaatgttctcccctccctccctctcccactcttctctttgctgttgtgttgttttcttttgttttctaattacatatcctctctcttgtctgtacactacctctagtgtttcttcttcaacatcaagtagttttttgtttggccgcatccttgcgctttccagcttaattgaagagaaaatataaacatccccacacacatctataaacatacaaacagatacaaattgaaagacacattgaaagacacattgaaacacccattgatatacacataaatttcaattaatcaaaagtacgtatctacagctaacccgagtgttttttttttttttgtttttcttggtttccagattctttctttttttgttttttttgagaagtgcttgtctactaacatacttgcaaaaacatcctgcctatttaccgttcgtataatgtatgctatacgaagttataaccggcgttgccagcgataaacgggaaacatcatgaaaactgtttcaccctctgggaagcataaacactagaaagccaatgaagagctctacaagcctcttatgggttcaatgggtctgcaatgaccgcatacgggcttggacaattaccttctattgaatttctgagaagagatacatctcaccagcaatgtaagcagacaatcccaattctgtaaacaacctctttgtccataattccccatcagaagagtgaaaaatgccctcaaaatgcatgcgccacacccacctctcaactgcactgcgccacctctgagggtcttttcaggggtcgactaccccggacacctcgcagaggagcgaggtcacgtacttttaaaatggcagagacgcgcagtttcttgaagaaaggataaaaatgaaatggtgcggaaatgcgaaaatgatgaaaaattttcttggtggcgaggaaattgagtgcaataattggcacgaggttgttgccacccgagtgtgagtatatatcctagtttctgcacttttcttcttcttttctttaccttttcttttcaacttttttttactttttccttcaacagacaaatctaacttatatatcacaatggcgtcatacaaagaaagatcagaatcacacacttcccctgttgctaggagacttttctccatcatggaggaaaagaagtctaacctttgtgcatcattggatattactgaaactgaaaagcttctctctattttggacactattggtccttacatctgtctagttaaaacacacatcgatattgtttctgattttacgtatgaaggaactgtgttgcctttgaaggagcttgccaagaaacataattttatgatttttgaagatagaaaatttgctgatattggtaacaccgttaaaaatcaatataaatctggtgtcttccgtattgccgaatgggctgacatcactaatgcacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgtcattggttttattgcgcaacacgatatgggcggtagagaagaaggttttgactggatcattatgactccaggggttggtttagatgacaaaggtgatgcacttggtcaacaatatagaactgttgatgaagttgtaaagactggaacggatatcataattgttggtagaggtttgtacggtcaaggaagagatcctatagagcaagctaaaagataccaacaagctggttggaatgcttatttaaacagatttaaatgagtgaatttactttaaatcttgcatttaaataaattttctttttatagctttatgacttagtttcaatttatatactattttaatgacattttcgattcattgattgaaagctttgtgttttttcttgatgcgctattgcattgttcttgtctttttcgccacatttaatatctgtagtagatacctgatacattgtggatcgcctggcagcagggcgataacctcataacttcgtataatgtatgctatacgaacggtatttaggtgtcagacatttgcacttgaaggataggagccccaacctgttgtaatttatgtttgatgttttgtaacgtttatctttatctttatcttgatctttgttttcgtttttgtttatgtttttgattttatacagttatacttatgctaagatctatatctttgtttggtcttacatataaatgtaccaatatgctttgcttccaagttatcccactttgaatgcgagctgacagtatgactccaaaaagcgtataaacgtgggtggtacaaattgaagcggttactgaatgtcagattgtcaatttttttcccttgtattatttttttttttcactcctgtttccttctgtattttgtcgttctctgtgcattactcgacagatctgtcgaaatccccacctagtcagtgcatttcttatttgaaaccatgcatatcctccatagtacattaggtctcaactcaaacaaaacgctgactgacgtatggttccaatacgttctccgaaattacaaatctccgagattcataatcacaacttttggtgtgttattgacatcatatatttttttcccgtcatcgttacttgcagtctctcacaaaccttctaaaaggccagataagtacacatgtgggttcaaaaacagcgggaatgactgttttgccaattctacactacagtcactgtcttcgctagatacactttatttgtatctagccgagatgctgagtttccaaatgccaccaggatacaccatctacccattaccattacatacgtctctatatcatatgc

SEQ ID NO: 50 - DNA, 8579 bp, Artificial Sequence
  DNA integration cassette s484, s485 and s486
gtatgataggtgtttccatgataaacaacatgattgggtgtatctttacattcacttgctccccatggttaaatgcaatgggtaacacaaacacatatgcaattttgactgccttccaagtcattgcatgtttatctgctgttccatttctcatttggggtaaaaagatgcgtttatggaccagaaaatactaccttgattttgtggaaaagagagatggagtcgaaaaatcaagctgacatatgcactgtcctatatacctcatcgaagctacttttttagtttcgttttctaagcactattctctttaattaatccgataattgtacaaaaaaaaacatgcttctttcaaaatcatgaatgggatactacagaacttagccaccaatattagtggttattttgtaatttttggagtaaacattataacgtaaagtaggtcagctctcctcctctgtgttgtctaaatgaaacaaatctgtatacatcatgctcatggctcgttgtgtggataaacacgtaatacattccatttttataaagggcgtcacgctgctcctaattgagaaaacactacttgcataaaggtgagatccatgatagcaaaatgtagggtaatgtacaaatagacaagcacatgggtcgatagattgtttatattaatctctaccagcctatcattggctttggttagagacaaatcaaattatccctccctcccttaattgtaatcatatccttttgtacaggattggaatctaaggcggggaacaaattctaaaatgcgaacaattctccgccacacttgccttatcaaggaataatttccaccacctgttacggtacgttgtcaaattgatgatggcctggtataaatgtttgttcattctatttgaaactctacctgttactggacctctagcatttcccattggtttttgatatatcaaccacatttccctaattgcgcggcgcgacttcgacagaaccagggctagatttcgatatggatatggatatggatatggatatggagatgaatttgaatttagatttgggtcttgatttggggttggaattaaaaggggataacaatgagggttttcctgttgatttaaacaatggacgtgggaggtgattgatttaacctgatccaaaaggggtatgtctattttttagagtgtgtctttgtgtcaaattatagtagaatgtgtaaagtagtataaactttcctctcaaatgacgaggtttaaaacaccccccgggtgagccgagccgagaatggggcaattgttcaatgtgaaatagaagtatcgagtgagaaacttgggtgttggccagccaaggggggggggaaggaaaatggcgcgaatgctcaggtgagattgttttggaattgggtgaagcgaggaaatgagcgacccggaggttgtgactttagtggcggaggaggacggaggaaaagccaagagggaagtgtatataaggggagcaatttgccaccaggatagaattggatgagttataattctactgtatttattgtataatttatttctccttttgtatcaaacacattacaaaacacacaaaacacacaaacaaacacaattacaaaaaatggaagataaagaaggacgatttcgagtggaatgcattgaaaatgtacattatgtaacagatatgttttgtaaatatccattaaaacttatcgctcctaaaacaaaacttgatttttctattctgtacatcatgagctatggaggtggcctggtatcaggggatcgtgtagcgctggatattatagttggaaaaaatgctacattgtgcatacagagtcaaggaaatacaaaattatataaacaaataccaggaaagcctgcaacacagcaaaagttggatgtagaagttggaacgaatgcattgtgcttgttattacaagatccagtgcaaccttttggagatagtaattacattcagactcaaaactttgtattagaagacgaaacttcttctcttgcattactggattggacattacatggtcgaagccatatcaatgaacaatggagtatgcgatcttatgtgtccaaaaattgtatccagatgaagattccagcttcaaaccagagaaaaacgcttttgagagatgtgttaaaaatattcgatgagcctaacctacatattggtttaaaagccgaacgaatgcatcactttgaatgtataggcaatttgtatcttataggaccaaaatttcttaaaactaaagaagcagttttgaaccaatataggaacaaggagaagaggatatcaaaaacaacggattcatctcaaatgaagaagattatctggactgcttgtgaaattcggtcggttacaataattaaattcgctgcttacaacactgaaactgcacgaaattttcttctgaaattattttcggactacgcaagctttctagatcatgaaactcttcgcgctttttggtactgagtgaatttactttaaatcttgcatttaaataaattttctttttatagctttatgacttagtttcaatttatatactattttaatgacattttcgattcattgattgaaagctttgtgttttttcttgatgcgctattgcattgttcttgtctttttcgccacatgtaatatctgtagtagatacctgatacattgtggatgaaacatcatgaaaactgtttcaccctctgtgaagcataaacactagaaagccaatgaagagctctacaagcctcttatgggttcaatgggtctgcaatgaccgcatacgggcttggacaattaccttctattgaatttctgagaagagatacatctcaccagcaatgtaagcagacaatcccaattctgtaaacaacctctttgtccataattccccatcagaagagtgaaaaatgccctcaaaatgcatgcgccacacccatctttcaactgcactgcgccacctctgagggtcttttcaggggtcgactaccccggacacctcgcagaggagcgaggtcacgtacttttaaaatggcagagacgcgcagtttcttgaagaaaggataaaaatgaaatggtgcggaaatgcgaaaatgatgaaaaattttcttggtggcgaggaaattgagtgcaataattggcacgaggttgttgccacccgagtgtgagtatatatcctagtttctgcacttttcttcttcttttctttaccttttcttttcaacttttttttactttttccttcaacagacaaatctaacttatatatcacaatgactgattcgcaaacggaaacacacttgtcgctaattctttcagacactgcgtttcctctgtcatctttttcttattcgtatgggttagagtcgtatttgtctcatcagcaggtgagagacgtcaatgcatttttcaactttttaccattgtccctcaattcagtgctacataccaatttgccaactgtcaaagcagcttgggagtcaccgcaacaatattccgaaatcgaagacttttttgaaagcacacagacatgcacaattgcccaaaaggtctccaccatgcagggtaaatctttgttaaatatttggacaaaatcactctcctttttcgttacatcaaccgatgtcttcaaatacttggatgagtacgaaagaagagttcgtagtaaaaaggcactcggtcatttcccagtggtttggggtgtggtatgtagagccttgggattatcgttagaaaggacatgttatctgttcttattggggcatgcaaaatcgatttgctcagcagctgttcgcttagatgttttgacctccttccagtacgtttccactttggctcatcctcaaaccgaaagtttacttagagattcgtcgcaactagctttgaacatgcaactagaggacactgctcagtcatggtatacgctggacctttggcagggtagacacagtttgttatatagtagaatatttaatagttaatccagccagtaaaatccatactcaacgacgatatgaacaaatttccctcattccgatgctgtatatgtgtataaatttttacatgctcttctgtttagacacagaacagctttaaataaaatgttggatatactttttctgcctgtggtgtaccgttcgtataatgtatgctatacgaagttataaccggcgttgccagcgataaacggctccatgctggacttactcgtcgaagatttcctgctactctctatataattagacacccatgttatagatttcagaaaacaatgtaataatatatggtagcctcctgaaactaccaagggaaaaatctcaacaccaagagctcatattcgttggaatagcgataatatctctttacctcaatcttatatgcatgttatttcgcctggcagcagggcgataacctcatttggttcattaacttttggttctgttcttggaaacgggtaccaactctctcagagtgcttcaaaaatttttcagcacatttggttagacatgaactttctctgctggttaaggattcagaggtgaagtcttgaacacaatcgttgaaacatctgtccacaagagatgtgtatagcctcatgaaatcagccatttgcttttgttcaacgatcttttgaaattgttgttgttcttggtagttaagttgatccatcttggcttatgttgtgtgtatgttgtagttattcttagtatattcctgtcctgagtttagtgaaacataatatcgccttgaaatgaaaatgctgaaattcgtcgacatacaatttttcaaacttttttttttgttggtgcacggacatgtttttaaaggaagtactctataccagttattcttcacaaatttaattgctggagaatagatcttcaacgctttaataaagtagtttgtttgttaaggatggcgtcatacaaagaaagatcagaatcacacacttcccctgttgctaggagacttttctccatcatggaggaaaagaagtctaacctttgtgcatcattggatattactgaaactgaaaagcttctctctattttggacactattggtccttacatctgtctagttaaaacacacatcgatattgtttctgattttacgtatgaaggaactgtgttgcctttgaaggagcttgccaagaaacataattttatgatttttgaagatagaaaatttgctgatattggtaacactgttaaaaatcaatataaatctggtgtcttccgtattgccgaatgggctgacatcactaatgcacatggtgtaacgggtgcaggtattgtttctggcttgaaggaggccgcccaagaaacaaccagtgaacctagaggtttgctaatgcttgctgagttatcatcaaagggttctttagcatatggtgaatatacagaaaaaacagtagaaattgctaaatctgataaagagtttgtcattggttttattgcgcaacacgatatgggcggtagagaagaaggttttgactggatcattatgactccaggggttggtttagatgacaaaggtgatgcacttggtcaacaatatagaactgttgatgaagttgtaaagactggaacggatatcataattgttggtagaggtttgtatggtcaaggaagagatcctgtagagcaagctaaaagataccaacaagctggttggaatgcttatttaaacagatttaaatgattcttacacaaagatttgatacatgtacactagtttaaataagcatgaaaagaattacacaagcaaaaaaaaaattaaatgaggtactttgagtaaaatcttatgatttagaaaaagttgtttaacaaaggctttagtatgtgaatttttaatgtagcaaagcgataactaataaacataaacaaaagtatggttttcttaaccggcgttgccagcgataaacggctccatgctggacttactcgtcgaagatttcctgctactctctatataattagacacccatgttatagatttcagaaaacaatgtaataatatatggtagcctcctgaaactaccaagggaaaaatctcaacaccaagagctcatattcgttggaatagcgataatatctctttacctcaatcttatatgcatgttatttcgcctggcagcagggcgataacctcataacttcgtataatgtatgctatacgaacggtagctacttagcttctatagttagttaatgcactcacgatattcaaaattgacacccttcaactactccctactattgtctactactgtctactactcctctttactatagctgctcccaataggctccaccaataggctctgccaatacattttgcgccgccacctttcaggttgtgtcactcctgaaggaccatattgggtaatcgtgcaatttctggaagagagtccgcgagaagtgaggcccccactgtaaatcctcgagggggcatggagtatggggcatggaggatggaggatggggggggggcgaaaaataggtagcaaaaggacccgctatcaccccacccggagaactcgttgccgggaagtcatatttcgacactccggggagtctataaaaggcgggttttgtcttttgccagttgatgttgctgaaaggacttgtttgccgtttcttccgatttaacagtatagaaatcaaccactgttaattatacacgttatactaacacaacaaaaacaaaaacaacgacaacaacaacaacaatggcgattccttttcttcacaagggaggttctgatgactcgactcatcaccatacacacgattacgaccatcataaccatgatcatcatggtcacgatcatcacagccatgattcatcttccaactcttccagcgaagctgccagattgcagttcatccaagagcatggccattctcacgatgctatggaaacgcctggcagctacttgaagcgtgaacttcctcagttcaatcatagagacttctctcgtcgtgcctttaccattggcgtcggaggaccggtcggttctggtaaaactgcacttttgcttcagctttgcaggctcttgggtgaaaaatatagcatcggagttgttaccaacgacatatttactcgtgaagatcaagaatttttaattcgtaacaaggcacttcccgaagagagaattcgcgcaatcgaaacaggcggttgtccacacgctgctattcgtgaagacgtctccggtaatttggtcgcattggaggagttgcaatccgagttcaacacagaattactactcgtggagtcaggaggtgataacttagctgcaaattactctcgtgatctcgctgatttcattatctatgtaattgatgtatctggaggcgacaagattccacgtaagggtggacctggtatcacggagtcagatctgttgattatcaacaaaacagatctagctaagttggtcggtgctgatttgtcggtcatggatcgtgatgcaaaaaagattcgtgagaatggacccattgtttttgcacaagtcaaaaatcaagttgggatggatgagatcaccgaacttattctaggcgccgctaagagtgctggtgctctcaagtaaatgagctatacaggcaatttatatcgaagtatgtaacatttggtaatccgccgaactgcagtaataacaagtactggccctaattacttgagcaatacattatcctttttcttctgccataacacagattgctttgtttttttgtgtcttggcacttaaacagtctggtagcatcagctttttccaaaatcacgaaatttcaaattttttaggctccatttagagcatcaataattaaaacaacttcatgttacaagtctataataaaccgtaaaatttacgtatccctagattacacacaaaaaaaactacataggtcccaattagcgggatttattaaagataagttccaacgtcagacatggcatactaactactatggtcgcccaagttaaagacgactcgctccacagctgtgcttaccgaaggggcaatcggttttgtttcttgcaagatgccaaatcagcgagtgatattctggctttttttttttttgcacaaacgaacaccatgaattccatgatgccgtagttgcagctttgcaggatatataactgccgactattgaccttctgataagcagaccgttaacatgttgttttctaaaaaggaagaaacgagtgaaccgccatctcgttcgaaacgtgagcaatgctgggcatcaagagatgcatactttgcttgccttgacaagcacaatatcgagaatccactagacccagaaaaggcgaagattgcatcaaaaaattgtgctgctgaagacaagcaattttctaaagattgtgttgcaagttgggtgaagtacttcaaagagaaaaggccattcgacattaaaaaggaaaggatgttgaaagaagctgcagaaaatgggcaagaaatcgttcaaatggaaggatatagaaagtagctggaatttccaataaaaaataccctttacagaaaaatatattcatgtaaatacaaatga

SEQ ID NO: 51 - DNA, 5181 bp, Artificial Sequence
  DNA integration cassette s481
acttggagaaattattaccgtttattgccttctcagtgtctgagttcctcattcgggcctttcctatcaagtttctcaacaatcgactgccttgtcttatcctcttatcagcttcatgccttcctatttgggacacggcgctttgtttcttgtaaggtaggtgaaagagagggacaaaaaaaagggggcaatatttcaaccaaagtgttgtatataaagacaatgttctcccctccctccctctcccactcttctctttgctgttgtgttgttttcttttgttttctaattacatatcctctctcttgtctgtacactacctctagtgtttcttcttcaacatcaagtagttttttgtttggccgcatccttgcgctttccagcttaattgaagagaaaatataaacatccccacacacatctataaacatacaaacagatacaaattgaaagacacattgaaagacacattgaaacacccattgatatacacataaatttcaattaatcaaaagtacgtatctacagctaacccgagtgttttttttttttttgtttttcttggtttccagattctttctttttttgttttttttgagaagtgcttgtctactaacatacttgcaaaaacatcctgcctattgggctagatttcgatatggatatggatatggatatggatatggagatgaatttgaatttagatttgggtcttgatttggggttggaattaaaaggggataacaatgagggttttcctgttgatttaaacaatggacgtgggaggtgattgatttaacctgatccaaaaggggtatgtctattttttagagtgtgtctttgtgtcaaattatagtagaatgtgtaaagtagtataaactttcctctcaaatgacgaggtttaaaacaccccccgggtgagccgagccgagaatggggcaattgttcaatgtgaaatagaagtatcgagtgagaaacttgggtgttggccagccaagggggaaggaaaatggcgcgaatgctcaggtgagattgttttggaattgggtgaagcgaggaaatgagcgacccggaggttgtgactttagtggcggaggaggacggaggaaaagccaagagggaagtgtatataaggggagcaatttgccaccaggatagaattggatgagttataattctactgtatttattgtataatttatttctccttttgtatcaaacacattacaaaacacacaaaacacacaaacaaacacaattacaaaaaatgcaacccagagagctacacaaattaacgcttcaccagctgggatctttagcccaaaaaaggctgtgtagaggggtaaagcttaacaagttagaggctacttcacttattgcatctcaaattcaagaatatgttcgcgacggtaatcattccgtagcagatttgatgagtcttggtaaagatatgctgggtaaacgccatgttcagcccaatgtcgttcatttgttacatgaaattatgattgaagcgactttccctgatggaacctatctaattaccattcatgatcccatttgcactacagatggtaatctcgaacatgctttatatggaagcttcctgcctacgccaagccaagaactgttccctctggaagaggaaaagttatatgctccggaaaatagccctggttttgttgaagtcttggagggcgagattgaactattgcctaatttacctcgtactcccatcgaggtacgaaacatgggtgacaggccaattcaagttggatcacactatcattttattgaaactaatgaaaaactatgcttcgatcgctcaaaggcttatggaaagcgcttggacattccgtcaggtactgctattcgatttgaacctggcgtaatgaaaattgtcaatttaatccctatcggtggtgcaaaactaattcaaggaggtaattcactttcgaagggtgtcttcgatgattctaggactcgggaaattgttgacaatttgatgaaacagggattcatgcatcaacctgaatctccgttgaatatgccattacaatctgcacgcccttttgttgttcctcgtaaattatacgctgtaatgtatggtccaacaacgaatgataaaattcgtctgggagatacaaatttgattgtgcgcgtggaaaaggactttactgaatatggaaatgaatctgttttcggcggcggaaaggttatacgtgatggtacgggacagtctagctcaaaatcgatggacgaatgcttggacactgtaattacaaatgctgtaatcattgatcataccggtatctacaaggctgacattggcattaaaaacggatatatcgtaggtataggtaaagcaggaaacccggatacaatggataacattggagaaaacatggtcattggatcttctacagatgttatttcagctgagaataaaattgttacttatggtggtatggacagccacgttcatttcatctgtcctcaacaaattgaagaggcattggcttccggtataactactatgtatggtggaggaactggccctagtacgggaactaatgctactacctgcaccccaaataaagacttaatccgttctatgcttcgttctactgattcttatcccatgaacattggtctcaccggaaaaggaaatgatagcggttcaagttctttgaaggagcaaatagaagcaggctgcagtggacttaagcttcacgaagattggggatctactcccgcagcaattgacagttgtttgtctgtttgtgatgagtatgacgttcagtgcctaattcataccgacaccctcaatgaatcctcttttgtagaaggtacatttaaagcttttaaaaataggaccattcacacgtatcacgttgaaggagccggtggtgggcatgcccccgatattatttctttagtccaaaatccaaatattcttccctctagcaccaatcccacacgaccatttactacaaatacgcttgatgaggaactggacatgttaatggtatgccatcatctttctaggaatgttcctgaagacgttgcatttgcagaatcccgtattcgtgctgaaacaattgctgctgaagatattttacaggatttgggagctattagtatgattagttcagactctcaagccatgggtcgttgtggtgaagtaatttcaagaacttggaaaaccgcccataaaaataagctacaacgaggagcacttcctgaggacgagggttcaggtgttgataatttccgtgtgaaacgttatgtatccaaatacactataaaccctgcaattactcatggaatttctcatattgttggttctgtggagataggcaagtttgctgatcttgtcttatgggactttgctgactttggggcaagacccagtatggtgctgaaaggaggaatgattgcattggcctctatgggtgatccaaatggatcgattccaacggtttctcccctcatgtcctggcaaatgtttggtgcacatgaccccgagaggagcattgcatttgtttccaaggcctctataacatccggtgttattgaaagctatggacttcataagagagttgaagccgtaaaatatacgagaaacattgggaagaaagacatggtttacaattcatatatgccaaaaatgactgttgatccagaagcttacacagttactgcagatggtaaagttatggaatgtgagcctgtagacaaacttccactttcccagtcttattttatcttttaatccagccagtaaaatccatactcaacgacgatatgaacaaatttccctcattccgatgctgtatatgtgtataaatttttacatgctcttctgtttagacacagaacagctttaaataaaatgttggatatactttttctgcctgtggtgtaccgttcgtataatgtatgctatacgaagttataaccggcgttgccagcgataaacgggaaacatcatgaaaactgtttcaccctctgggaagcataaacactagaaagccaatgaagagctctacaagcctcttatgggttcaatgggtctgcaatgaccgcatacgggcttggacaattaccttctattgaatttctgagaagagatacatctcaccagcaatgtaagcagacaatcccaattctgtaaacaacctctttgtccataattccccatcagaagagtgaaaaatgccctcaaaatgcatgcgccacacccacctctcaactgcactgcgccacctctgagggtcttttcaggggtcgactaccccggacacctcgcagaggagcgaggtcacgtacttttaaaatggcagagacgcgcagtttcttgaagaaaggataaaaatgaaatggtgcggaaatgcgaaaatgatgaaaaattttcttggtggcgaggaaattgagtgcaataattggcacgaggttgttgccacccgagtgtgagtatatatcctagtttctgcacttttcttcttcttttctttaccttttcttttcaacttttttttactttttccttcaacagacaaatctaacttatatatcacaatggcgtcatacaaagaaagatcagaatcacacacttcccctgttgctaggagacttttctccatcatggaggaaaagaagtctaacctttgtgcatcattggatattactgaaactgaaaagcttctctctattttggacactattggtccttacatctgtctagttaaaacacacatcgatattgtttctgattttacgtatgaaggaactgtgttgcctttgaaggagcttgccaagaaacataattttatgatttttgaagatagaaaatttgctgatattggtaacaccgttaaaaatcaatataaatctggtgtcttccgtattgccgaatgggctgacatcactaatgcacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgtcattggttttattgcgcaacacgatatgggcggtagagaagaaggttttgactggatcattatgactcca

SEQ ID NO: 52 - DNA, 3320 bp, Artificial Sequence
  DNA integration cassette s482
aatcaatataaatctggtgtcttccgtattgccgaatgggctgacatcactaatgcacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgtcattggttttattgcgcaacacgatatgggcggtagagaagaaggttttgactggatcattatgactccaggggttggtttagatgacaaaggtgatgcacttggtcaacaatatagaactgttgatgaagttgtaaagactggaacggatatcataattgttggtagaggtttgtacggtcaaggaagagatcctatagagcaagctaaaagataccaacaagctggttggaatgcttatttaaacagatttaaatgagtgaatttactttaaatcttgcatttaaataaattttctttttatagctttatgacttagtttcaatttatatactattttaatgacattttcgattcattgattgaaagctttgtgttttttcttgatgcgctattgcattgttcttgtctttttcgccacatttaatatctgtagtagatacctgatacattgtggatcgcctggcagcagggcgataacctcataacttcgtataatgtatgctatacgaacggtagctacttagcttctatagttagttaatgcactcacgatattcaaaattgacacccttcaactactccctactattgtctactactgtctactactcctctttactatagctgctcccaataggctccaccaataggctctgccaatacattttgcgccgccacctttcaggttgtgtcactcctgaaggaccatattgggtaatcgtgcaatttctggaagagagtccgcgagaagtgaggcccccactgtaaatcctcgagggggcatggagtatggggcatggaggatggaggatggggggggggcgaaaaataggtagcaaaaggacccgctatcaccccacccggagaactcgttgccgggaagtcatatttcgacactccggggagtctataaaaggcgggttttgtcttttgccagttgatgttgctgaaaggacttgtttgccgtttcttccgatttaacagtatagaaatcaaccactgttaattatacacgttatactaacacaacaaaaacaaaaacaacgacaacaacaacaacaatgaacagtatgtctgaatatgttaaacctagaaaaaatgaatttataaggaagtttgagaatttttatttcgaaataccctttctatcaaagcttccaccaaaggttagcgtgcctatcttttctttgatatcggtaaatatcgtagtttggataattgcggcaatagtcatcagtttagttaacagatcgttatttctctcagttttattatcttggacacttggtttaagacacgctctcgatgctgatcatattactgcaattgacaacttaacgcgccgtttattatcaacagacaaaccaatgtcaacagttggaacctggttcagcattggtcattcaactgtagtccttataacttgcatcgtagtagcagctacttccagtaagtttgcagatcgatgggataactttcaaaccataggaggaataattggaacttcagttagcatgggactattacttttgttggcaattggaaataccgttttactagtccggttatcgtattggctttggatgtatcgcaaatctggtgtcactaaagatgaaggggtcaccggattcttagctcgaaaaatgcagagattgtttagattggttgactctccgtggaagatttatgtacttggttttgttttcggtttgggatttgataccagtactgaggtttccttgctgggtatcgcaaccttgcaagccttaaaaggaacttctatatgggcaatcttacttttccccattgtatttcttgttggaatgtgcttagttgataccacagatggagcattaatgtattatgcttactcatattcttcgggtgaaaccaatccttatttctctaggctttattactccataattttaacatttgtttcggttatagcagcatttacaatcggtatcattcaaatgcttatgctaatcataagtgtccacccaatggaaagtacattttggaatggcctcaatagattatctgataattacgaaatagtcggtggatgtatatgcggtgcctttgttctagcaggtttgtttggtatttccatgcataattactttaagaaaaaattcacacctctagtgcaagtaggaaatgacagagaggacgaagttctagagaaaaataaagaattagaaaacgtatcaaaaaactcgatttctgttcaaatttccgaaagtgaaaaggtgagttacgatacagtggattctaaggtttgatttaggtgtcagacatttgcacttgaaggataggagccccaacctgttgtaatttatgtttgatgttttgtaacgtttatctttatctttatcttgatctttgttttcgtttttgtttatgtttttgattttatacagttatacttatgctaagatctatatctttgtttggtcttacatataaatgtaccaatatgctttgcttccaagttatcccactttgaatgcgagctgacagtatgactccaaaaagcgtataaacgtgggtggtacaaattgaagcggttactgaatgtcagattgtcaatttttttcccttgtattatttttttttttcactcctgtttccttctgtattttgtcgttctctgtgcattactcgacagatctgtcgaaatccccacctagtcagtgcatttcttatttgaaaccatgcatatcctccatagtacattaggtctcaactcaaacaaaacgctgactgacgtatggttccaatacgttctccgaaattacaaatctccgagattcataatcacaacttttggtgtgttattgacatcatatatttttttcccgtcatcgttacttgcagtctctcacaaaccttctaaaaggccagataagtacacatgtgggttcaaaaacagcgggaatgactgttttgccaattctacactacagtcactgtcttcgctagatacactttatttgtatctagccgagatgctgagtttccaaatgccaccaggatacaccatctacccattaccattacatacgtctctatatcatatgc

SEQ ID NO: 53 - DNA, 1547 bp, Artificial Sequence
  DNA integration cassette s483
aatcaatataaatctggtgtcttccgtattgccgaatgggctgacatcactaatgcacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgtcattggttttattgcgcaacacgatatgggcggtagagaagaaggttttgactggatcattatgactccaggggttggtttagatgacaaaggtgatgcacttggtcaacaatatagaactgttgatgaagttgtaaagactggaacggatatcataattgttggtagaggtttgtacggtcaaggaagagatcctatagagcaagctaaaagataccaacaagctggttggaatgcttatttaaacagatttaaatgagtgaatttactttaaatcttgcatttaaataaattttctttttatagctttatgacttagtttcaatttatatactattttaatgacattttcgattcattgattgaaagctttgtgttttttcttgatgcgctattgcattgttcttgtctttttcgccacatttaatatctgtagtagatacctgatacattgtggatcgcctggcagcagggcgataacctcataacttcgtataatgtatgctatacgaacggtatttaggtgtcagacatttgcacttgaaggataggagccccaacctgttgtaatttatgtttgatgttttgtaacgtttatctttatctttatcttgatctttgttttcgtttttgtttatgtttttgattttatacagttatacttatgctaagatctatatctttgtttggtcttacatataaatgtaccaatatgctttgcttccaagttatcccactttgaatgcgagctgacagtatgactccaaaaagcgtataaacgtgggtggtacaaattgaagcggttactgaatgtcagattgtcaatttttttcccttgtattatttttttttttcactcctgtttccttctgtattttgtcgttctctgtgcattactcgacagatctgtcgaaatccccacctagtcagtgcatttcttatttgaaaccatgcatatcctccatagtacattaggtctcaactcaaacaaaacgctgactgacgtatggttccaatacgttctccgaaattacaaatctccgagattcataatcacaacttttggtgtgttattgacatcatatatttttttcccgtcatcgttacttgcagtctctcacaaaccttctaaaaggccagataagtacacatgtgggttcaaaaacagcgggaatgactgttttgccaattctacactacagtcactgtcttcgctagatacactttatttgtatctagccgagatgctgagtttccaaatgccaccaggatacaccatctacccattaccattacatacgtctctatatcatatgc

SEQ ID NO: 54 - DNA, 3304 bp, Artificial Sequence
  DNA integration cassette s394
gcaggcttatggcagacaggtactttttttttgtctctgtataatgagtcaaattgtcaatattgaagggttgtatccaaactgcagttcttgacagtcagacacactcatctttcataaccttccctaaatagatgtgctcctatttcagccaagtatctttattgtcggtgaaaataatggaaacggtctaaatgcgcttgttactaaggctgttactttgataaacgcatttgactttgagatatataacttcaactctaacgacctaatttcaaacggaagagctacttagaccatagattaaaagtgaattctctctaacacactttgaggagcattaatttcacaccaaaacgtctatagatgctgactttagcggtttcaatgggaattgatcttgcaacaccaaggaattgccattgaagagaaacttactgatacatcattcaaccactccgatgatatacaccgggctagatttcgatatggatatggatatggatatggatatggagatgaatttgaatttagatttgggtcttgatttggggttggaattaaaaggggataacaatgagggttttcctgttgatttaaacaatggacgtgggaggtgattgatttaacctgatccaaaaggggtatgtctattttttagagtgtgtctttgtgtcaaattatagtagaatgtgtaaagtagtataaactttcctctcaaatgacgaggtttaaaacaccccccgggtgagccgagccgagaatggggcaattgttcaatgtgaaatagaagtatcgagtgagaaacttgggtgttggccagccaagggggaaggaaaatggcgcgaatgctcaggtgagattgttttggaattgggtgaagcgaggaaatgagcgacccggaggttgtgactttagtggcggaggaggacggaggaaaagccaagagggaagtgtatataaggggagcaatttgccaccaggatagaattggatgagttataattctactgtatttattgtataatttatttctccttttgtatcaaacacattacaaaacacacaaaacacacaaacaaacacaattacaaaaaatgttgcacgtttctatggttggttgtggtgctatcggtcgtggtgtcttagaattgttgaagtccgatccagacgttgttttcgatgttgttattgttccagaacatactatggatgaagctcgtggtgctgtctccgctttagccccaagagctagagttgccacccacttggatgatcaacgtccagatttgttagttgaatgcgccggtcatcacgctttagaagaacacattgtcccagccttagaaagaggtatcccttgtatggttgtctctgttggtgctttgtctgagcctggtatggctgaacgtttggaagccgctgctcgtagaggtggtacccaagtccaattgttgtccggtgctatcggtgccatcgatgctttagccgctgctcgtgtcggtggtttggacgaagttatctacaccggtagaaaaccagctagagcttggaccggtactccagctgagcaattgttcgacttggaagctttaactgaagccactgtcattttcgaaggtactgctagagatgccgctagattataccctaagaacgctaacgttgccgctaccgtttctttagctggtttgggtttggatagaaccgctgttaagttattggctgatcctcacgctgttgaaaacgtccaccatgtcgaagccagaggtgccttcggtggtttcgaattgaccatgagaggtaagccattggctgccaacccaaagacctctgctttaactgtcttttccgttgttagagctttgggtaatagagcccacgccgtttctatctaatccagccagtaaaatccatactcaacgacgatatgaacaaatttccctcattccgatgctgtatatgtgtataaatttttacatgctcttctgtttagacacagaacagctttaaataaaatgttggatatactttttctgcctgtggtgtaccgttcgtataatgtatgctatacgaagttataaccggcgttgccagcgataaacgggaaacatcatgaaaactgtttcaccctctgggaagcataaacactagaaagccaatgaagagctctacaagcctcttatgggttcaatgggtctgcaatgaccgcatacgggcttggacaattaccttctattgaatttctgagaagagatacatctcaccagcaatgtaagcagacaatcccaattctgtaaacaacctctttgtccataattccccatcagaagagtgaaaaatgccctcaaaatgcatgcgccacacccacctctcaactgcactgcgccacctctgagggtcttttcaggggtcgactaccccggacacctcgcagaggagcgaggtcacgtacttttaaaatggcagagacgcgcagtttcttgaagaaaggataaaaatgaaatggtgcggaaatgcgaaaatgatgaaaaattttcttggtggcgaggaaattgagtgcaataattggcacgaggttgttgccacccgagtgtgagtatatatcctagtttctgcacttttcttcttcttttctttaccttttcttttcaacttttttttactttttccttcaacagacaaatctaacttatatatcacaatggcgtcatacaaagaaagatcagaatcacacacttcccctgttgctaggagacttttctccatcatggaggaaaagaagtctaacctttgtgcatcattggatattactgaaactgaaaagcttctctctattttggacactattggtccttacatctgtctagttaaaacacacatcgatattgtttctgattttacgtatgaaggaactgtgttgcctttgaaggagcttgccaagaaacataattttatgatttttgaagatagaaaatttgctgatattggtaacaccgttaaaaatcaatataaatctggtgtcttccgtattgccgaatgggctgacatcactaatgcacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgtcattggttttattgcgcaacacgatatgggcggtagagaagaaggttttgactggatcattatgactcca

SEQ ID NO: 55 - DNA, 3307 bp, Artificial Sequence
  DNA integration cassette s396
gcaggcttatggcagacaggtactttttttttgtctctgtataatgagtcaaattgtcaatattgaagggttgtatccaaactgcagttcttgacagtcagacacactcatctttcataaccttccctaaatagatgtgctcctatttcagccaagtatctttattgtcggtgaaaataatggaaacggtctaaatgcgcttgttactaaggctgttactttgataaacgcatttgactttgagatatataacttcaactctaacgacctaatttcaaacggaagagctacttagaccatagattaaaagtgaattctctctaacacactttgaggagcattaatttcacaccaaaacgtctatagatgctgactttagcggtttcaatgggaattgatcttgcaacaccaaggaattgccattgaagagaaacttactgatacatcattcaaccactccgatgatatacaccgggctagatttcgatatggatatggatatggatatggatatggagatgaatttgaatttagatttgggtcttgatttggggttggaattaaaaggggataacaatgagggttttcctgttgatttaaacaatggacgtgggaggtgattgatttaacctgatccaaaaggggtatgtctattttttagagtgtgtctttgtgtcaaattatagtagaatgtgtaaagtagtataaactttcctctcaaatgacgaggtttaaaacaccccccgggtgagccgagccgagaatggggcaattgttcaatgtgaaatagaagtatcgagtgagaaacttgggtgttggccagccaagggggaaggaaaatggcgcgaatgctcaggtgagattgttttggaattgggtgaagcgaggaaatgagcgacccggaggttgtgactttagtggcggaggaggacggaggaaaagccaagagggaagtgtatataaggggagcaatttgccaccaggatagaattggatgagttataattctactgtatttattgtataatttatttctccttttgtatcaaacacattacaaaacacacaaaacacacaaacaaacacaattacaaaaaatgttgaagatcgctatgattggttgtggtgctatcggtgcctccgtcttggaattgttgcatggtgactctgacgttgttgttgatagagttatcaccgttccagaagctagagacagaactgaaatcgctgttgccagatgggctccaagagccagagttttggaagttttggctgctgacgatgccccagacttggttgttgaatgtgccggtcacggtgctatcgctgctcatgttgtcccagccttggaaagaggtattccatgtgttgttacctccgttggtgctttgtctgctccaggtatggctcaattattggagcaagccgccagaagaggtaagacccaagtccaattgttgtccggtgctatcggtggtatcgacgctttagctgccgctagagtcggtggtttggattccgtcgtttacactggtagaaagccaccaatggcctggaagggtactcctgctgaagctgtctgtgatttggactctttgaccgttgcccactgtattttcgacggttctgctgaacaagccgcccaattatacccaaagaacgctaacgttgctgctactttgtctttagccggtttgggtttgaagagaactcaagtccaattgttcgctgacccaggtgtttctgagaatgttcaccacgtcgctgctcatggtgctttcggttctttcgaattgactatgagaggtagaccattggctgccaaccctaagacctctgctttgaccgtctattctgttgtcagagctttgttaaacagaggtagagctttggttatttaatccagccagtaaaatccatactcaacgacgatatgaacaaatttccctcattccgatgctgtatatgtgtataaatttttacatgctcttctgtttagacacagaacagctttaaataaaatgttggatatactttttctgcctgtggtgtaccgttcgtataatgtatgctatacgaagttataaccggcgttgccagcgataaacgggaaacatcatgaaaactgtttcaccctctgggaagcataaacactagaaagccaatgaagagctctacaagcctcttatgggttcaatgggtctgcaatgaccgcatacgggcttggacaattaccttctattgaatttctgagaagagatacatctcaccagcaatgtaagcagacaatcccaattctgtaaacaacctctttgtccataattccccatcagaagagtgaaaaatgccctcaaaatgcatgcgccacacccacctctcaactgcactgcgccacctctgagggtcttttcaggggtcgactaccccggacacctcgcagaggagcgaggtcacgtacttttaaaatggcagagacgcgcagtttcttgaagaaaggataaaaatgaaatggtgcggaaatgcgaaaatgatgaaaaattttcttggtggcgaggaaattgagtgcaataattggcacgaggttgttgccacccgagtgtgagtatatatcctagtttctgcacttttcttcttcttttctttaccttttcttttcaacttttttttactttttccttcaacagacaaatctaacttatatatcacaatggcgtcatacaaagaaagatcagaatcacacacttcccctgttgctaggagacttttctccatcatggaggaaaagaagtctaacctttgtgcatcattggatattactgaaactgaaaagcttctctctattttggacactattggtccttacatctgtctagttaaaacacacatcgatattgtttctgattttacgtatgaaggaactgtgttgcctttgaaggagcttgccaagaaacataattttatgatttttgaagatagaaaatttgctgatattggtaacaccgttaaaaatcaatataaatctggtgtcttccgtattgccgaatgggctgacatcactaatgcacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgtcattggttttattgcgcaacacgatatgggcggtagagaagaaggttttgactggatcattatgactcca

SEQ ID NO: 56 - DNA, 3092 bp, Artificial Sequence
  DNA integration cassette s408
aatcaatataaatctggtgtcttccgtattgccgaatgggctgacatcactaatgcacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgtcattggttttattgcgcaacacgatatgggcggtagagaagaaggttttgactggatcattatgactccaggggttggtttagatgacaaaggtgatgcacttggtcaacaatatagaactgttgatgaagttgtaaagactggaacggatatcataattgttggtagaggtttgtacggtcaaggaagagatcctatagagcaagctaaaagataccaacaagctggttggaatgcttatttaaacagatttaaatgagtgaatttactttaaatcttgcatttaaataaattttctttttatagctttatgacttagtttcaatttatatactattttaatgacattttcgattcattgattgaaagctttgtgttttttcttgatgcgctattgcattgttcttgtctttttcgccacatttaatatctgtagtagatacctgatacattgtggatcgcctggcagcagggcgataacctcataacttcgtataatgtatgctatacgaacggtagctacttagcttctatagttagttaatgcactcacgatattcaaaattgacacccttcaactactccctactattgtctactactgtctactactcctctttactatagctgctcccaataggctccaccaataggctctgccaatacattttgcgccgccacctttcaggttgtgtcactcctgaaggaccatattgggtaatcgtgcaatttctggaagagagtccgcgagaagtgaggcccccactgtaaatcctcgagggggcatggagtatggggcatggaggatggaggatggggggggggcgaaaaataggtagcaaaaggacccgctatcaccccacccggagaactcgttgccgggaagtcatatttcgacactccggggagtctataaaaggcgggttttgtcttttgccagttgatgttgctgaaaggacttgtttgccgtttcttccgatttaacagtatagaaatcaaccactgttaattatacacgttatactaacacaacaaaaacaaaaacaacgacaacaacaacaacaatgaagggcggctctatggagaaaataaagcccatcttagcaattatttctttgcaattcggctacgcagggatgtacatcattacaatggtgagtttcaagcacggtatggaccattgggtgcttgcaacctatagacacgttgtggccaccgtagtcatggccccgtttgccctgatgtttgagcgtaaaatcagaccgaagatgacgttggctatcttctggagacttctggccctagggatcctagagcccttgatggatcagaatctgtattacatcggtttgaagaatacctctgcttcatacacgtccgcattcacaaacgccttgcctgctgtcacattcattctggccctgatcttccgtttggaaacggtcaatttcaggaaagtccatagtgtcgccaaggtagtcggtacagtgattacagtgggcggtgcaatgattatgacgctatacaaaggccccgcgatagagattgtcaaggcagcacacaactcctttcacgggggctcctcctccacgcctacaggtcagcactgggtgctaggcacaatcgccattatgggtagcattagcacttgggcagcgttttttatacttcaatcctatacattaaaagtctacccagctgagctgagcttggtaactcttatctgcggtattggaacgatcctaaacgctatagccagtttaatcatggttagggatccatccgcttggaaaataggcatggattctgggactttagctgctgtttattccggagtggtatgtagtggaatcgcgtattacatccagagcatcgtcattaagcaacgtggtcccgtattcacgacctccttctctccaatgtgtatgataataaccgccttcctgggcgccctggtactagctgagaagattcatcttggttcaatcattggagcggtgtttatcgtattgggcctgtacagtgttgtgtggggaaaaagtaaggatgaggttaatccattggacgaaaaaatagtagcaaagtctcaggagctgcccatcacaaacgttgtaaagcagacgaacggtcacgatgtaagcggtgccccaacaaatggagtagtgaccagtacctaagattaatataattatataaaaatattatcttcttttctttatatctagtgttatgtaaaataaattgatgactacggaaagcttttttatattgtttctttttcattctgagccacttaaatttcgtgaatgttcttgtaagggacggtagatttacaagtgatacaacaaaaagcaaggcgctttttctaataaaaagaagaaaagcatttaacaattgaacacctctatatcaacgaagaatattactttgtctctaaatccttgtaaaatgtgtacgatctctatatgggttactcagatagacatctgagtgagcgatagatagatagatagatagatagatgtatgggtagatagatgcatatatagatgcatggaatgaaaggaagatagatagagagaaatgcagaaataagcgtatgaggtttaattttaatgtacatacatgtatagataaacgatgtcgatataatttatttagtaaacagattccctgatatgtgtttttagttttatttttttttgttttttctatgttgaaaaacttgatgacatgatcgagtaaaattggagcttgatttcattcatcttgttgattcctttatcataatgcaaagctggggggggggagggtaaaaaaaagtgaagaaaaagaaagtatgatacaactgtggaagtggag

SEQ ID NO: 57 - DNA, 3530 bp, Artificial Sequence
  DNA integration cassette s409
aatcaatataaatctggtgtcttccgtattgccgaatgggctgacatcactaatgcacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgtcattggttttattgcgcaacacgatatgggcggtagagaagaaggttttgactggatcattatgactccaggggttggtttagatgacaaaggtgatgcacttggtcaacaatatagaactgttgatgaagttgtaaagactggaacggatatcataattgttggtagaggtttgtacggtcaaggaagagatcctatagagcaagctaaaagataccaacaagctggttggaatgcttatttaaacagatttaaatgagtgaatttactttaaatcttgcatttaaataaattttctttttatagctttatgacttagtttcaatttatatactattttaatgacattttcgattcattgattgaaagctttgtgttttttcttgatgcgctattgcattgttcttgtctttttcgccacatttaatatctgtagtagatacctgatacattgtggatcgcctggcagcagggcgataacctcataacttcgtataatgtatgctatacgaacggtagctacttagcttctatagttagttaatgcactcacgatattcaaaattgacacccttcaactactccctactattgtctactactgtctactactcctctttactatagctgctcccaataggctccaccaataggctctgccaatacattttgcgccgccacctttcaggttgtgtcactcctgaaggaccatattgggtaatcgtgcaatttctggaagagagtccgcgagaagtgaggcccccactgtaaatcctcgagggggcatggagtatggggcatggaggatggaggatggggggggggcgaaaaataggtagcaaaaggacccgctatcaccccacccggagaactcgttgccgggaagtcatatttcgacactccggggagtctataaaaggcgggttttgtcttttgccagttgatgttgctgaaaggacttgtttgccgtttcttccgatttaacagtatagaaatcaaccactgttaattatacacgttatactaacacaacaaaaacaaaaacaacgacaacaacaacaacaatggggctgggcggggatcagtccttcgtaccggtaatggatagcggacaggtaagattgaaggaactgggctataagcaggaactgaaaagggacttgtcagtgttctcaaacttcgcgatatcttttagcataataagcgtcttaacaggcattaccaccacgtacaatacaggcttgagattcggaggaactgtcaccctagtctacggttggtttttagccgggagtttcactatgtgcgtaggtcttagcatggctgaaatatgcagcagctatcctaccagcggcggtctttattactggagcgcaatgcttgctggaccgcgttgggctccattggcaagttggatgaccggttggtttaatatagtgggtcagtgggccgtaacagcctcagtggactttagtcttgcccaattgatccaggtcatcgtgcttttgtctacgggcgggaggaacgggggcggatataaggggagcgacttcgtcgtaatagggattcacgggggtatcttatttatccacgcccttctaaattcccttcctatcagcgtattgtccttcatcgggcaattggccgctctatggaatcttctaggggtcctagttcttatgatattgatccctctggtgagcacagaaagagctaccacaaaatttgtctttaccaatttcaataccgataatggacttgggattacttcttatgcttatatcttcgttcttggcctgctgatgagtcaatacacaataaccggctatgatgctagcgctcacatgacggaggaaactgtcgacgcggataaaaatgggcctaggggtattatcagtgccattgggatctccatattgttcggttgggggtacatcttgggtatatcctatgcagtcacagacattccttcccttctttccgaaactaataacagtggcggatacgcgatcgcagaaattttttatcttgcgtttaagaatcgtttcggttctgggactggtggtattgtctgtctgggggtagtagcggttgcggtgtttttctgtgggatgagtagcgtcacatcaaattccagaatggcatacgccttttctagagacggagcaatgcctatgtcccccctatggcataaggttaactcaagagaggtgcctataaacgcggtgtggctttctgctctgatttctttttgcatggcgttaacgtccttaggatcaatagtcgcgttccaggcgatggtcagtattgctaccatcgggttgtacatagcctatgcaatacccattatactaagggtaactttggcacgtaatacctttgttcccggtccattcagccttggcaaatatggtatggttgttggctgggtagcggttctgtgggtagttacaatttccgttttgttttctttacccgtggcctaccccataactgcggaaacgcttaattatacaccggtcgccgtagcagggctggttgccattacattaagttactggctgttttcagcgcgtcattggtttacaggtccaatatctaatattttgtcataagattaatataattatataaaaatattatcttcttttctttatatctagtgttatgtaaaataaattgatgactacggaaagcttttttatattgtttctttttcattctgagccacttaaatttcgtgaatgttcttgtaagggacggtagatttacaagtgatacaacaaaaagcaaggcgctttttctaataaaaagaagaaaagcatttaacaattgaacacctctatatcaacgaagaatattactttgtctctaaatccttgtaaaatgtgtacgatctctatatgggttactcagatagacatctgagtgagcgatagatagatagatagatagatagatgtatgggtagatagatgcatatatagatgcatggaatgaaaggaagatagatagagagaaatgcagaaataagcgtatgaggtttaattttaatgtacatacatgtatagataaacgatgtcgatataatttatttagtaaacagattccctgatatgtgtttttagttttatttttttttgttttttctatgttgaaaaacttgatgacatgatcgagtaaaattggagcttgatttcattcatcttgttgattcctttatcataatgcaaagctggggggggggagggtaaaaaaaagtgaagaaaaagaaagtatgatacaactgtggaagtggag

SEQ ID NO: 58 - protein, 1180 aa, Pichia kudriavzevii
MSTVEDHSSLHKLRKESEILSNANKILVANRGEIPIRIFRSAHELSMHTVAIYSHEDRLSMHRLKADEAYAIGKTGQYSPVQAYLQIDEIIKIAKEHDVSMIHPGYGFLSENSEFAKKVEESGMIWVGPPAEVIDSVGDKVSARNLAIKCDVPVVPGTDGPIEDIEQAKQFVEQYGYPVIIKAAFGGGGRGMRVVREGDDIVDAFQRASSEAKSAFGNGTCFIERFLDKPKHIEVQLLADNYGNTIHLFERDCSVQRRHQKVVEIAPAKTLPVEVRNAILKDAVTLAKTANYRNAGTAEFLVDSQNRHYFIEINPRIQVEHTITEEITGVDIVAAQIQIAAGASLEQLGLLQNKITTRGFAIQCRITTEDPAKNFAPDTGKIEVYRSAGGNGVRLDGGNGFAGAVISPHYDSMLVKCSTSGSNYEIARRKMIRALVEFRIRGVKTNIPFLLALLTHPVFISGDCWTTFIDDTPSLFEMVSSKNRAQKLLAYIGDLCVNGSSIKGQIGFPKLNKEAEIPDLLDPNDEVIDVSKPSTNGLRPYLLKYGPDAFSKKVREFDGCMIMDTTWRDAHQSLLATRVRTIDLLRIAPTTSHALQNAFALECWGGATFDVAMRFLYEDPWERLRQLRKAVPNIPFQMLLRGANGVAYSSLPDNAIDHFVKQAKDNGVDIFRVFDALNDLEQLKVGVDAVKKAGGVVEATVCYSGDMLIPGKKYNLDYYLETVGKIVEMGTHILGIKDMAGTLKPKAAKLLIGSIRSKYPDLVIHVHTHDSAGTGISTYVACALAGADIVDCAINSMSGLTSQPSMSAFIAALDGDIETGVPEHFARQLDAYWAEMRLLYSCFEADLKGPDPEVYKHEIPGGQLTNLIFQAQQVGLGEQWEETKKKYEDANMLLGDIVKVTPTSKVVGDLAQFMVSNKLEKEDVEKLANELDFPDSVLDFFEGLMGTPYGGFPEPLRTNVISGKRRKLKGRPGLELEPFNLEEIRENLVSRFGPGITECDVASYNMYPKVYEQYRKVVEKYGDLSVLPTKAFLAPPTIGEEVHVEIEQGKTLIIKLLAISDLSKSHGTREVYFELNGEMRKVTIEDKTAAIETVTRAKADGHNPNEVGAPMAGVVVEVRVKHGTEVKKGDPLAVLSAMKMEMVISAPVSGRVGEVFVNEGDSVDMGDLLVKIAKDEAPAA